(19) United States
(12) Patent Application Publication
(10) Pub. No.: US 2024/0377401 A1
(43) Pub. Date: Nov. 14, 2024

(54) COMPOSITIONS AND METHODS FOR DETECTION OF PROSTATE CANCER

(71) Applicant: Mercy Bioanalytics, Inc., Waltham, MA (US)

(72) Inventors: Joseph Charles SEDLAK, Waltham, MA (US); Emily Susan WINN-DEEN, Waltham, MA (US); Daniel GUSENLEITNER, Waltham, MA (US); Kelly BIETTE, Waltham, MA (US); Laura Teresa BORTOLIN, Waltham, MA (US); Daniel Parker SALEM, Waltham, MA (US); Anthony David COUVILLON, Waltham, MA (US); Jonian GROSHA, Waltham, MA (US)

(21) Appl. No.: 18/580,442

(22) PCT Filed: Jul. 21, 2022
(86) PCT No.: PCT/US2022/037938

§ 371 Date: Jan. 18, 2024

Related U.S. Application Data
(60) Provisional application No. 63/224,380, filed on Jul. 21, 2021.

(51) Int. Cl. G01N 33/574; C12Q 1/6886

(52) U.S. Cl. CPC G01N 33/57434; C12Q 1/6886; G01N 33/57488; C12Q 2600/158

(57) ABSTRACT

The present disclosure in one aspect provides technologies for detection of prostate cancer, e.g., early detection of prostate cancer. In another aspect, technologies provided herein are useful for selecting and/or monitoring and/or evaluating efficacy of, a treatment administered to a subject determined to have or susceptible to prostate cancer. In some embodiments, technologies provided herein are useful for development of companion diagnostics, e.g., by measuring tumor burdens and changes in tumor burdens in conjunction with therapeutics. In some embodiments, technologies provided herein are useful for development of companion diagnostics, e.g., by identifying biomarkers in subjects' bodily fluid samples (e.g., blood samples) that are associated with therapeutic response.

### CROSS-REFERENCE TO RELATED APPLICATIONS

This application claims the benefit of U.S. Provisional Application No. 63/224,380 filed Jul. 21, 2021, the content of which is hereby incorporated herein in its entirety.

### BACKGROUND

Early detection of cancer greatly increases the chance of successful treatment. However, many cancers including prostate cancer still lack effective screening recommendations. Typical challenges for cancer-screening tests include limited sensitivity and specificity. A high rate of false-positive results can be of particular concern, as it can create difficult management decisions for clinicians and patients who would not want to unnecessarily administer (or receive) anti-cancer therapy that may potentially have undesirable side effects. Conversely, a high rate of false-negative results fails to satisfy the purpose of the screening test, as patients who need therapy are missed, resulting in a treatment delay and consequently a reduced possibility of success.

### SUMMARY

The present disclosure, among other things, provides insights and technologies for achieving effective prostate cancer screening from a biological sample. In some embodiments, such a biological sample is or comprises a bodily fluid-derived sample, e.g., in some embodiments a blood-derived sample. In some embodiments, the present disclosure, among other things, provides insights and technologies that are particularly useful for prostate adenocarcinoma screening. In some embodiments, provided technologies are effective for detection of early-stage prostate cancer (e.g., prostate adenocarcinoma). In some embodiments, provided technologies are effective even when applied to populations comprising or consisting of asymptomatic individuals (e.g., due to sufficiently high sensitivity and/or low rates of false positive and/or false negative results). In some embodiments, provided technologies are effective when applied to populations comprising or consisting of individuals (e.g., asymptomatic individuals) without hereditary risk in developing prostate cancer (e.g., prostate adenocarcinoma). In some embodiments, provided technologies are effective when applied to populations comprising or consisting of symptomatic individuals (e.g., individuals suffering from one or more symptoms of prostate cancer). In some embodiments, provided technologies are effective when applied to populations comprising or consisting of individuals at risk for prostate cancer (e.g., individuals with hereditary and/or life-history associated risk factors for prostate cancer). In some embodiments, provided technologies may be or include one or more compositions (e.g., molecular entities or complexes, systems, cells, collections, combinations, kits, etc.) and/or methods (e.g., of making, using, assessing, etc.), as will be clear to one skilled in the art reading the disclosure provided herein.

In some embodiments, the present disclosure identifies the source of a problem with certain prior technologies including, for example, certain conventional approaches to detection and diagnosis of prostate cancer. For example, the present disclosure appreciates that many conventional diagnostic assays, e.g., digital rectal examination (DRE), urine test, CT scanning, and/or molecular tests based on cell-free nucleic acids, serum biomarkers (e.g., PSA), and/or bulk analysis of extracellular vesicles, can be time-consuming, costly, and/or lacking sensitivity and/or specificity sufficient to provide a reliable and comprehensive diagnostic assessment. In some embodiments, the present disclosure provides technologies (including systems, compositions, and methods) that solve such problems, among other things, by detecting co-localization of a target biomarker signature of prostate cancer in individual extracellular vesicles, which comprises at least one extracellular vesicle-associated surface biomarker and at least one target biomarker selected from the group consisting of surface biomarkers, internal biomarkers, and RNA biomarkers. In some embodiments, the present disclosure provides technologies (including systems, compositions, and methods) that solve such problems, among other things, by detecting such target biomarker signature of prostate cancer using a target entity detection approach that was developed by Applicant and described in U.S. application Ser. No. 16/805,637 (published as US2020/0299780; issued as U.S. Pat. No. 11,085,089), and International Application PCT/US2020/020529 (published as WO2020180741), both filed Feb. 28, 2020 and entitled “Systems, Compositions, and Methods for Target Entity Detection,” which are based on interaction and/or co-localization of at least two or more target entities (e.g., a target biomarker signature) in individual extracellular vesicles.

In some embodiments, extracellular vesicles for detection as described herein can be isolated from a bodily fluid of a subject by a size exclusion-based method. As will be understood by a skilled artisan, in some embodiments, a size exclusion-based method may provide a sample comprising nanoparticles having a size range of interest that includes extracellular vesicles. Accordingly, in some embodiments, provided technologies of the present disclosure encompass detection, in individual nanoparticles having a size range of interest (e.g., in some embodiments about 30 nm to about 1000 nm) that includes extracellular vesicles, of co-localization of at least two or more surface biomarkers (e.g., as described herein) that forms a target biomarker signature of prostate cancer. A skilled artisan reading the present disclosure will understand that various embodiments described herein in the context of “extracellular vesicle(s)” can be also applicable in the context of “nanoparticles” as described herein.

In some embodiments, the present disclosure, among other things, provides insights that screening of asymptotic individuals, e.g., regular screening prior to or otherwise in absence of developed symptom(s), can be beneficial, and even important for effective management (e.g., successful treatment) of prostate cancer (e.g., in some embodiments prostate adenocarcinoma. In some embodiments, the present disclosure provides prostate cancer screening systems that can be implemented to detect prostate cancer (e.g., in some embodiments prostate adenocarcinoma), including early-stage cancer, in some embodiments in asymptomatic individuals. In some embodiments, provided technologies are implemented to achieve regular screening of asymptomatic individuals. The present disclosure provides, for example, compositions (e.g., reagents, kits, components, etc.), and methods of providing and/or using them, including strategies that involve regular testing of one or more individuals (e.g., symptomatic or asymptomatic individuals). The present disclosure defines usefulness of such systems, and provides compositions and methods for implementing them.

In some embodiments, provided technologies achieve detection (e.g., early detection, e.g., in asymptomatic individual(s) and/or population(s)) of one or more features (e.g., incidence, progression, responsiveness to therapy, recurrence, etc.) of prostate cancer, with sensitivity and/or specificity (e.g., rate of false positive and/or false negative results) appropriate to permit useful application of provided technologies to single-time and/or regular (e.g., periodic) assessment. In some embodiments, provided technologies are useful in conjunction with regular medical examinations, such as but not limited to: physicals, general practitioner visits, cholesterol/lipid blood tests, diabetes (type 2) screening, blood pressure screening, thyroid function tests, prostate cancer screening, mammograms, HPV/Pap smears, and/or vaccinations. In some embodiments, provided technologies are useful in conjunction with treatment regimen(s); in some embodiments, provided technologies may improve one or more characteristics (e.g., rate of success according to an accepted parameter) of such treatment regimen(s).

In some aspects, provided are technologies for use in classifying a subject (e.g., an asymptomatic subject) as having or being susceptible to prostate cancer (e.g., in some embodiments prostate adenocarcinoma). In some embodiments, the present disclosure provides methods or assays for classifying a subject (e.g., an asymptomatic subject) as having or being susceptible to prostate cancer (e.g., in some embodiments prostate adenocarcinoma). In some embodiments, a provided method or assay comprises (a) detecting, in a bodily fluid-derived sample (e.g., but not limited to a blood-derived sample, a urine-derived sample, etc.) from a subject in need thereof, extracellular vesicles expressing a target biomarker signature of prostate cancer (e.g., in some embodiments prostate adenocarcinoma), the target biomarker signature comprising: at least one extracellular vesicle-associated surface biomarker and at least one target biomarker selected from the group consisting of: surface biomarkers (as described herein), intravesicular biomarkers (as described herein), and intravesicular RNA biomarkers (as described herein); (b) comparing sample information indicative of level of the target biomarker signature-expressing extracellular vesicles in the bodily fluid-derived sample (e.g., but not limited to a blood-derived sample, a urine-derived sample, etc.) to reference information including a reference threshold level; and (c) classifying the subject as having or being susceptible to prostate cancer (e.g., in some embodiments prostate adenocarcinoma) when the bodily fluid-derived sample (e.g., but not limited to a blood-derived sample, a urine-derived sample, etc.) shows an elevated level of target biomarker signature-expressing extracellular vesicles relative to a classification cutoff referencing the reference threshold level.

In some embodiments, one or more surface biomarkers that can be included in a target biomarker signature are selected from (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, and combinations thereof; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof.

In some embodiments, one or more surface biomarkers that can be included in a target biomarker signature are selected from (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, one or more intravesicular biomarkers that can be included in a target biomarker signature are selected from polypeptides encoded by human genes as follows: ACP5, ACPP, ACSM1, ACTA2, ACTG2, ADAMTS1, AGR2, AIM1, ALCAM, ALDH1A3, ALOX15B, ANTXR1, ANTXR2, AP1M2, AR, ARG2, ARHGAP6, ARHGEF26, ARHGEF37, ASAP3, ATP8B1, ATP9A, BAIAP2L1, BCAM, BHLHA15, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CGN, CGNL1, CHMP4C, CLGN, CMTM4, COLCA1, CORO2A, CPE, CPNE4, CPQ, CRYM, CTSF, CX3CL1, CXADR, CXCR4, CYB561, DPYSL3, DSP, EEF1A2, EFNA1, EHD2, EHF, ELF3, EMP2, EPHX1, EPN3, ERBB3, ERG, ESRP1, ESRP2, F3, FAAH, FAM129A, FAM129B, FAM210B, FAM3B, FAM3D, FAM83H, FAM84A, FAM84B, FAT1, FBLIM1, FBP1, FLNC, FNBP1L, FOS, FOXA1, GADD45G, GATA2, GGT1, GJB1, GLYATL1, GNG4, GNMT, GRHL2, GUCY1A3, HGD, HID1, HIST1H2BK, HIST3H2A, HMGCS2, HOMER2, HOXB13, HSD17B6, HSPB6, HSPB8, ICA1, ID1, IQGAP2, IRF6, ITGA6, KIAA1522, KIF5C, KLK2, KRT14, KRT15, KRT17, KRT18, KRT19, KRT5, KRT7, KRT8, LCP1, LIFR, LIMCH1, LMOD1, LRP11, LRP3, LSR, MAL2, MAOA, MAP7, MARC1, MARCKSL1, MB, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, PALLD, PARVA, PCDH1, PCP4, PDIA5, PEBP4, PGM5, PKP1, PKP3, PPL, PPM1H, PPP1R1B, PPP3CA, PRAC1, PRR15L, PSAT1, PTPRF, PYCR1, RAB3B, RAMP1, RAP1GAP, RASEF, RBM47, RCAN3, RDH11, REEP6, REPS2, RGS10, RHPN2, RNF144B, RORC, RPS4Y1, S100A16, SELENBP1, SERINC5, SH3BGRL2, SH3BP4, SHC2, SLC1A5, SLC22A3, SLC2A12, SLC40A1, SLC4A4, SLC7A8, SLC9A3R2, SMOC2, SORD, SPDEF, SPINT1, SPINT2, SPTBN2, ST14, STAP2, STK39, SULT2B1, SYNE4, SYT7, SYTL1, TAGLN, TBC1D16, TBX3, TC2N, TCEA3, TEAD3, TESC, THBS4, TM4SF1, TMED3, TMEM150C, TMEM54, TMEM98, TMSB15A, TP53111, TPD52, TPM1, TSPAN13, TSPAN6, TSPAN8, VAMP8, VSTM2L, WLS, WWC1, YAP1, ZBTB42, ZC3H11A, ZNF217, ACSL3, CADH7, KCNN2, OPRK1, PDE9A, PLPP1, SPON2, TMEM121B, and combinations thereof. In some embodiments, an intravesicular biomarker described herein may comprise at least one post-translational modification.

In some embodiments, one or more intravesicular RNAs (e.g., mRNAs) that can be included in a target biomarker signature are selected from RNA transcripts (e.g., mRNA transcripts) encoded by human genes as follows: ABCC4, ACP5, ACPP, ACSM1, ACTA2, ACTG2, ADAMTS1, AGR2, AIM1, ALCAM, ALDH1A3, ALOX15B, ANO7, ANPEP, ANTXR1, ANTXR2, AP1M2, APCDD1, AQP3, AR, ARG2, ARHGAP6, ARHGEF26, ARHGEF37, ASAP3, ASTN2, ATP8B1, ATP9A, BAIAP2L1, BCAM, BHLHA15, BIK, BMPR1B, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CDH1, CGN, CGNL1, CHMP4C, CHRM1, CHRNA2, CLDN3, CLDN4, CLDN7, CLDN8, CLGN, CMTM4, COLCA1, COLEC12, CORO2A, CPE, CPNE4, CPQ, CRB3, CREB3L1, CREB3L4, CRYM, CTSF, CWH43, CX3CL1, CXADR, CXCR4, CYB561, DPP4, DPYSL3, DSG2, DSP, EEF1A2, EFNA1, EHD2, EHF, ELF3, ELOVL7, EMB, EMP2, ENPP5, EPCAM, EPHX1, EPN3, ERBB3, ERG, ESRP1, ESRP2, F3, FAAH, FAM129A, FAM129B, FAM189A2, FAM210B, FAM3B, FAM3D, FAM83H, FAM84A, FAM84B, FAT1, FBLIM1, FBP1, FLNC, FNBP1L, FOLH1, FOS, FOXA1, FXYD3, GADD45G, GALNT3, GALNT7, GATA2, GCNT1, GGT1, GJB1, GLYATL1, GNG4, GNMT, GOLM1, GPR160, GREB1, GRHL2, GUCY1A3, HGD, HID1, HIST1H2BK, HIST3H2A, HMGCS2, HOMER2, HOXB13, HPN, HSD17B6, HSPB6, HSPB8, ICA1, ID1, IQGAP2, IRF6, ITGA6, KCNN4, KIAA1324, KIAA1522, KIF5C, KLK2, KRT14, KRT15, KRT17, KRT18, KRT19, KRT5, KRT7, KRT8, KRTCAP3, LCP1, LIFR, LIMCH1, LMOD1, LPAR3, LRP11, LRP3, LRRC26, LSR, MAL2, MAOA, MAP7, MARC1, MARCKSL1, MB, MBOAT2, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, OR51E1, OR51E2, PALLD, PARM1, PARVA, PCDH1, PCP4, PDIA5, PDLIM5, PDZK1IP1, PDZRN3, PEBP4, PGM5, PIGR, PKP1, PKP3, PLA2G2A, PMEPA1, PODXL2, PPL, PPM1H, PPP1R1B, PPP3CA, PRAC1, PROM2, PRR15L, PRSS8, PSAT1, PSCA, PTPRF, PTPRN2, PYCR1, RAB25, RAB27B, RAB3B, RAMP1, RAP1GAP, RASEF, RBM47, RCAN3, RDH11, REEP6, REPS2, RGS10, RHPN2, RNF144B, RORC, RPS4Y1, S100A16, SCNN1A, SDC1, SELENBP1, SERINC2, SERINC5, SEZ6L2, SH3BGRL2, SH3BP4, SHC2, SLC15A2, SLC1A5, SLC22A3, SLC2A10, SLC2A12, SLC30A4, SLC35F2, SLC40A1, SLC44A4, SLC45A3, SLC4A4, SLC7A8, SLC9A3R2, SMIM22, SMOC2, SORD, SPDEF, SPINT1, SPINT2, SPTBN2, ST14, STAP2, STEAP1, STEAP2, STEAP4, STK39, SULT2B1, SYNE4, SYT7, SYTL1, TACSTD2, TAGLN, TBC1D16, TBX3, TC2N, TCEA3, TEAD3, TESC, THBS4, TM4SF1, TMC4, TMC5, TMED3, TMEFF2, TMEM125, TMEM150C, TMEM30B, TMEM45B, TMEM54, TMEM98, TMPRSS2, TMSB15A, TP53111, TPD52, TPM1, TRPM4, TRPM8, TRPV6, TSPAN1, TSPAN13, TSPAN6, TSPAN8, TUSC3, UPK3A, VAMP8, VSIG2, VSTM2L, WLS, WWC1, YAP1, ZBTB42, ZC3H11A, ZNF217, and combinations thereof.

In some embodiments, methods or assays described herein may be performed for one more additional target biomarker signature (including, e.g., at least one, at least two, at least three, or more additional target biomarker signatures). In some such embodiments, a classification cutoff may reference additional reference threshold level(s) corresponding to each additional target biomarker signature.

In some embodiments, an extracellular vesicle-associated surface biomarker for use in a target biomarker signature of prostate cancer used and/or described herein may be or comprise a tumor-specific biomarker and/or a tissue-specific biomarker (e.g., a prostate tissue-specific biomarker). In some embodiments, such an extracellular vesicle-associated surface biomarker may be or comprise a non-specific marker, e.g., it is present in one or more non-target tumors, and/or in one or more non-target tissues. In some embodiments, such an extracellular vesicle-associated surface biomarker may be or comprise (i) one or more surface proteins encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, or any combinations thereof; and/or (ii) one or more carbohydrate-dependent markers as follows: Tn antigen, SialylTn (sTn) antigen, Thomsen-Friedenreich (T. TF) antigen, Lewis Y antigen (also known as CD174), Sialyl Lewis X (sLex) antigen (also known as Sialyl SSEA-1 (SLX)), or combinations thereof.

In some embodiments, an extracellular vesicle-associated surface biomarker may be or comprise (i) a polypeptide encoded by human gene MUC1; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), or combinations thereof.

In some embodiments, a target biomarker signature of prostate cancer (e.g., prostate adenocarcinoma) may comprise an extracellular vesicle-associated surface biomarker (e.g., ones described herein) and at least one (including, e.g., 1, 2, 3, or more) additional target surface biomarker, which, in some embodiments, may be or comprise one or more polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, or combinations thereof; and/or one or more carbohydrate markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), or combinations thereof.

In some embodiments, a target biomarker signature of prostate cancer (e.g., prostate adenocarcinoma) may comprise an extracellular vesicle-associated surface biomarker (e.g., ones described herein) and at least one (including, e.g., 1, 2, 3, or more) additional surface biomarker, which are selected from (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)). T antigen, Tn antigen, and combinations thereof.

In some embodiments, a target biomarker signature of prostate cancer (e.g., prostate adenocarcinoma) may comprise an extracellular vesicle-associated surface biomarker (e.g., ones described herein) and at least one target intravesicular RNA biomarker, which, in some embodiments, may be or comprise at least one RNA transcript (e.g., mRNA transcript) encoded by a human gene as follows: ABCC4, ACP5, ACPP, ACSM1, ACTA2, ACTG2, ADAMTS1, AGR2, AIM1, ALCAM, ALDH1A3, ALOX15B, ANO7, ANPEP, ANTXR1, ANTXR2, AP1M2, APCDD1, AQP3, AR, ARG2, ARHGAP6, ARHGEF26, ARHGEF37, ASAP3, ASTN2, ATP8B1, ATP9A, BAIAP2L1, BCAM, BHLHA15, BIK, BMPR1B, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CDH1, CGN, CGNL1, CHMP4C, CHRM1, CHRNA2, CLDN3, CLDN4, CLDN7, CLDN8, CLGN, CMTM4, COLCA1, COLEC12, CORO2A, CPE, CPNE4, CPQ, CRB3, CREB3L1, CREB3L4, CRYM, CTSF, CWH43, CX3CL1, CXADR, CXCR4, CYB561, DPP4, DPYSL3, DSG2, DSP, EEF1A2, EFNA1, EHD2, EHF, ELF3, ELOVL7, EMB, EMP2, ENPP5, EPCAM, EPHX1, EPN3, ERBB3, ERG, ESRP1, ESRP2, F3, FAAH, FAM129A, FAM129B, FAM189A2, FAM210B, FAM3B, FAM3D, FAM83H, FAM84A, FAM84B, FAT1, FBLIM1, FBP1, FLNC, FNBP1L, FOLH1, FOS, FOXA1, FXYD3, GADD45G, GALNT3, GALNT7, GATA2, GCNT1, GGT1, GJB1, GLYATL1, GNG4, GNMT, GOLM1, GPR160, GREB1, GRHL2, GUCY1A3, HGD, HID1, HIST1H2BK, HIST3H2A, HMGCS2, HOMER2, HOXB13, HPN, HSD17B6, HSPB6, HSPB8, ICA1, ID1, IQGAP2, IRF6, ITGA6, KCNN4, KIAA1324, KIAA1522, KIF5C, KLK2, KRT14, KRT15, KRT17, KRT18, KRT19, KRT5, KRT7, KRT8, KRTCAP3, LCP1, LIFR, LIMCH1, LMOD1, LPAR3, LRP11, LRP3, LRRC26, LSR, MAL2, MAOA, MAP7, MARC1, MARCKSL1, MB, MBOAT2, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, OR51E1, OR51E2, PALLD, PARM1, PARVA, PCDH1, PCP4, PDIA5, PDLIM5, PDZK1IP1, PDZRN3, PEBP4, PGM5, PIGR, PKP1, PKP3, PLA2G2A, PMEPA1, PODXL2, PPL, PPM1H, PPP1R1B, PPP3CA, PRAC1, PROM2, PRR15L, PRSS8, PSAT1, PSCA, PTPRF, PTPRN2, PYCR1, RAB25, RAB27B, RAB3B, RAMP1, RAP1GAP, RASEF, RBM47, RCAN3, RDH11, REEP6, REPS2, RGS10, RHPN2, RNF144B, RORC, RPS4Y1, S100A16, SCNN1A, SDC1, SELENBP1, SERINC2, SERINC5, SEZ6L2, SH3BGRL2, SH3BP4, SHC2, SLC15A2, SLC1A5, SLC22A3, SLC2A10, SLC2A12, SLC30A4, SLC35F2, SLC40A1, SLC44A4, SLC45A3, SLC4A4, SLC7A8, SLC9A3R2, SMIM22, SMOC2, SORD, SPDEF, SPINT1, SPINT2, SPTBN2, ST14, STAP2, STEAP1, STEAP2, STEAP4, STK39, SULT2B1, SYNE4, SYT7, SYTL1, TACSTD2, TAGLN, TBC1D16, TBX3, TC2N, TCEA3, TEAD3, TESC, THBS4, TM4SF1, TMC4, TMC5, TMED3, TMEFF2, TMEM125, TMEM150C, TMEM30B, TMEM45B, TMEM54, TMEM98, TMPRSS2, TMSB15A, TP53111, TPD52, TPM1, TRPM4, TRPM8, TRPV6, TSPAN1, TSPAN13, TSPAN6, TSPAN8, TUSC3, UPK3A, VAMP8, VSIG2, VSTM2L, WLS, WWC1, YAP1, ZBTB42, ZC3H11A, ZNF217, or combinations thereof.

In some embodiments, a target biomarker signature of prostate cancer may comprise an extracellular vesicle-associated surface biomarker (e.g., ones described herein) and at least one additional target intravesicular biomarker, which, in some embodiments, may be or comprise at least one polypeptide encoded by a human gene as follows: ACP5, ACPP, ACSM1, ACTA2, ACTG2, ADAMTS1, AGR2, AIM1, ALCAM, ALDH1A3, ALOX15B, ANTXR1, ANTXR2, AP1M2, AR, ARG2, ARHGAP6, ARHGEF26, ARHGEF37, ASAP3, ATP8B1, ATP9A, BAIAP2L1, BCAM, BHLHA15, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CGN, CGNL1, CHMP4C, CLGN, CMTM4, COLCA1, CORO2A, CPE, CPNE4, CPQ, CRYM, CTSF, CX3CL1, CXADR, CXCR4, CYB561, DPYSL3, DSP, EEF1A2, EFNA1, EHD2, EHF, ELF3, EMP2, EPHX1, EPN3, ERBB3, ERG, ESRP1, ESRP2, F3, FAAH, FAM129A, FAM129B, FAM210B, FAM3B, FAM3D, FAM83H, FAM84A, FAM84B, FAT1, FBLIM1, FBP1, FLNC, FNBP1L, FOS, FOXA1, GADD45G, GATA2, GGT1, GJB1, GLYATL1, GNG4, GNMT, GRHL2, GUCY1A3, HGD, HID1, HIST1H2BK, HIST3H2A, HMGCS2, HOMER2, HOXB13, HSD17B6, HSPB6, HSPB8, ICA1, ID1, IQGAP2, IRF6, ITGA6, KIAA1522, KIF5C, KLK2, KRT14, KRT15, KRT17, KRT18, KRT19, KRT5, KRT7, KRT8, LCP1, LIFR, LIMCH1, LMOD1, LRP11, LRP3, LSR, MAL2, MAOA, MAP7, MARC1, MARCKSL1, MB, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, PALLD, PARVA, PCDH1, PCP4, PDIA5, PEBP4, PGM5, PKP1, PKP3, PPL, PPM1H, PPP1R1B, PPP3CA, PRAC1, PRR15L, PSAT1, PTPRF, PYCR1, RAB3B, RAMP1, RAP1GAP, RASEF, RBM47, RCAN3, RDH11, REEP6, REPS2, RGS10, RHPN2, RNF144B, RORC, RPS4Y1, S100A16, SELENBP1, SERINC5, SH3BGRL2, SH3BP4, SHC2, SLC1A5, SLC22A3, SLC2A12, SLC40A1, SLC4A4, SLC7A8, SLC9A3R2, SMOC2, SORD, SPDEF, SPINT1, SPINT2, SPTBN2, ST14, STAP2, STK39, SULT2B1, SYNE4, SYT7, SYTL1, TAGLN, TBC1D16, TBX3, TC2N, TCEA3, TEAD3, TESC, THBS4, TM4SF1, TMED3, TMEM150C, TMEM54, TMEM98, TMSB15A, TP53111, TPD52, TPM1, TSPAN13, TSPAN6, TSPAN8, VAMP8, VSTM2L, WLS, WWC1, YAP1, ZBTB42, ZC3H11A, ZNF217, ACSL3, CADH7, KCNN2, OPRK1, PDE9A, PLPP1, SPON2, TMEM121B, or combinations thereof. In some embodiments, an intravesicular biomarker described herein may comprise at least one post-translational modification.

In some embodiments, a reference threshold level for use in a provided method or assay described herein is determined by levels of target biomarker signature-expressing extracellular vesicles observed in comparable samples from a population of non-prostate cancer subjects.

In some embodiments, an extracellular vesicle-associated surface biomarker included in a target biomarker signature may be detected using affinity agents (e.g., but not limited to antibody-based agents). In some embodiments, an extracellular vesicle-associated surface biomarker may be detected using a capture assay comprising an antibody-based agent. For example, in some embodiments, a capture assay for detecting the presence of an extracellular vesicle-associated surface biomarker in an extracellular vesicle may involve contacting a bodily fluid-derived sample (e.g., but not limited to a blood-derived sample, a urine-derived sample, etc.) comprising extracellular vesicles with a capture agent directed to such an extracellular vesicle-associated surface biomarker. In some embodiments, such a capture agent may comprise a binding moiety directed to an extracellular vesicle-associated surface biomarker (e.g., ones described herein), which may be optionally conjugated to a solid substrate. Without limitations, an exemplary capture agent for an extracellular vesicle-associated surface biomarker may be or comprising a solid substrate (e.g., a magnetic bead) and a binding moiety (e.g., an antibody agent) directed to an extracellular vesicle-associated surface biomarker.

In some embodiments, a target biomarker included in a target biomarker signature may be detected using appropriate methods known in the art, which may vary with types of analytes to be detected (e.g., surface analytes vs. intravesicular analytes; and/or polypeptides and/or glycoforms vs. carbohydrates vs. RNAs). For example, a person skilled in the art, reading the present disclosure, will appreciate that a surface biomarker and/or an intravesicular biomarker may be detected using affinity agents (e.g., antibody-based agents) in some embodiments, while in some embodiments, an intravesicular RNA (e.g., but not limited to mRNA and noncoding RNA such as, e.g., orphan noncoding RNA, long noncoding RNA, piwi-interacting RNA, microRNA, circular RNA, etc.) biomarker may be detected using nucleic acid-based agents, e.g., using quantitative reverse transcription PCR.

For example, in some embodiments where a target biomarker is or comprises a surface biomarker and/or an intravesicular marker, such a target biomarker may be detected involving a proximity ligation assay, e.g., following a capture assay (e.g., ones as described herein) to capture extracellular vesicles that display an extracellular vesicle-associated surface biomarker (e.g., ones as used and/or described herein). In some embodiments, such a proximity ligation assay may comprise contacting a bodily fluid-derived sample (e.g., but not limited to a blood-derived sample, a urine-derived sample, etc.) comprising extracellular vesicles with a set of detection probes, each directed to a target biomarker, which set comprises at least two distinct detection probes, so that a combination comprising the extracellular vesicles and the set of detection probes is generated, wherein the two detection probes each comprise: (i) a binding moiety directed to a surface biomarker and/or an intravesicular biomarker; and (ii) an oligonucleotide domain coupled to the binding moiety, the oligonucleotide domain comprising a double-stranded portion and a single-stranded overhang portion extended from one end of the oligonucleotide domain. Such single-stranded overhang portions of the detection probes are characterized in that they can hybridize with each other when the detection probes are bound to the same extracellular vesicle. Such a combination comprising the extracellular vesicles and the set of detection probes is then maintained under conditions that permit binding of the set of detection probes to their respective targets on the extracellular vesicles such that their oligonucleotide domains are in close enough proximity to anneal to form a double-stranded complex. Such a double-stranded complex can be detected by contacting the double-stranded complex with a nucleic acid ligase to generate a ligated template; and detecting the ligated template. In some embodiments, a ligated template can be detected using quantitative PCR. The presence of such a ligated template is indicative of presence of extracellular vesicles that are positive for a target biomarker signature of prostate cancer (e.g., prostate adenocarcinoma). While such a proximity ligation assay may perform better, e.g., with higher specificity and/or sensitivity, than other existing proximity ligation assays, a person skilled in the art reading the present disclosure will appreciate that other forms of proximity ligation assays that are known in the art may be used instead.

In some embodiments where a target biomarker is or comprises an intravesicular RNA (e.g., but not limited to mRNA and noncoding RNA such as, e.g., orphan noncoding RNA, long noncoding RNA, piwi-interacting RNA, microRNA, circular RNA, etc.) marker, such a target biomarker may be detected involving a nucleic acid detection assay. In some embodiments, an exemplary nucleic acid detection assay may be or comprise reverse-transcription PCR.

In some embodiments where a target biomarker is or comprises an intravesicular biomarker and/or an intravesicular RNA (e.g., but not limited to mRNA and noncoding RNA such as, e.g., orphan noncoding RNA, long noncoding RNA, piwi-interacting RNA, microRNA, circular RNA, etc.) biomarker, such a target biomarker may be detected involving, prior to a detection assay (e.g., a proximity ligation assay as described herein), a sample treatment (e.g., fixation and/or permeabilization) to expose such biomarker(s) within extracellular vesicles for subsequent detection.

The present disclosure, among other things, recognizes that detection of a single prostate cancer-associated serum protein (e.g. prostate-specific antigen (PSA)) or a plurality of prostate cancer-associated biomarkers based on a bulk sample (e.g., a bulk sample of extracellular vesicles), rather than at a resolution of a single extracellular vesicle, typically does not provide sufficient specificity and/or sensitivity in determination of whether a subject from whom the sample is obtained is likely to be suffering from or susceptible to prostate cancer. The present disclosure, among other things, provides technologies, including systems, compositions, and/or methods, that solve such problems, including for example by specifically requiring that individual extracellular vesicles for detection be characterized by presence of a target biomarker signature comprising a combination of at least one or more extracellular vesicle-associated surface biomarkers and at least one or more target biomarkers. In particular embodiments, the present disclosure teaches technologies that require such individual extracellular vesicles be characterized by presence (e.g., by expression) of such a target biomarker signature of prostate cancer (e.g., prostate adenocarcinoma), while extracellular vesicles that do not comprise the target biomarker signature do not produce a detectable signal (e.g., a level that is above a reference level, e.g., by at least 10% or more, where in some embodiments, a reference level may be a level observed in a negative control sample, such as a sample in which individual extracellular vesicles comprising such a target biomarker signature are absent).

As will be understood by a skilled artisan, in some embodiments, a sample comprising extracellular vesicles may also comprise nanoparticles having a size range of interest that includes extracellular vesicles. Thus, in some embodiments, provided technologies of the present disclosure in the context of extracellular vesicles are also applicable to detection of nanoparticles having a size range interest that includes extracellular vesicles. Accordingly, in some embodiments, the present disclosure, among other things, provides technologies for detection, in individual nanoparticles having a size range of interest (e.g., in some embodiments about 30 nm to about 1000 nm) that includes extracellular vesicles, of co-localization of at least two or more surface biomarkers (e.g., as described herein) that forms a target biomarker signature of prostate cancer.

In some embodiments, the present disclosure describes a method comprising steps of: (a) providing or obtaining a sample comprising nanoparticles having a size within the range of about 30 nm to about 1000 nm, which are isolated from a bodily fluid-derived sample (e.g., but not limited to a blood-derived sample, a urine-derived sample, etc.) of a subject; (b) detecting on surfaces of the nanoparticles co-localization of at least two surface biomarkers whose combined expression level has been determined to be associated with prostate cancer, wherein the surface biomarkers are selected from (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof; (c) comparing the detected co-localization level with the determined level; and (d) classifying the subject as having or being susceptible to prostate cancer when the detected co-localization level is at or above the determined level.

In some embodiments, the first surface biomarker and the second surface biomarker(s) are each independently selected from: (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

Accordingly, in some embodiments, technologies provided herein can be useful for detection of incidence or recurrence of prostate cancer in a subject and/or across a population of subjects. In some embodiments, a target biomarker signature may be selected for detection of prostate cancer. In some embodiments, a target biomarker signature may be selected for detection of a specific category of prostate cancer, including, e.g., but not limited to prostate adenocarcinoma. In some embodiments, a target biomarker signature may be selected for detection of early-stage (e.g., stage I and/or stage II) prostate cancer, including, e.g., but not limited to prostate adenocarcinoma. In some embodiments, a target biomarker signature may be selected for detection of late-stage (e.g., stage III and/or stage IV) prostate cancer, including, e.g., but not limited to prostate adenocarcinoma. In some embodiments, a target biomarker signature may be selected for detection of low grade (e.g., Gleason score of 6) prostate cancer, including, e.g., but not limited to prostate adenocarcinoma. In some embodiments, a target biomarker signature may be selected for detection of intermediate grade (e.g., Gleason score of 7) prostate cancer, including, e.g., but not limited to prostate adenocarcinoma. In some embodiments, a target biomarker signature may be selected for detection of high grade (e.g., Gleason score of 8-10) prostate cancer, including, e.g., but not limited to prostate adenocarcinoma. In some embodiments, technologies provided herein can be used periodically (e.g., every year) to screen a human subject or across a population of human subjects for early-stage prostate cancer or prostate cancer recurrence.

In some embodiments, a subject that is amenable to technologies provided herein for detection of incidence or recurrence of prostate adenocarcinoma may be an asymptomatic human subject and/or across an asymptomatic population. Such an asymptomatic subject may be a subject who has a family history of prostate cancer, who has a life history which places them at increased risk for prostate cancer, who has been previously treated for prostate cancer, who is at risk of prostate cancer recurrence after cancer treatment, who is in remission after prostate cancer treatment, and/or who has been previously or periodically screened for the presence of at least one prostate cancer serum biomarker (e.g., PSA). In some embodiments, such an asymptomatic subject may be a subject who is determined to have a normal medical diagnosis result from, e.g., digital rectal examination (DRE), urine test, CT scanning, and/or molecular tests, for example, and/or based on cell-free nucleic acids. In some embodiments, such an asymptomatic subject may be a subject who is determined to have an abnormal medical diagnosis result from, e.g., digital rectal examination (DRE), urine test, CT scanning, and/or molecular tests, for example, based on cell-free nucleic acids, when compared to results as typically observed in non-prostate cancer subjects and/or normal healthy subjects. Alternatively, in some embodiments, an asymptomatic subject may be a subject who has not been previously screened for prostate cancer, who has not been diagnosed for prostate cancer, and/or who has not previously received prostate cancer therapy.

In some embodiments, a subject or population of subjects may be selected based on one or more characteristics such as age, race, geographic location, genetic history, personal and/or medical history (e.g., smoking, alcohol, drugs, carcinogenic agents, diet, obesity, diabetes, physical activity, sun exposure, radiation exposure, chronic inflammation of the prostate, chronic urinary tract infections, and/or occupational hazard).

In some embodiments, technologies provided herein can be useful for selecting surgery or therapy for a subject who is suffering from or susceptible to prostate adenocarcinoma. In some embodiments, prostate cancer surgery, therapy, and/or an adjunct therapy can be selected in light of findings based on technologies provided herein.

In some embodiments, technologies provided herein can be useful for monitoring and/or evaluating efficacy of therapy administered to a subject (e.g., prostate cancer subject).

In some embodiments, the present disclosure provides technologies for managing patient care, e.g., for one or more individual subjects and/or across a population of subjects. To give but a few examples, in some embodiments, the present disclosure provides technologies that may be utilized in screening (e.g., temporally or incidentally motivated screening and/or non-temporally or incidentally motivated screening, e.g., periodic screening such as annual, semi-annual, bi-annual, or with some other frequency). For example, in some embodiments, provided technologies for use in temporally motivated screening can be useful for screening one or more individual subjects or across a population of subjects (e.g., asymptomatic subjects) who are older than a certain age (e.g., over 40, 45, 50, 55, 60, 65, 70, or older). In some embodiments, provided technologies for use in temporally motivated screening can be useful for screening one or more individual subjects or across a population of subjects (e.g., asymptomatic subjects) who are between an age range from 50 to 70. In some embodiments, provided technologies for use in temporally motivated screening can be useful for screening one or more individual subjects or across a population of subjects (e.g., asymptomatic subjects) who are between an age range from 55 to 69. In some embodiments, provided technologies for use in incidentally motivated screening can be useful for screening individual subjects who may have experienced an incident or event that motivates screening for prostate cancer as described herein. For example, in some embodiments, an incidental motivation relating to determination of one or more indicators of cancer or susceptibility thereto may be or comprise, e.g., an incident based on their family history (e.g., a close relative such as blood-related relative was previously diagnosed for prostate cancer), identification of one or more risk factors associated with prostate cancer (e.g., life history risk factors including, but not limited to smoking, alcohol, diet, obesity, occupational hazard, etc.) and/or prior incidental findings from genetic tests (e.g., genome sequencing), and/or imaging diagnostic tests (e.g., ultrasound, computerized tomography (CT) and/or magnetic resonance imaging (MRI) scans), development of one or more signs or symptoms characteristic of prostate cancer (e.g., abnormal medical results such as difficulty urinating, blood in the urine or semen, erectile dysfunction (ED), pain in the hips, back (spine), chest (ribs), weakness or numbness in the legs or feet, loss of bladder control, and/or symptoms potentially indicative of prostate cancer, etc.).

In some embodiments, provided technologies for managing patient care can inform treatment and/or payment (e.g., reimbursement for treatment) decisions and/or actions. For example, in some embodiments, provided technologies can provide determination of whether individual subjects have one or more indicators of incidence or recurrence of prostate cancer, thereby informing physicians and/or patients when to initiate therapy in light of such findings. Additionally or alternatively, in some embodiments, provided technologies can inform physicians and/or patients of treatment selection, e.g., based on findings of specific responsiveness biomarkers (e.g., prostate cancer responsiveness biomarkers). In some embodiments, provided technologies can provide determination of whether individual subjects are responsive to current treatment, e.g., based on findings of changes in one or more levels of molecular targets associated with prostate cancer, thereby informing physicians and/or patients of efficacy of such therapy and/or decisions to maintain or alter therapy in light of such findings.

In some embodiments, provided technologies can inform decision making relating to whether health insurance providers reimburse (or not), e.g., for (1) screening itself (e.g., reimbursement available only for periodic/regular screening or available only for temporally and/or incidentally motivated screening); and/or for (2) initiating, maintaining, and/or altering therapy in light of findings by provided technologies. For example, in some embodiments, the present disclosure provides methods relating to (a) receiving results of a screening as described herein and also receiving a request for reimbursement of the screening and/or of a particular therapeutic regimen; (b) approving reimbursement of the screening if it was performed on a subject according to an appropriate schedule or response to a relevant incident and/or approving reimbursement of the therapeutic regimen if it represents appropriate treatment in light of the received screening results; and, optionally (c) implementing the reimbursement or providing notification that reimbursement is refused. In some embodiments, a therapeutic regimen is appropriate in light of received screening results if the received screening results detect a biomarker that represents an approved biomarker for the relevant therapeutic regimen (e.g., as may be noted in a prescribing information label and/or via an approved companion diagnostic). Alternatively or additionally, the present disclosure contemplates reporting systems (e.g., implemented via appropriate electronic device(s) and/or communications system(s)) that permit or facilitate reporting and/or processing of screening results, and/or of reimbursement decisions as described herein.

Some aspects provided herein relate to systems and kits for use in provided technologies. In some embodiments, a system or kit may comprise detection agents for a tumor biomarker signature of prostate cancer (e.g., ones described herein).

In some embodiments, such a system or kit may comprise a capture agent for an extracellular vesicle-associated surface biomarker present in extracellular vesicles associated with prostate cancer (e.g., ones used and/or described herein); and (b) at least one or more detection agents directed to one or more target biomarkers of a target biomarker signature of prostate cancer, which may be or comprise additional surface biomarker(s) (e.g., ones as used and/or described herein), intravesicular biomarker(s) (e.g., ones as used and/or described herein), and/or intravesicular RNA (e.g., but not limited to mRNA and noncoding RNA such as, e.g., orphan noncoding RNA, long noncoding RNA, piwi-interacting RNA, microRNA, circular RNA, etc.) biomarker(s) (e.g., ones as used and/or described herein).

In some embodiments, a capture agent included in a system and/or kit may comprise a binding moiety directed to an extracellular vesicle-associated surface biomarker (e.g., ones described herein). In some embodiments, such a binding moiety may be conjugated to a solid substrate, which in some embodiments may be or comprise a solid substrate. In some embodiments, such a solid substrate may be or comprise a magnetic bead. In some embodiments, an exemplary capture agent included in a provided system and/or kit may be or comprise a solid substrate (e.g., a magnetic bead) and an affinity reagent (e.g., but not limited to an antibody agent) directed to an extracellular vesicle-associated surface biomarker conjugated thereto.

In some embodiments where a target biomarker includes a surface biomarker and/or an intravesicular biomarker, a system and/or kit may include detection agents for performing a proximity ligation assay (e.g., ones as described herein). In some embodiments, such detection agents for performing a proximity ligation assay may comprise a set of detection probes, each directed to a target biomarker of a target biomarker signature, which set comprises at least two detection probes, wherein the two detection probes each comprise: (i) a polypeptide-binding moiety directed to a target biomarker; and (ii) an oligonucleotide domain coupled to the binding moiety, the oligonucleotide domain comprising a double-stranded portion and a single-stranded overhang portion extended from one end of the oligonucleotide domain, wherein the single-stranded overhang portions of the detection probes are characterized in that they can hybridize to each other when the detection probes are bound to the same extracellular vesicle.

In some embodiments, a provided system and/or kit may comprise a plurality (e.g., 2, 3, 4, 5, or more) of sets of detection probes, each set of which comprises two or more (e.g., 3, 4, 5, 6, 7, 8, 9, 10, 11, 12, 13, 14, 15, 16, 17, 18, 19, 20 or more) detection probes. In some embodiments, at least one set of detection probes may be directed to detection for prostate cancer. For example, in some embodiments, a provided system and/kit may comprise at least one set for detection probes for detection of prostate cancer and at least one set of detection probes for detection of a different cancer (e.g., pancreatic cancer). In some embodiments, two or more detection probes maybe directed to different categories of prostate cancer (including, e.g., prostate adenocarcinoma). In some embodiments, two or more sets may be directed to detection of prostate cancer of different stages. In some embodiments, two or more sets may be directed to detection of prostate cancer of the same stage.

In some embodiments, detection probes in a provided kit may be provided as a single mixture in a container. In some embodiments, multiple sets of detection probes may be provided as individual mixtures in separate containers. In some embodiments, each detection probe is provided individually in a separate container.

In some embodiments where a target biomarker includes an intravesicular RNA (e.g., but not limited to mRNA and noncoding RNA such as, e.g., orphan noncoding RNA, long noncoding RNA, piwi-interacting RNA, microRNA, circular RNA, etc.) biomarker, such a system and/or kit may include detection agents for performing a nucleic acid detection assay. In some embodiments, such a system and/or kit may include detection agents for performing a quantitative reverse-transcription PCR, for example, which may comprise primers directed to intravesicular RNA (e.g., but not limited to mRNA and noncoding RNA such as, e.g., orphan noncoding RNA, long noncoding RNA, piwi-interacting RNA, microRNA, circular RNA, etc.) target(s).

A skilled artisan reading the present disclosure will understand that a system or kit for detection of extracellular vesicles can also be employed to detect nanoparticles having a size range of interest that includes extracellular vesicles. Accordingly, in some embodiments, a system or kit may comprise (i) a capture agent for a first surface biomarker of a prostate cancer-associated biomarker signature (e.g., as described herein) present on the surface of nanoparticles having a size range of interest that includes extracellular vesicles; and (ii) at least one or more detection agents directed to a second surface biomarker of the prostate cancer-specific biomarker signature. In some embodiments, such nanoparticles have a size within the range of about 30 nm to about 1000 nm.

In some embodiments, the present disclosure describes a kit for detection of prostate cancer comprising: (a) a capture agent comprising a target-capture moiety directed to a first surface biomarker; and (b) at least one set of detection probes, which set comprises at least two detection probes each directed to a second surface biomarker, wherein the detection probes each comprise: (i) a target binding moiety directed at the second surface biomarker; and (ii) an oligonucleotide domain coupled to the target binding moiety, the oligonucleotide domain comprising a double-stranded portion and a single-stranded overhang portion extended from one end of the oligonucleotide domain, wherein the single-stranded overhang portions of the at least two detection probes are characterized in that they can hybridize to each other when the at least two detection probes are bound to the same nanoparticle having the size within the range of about 30 nm to about 1000 nm; wherein at least the first surface biomarker and the second surface biomarker form a target biomarker signature determined to be associated with prostate cancer, and wherein the first and second surface biomarkers are each independently selected from: (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof.

In some embodiments, the first surface biomarker and the second surface biomarker(s) are each independently selected from: (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, a provided system and/or kit may comprise at least one chemical reagent, e.g., to process a sample and/or nanoparticles (including, e.g., in some embodiments extracellular vesicles) therein. In some embodiments, a provided system and/or kit may comprise at least one chemical reagent to process nanoparticles (including, e.g., in some embodiments extracellular vesicles) in a sample, including, e.g., but not limited to a fixation agent, a permeabilization agent, and/or a blocking agent. In some embodiments, a provided system and/or kit may comprise a nucleic acid ligase and/or a nucleic acid polymerase. In some embodiments, a provided system and/or kit may comprise one or more primers and/or probes. In some embodiments, a provided system and/or kit may comprise one or more pairs of primers, for example for PCR, e.g., quantitative PCR (qPCR) reactions. In some embodiments, a provided system and/or kit may comprise one or more probes such as, for example, hydrolysis probes which may in some embodiments be designed to increase the specificity of qPCR (e.g., TaqMan probes). In some embodiments, a provided system and/or kit may comprise one or more multiplexing probes, for example as may be useful when simultaneous or parallel qPCR reactions are employed (e.g., to facilitate or improve readout).

In some embodiments, a provided system and/or kit can be used for screening (e.g., regular screening) and/or other assessment of individuals (e.g., asymptomatic or symptomatic subjects) for detection (e.g., early detection) of prostate cancer. In some embodiments, a provided system and/or kit can be used for screening and/or other assessment of individuals susceptible to prostate cancer (e.g., individuals with a known genetic, environmental, or experiential risk, etc.). In some embodiments, provided system and/or kits can be used for monitoring recurrence of prostate cancer in a subject who has been previously treated. In some embodiments, provided systems and/or kits can be used as a companion diagnostic in combination with a therapy for a subject who is suffering from prostate cancer. In some embodiments, provided systems and/or kits can be used for monitoring or evaluating efficacy of a therapy administered to a subject who is suffering from prostate cancer. In some embodiments, provided systems and/or kits can be used for selecting a therapy for a subject who is suffering from prostate cancer. In some embodiments, provided systems and/or kits can be used for making a therapy decision and/or selecting a therapy for a subject with one or more symptoms (e.g., non-specific symptoms) associated with prostate cancer.

Complexes formed by performing methods described herein and/or using systems and/or kits described herein are also within the scope of disclosure. For example, in some embodiments, a complex comprises: an extracellular vesicle expressing a target biomarker signature, which includes at least one extracellular vesicle-associated surface biomarker and at least one target biomarker selected from the group consisting of: surface biomarkers (e.g., ones described herein), intravesicular biomarkers (e.g., ones described herein), and intravesicular RNA biomarkers (e.g., ones described herein), wherein the extracellular vesicle is immobilized onto a solid substrate comprising a binding moiety directed to such a extracellular vesicle-associated surface biomarker. In some embodiments, such a complex further comprises at least two detection probes directed to at least one target biomarker of a target biomarker signature present in the extracellular vesicle, wherein each detection probe is bound to a respective target biomarker and each comprises: (i) a binding moiety directed to the target biomarker; and (ii) an oligonucleotide domain coupled to the binding moiety, the oligonucleotide domain comprising a double-stranded portion and a single-stranded overhang portion extended from one end of the oligonucleotide domain, wherein the single-stranded overhang portions of the detection probes are hybridized to each other.

In some embodiments, an extracellular vesicle-associated surface biomarker present in an extracellular vesicle that forms a complex may comprise one or more surface biomarkers described herein. In some embodiments, such an extracellular vesicle-associated biomarker may be or comprise (i) at least one polypeptide encoded by a human gene as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, or combinations thereof; and/or (ii) at least one carbohydrate-dependent marker as follows: Tn antigen, SialylTn (sTn) antigen, Thomsen-Friedenreich (T. TF) antigen, Lewis Y antigen (also known as CD174), Sialyl Lewis X (sLex) antigen (also known as Sialyl SSEA-1 (SLX)), or combinations thereof.

In some embodiments, an extracellular vesicle-associated biomarker may be or comprise (i) a polypeptide encoded by human gene MUC1; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, a surface biomarker present in an extracellular vesicle that forms a complex may be or comprise (i) at least one polypeptide encoded by a human gene as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, or combinations thereof; and/or (ii) at least one carbohydrate-dependent marker as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), or combinations thereof.

In some embodiments, a surface biomarker present in an extracellular vesicle that forms a complex may be or comprise (i) one or more polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) one or more carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, or combinations thereof.

In some embodiments, an intravesicular biomarker present in an extracellular vesicle that forms a complex may be or comprise at least one polypeptide encoded by a human gene: ACP5, ACPP, ACSM1, ACTA2, ACTG2, ADAMTS1, AGR2, AIM1, ALCAM, ALDH1A3, ALOX15B, ANTXR1, ANTXR2, AP1M2, AR, ARG2, ARHGAP6, ARHGEF26, ARHGEF37, ASAP3, ATP8B1, ATP9A, BAIAP2L1, BCAM, BHLHA15, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CGN, CGNL1, CHMP4C, CLGN, CMTM4, COLCA1, CORO2A, CPE, CPNE4, CPQ, CRYM, CTSF, CX3CL1, CXADR, CXCR4, CYB561, DPYSL3, DSP, EEF1A2, EFNA1, EHD2, EHF, ELF3, EMP2, EPHX1, EPN3, ERBB3, ERG, ESRP1, ESRP2, F3, FAAH, FAM129A, FAM129B, FAM210B, FAM3B, FAM3D, FAM83H, FAM84A, FAM84B, FAT1, FBLIM1, FBP1, FLNC, FNBP1L, FOS, FOXA1, GADD45G, GATA2, GGT1, GJB1, GLYATL1, GNG4, GNMT, GRHL2, GUCY1A3, HGD, HID1, HIST1H2BK, HIST3H2A, HMGCS2, HOMER2, HOXB13, HSD17B6, HSPB6, HSPB8, ICA1, ID1, IQGAP2, IRF6, ITGA6, KIAA1522, KIF5C, KLK2, KRT14, KRT15, KRT17, KRT18, KRT19, KRT5, KRT7, KRT8, LCP1, LIFR, LIMCH1, LMOD1, LRP11, LRP3, LSR, MAL2, MAOA, MAP7, MARC1, MARCKSL1, MB, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, PALLD, PARVA, PCDH1, PCP4, PDIA5, PEBP4, PGM5, PKP1, PKP3, PPL, PPM1H, PPP1R1B, PPP3CA, PRAC1, PRR15L, PSAT1, PTPRF, PYCR1, RAB3B, RAMP1, RAP1GAP, RASEF, RBM47, RCAN3, RDH11, REEP6, REPS2, RGS10, RHPN2, RNF144B, RORC, RPS4Y1, S100A16, SELENBP1, SERINC5, SH3BGRL2, SH3BP4, SHC2, SLC1A5, SLC22A3, SLC2A12, SLC40A1, SLC4A4, SLC7A8, SLC9A3R2, SMOC2, SORD, SPDEF, SPINT1, SPINT2, SPTBN2, ST14, STAP2, STK39, SULT2B1, SYNE4, SYT7, SYTL1, TAGLN, TBC1D16, TBX3, TC2N, TCEA3, TEAD3, TESC, THBS4, TM4SF1, TMED3, TMEM150C, TMEM54, TMEM98, TMSB15A, TP53111, TPD52, TPM1, TSPAN13, TSPAN6, TSPAN8, VAMP8, VSTM2L, WLS, WWC1, YAP1, ZBTB42, ZC3H11A, ZNF217, ACSL3, CADH7, KCNN2, OPRK1, PDE9A, PLPP1, SPON2, TMEM121B, or combinations thereof. In some embodiments, an intravesicular biomarker described herein may comprise at least one post-translational modification.

In some embodiments, an intravesicular RNA biomarker present in an extracellular vesicle that forms a complex may be or comprise at least one RNA transcript (e.g., mRNA transcript) encoded by a human gene: ABCC4, ACP5, ACPP, ACSM1, ACTA2, ACTG2, ADAMTS1, AGR2, AIM1, ALCAM, ALDH1A3, ALOX15B, ANO7, ANPEP, ANTXR1, ANTXR2, AP1M2, APCDD1, AQP3, AR, ARG2, ARHGAP6, ARHGEF26, ARHGEF37, ASAP3, ASTN2, ATP8B1, ATP9A, BAIAP2L1, BCAM, BHLHA15, BIK, BMPR1B, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CDH1, CGN, CGNL1, CHMP4C, CHRM1, CHRNA2, CLDN3, CLDN4, CLDN7, CLDN8, CLGN, CMTM4, COLCA1, COLEC12, CORO2A, CPE, CPNE4, CPQ, CRB3, CREB3L1, CREB3L4, CRYM, CTSF, CWH43, CX3CL1, CXADR, CXCR4, CYB561, DPP4, DPYSL3, DSG2, DSP, EEF1A2, EFNA1, EHD2, EHF, ELF3, ELOVL7, EMB, EMP2, ENPP5, EPCAM, EPHX1, EPN3, ERBB3, ERG, ESRP1, ESRP2, F3, FAAH, FAM129A, FAM129B, FAM189A2, FAM210B, FAM3B, FAM3D, FAM83H, FAM84A, FAM84B, FAT1, FBLIM1, FBP1, FLNC, FNBP1L, FOLH1, FOS, FOXA1, FXYD3, GADD45G, GALNT3, GALNT7, GATA2, GCNT1, GGT1, GJB1, GLYATL1, GNG4, GNMT, GOLM1, GPR160, GREB1, GRHL2, GUCY1A3, HGD, HID1, HIST1H2BK, HIST3H2A, HMGCS2, HOMER2, HOXB13, HPN, HSD17B6, HSPB6, HSPB8, ICA1, ID1, IQGAP2, IRF6, ITGA6, KCNN4, KIAA1324, KIAA1522, KIF5C, KLK2, KRT14, KRT15, KRT17, KRT18, KRT19, KRT5, KRT7, KRT8, KRTCAP3, LCP1, LIFR, LIMCH1, LMOD1, LPAR3, LRP11, LRP3, LRRC26, LSR, MAL2, MAOA, MAP7, MARC1, MARCKSL1, MB, MBOAT2, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, OR51E1, OR51E2, PALLD, PARM1, PARVA, PCDH1, PCP4, PDIA5, PDLIM5, PDZK1IP1, PDZRN3, PEBP4, PGM5, PIGR, PKP1, PKP3, PLA2G2A, PMEPA1, PODXL2, PPL, PPM1H, PPP1R1B, PPP3CA, PRAC1, PROM2, PRR15L, PRSS8, PSAT1, PSCA, PTPRF, PTPRN2, PYCR1, RAB25, RAB27B, RAB3B, RAMP1, RAP1GAP, RASEF, RBM47, RCAN3, RDH11, REEP6, REPS2, RGS10, RHPN2, RNF144B, RORC, RPS4Y1, S100A16, SCNN1A, SDC1, SELENBP1, SERINC2, SERINC5, SEZ6L2, SH3BGRL2, SH3BP4, SHC2, SLC15A2, SLC1A5, SLC22A3, SLC2A10, SLC2A12, SLC30A4, SLC35F2, SLC40A1, SLC44A4, SLC45A3, SLC4A4, SLC7A8, SLC9A3R2, SMIM22, SMOC2, SORD, SPDEF, SPINT1, SPINT2, SPTBN2, ST14, STAP2, STEAP1, STEAP2, STEAP4, STK39, SULT2B1, SYNE4, SYT7, SYTL1, TACSTD2, TAGLN, TBC1D16, TBX3, TC2N, TCEA3, TEAD3, TESC, THBS4, TM4SF1, TMC4, TMC5, TMED3, TMEFF2, TMEM125, TMEM150C, TMEM30B, TMEM45B, TMEM54, TMEM98, TMPRSS2, TMSB15A, TP53111, TPD52, TPM1, TRPM4, TRPM8, TRPV6, TSPAN1, TSPAN13, TSPAN6, TSPAN8, TUSC3, UPK3A, VAMP8, VSIG2, VSTM2L, WLS, WWC1, YAP1, ZBTB42, ZC3H11A, ZNF217, or combinations thereof.

In some embodiments, an extracellular vesicle-associated surface biomarker and/or a surface biomarker included in a target biomarker signature may be or comprise a CLDN3 polypeptide. In some embodiments, an extracellular vesicle-associated surface biomarker and/or a surface biomarker included in a target biomarker signature may be or comprise an ABCC4 polypeptide. In some embodiments, an extracellular vesicle-associated surface biomarker and/or a surface biomarker included in a target biomarker signature may be or comprise a RAB3B polypeptide. In some embodiments, an extracellular vesicle-associated surface biomarker and/or a surface biomarker included in a target biomarker signature may be or comprise a FOLH1 polypeptide. In some embodiments, an extracellular vesicle-associated surface biomarker and/or a surface biomarker included in a target biomarker signature may be or comprise a PMEPA1 polypeptide. In some embodiments, an extracellular vesicle-associated surface biomarker and/or a surface biomarker included in a target biomarker signature may be or comprise a TMPRSS2 polypeptide. In some embodiments, an extracellular vesicle-associated surface biomarker and/or a surface biomarker included in a target biomarker signature may be or comprise a TSPAN1 polypeptide. In some embodiments, an extracellular vesicle-associated surface biomarker and/or a surface biomarker included in a target biomarker signature may be or comprise a PODXL2 polypeptide. In some embodiments, an extracellular vesicle-associated surface biomarker and/or a surface biomarker included in a target biomarker signature may be or comprise a GOLM1 polypeptide. In some embodiments, an extracellular vesicle-associated surface biomarker and/or a surface biomarker included in a target biomarker signature may be or comprise a SORD polypeptide. In some embodiments, an extracellular vesicle-associated surface biomarker and/or a surface biomarker included in a target biomarker signature may be or comprise a CLGN polypeptide. In some embodiments, an extracellular vesicle-associated surface biomarker and/or a surface biomarker included in a target biomarker signature may be or comprise a GRHL2 polypeptide. In some embodiments, an extracellular vesicle-associated surface biomarker and/or a surface biomarker included in a target biomarker signature may be or comprise a CANT1 polypeptide. In some embodiments, an extracellular vesicle-associated surface biomarker and/or a surface biomarker included in a target biomarker signature may be or comprise a SYT7 polypeptide. In some embodiments, an extracellular vesicle-associated surface biomarker and/or a surface biomarker included in a target biomarker signature may be or comprise a PPP3CA polypeptide. In some embodiments, an extracellular vesicle-associated surface biomarker and/or a surface biomarker included in a target biomarker signature may be or comprise a CDH1 polypeptide. In some embodiments, an extracellular vesicle-associated surface biomarker and/or a surface biomarker included in a target biomarker signature may be or comprise a RDH11 polypeptide. In some embodiments, an extracellular vesicle-associated surface biomarker and/or a surface biomarker included in a target biomarker signature may be or comprise a CLDN4 polypeptide. In some embodiments, an extracellular vesicle-associated surface biomarker and/or a surface biomarker included in a target biomarker signature may be or comprise an EPCAM polypeptide. In some embodiments, an extracellular vesicle-associated surface biomarker and/or a surface biomarker included in a target biomarker signature may be or comprise a TRPM4 polypeptide. In some embodiments, an extracellular vesicle-associated surface biomarker and/or a surface biomarker included in a target biomarker signature may be or comprise a FAAH polypeptide.

In some embodiments, an extracellular vesicle-associated surface biomarker and/or a surface biomarker included in a target biomarker signature may be or comprise a MUC1 polypeptide. In some embodiments, an extracellular vesicle-associated surface biomarker and/or a surface biomarker included in a target biomarker signature may be or comprise a Lewis Y antigen. In some embodiments, an extracellular vesicle-associated surface biomarker and/or a surface biomarker included in a target biomarker signature may be or comprise a sTn antigen. In some embodiments, an extracellular vesicle-associated surface biomarker and/or a surface biomarker included in a target biomarker signature may be or comprise a sLex antigen. In some embodiments, an extracellular vesicle-associated surface biomarker and/or a surface biomarker included in a target-biomarker signature may be or comprise a T antigen. In some embodiments, an extracellular vesicle-associated surface biomarker and/or a surface biomarker included in a target-biomarker signature may be or comprise a Tn antigen.

Also within the scope of the present disclosure is a complex comprising: a nanoparticle having a size range of interest that includes extracellular vesicles, and comprising a prostate cancer-specific biomarker signature, which includes at least two surface biomarkers described herein, wherein the nanoparticle is immobilized onto a solid substrate comprising a binding moiety directed to a first surface biomarker of a prostate cancer-specific biomarker signature. In some embodiments, such a complex is also bound to at least two detection probes each directed to a surface biomarker (which can be the same or different surface biomarker(s)) of the prostate cancer-specific biomarker signature, wherein each detection probe is bound to a respective surface biomarker and each comprises: (i) a binding moiety directed to the surface biomarker; and (ii) an oligonucleotide domain coupled to the binding moiety, the oligonucleotide domain comprising a double-stranded portion and a single-stranded overhang portion extended from one end of the oligonucleotide domain, wherein the single-stranded overhang portions of the detection probes are hybridized to each other.

In some embodiments, the present disclosure describes a complex comprising: (a) a nanoparticle having a size within the range of about 30 nm to about 1000 nm, and comprising at least a first surface biomarker and a second surface biomarker, which combination is determined to be a target biomarker signature for prostate cancer, wherein the first surface biomarker and the second surface biomarker are each independently selected from: (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof; (b) a solid substrate comprising a target-capture moiety directed to the first surface biomarker; wherein the target-capture moiety binds to the first surface biomarker of the nanoparticle such that the nanoparticle is immobilized on the solid substrate; and (c) at least a first detection probe and a second detection probe each bound to the nanoparticle, wherein each detection probe comprises: (i) a target binding moiety directed to the second surface biomarker; and (ii) an oligonucleotide domain coupled to the target binding moiety, the oligonucleotide domain comprising a double-stranded portion and a single-stranded overhang portion extended from one end of the oligonucleotide domain, wherein the single-stranded overhang portions of the first and second detection probes are hybridized to each other.

In some embodiments, the first surface biomarker and the second surface biomarker(s) are each independently selected from: (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

These, and other aspects encompassed by the present disclosure, are described in more detail below and in the claims.

### BRIEF DESCRIPTION OF THE DRAWINGS

FIG. 1 is a schematic diagram illustrating an exemplary workflow of profiling individual extracellular vesicles (EVs). The figure shows purification of EVs from plasma using size exclusion chromatography (SEC) and immunoaffinity capture of EVs displaying a specific EV-associated surface marker (Panel A); detection of co-localized target markers (e.g., intravesicular biomarkers or surface biomarkers) on captured EVs using a target entity detection assay according to some embodiments described herein (Panel B).

FIG. 2 is a schematic diagram illustrating a target entity detection assay according to some embodiments described herein. In some embodiments, a target entity detection assay uses a combination of detection probes, which combination is specific for detection of cancer. In some embodiments, a duplex system includes a first detection probe for a target biomarker 1 and a second detection probe for a target biomarker 2 are added to a sample comprising a biological entity (e.g., extracellular vesicle). In some embodiments, detection probes each comprise a target binding moiety (e.g., an affinity agent such as, e.g., an antibody agent against a target biomarker) coupled to an oligonucleotide domain, which comprises a double-stranded portion and a single-stranded overhang extended from one end of the oligonucleotide domain. A detection signal is generated when distinct target binding moieties (e.g., affinity agents such as, e.g., antibody agents against target biomarker 1 and target biomarker 2, respectively) of the first and second detection probes are localized to the same biological entity (e.g., an extracellular vesicle) in close proximity such that the corresponding single-stranded overhangs hybridize to each other, thus allowing ligation of their oligonucleotide domains to occur. For example, a control entity (e.g., a biological entity from a healthy subject sample) does not express one or both of target biomarker 1 and target biomarker 2, so no detection of signal can be generated. However, when a biological entity from a cancer sample (e.g., a prostate cancer sample) expresses target biomarker 1 and target biomarker 2, and the target biomarkers are present within a short enough distance of each other in the same biological entity (e.g., extracellular vesicle), a detection signal is generated.

FIG. 3 is a schematic diagram illustrating a target entity detection assay according to some embodiments described herein. The figure shows an exemplary triplex target entity detection system, in which in some embodiments, three or more detection probes, each for a target biomarker, can be added to a sample comprising a biological entity (e.g., extracellular vesicle). In some embodiments, detection probes each comprise a target binding moiety (e.g., an affinity agent such as, e.g., an antibody agent against a target biomarker) coupled to an oligonucleotide domain, which comprises a double-stranded portion and a single-stranded overhang extended from one end of the oligonucleotide domain. A detection signal is generated when the corresponding single-stranded overhangs of all three or more detection probes hybridize to each other to form a linear double-stranded complex, and ligation of at least one strand of the double-stranded complex occurs, thus allowing a resulting ligated product to be detected.

FIG. 4 is a non-limiting example of a double-stranded complex comprising four detection probes connected to each other in a linear arrangement through hybridization of their respective single-stranded overhangs.

FIG. 5 is a schematic diagram illustrating a target entity detection assay of an exemplary embodiment described herein. In some embodiments, a plurality of detection probes, each for a distinct target, are added to a sample comprising a biological entity (e.g., extracellular vesicle). In some embodiments, detection probes each comprise a target binding moiety (e.g., an antibody agent) coupled to an oligonucleotide domain, which comprises a double-stranded portion and a single-stranded overhang extended from one end of the oligonucleotide domain. A detection signal is generated when all detection probes are localized to the same biological entity (e.g., an extracellular vesicle or analyte) in close proximity such that the corresponding single-stranded overhangs hybridize to form a linear double-stranded complex, and ligation of at least one strand of the resulting linear double-stranded complex occurs, thereby allowing a ligated product to be detected.

FIG. 6 is a depiction of a bar chart showing the 5-year relative survival rates by stage of diagnosis of prostate cancer taken from SEER 18 2010-2016, All Races, Both Sexes by SEER Summary Stage 2000.

FIG. 7 is a depiction of a pie chart showing at which point diagnosis occurs by percentage (localized, regional, distant, and unknown) for prostate cancer. Commonly, diagnosis occurs in the distant stage when cancer is most lethal. SEER 18 2010-2016, All Races, Both Sexes by SEER Summary Stage 2000.

FIG. 8 shows Ct values from characterization of certain exemplary biomarker combinations using methods and/or assays described herein (e.g., target entity detection systems as described herein) in prostate cancer-specific cell lines that express at least two surface biomarkers and in a negative control group. Panel A shows biomarker combination of EPCAM and T antigen Panel B shows biomarker combination of LEY and VWA1. Panel C shows biomarker combination of MARCKSL1 and T antigen, Panel D shows biomarker combination of T antigen and Tn antigen, Panel E shows biomarker combination of ABCC4 and TSPAN1, Panel F shows biomarker combination of FOLH1 and TSPAN1, and Panel G shows biomarker combination of MUC1 and TSPAN1.

FIG. 9 shows MIF RT-PCR signal (45-Ct) following EPCAM-targeted immunoaffinity capture for OVCAR-3 (positive cell line) and SK-MEL-1 (negative cell line) EVs. Multiple detergent (Tween-20) concentrations were evaluated, with 0% Tween showing greater delta values.

### CERTAIN DEFINITIONS

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be parenteral. In some embodiments, administration may be oral. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity Agent: The term “affinity agent” as used herein refers to an entity that is or comprises a target-binding moiety as described herein, and therefore binds to a target of interest (e.g., molecular target of interest such as a biomarker or an epitope). In many embodiments, an affinity agent in accordance with the present disclosure binds specifically with a biomarker as described herein. In many embodiments, an affinity agent in accordance with the present disclosure binds specifically with a surface protein biomarker as described herein. In some embodiments, an affinity agent in accordance with the present disclosure binds specifically with a carbohydrate-dependent marker as described herein. In some embodiments, an affinity agent may be or comprise an antibody agent (e.g., an antibody or other entity that is or includes an antigen-binding portion thereof). Alternatively or additionally, in some embodiments, an affinity agent may selected from the group consisting of affimers, aptamers, lectins, sialic acid-binding immunoglobulin-type lectins (siglecs), and combinations thereof, and/or another binding agent that may be considered a ligand. In some embodiments, a target (e.g., a biomarker target) of an affinity agent is or comprises one or more polypeptide, nucleic acid, carbohydrate, and/or lipid moieties and/or entities).

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amplification: The terms “amplification” and “amplify” refers to a template-dependent process that results in an increase in the amount and/or levels of a nucleic acid molecule relative to its initial amount and/or level. A template-dependent process is generally a process that involves template-dependent extension of a primer molecule, wherein the sequence of the newly synthesized strand of nucleic acid is dictated by the well-known rules of complementary base pairing (see, for example, Watson, J. D, et al., In: Molecular Biology of the Gene, 4th Ed., W. A. Benjamin, Inc., Menlo Park, Calif. (1987); which is incorporated herein by reference for the purpose described herein).

Antibody agent: As used herein, the term “antibody agent” refers to an agent that specifically binds to a particular antigen. In some embodiments, an antibody agent refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of rabbit, rodent (e.g., mouse, rat, hamster, etc.), camelid (e.g., llama, alpaca), sheep, goat, bovine, horse, chicken, donkey, shark, primate, human, or in vitro-derived (e.g., yeast, phage) antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, IgA, IgG, IgE or IgM antibodies; bi- or multi-specific antibodies (e.g., Zybodies®, etc); antibody fragments such as Fab fragments, Fab′ fragments, F(ab′) 2 fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, Affibodies, Affilins, Affimers, Affitins, Alphabodies, Avimers, Fynomers, Im7, VLR, VNAR, Trimab, CrossMab, Trident); nanobodies, binanobodies, di-sdFv, single domain antibodies, trifunctional antibodies, diabodies, and minibodies, etc. In some embodiments, relevant formats may be or include: Adnectins®; Affibodies®; Affilins®; Anticalins®; Avimers®; BiTERs; cameloid antibodies; Centyrins®; ankyrin repeat proteins or DARPINs®; dual-affinity re-targeting (DART) agents; Fynomers®; shark single domain antibodies such as IgNAR; immune mobilizing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., polyethylene glycol, etc.]).

Antigen: As used herein, the term “antigen” refers to an entity (e.g., a molecule or a molecular structure such as, e.g., a peptide or protein, carbohydrate, lipoparticle, oligonucleotide, chemical molecule, or combinations thereof) that includes one or more epitopes and therefore is recognized and bound by an affinity agent (e.g., an antibody, affimer, or aptamer).

Approximately or about: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Aptamer: As used herein, the term “aptamer” typically refers to a nucleic acid molecule or a peptide molecule that binds to a specific target molecule (e.g., an epitope). In some embodiments, a nucleic acid aptamer may be described by a nucleotide sequence and is typically about 15-60 nucleotides in length. A nucleic acid aptamer may be or comprise a single stranded and/or double-stranded structure. In some embodiments, a nucleic acid aptamer may be or comprise DNA. In some embodiments, a nucleic acid aptamer may be or comprise RNA. Without wishing to be bound by any theory, it is contemplated that the chain of nucleotides in an aptamer form intramolecular interactions that fold the molecule into a complex three-dimensional shape, and this three-dimensional shape allows the aptamer to bind tightly to the surface of its target molecule. In some embodiments, a peptide aptamer may be described to have one or more peptide loops of variable sequence displayed by a protein scaffold. Peptide aptamers can be isolated from combinatorial libraries and often subsequently improved by directed mutation or rounds of variable region mutagenesis and selection. Given the extraordinary diversity of molecular shapes that exist within the universe of all possible nucleotide and/or peptide sequences, aptamers may be obtained for a wide array of molecular targets, including proteins and small molecules. In addition to high specificity, aptamers typically have very high affinities for their targets (e.g., affinities in the picomolar to low nanomolar range for proteins or polypeptides). Because aptamers are typically synthetic molecules, aptamers are amenable to a variety of modifications, which can optimize their function for particular applications.

Associated with: Two events or entities are “associated” with one another, as that term is used herein, if the presence, level and/or form of one is correlated with that of the other. For example, a particular biological phenomenon (e.g., expression of a specific biomarker) is considered to be associated with prostate cancer (e.g., a specific type of prostate cancer (e.g., prostate adenocarcinoma) and/or stage of prostate cancer), if its presence correlates with incidence of and/or susceptibility of the prostate cancer (e.g., across a relevant population).

Biological entity: In appropriate circumstances, as will be clear from context to those skilled in the art, the term “biological entity” may be utilized to refer to an entity or component that is present in a biological sample, e.g., in some embodiments derived or obtained from a subject, which, in some embodiments, may be or comprise a cell or an organism, such as an animal or human, or, in some embodiments, may be or comprise a biological tissue or fluid. In some embodiments, a biological entity is or comprises a cell or microorganism, or a fraction, extract, or component thereof (including, e.g., intracellular components and/or molecules secreted by a cell or microorganism). For example, in some embodiments, a biological entity is or comprises a cell. In some embodiments, a biological entity is or comprises a nanoparticle having a size within the range of about 30 nm to about 1000 nm, which in some embodiments are obtained from a bodily fluid sample (e.g., but not limited to a blood sample, a urine sample, etc.) of a subject. In some embodiments, such a nanoparticle may be or comprise a protein aggregate, including, e.g., in some embodiments comprising a glycan, and/or an extracellular vesicle. In some embodiments, such a nanoparticle may have a size within the range of about 30 nm to about 1000 nm, about 50 nm to about 500 nm, or about 75 nm to about 500 nm. In some embodiments, a biological entity is or comprises an extracellular vesicle. In some embodiments, a biological entity is or comprises a biological analyte (e.g., a metabolite, carbohydrate, protein or polypeptide, enzyme, lipid, organelle, cytokine, receptor, ligand, and any combinations thereof). In some embodiments, a biological entity present in a sample is in a native state (e.g., proteins or polypeptides remain in a naturally occurring conformational structure). In some embodiments, a biological entity is processed, e.g., by isolating from a sample or deriving from a naturally occurring biological entity. For example, a biological entity can be processed with one or more chemical agents such that it is more desirable for detection utilizing technologies provided herein. As an example only, a biological entity may be a cell or extracellular vesicle that is contacted with a fixative agent (e.g., but not limited to methanol and/or formaldehyde) to cause proteins and/or peptides present in the cell or extracellular vesicle to form crosslinks. In some embodiments, a biological entity is in an isolated or pure form (e.g., isolated from a bodily fluid sample such as, e.g., a blood, serum, plasma, or urine sample, etc.). In some embodiments, a biological entity may be present in a complex matrix (e.g., a bodily fluid sample such as, e.g., a blood, serum, plasma, or urine sample, etc.).

Biomarker: The term “biomarker” typically refers to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. In some embodiments, a biomarker may be or comprise a marker for a particular tissue (e.g., but not limited to brain, breast, colon, pancreas, prostate and/or other tissues associated with a male reproductive system, liver, lung, and skin). Such a marker for a particular tissue, in some embodiments, may be specific for a healthy tissue, specific for a diseased tissue, or in some embodiments may be present in a normal healthy tissue and diseased tissue (e.g., a tumor); those skilled in the art, reading the present disclosure, will appreciate appropriate contexts for each such type of biomarker. In some embodiments, a biomarker may be or comprise a cancer-specific marker (e.g., a marker that is specific to a particular cancer). In some embodiments, a biomarker may be or comprise a non-specific cancer marker (e.g., a marker that is present in at least two or more cancers). A non-specific cancer marker may be or comprise, in some embodiments, a generic marker for cancers (e.g., a marker that is typically present in cancers, regardless of tissue types), or in some embodiments, a marker for cancers of a specific tissue (e.g., but not limited to brain, breast, colon, pancreas, prostate and/or other tissues associated with a male reproductive system, liver, lung, and skin). Thus, in some embodiments, a biomarker is predictive; in some embodiments, a biomarker is prognostic; in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is or comprises a portion of a particular molecule, complex, or structure; e.g., in some embodiments, a biomarker may be or comprise an epitope. In some embodiments, a biomarker is a surface marker (e.g., a surface protein marker) of an extracellular vesicle associated with prostate cancer (e.g., prostate adenocarcinoma). In some embodiments, a biomarker is intravesicular (e.g., a protein or RNA marker that is present within an extracellular vesicle). In some embodiments, a biomarker may be or comprise a genetic or epigenetic signature. In some embodiments, a biomarker may be or comprise a gene expression signature. In some embodiments, a “biomarker” appropriate for use in accordance with the present disclosure may refer to presence, level, and/or form of a molecular entity (e.g., epitope) present in a target marker. For example, in some embodiments, two or more “biomarkers” as molecular entities (e.g., epitopes) may be present on the same target marker (e.g., a marker protein such as a surface protein present in an extracellular vesicle).

Blood-derived sample: The term “blood-derived sample.” as used herein, refers to a sample derived from a blood sample (i.e., a whole blood sample) of a subject in need thereof. Examples of blood-derived samples include, but are not limited to, blood plasma (including, e.g., fresh frozen plasma), blood serum, blood fractions, plasma fractions, serum fractions, blood fractions comprising red blood cells (RBC), platelets, leukocytes, etc., and cell lysates including fractions thereof (for example, cells, such as red blood cells, white blood cells, etc., may be harvested and lysed to obtain a cell lysate). In some embodiments, a blood-derived sample that is used with methods, systems, and/or kits described herein is a plasma sample.

Cancer: The term “cancer” is used herein to generally refer to a disease or condition in which cells of a tissue of interest exhibit relatively abnormal, uncontrolled, and/or autonomous growth, so that they exhibit an aberrant growth phenotype characterized by a significant loss of control of cell proliferation. In some embodiments, cancer may comprise cells that are precancerous (e.g., benign), malignant, pre-metastatic, metastatic, and/or non-metastatic. The present disclosure provides technologies for detection of prostate cancer (including, for example, prostate adenocarcinoma).

Capture assay: As used herein, the term “capture assay” refers to a process of isolating or separating a biological entity of interest from a sample (e.g., in some embodiments a bodily fluid-derived sample). In some embodiments, a biological entity of interest is isolated or separated from a sample (e.g., in some embodiments a bodily fluid-derived sample) using a capture probe described herein. In some embodiments, a biological entity of interest that binds to a capture probe described herein is subject to a detection assay described herein. In some embodiments, a biological entity of interest amenable to a capture assay described herein is or comprises nanoparticles having a size range of interest that includes extracellular vesicles. In some embodiments, such a nanoparticle may have a size within the range of about 30 nm to about 1000 nm, about 50 nm to about 500 nm, or about 75 nm to about 500 nm. In some embodiments, a biological entity of interest amenable to a capture assay described herein is or comprises extracellular vesicles (e.g., in some embodiments exosomes) of interest.

Capture probe: As used herein, the term “capture probe” refers to a capture agent for capturing a biological entity of interest from a sample (e.g., in some embodiments a bodily fluid-derived sample). In many embodiments described herein, a capture agent comprises at least one target-capture moiety that binds to a surface polypeptide of a biological entity of interest. In some embodiments, such a biological entity of interest is or comprises nanoparticles having a size range of interest that includes extracellular vesicles. In some embodiments, such nanoparticles may have a size within the range of about 30 nm to about 1000 nm, about 50 nm to about 500 nm, or about 75 nm to about 500 nm. In some embodiments, such a biological entity of interest comprises extracellular vesicles (e.g., in some embodiments exosomes). In some embodiments, a capture agent comprises at least one target moiety that binds to a surface biomarker (e.g., ones described herein) of nanoparticles having a size within the range of about 30 nm to about 1000 nm, including, e.g., extracellular vesicles (e.g., in some embodiments exosomes). In some embodiments, a target-capture moiety of a capture agent is or comprises an affinity agent described herein. In some embodiments, a target-capture moiety of a capture agent is or comprises an antibody agent. In some embodiments, a target-capture moiety of a capture agent is or comprises a lectin or a sialic acid-binding immunoglobulin-type lectin. In some embodiments, a capture agent may comprise a solid substrate such that its target-capture moiety is immobilized thereonto. In some embodiments, an exemplary solid substrate is a bead (e.g., a magnetic bead). In some embodiments, a capture probe is or comprises a population of magnetic beads comprising a target-capture moiety that specifically binds to a surface biomarker described herein.

Classification cutoff: As used herein, the term “classification cutoff” refers to a level, value, or score, or a set of values, or an indicator that is used to predict a subject's risk for a disease or condition (e.g., prostate adenocarcinoma), for example, by defining one or more dividing lines among two or more subsets of a population (e.g., normal healthy subjects and subjects with inflammatory conditions vs. prostate adenocarcinoma subjects). In some embodiments, a classification cutoff may be determined referencing at least one reference threshold level (e.g., reference cutoff) for a target biomarker signature described herein, optionally in combination with other appropriate variables, e.g., age, life-history-associated risk factors, hereditary factors, physical and/or medical conditions of a subject. In some embodiments where a classification is based on a single target biomarker signature (e.g., as described herein), a classification cutoff may be the same as a reference threshold (e.g., cutoff) pre-determined for the single target biomarker signature. In some embodiments where a classification is based on two or more (e.g., 2, 3, 4, or more) target biomarker signatures, a classification cutoff may reference two or more reference thresholds (e.g., cutoffs) each individually pre-determined for the corresponding target biomarker signatures, and optionally incorporate one or more appropriate variables, e.g., age, life-history-associated risk factors, hereditary factors, physical and/or medical conditions of a subject. In some embodiments, a classification cutoff may be determined via a computer algorithm-mediated analysis that references at least one reference threshold level (e.g., reference cutoff) for a target biomarker signature described herein, optionally in combination with other appropriate variables, e.g., age, life-history-associated risk factors, hereditary factors, physical and/or medical conditions of a subject.

Close proximity: The term “close proximity” as used herein, refers to a distance between two detection probes (e.g., two detection probes in a pair) that is sufficiently close enough such that an interaction between the detection probes (e.g., through respective oligonucleotide domains) is expected to likely occur. For example, in some embodiments, probability of two detection probes interacting with each other (e.g., through respective oligonucleotide domains) over a period of time when they are in sufficiently close proximity to each other under a specified condition (e.g., when detection probes are bound to respective targets in an extracellular vesicle is at least 50% or more, including, e.g., at least 60%, at least 70%, at least 80%, at least 90% or more. In some embodiments, a distance between two detection probes when they are in sufficiently close proximity to each other may range between approximately 0.1-1000 nm, or 0.5-500 nm, or 1-250 nm. In some embodiments, a distance between two detection probes when they are in sufficiently close proximity to each other may range between approximately 0.1-10 nm or between approximately 0.5-5 nm. In some embodiments, a distance between two detection probes when they are in sufficiently close proximity to each other may be less than 100 nm or shorter, including, e.g., less than 90 nm, less than 80 nm, less than 70 nm, less than 60 nm, less than 50 nm, less than 40 nm, less than 30 nm, less than 20 nm, less than 10 nm, less than 5 nm, less than 1 nm, or shorter. In some embodiments, a distance between two detection probes when they are in sufficiently close proximity to each other may range between approximately 40-1000 nm or 40 nm-500 nm.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Complementary: As used herein, the term “complementary” in the context of nucleic acid base-pairing refers to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules. In the context of identifying biomarker combinations for detection of a particular cancer, the term “complementary” is used herein in reference to sets of biomarkers having different information content (e.g., ability to detect cancer in distinct, substantially non-overlapping subgroups of subjects). For example, two sets of biomarkers-set 1 and set 2—are said to be “complementary” to each other if, for example, set 1 detects cancer in a group (e.g., group A) of subjects in a population, and set 2 detects cancer in a substantially separate and non-overlapping group of subjects in the same population (e.g., group B), but not in Group A. Similarly, set 1 does not detect cancer in a substantial number of subjects in Group B.

Detecting: The term “detecting” is used broadly herein to include appropriate means of determining the presence or absence of an extracellular vesicle expressing a target biomarker signature of prostate cancer (e.g., prostate adenocarcinoma) or any form of measurement indicative of such an extracellular vesicle. Thus, “detecting” may include determining, measuring, assessing, or assaying the presence or absence, level, amount, and/or location of an entity of interest (e.g., a surface biomarker, an intravesicular biomarker, or an intravesicular RNA biomarker) that corresponds to part of a target biomarker signature in any way. In some embodiments, “detecting” may include determining, measuring, assessing, or quantifying a form of measurement indicative of an entity of interest (e.g., a ligated template indicative of a surface biomarker and/or an intravesicular biomarker, or a PCR amplification product indicative of an intravesicular mRNA). Quantitative and qualitative determinations, measurements or assessments are included, including semi-quantitative. Such determinations, measurements or assessments may be relative, for example when an entity of interest (e.g., a surface biomarker, an intravesicular biomarker, or an intravesicular RNA biomarker) or a form of measurement indicative thereof is being detected relative to a control reference, or absolute. As such, the term “quantifying” when used in the context of quantifying an entity of interest (e.g., a surface biomarker, an intravesicular biomarker, or an intravesicular RNA biomarker) or a form of measurement indicative thereof can refer to absolute or to relative quantification. Absolute quantification may be accomplished by correlating a detected level of an entity of interest (e.g., a surface biomarker, an intravesicular biomarker, or an intravesicular RNA biomarker) or a form of measurement indicative thereof to known control standards (e.g., through generation of a standard curve). Alternatively, relative quantification can be accomplished by comparison of detected levels or amounts between two or more different entities of interest (e.g., different surface biomarkers, intravesicular biomarkers, or intravesicular RNA biomarkers) to provide a relative quantification of each of the two or more different entities of interest, i.e., relative to each other.

Detection label: The term “detection label” as used herein refers to any element, molecule, functional group, compound, fragment or moiety that is detectable. In some embodiments, a detection label is provided or utilized alone. In some embodiments, a detection label is provided and/or utilized in association with (e.g., joined to) another agent. Examples of detection labels include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr, etc.), fluorescent dyes, chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, digoxigenin, haptens, and proteins for which antisera or monoclonal antibodies are available.

Detection probe: The term “detection probe” typically refers to a probe directed to detection and/or quantification of a specific target. In some embodiments, a detection probe is a quantification probe, which provides an indicator representing level of a specific target. In accordance with the present disclosure, a detection probe refers to a composition comprising a target binding entity, directly or indirectly, coupled to an oligonucleotide domain, wherein the target binding entity specifically binds to a respective target (e.g., molecular target), and wherein at least a portion of the oligonucleotide domain is designed to permit hybridization with a portion of an oligonucleotide domain of another detection probe for a distinct target. In many embodiments, an oligonucleotide domain appropriate for use in the accordance with the present disclosure comprises a double-stranded portion and at least one single-stranded overhang. In some embodiments, an oligonucleotide domain may comprise a double-stranded portion and a single-stranded overhang at each end of the double-stranded portion. In some embodiments, a target binding entity of a detection probe is or comprises an affinity agent described herein. In some embodiments, a target binding entity of a detection probe is or comprises an antibody agent. In some embodiments, a target binding entity of a detection probe is or comprises a lectin or a sialic acid-binding immunoglobulin-type lectin (siglec).

Double-stranded: As used herein, the term “double-stranded” in the context of oligonucleotide domain is understood by those of skill in the art that a pair of oligonucleotides exist in a hydrogen-bonded, helical arrangement typically associated with, for example, nucleic acid such as DNA. In addition to the 100% complementary form of double-stranded oligonucleotides, the term “double-stranded” as used herein is also meant to refer to those forms which include mismatches (e.g., partial complementarity) and/or structural features as bulges, loops, or hairpins.

Double-stranded complex: As used herein, the term “double-stranded complex” typically refers to a complex comprising at least two or more (including, e.g., 2, 3, 4, 5, 6, 7, 8, 9, 10, 11, 12, 13, 14, 15, 16, 17, 18, 19, 20, or more) detection probes (e.g., as provided and/or utilized herein), each directed to a target (which can be the same target or a distinct target), connected or coupled to one another in a linear arrangement through hybridization of complementary single-stranded overhangs of the detection probes. In some embodiments, such a double-stranded complex may comprise an extracellular vesicle, wherein respective target binding moieties of the detection probes are simultaneously bound to the extracellular vesicle.

Epitope: As used herein, the term “epitope” includes any moiety that is specifically recognized by an affinity agent (e.g., but not limited to an antibody, affimer, and/or aptamer). In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Extracellular vesicle: As used herein, the term “extracellular vesicle” typically refers to a vesicle outside of a cell, e.g., secreted by a cell. Examples of secreted vesicles include, but are not limited to exosomes, microvesicles, microparticles, ectosomes, oncosomes, and apoptotic bodies. Without wishing to be bound by theory, exosomes are nanometer-sized vesicles (e.g., between 40 nm and 120 nm) of endocytic origin that may form by inward budding of the limiting membrane of multivesicular endosomes (MVEs), while microvesicles typically bud from the cell surface and their size may vary between 50 nm and 1000 nm. In some embodiments, an extracellular vesicle is or comprises an exosome and/or a microvesicle. In some embodiments, a sample comprising an extracellular vesicle is substantially free of apoptotic bodies. In some embodiments, a sample comprising extracellular vesicles may comprise extracellular vesicles shed or derived from one or more tissues (e.g., cancerous tissues and/or non-cancerous or healthy tissues). In some embodiments, an extracellular vesicle in a sample may be shed or derived from a prostate adenocarcinoma tumor; in some embodiments, an extracellular vesicle is shed or derived from a tumor of a non-prostate adenocarcinoma. In some embodiments, an extracellular vesicle is shed or derived from a healthy tissue. In some embodiments, an extracellular vesicle is shed or derived from a benign prostate tumor. In some embodiments, an extracellular vesicle is shed or derived from a tissue of a subject with symptoms (e.g., non-specific symptoms) associated with prostate adenocarcinoma.

Extracellular vesicle-associated membrane-bound polypeptide: As used herein, such a term refers to a polypeptide that is present in the membrane of an extracellular vesicle. In some embodiments, such a biomarker may be associated with the extracellular side of the membrane. In some embodiments, such a polypeptide may be tumor specific. In some embodiments, such a polypeptide may be tissue-specific (e.g., prostate tissue-specific). In some embodiments, such a polypeptide may be non-specific, e.g., it is present in one or more non-target tumors, and/or in one or more non-target tissues.

Hybridization: As used herein, the term “hybridizing”, “hybridize”, “hybridization”, “annealing”, or “anneal” are used interchangeably in reference to pairing of complementary nucleic acids using any process by which a strand of nucleic acid joins with a complementary strand through base pairing to form a hybridization complex. Hybridization and the strength of hybridization (e.g., strength of the association between the nucleic acids) is impacted by various factors including, e.g., the degree of complementarity between the nucleic acids, stringency of the conditions involved, the melting temperature (T) of the formed hybridization complex, and the G: C ratio within the nucleic acids.

Intravesicular protein biomarker: As used herein, the term “intravesicular protein biomarker” refers to a marker indicative of the state (e.g., presence, level, and/or activity) of a polypeptide that is present within a biological entity (e.g., a cell or an extracellular vesicle). In many embodiments, an intravesicular protein biomarker is associated with or present within an extracellular vesicle. In many embodiments, an intravesicular protein biomarker may be post-translationally modified in a reversible (e.g. phosphorylation) or irreversible (e.g. cleavage) manner. In some embodiments, an intravesicular protein biomarker may be or comprise a phosphorylated polypeptide. In some embodiments, an intravesicular protein biomarker may be or comprise a mutated polypeptide.

Intravesicular RNA biomarker: As used herein, the term “intravesicular RNA biomarker” refers to a marker indicative of the state (e.g., presence and/or level) of a RNA that is present within a biological entity (e.g., a cell or an extracellular vesicle). In many embodiments, an intravesicular RNA biomarker is associated with or present within an extracellular vesicle. In some embodiments, an intravesicular RNA biomarker is associated or specific to cancer. In some embodiments, an intravesicular RNA biomarker is or comprises an mRNA transcript. In some embodiments, an intravesicular RNA biomarker is or comprises a noncoding RNA. Exemplary noncoding RNAs may include, but are not limited to small nuclear RNA, microRNA (miRNA), small nucleolar RNA (snoRNA), circular RNA (circRNA), long noncoding RNA (lncRNA), small noncoding RNA, piwi-interacting RNA, etc.). Certain RNA biomarkers for cancer are described in the art, e.g., as described in Xi et al. “RNA Biomarkers: Frontier of Precision Medicine for Cancer” Noncoding RNA (2017) 3:9, the contents of which are incorporated herein by reference for purposes described herein. In some embodiments, an intravesicular RNA biomarker is or comprise an orphan noncoding RNA (oncRNA). Certain oncRNAs that are cancer-specific were identified and described in the art, e.g., as described in Teng et al. “Orphan noncoding RNAs: novel regulators and cancer biomarkers” Ann Transl Med (2019) 7: S21; Fish et al. “Cancer cells exploit an orphan RNA to drive metastatic progression” Nature Medicine (2018) 24:1743-1751; International Patent Publication WO 2019/094780, each of which are incorporated herein by reference for purposes described herein. In some embodiments, an intravesicular RNA biomarker is or comprises a long non-coding RNA. Certain non-coding RNA biomarkers for cancer are described in the art, e.g., as described in Qian et al. “Long Non-coding RNAs in Cancer: Implications for Diagnosis, Prognosis, and Therapy” Front. Med. (2020) Volume 7, Article 612393, the contents of which are incorporated herein by reference for purposes described herein. In some embodiments, an intravesicular RNA biomarker is or comprises piwiRNA. In some embodiments, an intravesicular RNA biomarker is or comprises miRNA. In some embodiments, an intravesicular RNA biomarker is or comprises snoRNA. In some embodiments, an intravesicular RNA biomarker is or comprises circRNA.

Ligase: As used herein, the term “ligase” or “nucleic acid ligase” refers to an enzyme for use in ligating nucleic acids. In some embodiments, a ligase is enzyme for use in ligating a 3′-end of a polynucleotide to a 5′-end of a polynucleotide. In some embodiments, a ligase is an enzyme for use to perform a sticky-end ligation. In some embodiments, a ligase is an enzyme for use to perform a blunt-end ligation. In some embodiments, a ligase is or comprises a DNA ligase.

Life-history-associated risk factors: As used herein, the term “life-history risk factors” refers to individuals' actions, experiences, medical history, and/or exposures in their lives which may directly or indirectly increase such individuals' risk for a condition, e.g., cancer such as, e.g., prostate adenocarcinoma, relative to individuals who do not have such actions, experiences, medical history, and/or exposures in their lives. In some embodiments, non-limiting examples of life-history-associated risk factors include smoking, alcohol, drugs, carcinogenic agents, diet, obesity, diabetes, physical activity, sun exposure, radiation exposure, bituminous smoke exposure, exposure to infectious agents such as viruses and bacteria, and/or occupational hazard (Reid et al., 2017; which is incorporated herein by reference for the purpose described herein). One skilled in the art recognizes that the above list of life-history-associated risk factors contributing to cancer (e.g., prostate adenocarcinoma) susceptibility is not exhaustive but constantly evolving.

Ligation: As used herein, the term “ligate”, “ligating or “ligation” refers to a method or composition known in the art for joining two oligonucleotides or polynucleotides. A ligation may be or comprise a sticky-end ligation or a blunt-end ligation. In some embodiments, ligation involved in provided technologies is or comprises a sticky-end ligation. In some embodiments, ligation refers to joining a 3′ end of a polynucleotide to a 5′ end of a polynucleotide. In some embodiments, ligation is facilitated by use of a nucleic acid ligase.

Nanoparticles: The term “nanoparticles” as used in the context of a sample for a detection assay (e.g., as described herein) refers to particles having a size range of about 30 nm to about 1000 nm. In some embodiments, nanoparticles have a size range of about 30 nm to about 750 nm. In some embodiments, nanoparticles have a size range of about 50 nm to about 750 nm. In some embodiments, nanoparticles have a size range of about 30 nm to about 500 nm. In some embodiments, nanoparticles have a size range of about 50 nm to about 500 nm. In some embodiments, nanoparticles are obtained from a bodily fluid sample of a subject, for example, in some embodiments by a size exclusion-based method (e.g., in some embodiments size exclusion chromatography). In some embodiments, nanoparticles are or comprise analyte aggregates, which in some embodiments may be or comprise protein or mucin aggregates. In some embodiments, nanoparticles are or comprise protein multimers. In some embodiments, nanoparticles are or comprise extracellular vesicles.

Non-cancer subjects: As used herein, the term “non-cancer subjects” generally refers to subjects who do not have non-benign prostate cancer, and more specifically prostate adenocarcinoma. For example, in some embodiments, a non-cancer subject is a healthy subject. In some embodiments, a non-cancer subject is a healthy subject below age 55. In some embodiments, a non-cancer subject is a healthy subject of age 55 or above. In some embodiments, a non-cancer subject is a subject with non-prostate related health diseases, disorders, or conditions. In some embodiments, a non-cancer subject is a subject having benign prostatic hyperplasia.

Nucleic acid/Oligonucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of an oligonucleotide, a certain number of nucleotides refers to the number of nucleotides on a single strand, e.g., of an oligonucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes cancer, or presence of one or more tumors. In some embodiments, a patient is receiving or has received certain therapy to diagnose and/or to treat a disease, disorder, or condition.

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, glycosylation, methylation, etc. In some embodiments, polypeptides may comprise natural amino acids, non-natural amino acids, synthetic amino acids, and combinations thereof (e.g., may be or comprise peptidomimetics).

Prevent or prevention: As used herein, “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Primer: As used herein, the term “primer” refers to an oligonucleotide capable of acting as a point of initiation of synthesis when placed under conditions in which synthesis of a primer extension product which is complementary to a nucleic acid strand is induced (e.g., in the presence of nucleotides and an inducing agent such as DNA polymerase and at a suitable temperature and pH). A primer is preferably single stranded for maximum efficiency in amplification. A primer must be sufficiently long to prime the synthesis of extension products in the presence of the inducing agent. The exact lengths of a primer can depend on many factors, e.g., desired annealing temperature, etc.

Reference: As used herein, “reference” describes a standard or control relative to which a comparison is performed. For example, in some embodiments, an agent, animal, individual, population, sample, sequence or value of interest is compared with a reference or control agent, animal, individual, population, sample, sequence, or value. In some embodiments, a reference or control is tested and/or determined substantially simultaneously with the testing or determination of interest. In some embodiments, a reference or control is a historical reference or control, optionally embodied in a tangible medium. In some embodiments, a reference or control in the context of a reference level of a target refers to a level of a target in a normal healthy subject or a population of normal healthy subjects. In some embodiments, a reference or control in the context of a reference level of a target refers to a level of a target in a subject prior to a treatment. Typically, as would be understood by those skilled in the art, a reference or control is determined or characterized under comparable conditions or circumstances to those under assessment. In some embodiments, cell-line-derived extracellular vesicles are used as a reference or control. Those skilled in the art will appreciate when sufficient similarities are present to justify reliance on and/or comparison to a particular possible reference or control.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

Sample: As used herein, the term “sample” typically refers to an aliquot of material obtained or derived from a source of interest. In some embodiments, a sample is obtained or derived from a biological source (e.g., a tissue or organism or cell culture) of interest. In some embodiments, a source of interest may be or comprise a cell or an organism, such as an animal or human. In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravesicular fluid (blood plasma), an interstitial fluid, a lymphatic fluid, and/or a transcellular fluid. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises a bodily fluid sample or a bodily fluid-derived sample. Examples of a bodily fluid include, but are not limited to an amniotic fluid, bile, blood, breast milk, bronchoalveolar lavage fluid (BAL), cerebrospinal fluid, dialysate, feces, saliva, semen, synovial fluid, tears, urine, etc. In some embodiments, a biological sample is or comprises a liquid biopsy. In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, a sample is a preparation that is processed by using a semi-permeable membrane or an affinity-based method such antibody-based method to separate a biological entity of interest from other non-target entities. Such a “processed sample” may comprise, for example, in some embodiments extracellular vesicles, while, in some embodiments, nucleic acids and/or proteins, etc., extracted from a sample. In some embodiments, a processed sample can be obtained by subjecting a primary sample to one or more techniques such as amplification or reverse transcription of nucleic acid, isolation and/or purification of certain components, etc.

Selective or specific: The term “selective” or “specific”, when used herein with reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety. In some embodiments, a target-binding moiety does not detectably bind to the competing alternative target under conditions of binding to its target entity. In some embodiments, a target-binding moiety binds with higher on-rate, lower off-rate, increased affinity, decreased dissociation, and/or increased stability to its target entity as compared with the competing alternative target(s).

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is biologically active. In some embodiments, suitable small molecules may be identified by methods such as screening large libraries of compounds (Beck-Sickinger & Weber (2001) Combinational Strategies in Biology and Chemistry (John Wiley & Sons, Chichester, Sussex); by structure-activity relationship by nuclear magnetic resonance (Shuker et al. (1996) “Discovering high-affinity ligands for proteins: SAR by NMR.” Science 274:1531-1534); encoded self-assembling chemical libraries (Melkko et al. (2004) “Encoded self-assembling chemical libraries.” Nature Biotechnol. 22:568-574); DNA-templated chemistry (Gartner et al. (2004) “DNA-templated organic synthesis and selection of a library of macrocycles.” Science 305:1601-1605); dynamic combinatorial chemistry (Ramstrom & Lehn (2002) “Drug discovery by dynamic combinatorial libraries.” Nature Rev. Drug Discov. 1:26-36); tethering (Arkin & Wells (2004) “Small-molecule inhibitors of protein-protein interactions: progressing towards the dream.” Nature Rev. Drug Discov. 3:301-317); and speed screen (Muckenschnabel et al. (2004) “SpeedScreen: label-free liquid chromatography-mass spectrometry-based high-throughput screening for the discovery of orphan protein ligands.” Anal. Biochem. 324:241-249). In some embodiments, a small molecule may have a dissociation constant for a target in the nanomolar range.

Specific binding: As used herein, the term “specific binding” refers to an ability to discriminate between possible binding partners in the environment in which binding is to occur. A target-binding moiety that interacts with one particular target when other potential targets are present is said to “bind specifically” to the target with which it interacts. In some embodiments, specific binding is assessed by detecting or determining degree of association between a target-binding moiety and its partner; in some embodiments, specific binding is assessed by detecting or determining degree of dissociation of a target-binding moiety-partner complex; in some embodiments, specific binding is assessed by detecting or determining ability of a target-binding moiety to compete an alternative interaction between its partner and another entity. In some embodiments, specific binding is assessed by performing such detections or determinations across a range of concentrations.

Stage of cancer: As used herein, the term “stage of cancer” refers to a qualitative or quantitative assessment of the level of advancement of a cancer (e.g., prostate adenocarcinoma). In some embodiments, criteria used to determine the stage of a cancer may include, but are not limited to, one or more of where the cancer is located in a body, tumor size, whether the cancer has spread to lymph nodes, whether the cancer has spread to one or more different parts of the body, etc. In some embodiments, cancer may be staged using the AJCC staging system. The AJCC staging system is a classification system, developed by the American Joint Committee on Cancer for describing the extent of disease progress in cancer patients, which utilizes in part the TNM scoring system: Tumor size, Lymph Nodes affected, Metastases. In some embodiments, cancer may be staged using a classification system that in part involves the TNM scoring system, according to which T refers to the size and extent of the main tumor, usually called the primary tumor; N refers to the number of nearby lymph nodes that have cancer; and M refers to whether the cancer has metastasized. In some embodiments, a cancer may be referred to as Stage 0 (abnormal cells are present but have not spread to nearby tissue, also called carcinoma in situ, or C1S; C1S is not cancer, but it may become cancer), Stage I-III (cancer is present; the higher the number, the larger the tumor and the more it has spread into nearby tissues), or Stage IV (the cancer has spread to distant parts of the body). In some embodiments, a cancer may be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to figure out the stage). In some embodiments, a prostate cancer (e.g., prostate adenocarcinoma) may be assigned a Gleason score which is a measure of how likely the cancer is to grow and spread. In some embodiments, a Gleason score is determined from a prostate biopsy or surgery. In some embodiments, a Gleason score may comprise a score between 1 and 5, and one of skill in the art would know how to assign a Gleason score to a prostate cancer (e.g., prostate adenocarcinoma) based on histological patterns.

Subject: As used herein, the term “subject” refers to an organism from which a sample is obtained, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject, e.g., a human male subject. In some embodiments, a subject is suffering from prostate adenocarcinoma. In some embodiments, a subject is susceptible to prostate adenocarcinoma. In some embodiments, a subject displays one or more symptoms or characteristics of prostate adenocarcinoma. In some embodiments, a subject displays one or more non-specific symptoms of prostate adenocarcinoma. In some embodiments, a subject does not display any symptom or characteristic of prostate adenocarcinoma. In some embodiments, a subject is someone with one or more features characteristic of susceptibility to or risk of prostate adenocarcinoma. In some embodiments, a subject is a patient. In some embodiments, a subject is an individual to whom diagnosis and/or therapy is and/or has been administered. In some embodiments, a subject is an asymptotic subject. Such an asymptomatic subject may be a subject at average population risk or with hereditary risk. For example, such an asymptomatic subject may be a subject who has a family history of cancer, who has been previously treated for cancer, who is at risk of cancer recurrence after cancer treatment, who is in remission after cancer treatment, and/or who has been previously or periodically screened for the presence of at least one cancer biomarker. Alternatively, in some embodiments, an asymptomatic subject may be a subject who has not been previously screened for cancer, who has not been diagnosed for cancer, and/or who has not previously received cancer therapy. In some embodiments, a subject amenable to provided technologies is an individual selected based on one or more characteristics such as age, race, geographic location, genetic history, medical history, personal history (e.g., smoking, alcohol, drugs, carcinogenic agents, diet, obesity, physical activity, sun exposure, radiation exposure, exposure to infectious agents such as viruses, and/or occupational hazard).

Suffering from: An individual who is “suffering from” a disease, disorder, and/or condition has been diagnosed with and/or displays one or more symptoms of a disease, disorder, and/or condition.

Surface analyte: As used herein, a “surface analyte” refers to an analyte present on the surface of a biological entity (e.g., a cell or a nanoparticle from a biological sample). In some embodiments, a surface analyte is or comprises a surface polypeptide or surface protein. In some embodiments, a surface analyte is or comprises a glycan.

Surface biomarker: As used herein, a “surface biomarker” refers to a marker indicative of the state (e.g., presence, level, and/or activity) of a surface analyte (e.g., as described herein) of a biological entity (e.g., a cell or a nanoparticle including, e.g., in some embodiments an analyte aggregate (e.g., a protein or mucin aggregate) and/or an extracellular vesicle). In some embodiments, a surface biomarker is or comprises a surface protein biomarker. In some embodiments, a surface biomarker is or comprises a carbohydrate-dependent marker.

Surface polypeptide or surface protein: As used interchangeably herein, the terms “surface polypeptide” and “surface protein” refer to a polypeptide or protein present in and/or on the surface of a biological entity (e.g., a cell or a nanoparticle including, e.g., in some embodiments an analyte aggregate (e.g., a protein or mucin aggregate) and/or an extracellular vesicle, etc.) through direct or indirect interactions. As will be understood by a skilled artisan, a surface protein, in some embodiments, may comprise a post-translational modification, including, e.g., but not limited to glycosylation. In some embodiments, a surface polypeptide or protein may be or comprise a membrane-bound polypeptide. In some embodiments, a membrane-bound polypeptide refers to a polypeptide or protein with one or more domains or regions present in and/or on the surface of the membrane of a biological entity (e.g., a cell, an extracellular vesicle, etc.). In some embodiments, a membrane-bound polypeptide may comprise one or more domains or regions spanning and/or associated with the plasma membrane of a biological entity (e.g., a cell, an extracellular vesicle, etc.). In some embodiments, a membrane-bound polypeptide may comprise one or more domains or regions spanning and/or associated with the plasma membrane of a biological entity (e.g., a cell, an extracellular vesicle, etc.) and also protruding into the intracellular and/or intravesicular space. In some embodiments, a membrane-bound polypeptide may comprise one or more domains or regions associated with the plasma membrane of a biological entity (e.g., a cell, an extracellular vesicle, etc.), for example, via one or more non-peptidic linkages (e.g., through a glycosylphosphatidylinositol (GPI) anchor or lipidification or through non-covalent interaction). In some embodiments, a membrane-bound polypeptide may comprise one or more domains or regions that is/are anchored into either side of plasma membrane of a biological entity (e.g., a cell, an extracellular vesicle, etc.). In some embodiments, a surface protein is associated with or present on the surface of a nanoparticle (e.g., as described herein). In some embodiments, a surface protein is associated with or present within an extracellular vesicle. In some embodiments, a surface protein may be associated with or present within a prostate adenocarcinoma-associated extracellular vesicle (e.g., an extracellular vesicle obtained or derived from a bodily fluid-derived sample (e.g., but not limited to a blood-derived sample, a urine-derived sample, etc.) of a subject suffering from or susceptible to prostate adenocarcinoma). As will be understood by a skilled artisan, detection of the presence of at least a portion of a surface polypeptide or surface protein on/within extracellular vesicles can facilitate separation and/or isolation of prostate adenocarcinoma-associated extracellular vesicles from a biological sample (e.g., a blood or blood-derived sample) from a subject. In some embodiments, detection of the presence of a surface polypeptide or surface protein may be or comprise detection of an intravesicular portion (e.g., an intravesicular epitope) of such a surface polypeptide or surface protein. In some embodiments, detection of the presence of a surface polypeptide or surface protein may be or comprise detection of a membrane-spanning portion of such a surface polypeptide or surface protein. In some embodiments, detection of the presence of a surface polypeptide or surface protein may be or comprise detection of an extravesicular portion of such a surface polypeptide or surface protein.

Surface protein biomarker: As used herein, the term “surface protein biomarker” refers to a marker indicative of the state (e.g., presence, level, and/or activity) of a surface protein (e.g., as described herein) of a biological entity (e.g., a cell or a nanoparticle including, e.g., in some embodiments an analyte aggregate (e.g., a protein or mucin aggregate) and/or an extracellular vesicle). In some embodiments, a surface protein refers to a polypeptide or protein with one or more domains or regions located in or on the surface of the membrane of a biological entity (e.g., a cell or an extracellular vesicle). In some embodiments, a surface protein biomarker may be or comprise an epitope that is present on the interior side (intravesicular) or the exterior side (extravesicular) of the membrane. In some embodiments, a surface protein biomarker is associated with or present in an extracellular vesicle. In some embodiments, a surface protein biomarker may be or comprise a mutated polypeptide. In some embodiments, a surface protein biomarker may be post-translationally modified (e.g., but not limited to glycosylated, phosphorylated, etc.). In some embodiments, a surface protein biomarker may be post-translationally processed and present in the form of a truncated polypeptide, for example, as a result of proteolytic cleavage). In some embodiments, a surface-protein biomarker may be or comprise an epitope that is present on the exterior surface of a nanoparticle.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder, and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target-binding moiety: In general, the terms “target-binding moiety” and “binding moiety” are used interchangeably herein to refer to any entity or moiety that binds to a target of interest (e.g., molecular target of interest such as a biomarker or an epitope). In many embodiments, a target-binding moiety of interest is one that binds specifically with its target (e.g., a target biomarker) in that it discriminates its target from other potential binding partners in a particular interaction context. In general, a target-binding moiety may be or comprise an entity or moiety of any chemical class (e.g., polymer, non-polymer, small molecule, polypeptide, carbohydrate, lipid, nucleic acid, etc.). In some embodiments, a target-binding moiety is a single chemical entity. In some embodiments, a target-binding moiety is a complex of two or more discrete chemical entities associated with one another under relevant conditions by non-covalent interactions. For example, those skilled in the art will appreciate that in some embodiments, a target-binding moiety may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple target binding moieties through linkage of different specific binding moieties with a generic binding moiety partner.

Target biomarker signature: The term “target biomarker signature”, as used herein, refers to a combination of (e.g., at least 2 or more, including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, or more) biomarkers, which combination correlates with a particular biological event or state of interest, so that one skilled in the art will appreciate that it may appropriately be considered to be a “signature” of that event or state. To give but a few examples, in some embodiments, a target biomarker signature may correlate with a particular disease or disease state, and/or with likelihood that a particular disease, disorder or condition may develop, occur, or reoccur. In some embodiments, a target biomarker signature may correlate with a particular disease or therapeutic outcome, or likelihood thereof. In some embodiments, a target biomarker signature may correlate with a specific cancer and/or stage thereof. In some embodiments, a target biomarker signature may correlate with prostate adenocarcinoma and/or a stage and/or a subtype thereof. In some embodiments, a target biomarker signature comprises a combination of (e.g., at least 2 or more, including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, or more) biomarkers that together are specific for a prostate adenocarcinoma or a subtype and/or a disease stage thereof), though one or more biomarkers in such a combination may be directed to a target (e.g., a surface biomarker, an intravesicular biomarker, and/or an intravesicular RNA) that is not specific to the prostate adenocarcinoma. For example, in some embodiments, a target biomarker signature may comprise at least one biomarker specific to a prostate adenocarcinoma or a stage and/or subtype thereof (i.e., a prostate adenocarcinoma-specific target), and may further comprise a biomarker that is not necessarily or completely specific for the prostate adenocarcinoma (e.g., that may also be found on some or all biological entities such as, e.g., cells, extracellular vesicles, etc., that are not cancerous, are not of the relevant cancer, and/or are not of the particular stage and/or subtype of interest). That is, as will be appreciated by those skilled in the art reading the present specification, so long as a combination of biomarkers utilized in a target biomarker signature is or comprises a plurality of biomarkers that together are specific for the relevant target biological entities of interest (e.g., prostate adenocarcinoma cells of interest or extracellular vesicles secreted by prostate adenocarcinoma cells) (i.e., sufficiently distinguish the relevant target biological entities (e.g., prostate adenocarcinoma cells of interest or extracellular vesicles secreted by prostate adenocarcinoma cells) for detection from other biological entities not of interest for detection), such a combination of biomarkers is a useful target biomarker signature in accordance with certain embodiments of the present disclosure.

Therapeutic agent: As used interchangeably herein, the phrase “therapeutic agent” or “therapy” refers to an agent or intervention that, when administered to a subject or a patient, has a therapeutic effect and/or elicits a desired biological and/or pharmacological effect. In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Threshold level (e.g., cutoff): As used herein, the term “threshold level” refers to a level that are used as a reference to attain information on and/or classify the results of a measurement, for example, the results of a measurement attained in an assay. For example, in some embodiments, a threshold level (e.g., a cutoff) means a value measured in an assay that defines the dividing line between two subsets of a population (e.g., normal and/or non-prostate adenocarcinoma vs. prostate adenocarcinoma). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for the purpose described herein.

### DETAILED DESCRIPTION OF CERTAIN EMBODIMENTS

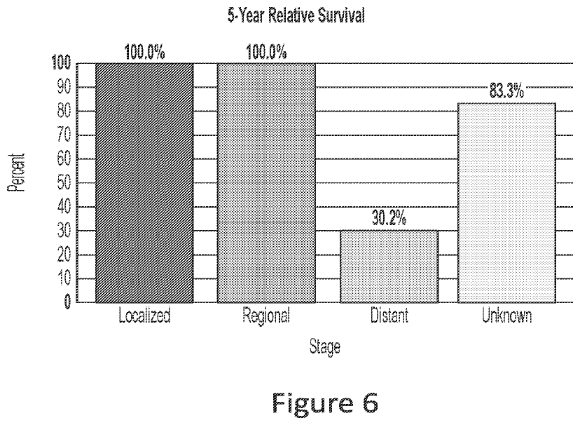

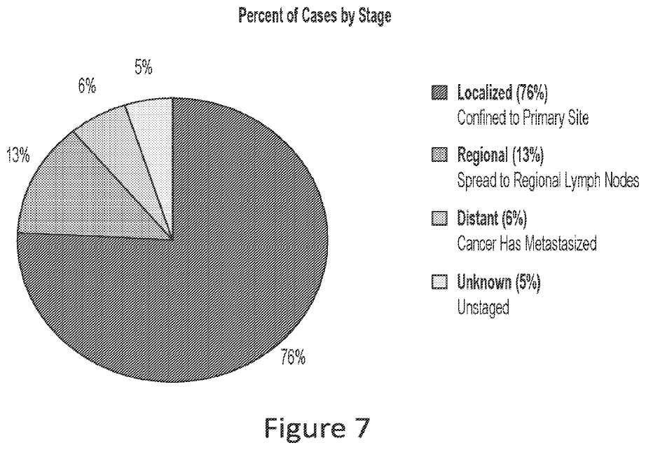

Prostate cancer was responsible for an estimated 191,930 deaths in 2020 in the United States (Estimates of new cases and deaths for 2020 are projections made by the American Cancer Society (ACS), based on earlier reported data; which is incorporated herein by reference for the purpose described herein). The majority of these deaths were attributable to late diagnosis; the 5-year total survival rate for prostate cancer in the United States from 2010 to 2016 was 30.2% in the distant stage. Patients with localized disease at diagnosis had a 5-year survival rate of 100% and 76% of prostate cancer was detected at the localized stage (FIGS. 6 and 7). Additionally, 13% of prostate cancers were detected in the regional stage and the 5-year survival was 100% between 2010 and 2016. This means that any cases detected earlier than in the distant stage will almost guarantee a 5-year survival for that individual. Therefore, it is imperative to use technologies disclosed herein for early-stage cancer detection and, more specifically, prostate cancer detection.

Prostate adenocarcinoma (PRAD) refers to cancer in the prostate, which is a walnut shaped organ responsible for producing seminal fluid in males. Prostate cancer varies greatly in speed to metastasize, making it difficult to predict which men will benefit from aggressive treatment. Symptoms of prostate cancer include incontinence and erectile dysfunction.

A common type of screenings for prostate cancer includes a digital rectal examination (DRE), in which a physician inserts their finger(s) into a patient's rectum to examine the texture, shape, and size of the prostate gland. Conducting a prostate-specific antigen (PSA) quantification test from a blood sample is an additional common type of screening in which increased PSA may indicate prostate infection, enlargement, and/or cancer. Tests for prostate-cancer-specific markers in the urine (e.g., PCA3) have also been used to help diagnose patients for prostate cancer

Additionally, common post initial screening technologies and diagnostic methods may include ultrasound, magnetic resonance imaging (MRI), and/or biopsy. The present technologies include higher accuracy and precision (e.g. higher signal to noise ratio) in the initial screening compared to common/existing screening methods, which may allow for reduced use of costly imaging systems by reducing the number of false positives.

The present disclosure, among other things, identifies the source of a problem with certain prior technologies including, for example, certain conventional approaches to detection and diagnosis of prostate cancer. For example, the present disclosure appreciates that many conventional diagnostic assays, e.g., digital rectal examination (DRE), urine test, and/or CT scanning, can be time-consuming, costly, and/or lacking sensitivity and/or specificity sufficient to provide a reliable and comprehensive diagnostic assessment. In some embodiments, the present disclosure provides technologies (including systems, compositions, and methods) that solve such problems, among other things, by identification of biomarker combinations that are predicted to exhibit high sensitivity and specificity for prostate cancer based on bioinformatics analysis. In some embodiments, the present disclosure provides technologies (including systems, compositions, and methods) that solve such problems, by detecting co-localization of a target biomarker signature of prostate cancer (e.g., identified by bioinformatics analysis) in individual extracellular vesicles, which comprises at least one extracellular vesicle-associated surface biomarker and at least one target biomarker selected from the group consisting of surface biomarkers, internal protein biomarkers, and RNA biomarkers present in extracellular vesicles associated with prostate cancer. In some embodiments, the present disclosure provides technologies (including systems, compositions, and methods) that solve such problems, among other things, by detecting such target biomarker signature of prostate cancer using a target entity detection approach that was developed by Applicant and described in U.S. application Ser. No. 16/805,637 (published as US2020/0299780; issued as U.S. Pat. No. 11,085,089), and International Application PCT/US2020/020529 (published as WO2020180741), both filed Feb. 28, 2020 and entitled “Systems, Compositions, and Methods for Target Entity Detection,” which are based on interaction and/or co-localization of a target biomarker signature in individual extracellular vesicles. The contents of each of the aforementioned disclosures is incorporated herein by reference in their entirety.

In some embodiments, extracellular vesicles for detection as described herein can be isolated from a bodily fluid of a subject by a size exclusion-based method. As will be understood by a skilled artisan, in some embodiments, a size exclusion-based method may provide a sample comprising nanoparticles having a size range of interest that includes extracellular vesicles. Accordingly, in some embodiments, provided technologies of the present disclosure encompass detection, in individual nanoparticles having a size range of interest (e.g., in some embodiments about 30 nm to about 1000 nm) that includes extracellular vesicles, of co-localization of at least two or more surface biomarkers (e.g., as described herein) that forms a target biomarker signature of prostate cancer. A skilled artisan reading the present disclosure will understand that various embodiments described herein in the context of “extracellular vesicle(s)” (e.g., assays for detecting individual extracellular vesicles and/or provided “extracellular vesicle-associated surface biomarkers”) can be also applicable in the context of “nanoparticles” as described herein.

The present disclosure, among other things, provides insights and technologies for achieving effective prostate cancer screening, e.g., for early detection of prostate cancer, e.g., including but not limited to prostate adenocarcinoma. In some embodiments, the present disclosure provides technologies for early detection of prostate cancer in subjects who may be experiencing one more symptoms associated with prostate cancer. In some embodiments, the present disclosure provides technologies for early detection of prostate cancer in subjects who are at hereditary risks for prostate cancer. In some embodiments, the present disclosure provides technologies for early detection of prostate cancer in subjects who may be at hereditary risk and/or experiencing one or more symptoms associated with prostate cancer. In some embodiments, the present disclosure provides technologies for early detection of prostate cancer in subjects who may have life-history risk factors. In some embodiments, the present disclosure provides technologies for screening individuals, e.g., individuals with certain risks (e.g., hereditary risk, life history associated risk, or average risk) for early-stage prostate cancer (e.g., prostate adenocarcinoma). In some embodiments, provided technologies are effective for detection of early-stage prostate cancer (e.g., prostate adenocarcinomas). In some embodiments, provided technologies are effective when applied to populations comprising or consisting of individuals having one or more symptoms that may be associated with prostate cancer. In some embodiments, provided technologies are effective even when applied to populations comprising or consisting of asymptomatic or symptomatic individuals (e.g., due to sufficiently high sensitivity and/or low rates of false positive and/or false negative results). In some embodiments, provided technologies are effective when applied to populations comprising or consisting of individuals (e.g., asymptomatic or symptomatic individuals) without hereditary risk, and/or life-history related risk of developing prostate cancer. In some embodiments, provided technologies are effective when applied to populations comprising or consisting of individuals (e.g., asymptomatic or symptomatic individuals) with hereditary risk for developing prostate cancer. In some embodiments, provided technologies are effective when applied to populations comprising or consisting of individuals susceptible to prostate cancer (e.g., individuals with a known genetic, environmental, or experiential risk, etc.). In some embodiments, provided technologies may be or include one or more compositions (e.g., molecular complexes, systems, collections, combinations, kits, etc.) and/or methods (e.g., of making, using, assessing, etc.), as will be clear to one skilled in the art reading the disclosure provided herein.

In some embodiments, provided technologies achieve detection (e.g., early detection, e.g., in asymptomatic individual(s) and/or population(s)) of one or more features (e.g., incidence, progression, responsiveness to therapy, recurrence, etc.) of prostate cancer, with sensitivity and/or specificity (e.g., rate of false positive and/or false negative results) appropriate to permit useful application of provided technologies to single-time and/or regular (e.g., periodic) assessment. In some embodiments, provided technologies are useful in conjunction with an individual's regular medical examinations, such as but not limited to: physicals, general practitioner visits, cholesterol/lipid blood tests, urine tests, diabetes (type 2) screening, colonoscopies, blood pressure screening, thyroid function tests, prostate cancer screening, and/or vaccinations. In some embodiments, provided technologies are useful in conjunction with treatment regimen(s); in some embodiments, provided technologies may improve one or more characteristics (e.g., rate of success according to an accepted parameter) of such treatment regimen(s).

In some embodiments, the present disclosure, among other things, provides insights that screening of asymptotic individuals, e.g., regular screening prior to or otherwise in absence of developed symptom(s), can be beneficial, and even important for effective management (e.g., successful treatment) of prostate cancer. In some embodiments, the present disclosure provides prostate cancer screening systems that can be implemented to detect prostate cancer, including early-stage cancer, in some embodiments in asymptomatic individuals (e.g., without hereditary, and/or life-history associated risks in prostate cancer). In some embodiments, provided technologies are implemented to achieve regular screening of asymptomatic individuals (e.g., with or without hereditary risk(s) in prostate cancer). In some embodiments, provided technologies are implemented to achieve regular screening of symptomatic individuals (e.g., with or without hereditary and/or life-history associated risk(s) in prostate cancer). The present disclosure provides, for example, compositions (e.g., reagents, kits, components, etc.), and methods of providing and/or using them, including strategies that involve regular testing of one or more individuals (e.g., asymptomatic individuals). The present disclosure defines usefulness of such systems, and provides compositions and methods for implementing them.

### 1. Prostate Cancer Detection

Prostate cancer represents 10.6% of all new cancer cases in the U.S. per year, and was ranked third in new case counts in 2020. In the USA, the annual rate of new cases for prostate cancer was 109.8 per 100,000 for men between 2013 and 2017. The death rate was 19 per 100,000 men between 2014 and 2018. The lifetime risk for men developing prostate cancer at any point in their lives was 12.1% between 2015 and 2017. Fortunately, the death rate has decreased by approximately 30% over the past ˜30 years. However, prostate cancer still accounts for 7.0% of all cancer-related deaths. In 2017, there were an estimated 3,170,339 men living with prostate cancer in the United States (Data taken from seer.cancer.gov, which is incorporated herein by reference in its entirety).

The Surveillance, Epidemiology and End Results (SEER) data from 2000-2017 has reported extensively on the prevalence and epidemiology of prostate cancer in the United States of America. SEER reported that in 2017, there were 615.3 cases of prostate cancer per 100,000 men aged 65+ in the USA. Additionally, there were 252.5 cases per 100,000 men aged 50-64, and 3.7 cases per 100,000 men aged <50 (SEER*Explorer: An interactive website for SEER cancer statistics. Surveillance Research Program, National Cancer Institute. [Cited 2020 Sep. 14]. Available from https://seer.cancer.gov/explorer/, which is incorporated herein by reference in their entirety). Technologies disclosed herein are designed to address the current short-comings in prostate cancer screening technologies.

According to the CDC and the American Cancer Society, risk factors for prostate cancer (e.g. prostate adenocarcinoma) include age (e.g., increased risk after the age of 50 years), race (e.g., prostate cancer develops more often in men of African-American ancestry, and is less common in men of Asian or Hispanic ancestry), geographic location (e.g., prostate cancer is more common in North America, Europe and Australia, and is less common in Asia, Africa, and Central/South America), diet (e.g., consumption of large amounts of red meat and high-fat foods increases risk), obesity, and family history.

In general, consuming tobacco and tobacco smoke increases rates of all cancer types. The International Agency for Research on Cancer (IARC) has identified at least 50 known carcinogens in tobacco smoke. Examples of such carcinogens include, but are not limited to, tobacco-specific N-nitrosamines (TSNAs) formed by nitrosation of nicotine during tobacco processing and during smoking. The chemical 4-(methylnitrosamino)-1 (3-pyridyl)-1-butanone (NNK) is known to induce cancer experimental animals. NNK is known to bind to DNA and create DNA adducts, leading to DNA damage. Failure to repair this damage can lead to permanent mutations. NNK is associated with DNA mutations resulting in the activation of K-Ras oncogenes, which is detected in humans.

In some embodiments, the present disclosure provides technologies for effective screening of prostate cancer in individuals at hereditary risk, or in individuals with life-history associated-risks. In some embodiments, the present disclosure provides technologies for effective screening of prostate cancer in average-risk individuals. In some embodiments, the present disclosure provides technologies for effective screening of prostate cancer in individuals with one or more symptoms associated with prostate cancer. In some embodiments, the present disclosure provides technologies for effective screening of prostate cancer in asymptomatic individuals. Despite being relatively common in men, there is currently no recommended prostate cancer screening tool that is non-invasive based on a subject's blood sample and intended for screening asymptomatic and/or average-risk individuals (e.g., individuals under the age of 50 years, or individuals over the age of 50 years. This is due, in part, to the cost, limited availability, potential side effects, and/or poor performance (e.g., high false positive rate, or ineffectualness) of existing prostate cancer and prostate cancer screening technologies. Given the incidence of prostate cancer in average-risk individuals, inadequate test specificities (<99.5%) can result in false positive results that outnumber true positives by more than an order of magnitude. This places a significant burden on the healthcare system and on the individuals being screened as false positive results lead to additional tests, unnecessary surgeries, and emotional/physical distress (Wu et al., 2016). In some embodiments, the present disclosure provides an insight that a particularly useful prostate cancer screening test would be characterized by: (1) ultrahigh specificity (>99%) to minimize the number of false positives, and (2) high sensitivity (>40%) for stage I and II with the ability to differentiate between indolent (e.g., Gleason score <7) and aggressive (e.g., Gleason score >=7) prostate cancer (i.e., when prognosis is most favorable and treatment is necessary).

In some embodiments, the present disclosure provides an insight that a particularly useful prostate cancer screening test may be characterized by: (1) ultrahigh specificity (>98%) to minimize the number of false positives, and (2) high sensitivity (>40%) for stage I and II (Gleason score <7) prostate cancer (i.e., when prognosis is most favorable). For example, in some embodiments, a particularly useful prostate cancer screening test may be characterized by a specificity of >98% and a sensitivity of >50%, for example, for stage I and II (Gleason score <7) prostate cancer. In some embodiments, a particularly useful prostate cancer screening test may be characterized by a specificity of >98% and a sensitivity of >60%, for example, for stage I and II (Gleason score <7) prostate cancer. In some embodiments, a particularly useful prostate cancer screening test may be characterized by a specificity of >98% and a sensitivity of >70%, for example, for stage I and II (Gleason score <7) prostate cancer. In some embodiments, a particularly useful prostate cancer screening test may be characterized by a specificity of >99.5% and a sensitivity of >65%, for example, for stage I and II (Gleason score <7) prostate cancer. In some embodiments, a particularly useful prostate cancer screening test may be characterized by a specificity of >99.5% and a sensitivity of >60%, for example, for stage I and II (Gleason score <7) prostate cancer. In some embodiments, a particularly useful prostate cancer screening test may be characterized by a specificity of 99% or higher and a sensitivity of >10% or higher (including, e.g., >15%, >20%, >25%). In some embodiments, a particularly useful prostate cancer screening test may be characterized by a specificity of 99% or higher and a sensitivity of 50% or higher. In some embodiments, a particularly useful prostate cancer screening test may be characterized by a specificity of 90% or higher and a sensitivity of 50% or higher.

In some embodiments, the present disclosure provides an insight that a prostate cancer screening test involving more than one set of biomarker combinations (e.g., at least two orthogonal biomarker combinations as described herein) can increase specificity and/or sensitivity of such an assay, as compared to that is achieved by one set of biomarker combination. For example, in some embodiments, a prostate cancer screening test involving at least two orthogonal biomarker combinations can achieve a specificity of at least 98% and a sensitivity of at least 50%. In some embodiments, a prostate cancer screening test involving at least two orthogonal biomarker combinations can achieve a specificity of at least 98% and a sensitivity of at least 60%. In some embodiments, a prostate cancer screening test involving at least two orthogonal biomarker combinations can achieve a specificity of 99% and a sensitivity of 50% or higher.

In some embodiments, the present disclosure provides an insight that a particularly useful prostate cancer screening test may be characterized by an acceptable positive predictive value (PPV) at an economically justifiable cost. PPV is the likelihood a patient has the disease following a positive test, and is influenced by sensitivity, specificity, and/or disease prevalence. In some embodiments, assays described herein can be useful for early prostate cancer detection that achieves a PPV of greater than 10% or higher, including, e.g., greater than 15%, greater than 20%, or greater than 25% or higher, with a specificity cutoff of at least 70% or higher, including, e.g., at least 75%, at least 80%, at least 85%, or higher. In some embodiments, assays described herein are particularly useful for early prostate cancer detection that achieves a PPV of greater than 10% or higher, including, e.g., greater than 15%, greater than 20%, or greater than 25% or higher, with a specificity cutoff of at least 85% or higher, including, e.g., at least 90%, at least 95%, or higher (e.g., a specificity cutoff of at least 98% for subjects at hereditary risk for prostate cancer, or a specificity cutoff of at least 99.5% for subjects experiencing one or more symptoms associated with prostate cancer).

In some embodiments, assays described herein can be useful for early prostate cancer detection that achieves a PPV of greater than 30% or higher, including, e.g., greater than 35%, greater than 40%, or greater than 45% or higher, with a specificity cutoff of at least 70% or higher, including, e.g., at least 75%, at least 80%, at least 85%, or higher. In some embodiments, assays described herein are particularly useful for early prostate cancer detection that achieves a PPV of greater than 30% or higher, including, e.g., greater than 35%, greater than 40%, or greater than 45% or higher, with a specificity cutoff of at least 85% or higher, including, e.g., at least 90%, at least 95%, or higher (e.g., a specificity cutoff of at least 98% for subjects at hereditary risk for prostate cancer, or a specificity cutoff of at least 99.5% for subjects experiencing one or more symptoms associated with prostate cancer).

In some embodiments, assays described herein are particularly useful as a first screening test for early prostate cancer detection. In some embodiments, subjects who have received a positive test result from assays described herein are recommended to receive a follow-up test, e.g., digital rectal examination (DRE). In some such embodiments, assays described herein can be useful for early prostate cancer detection that achieves a PPV of greater than 20%, or greater than 25%, or greater than 30%, or greater than 35%, or greater than 40% or higher. In some embodiments, assays described herein can achieve a specificity cutoff of at least 70% or higher, including, e.g., at least 75%, at least 80%, at least 85%, or higher. In some such embodiments, assays described herein can achieve a specificity cutoff of at least 85% or higher, including, e.g., at least 90%, at least 95% or higher (e.g., a specificity cutoff of at least 98% for subjects at hereditary risk for prostate cancer, or with a specificity cutoff of at least 99.5% for subjects experiencing one or more symptoms associated with prostate cancer). In some embodiments, assays described herein can achieve a sensitivity cutoff of at least 75% or higher, including, e.g., at least 80%, at least 85%, at least 90%, or higher.

Several different biomarker classes have been studied for a prostate cancer liquid biopsy assay including circulating tumor DNA (ctDNA), circulating tumor cells (CTCs), bulk proteins, and extracellular vesicles (EVs). EVs are particularly promising due to their abundance and stability in the bloodstream relative to ctDNA and CTCs, suggesting improved sensitivity for early-stage cancers. Moreover, EVs contain cargo (i.e., proteins, RNA, metabolites) that originated from the same cell, providing superior specificity over bulk protein measurements. While the diagnostic utility EVs has been studied, much of this work has pertained to bulk EV measurements or low-throughput single-EV analyses.

II. Provided Biomarkers and/or Target Biomarker Signatures for Detection of Prostate Cancer

The present disclosure, among other things, provides various target biomarkers or combinations thereof (e.g., target biomarker signatures) for prostate cancer. Such target biomarker signatures that are predicted to exhibit high sensitivity and specificity for prostate cancer were discovered by a multi-pronged bioinformatics analysis and biological approach, which for example, in some embodiments involve computational analysis of a diverse set of data, e.g., in some embodiments comprising one or more of sequencing data, expression data, mass spectrometry, histology, post-translational modification data, and/or in vitro and/or in vivo experimental data through machine learning and/or computational modeling.

In some embodiments, a target biomarker signature of prostate cancer comprises at least one or more (e.g., 1, 2, 3, 4, 5, 6, 7, 8, or more) surface biomarker (e.g., in some embodiments surface polypeptide present in extracellular vesicles associated with prostate cancer; “extracellular vesicle-associated surface biomarker”) and at least one or more (e.g., 1, 2, 3, 4, 5, 6, 7, 8, or more) target biomarkers selected from the group consisting of surface biomarker(s), intravesicular biomarker(s), and intravesicular RNA biomarker(s), such that the combination of such surface biomarker(s) and such target biomarker(s) present a target biomarker signature of prostate cancer that provides (a) high specificity (e.g., greater than 98% or higher such as greater than 99%, or greater than 99.5%) to minimize the number of false positives, and (b) high sensitivity (e.g., greater than 40%, greater than 50%, greater than 60%, greater than 70%, greater than 80%) for stage I and II prostate cancer when prognosis is most favorable.

In some embodiments, the present disclosure recognizes that in certain embodiments, sensitivity and specificity rates for subjects with different prostate cancer risk levels may vary depending upon the risk tolerance of the attending physician and/or the guidelines set forth by interested medical consortia. In some embodiments, lower specificity and/or sensitivity may be used for screening patients at higher risk of prostate cancer (e.g., patients with life-history-associated risk factors, symptomatic patients, patients with a family history of prostate cancer, or patients with abnormal serum PSA, etc.) as compared to that for patients with lower risk for prostate cancer. For example, in some embodiments, biomarker combinations described herein that are useful for detection of prostate cancer may provide a specificity of at least 70% including, e.g., at least 75%, at least 80%, at least 85%, at least 90%, at least 95%, at least 98%, at least 99.5%, or higher. Additionally or alternatively, in some embodiments, biomarker combinations described herein that are useful for detection of prostate cancer may provide a sensitivity of at least 50% including, e.g., at least 55%, at least 60%, at least 65%, at least 70%, at least 75%, at least 80%, at least 85%, at least 90%, at least 95%, at least 98%, at least 99.5%, or higher.

In certain embodiments, subjects at risk of prostate cancer may be served with an 85% specificity rate or higher (including, e.g., at least 90%, at least 95% or higher specificity rate) with 50% sensitivity or higher (including, e.g., at least 60%, at least 70%, at least 80%, or higher sensitivity). In certain embodiments, at risk subjects with life-history-associated risk factors may be served with an 85% specificity rate or higher (including, e.g., at least 90%, at least 95% or higher specificity rate) with 50% sensitivity or higher (including, e.g., at least 60%, at least 70%, at least 80%, or higher sensitivity). In certain embodiments, symptomatic subjects may be served with an 85% specificity rate or higher (including, e.g., at least 90%, at least 95% or higher specificity rate) with 50% sensitivity or higher (including, e.g., at least 60%, at least 70%, at least 80%, or higher sensitivity). In certain embodiments, non-symptomatic subjects may be served with an 85% specificity rate or higher (including, e.g., at least 90%, at least 95% or higher specificity rate) with 50% sensitivity or higher (including, e.g., at least 60%, at least 70%, at least 80%, or higher sensitivity). In certain embodiments, subjects at risk of prostate cancer may be served with a 99.5% specificity rate with 70% sensitivity or a 98% specificity rate with 80% sensitivity. In certain embodiments, at risk subjects with life-history-associated risk factors may be served with a 99.5% specificity rate with 70% sensitivity or a 98% specificity rate with 80% sensitivity. In some embodiments, an assay described herein for detection of prostate cancer in at-risk subjects (e.g., with life-history-associated risk factors) may have a set sensitivity rate that is lower than 80% sensitivity, including e.g., less than 70%, less than 60%, less than 50% or lower sensitivity rate. In certain embodiments, non-symptomatic subjects may be served with a 99.5% specificity rate with 70% sensitivity or a 98% specificity rate with 80% sensitivity. In some embodiments, an assay described herein for detection of prostate cancer in non-symptomatic subjects may have a set sensitivity rate that is lower than 80% sensitivity, including e.g., less than 70%, less than 60%, less than 50% or lower sensitivity rate. In some embodiments, technologies and/or assays described herein for detection of prostate cancer in a symptomatic subject may have a lower sensitivity and/or specificity requirement than those for detection of prostate cancer in an asymptomatic subject. In some embodiments, an assay described herein for detection of prostate cancer in a symptomatic subject may have a set specificity rate that is lower than 99.5% specificity, including e.g., less than 99% sensitivity, less than 95%, less than 90%, or less than 85% specificity rate. In some embodiments, an assay described herein for detection of prostate cancer in a symptomatic subject may have a set sensitivity rate that is lower than 80% sensitivity, including e.g., less than 70%, or less than 60% sensitivity rate.

In some embodiments, the present disclosure, among other things, appreciates that a biomarker signature of prostate cancer that provides a positive predictive value (PPV) of 30% or higher is useful for screening individuals at risk for prostate cancer. In some embodiments, a target biomarker signature of prostate cancer comprises at least one surface biomarker (e.g., surface biomarker present on the surfaces of extracellular vesicles associated with prostate cancer) and at least one target biomarker selected from the group consisting of surface biomarker(s), intravesicular biomarker(s), and intravesicular RNA biomarker(s), such that the combination of such surface biomarker(s) and such target biomarker(s) present a target biomarker signature of prostate cancer that provides a positive predictive value (PPV) of at least 25% or higher, including, e.g., at least 30% or higher, at least 35% or higher, at least 40% or higher, and/or at least 45% or higher, in high-risk population.

In general, gene identifiers used herein refer to the Gene Identification catalogued by the UniProt Consortium (UniProt.org); one skilled in the art will understand that certain genes can be known by multiple names and will also readily recognize such multiple names.

In general, carbohydrate identifiers used herein refer to Kegg Cancer-associated Carbohydrates database (genome.jp/kegg/disease/br08441.html); one skilled in the art will understand that certain carbohydrates can be known by multiple names and will also readily recognize such multiple names.

In some embodiments, a target biomarker included in a target biomarker signature of prostate cancer is or comprises a surface biomarker selected from the group consisting of: ATP-binding cassette sub-family C member 4 (ABCC4) polypeptide, Alpha/beta hydrolase domain-containing protein 17C (ABHD17C) polypeptide, 1,2-dihydroxy-3-keto-5-methylthiopentene dioxygenase (AD11) polypeptide, Type-1 angiotensin II receptor-associated protein (AGTRAP) polypeptide, AP-1 complex subunit mu-2 (AP1M2) polypeptide, MICOS complex subunit MIC26 (APOO) polypeptide, Brefeldin A-inhibited guanine nucleotide-exchange protein 3 (ARFGEF3) polypeptide, Calcium-transporting ATPase type 2C member 1 (ATP2C1) polypeptide, Basal cell adhesion molecule (BCAM) polypeptide, Cell adhesion molecule 4 (CADM4) polypeptide, Soluble calcium-activated nucleotidase 1 (CANT1) polypeptide, Cadherin-1 (CDH1) polypeptide, Charged multivesicular body protein 4c (CHMP4C) polypeptide, Claudin-3 (CLDN3) polypeptide, Claudin-4 (CLDN4) polypeptide, Calmegin (CLGN) polypeptide, Ceroid-lipofuscinosis neuronal protein 5 (CLN5) polypeptide, Transmembrane ascorbate-dependent reductase CYB561 (CYB561) polypeptide, DnaJ homolog subfamily C member 30, mitochondrial (DNAJC30) polypeptide, Ectonucleotide pyrophosphatase/phosphodiesterase family member 5 (ENPP5) polypeptide, Epithelial cell adhesion molecule (EPCAM) polypeptide, Endoplasmic reticulum-Golgi intermediate compartment protein 1 (ERGIC1) polypeptide, Fatty-acid amide hydrolase 1 (FAAH) polypeptide, Glutamate carboxypeptidase 2 (FOLH1) polypeptide, Polypeptide N-acetylgalactosaminyltransferase 3 (GALNT3) polypeptide, Guanine nucleotide-binding protein G (I)/G(S)/G (O) subunit gamma-4 (GNG4) polypeptide, Glucosamine 6-phosphate N-acetyltransferase (GNPNAT1) polypeptide, Golgi membrane protein 1 (GOLM1) polypeptide, Grainyhead-like protein 2 homolog (GRHL2) polypeptide, Protein HID1 (HID1) polypeptide, Homer protein homolog 2 (HOMER2) polypeptide, Serine protease hepsin (HPN) polypeptide, Plastin-2 (LCP1) polypeptide, Leucine-rich repeats and immunoglobulin-like domains protein 1 (LRIG1) polypeptide, Ensconsin (MAP7) polypeptide, MARCKS-related protein (MARCKSL1) polypeptide, MARVEL domain-containing protein 2 (MARVELD2) polypeptide, Lysophospholipid acyltransferase 2 (MBOAT2) polypeptide, Transport and Golgi organization protein 1 homolog (MIA3) polypeptide, Mucin-1 (MUC1) polypeptide, N-acylethanolamine-hydrolyzing acid amidase (NAAA) polypeptide, NADH dehydrogenase [ubiquinone] 1 alpha subcomplex subunit 2 (NDUFA2) polypeptide, Protein TMEPAI (PMEPA1) polypeptide, Podocalyxin-like protein 2 (PODXL2) polypeptide, Serine/threonine-protein phosphatase 2B catalytic subunit alpha isoform (PPP3CA) polypeptide, Prostasin (PRSS8) polypeptide, Ras-related protein Rab-3B (RAB3B) polypeptide, Ras-related protein Rab-3D (RAB3D) polypeptide, Rap1 GTPasc-activating protein 1 (RAP1GAP) polypeptide, Retinol dehydrogenase 11 (RDH11) polypeptide, Lysosome membrane protein 2 (SCARB2) polypeptide, Serine incorporator 5 (SERINC5) polypeptide, Sideroflexin-2 (SFXN2) polypeptide, Protein Shroom2 (SHROOM2) polypeptide, Protein Shroom3 (SHROOM3) polypeptide, Solute carrier family 35 member F2 (SLC35F2) polypeptide, Zinc transporter ZIP6 (SLC39A6) polypeptide, Zinc transporter SLC39A7 (SLC39A7) polypeptide, Electrogenic sodium bicarbonate cotransporter 1 (SLC4A4) polypeptide, Acid sphingomyelinase-like phosphodiesterase 3b (SMPDL3B) polypeptide, Sorbitol dehydrogenase (SORD) polypeptide, Metalloreductase STEAP1 (STEAP1) polypeptide, Metalloreductase STEAP2 (STEAP2) polypeptide, Synaptogyrin-2 (SYNGR2) polypeptide, Synaptotagmin-7 (SYT7) polypeptide, Transmembrane channel-like protein 5 (TMC5) polypeptide, Transmembrane emp24 domain-containing protein 3 (TMED3) polypeptide, Transmembrane protein 141 (TMEM141) polypeptide, Transmembrane protein 192 (TMEM192) polypeptide, Proton-transporting V-type ATPase complex assembly regulator TMEM9 (TMEM9) polypeptide, Transmembrane protease serine 2 (TMPRSS2) polypeptide, Transient receptor potential cation channel subfamily M member 4 (TRPM4) polypeptide, Tetraspanin-1 (TSPAN1) polypeptide, Protein unc-13 homolog B (UNC13B) polypeptide, von Willebrand factor A domain-containing protein 1 (VWA1) polypeptide, Protein YIPF1 (YIPF1) polypeptide, Disintegrin and metalloproteinase domain-containing protein 17 (ADAM17) polypeptide, C—C motif chemokine 2 (CCL2) polypeptide, Programmed cell death 1 ligand 1 (CD274) polypeptide, ADP-ribosyl cyclase/cyclic ADP-ribose hydrolase 1 (CD38) polypeptide, C-type lectin domain family 2 member D (CLEC2D) polypeptide, Receptor tyrosine-protein kinase erbB-2 (ERBB2) polypeptide, Filamin-A (FLNA) polypeptide, Filamin-B (FLNB) polypeptide, Glypican-1 (GPC1) polypeptide, Interleukin-6 (IL6) polypeptide, Integrin alpha-V (ITGAV) polypeptide, Prostate-specific antigen (KLK3) polypeptide, Plasma kallikrein (KLKB1) polypeptide, Placenta-specific protein 1 (PLAC1) polypeptide, Protein phosphatase 1 regulatory subunit 3A (PPP1R3A) polypeptide, Prostate stem cell antigen (PSCA) polypeptide, Poliovirus receptor (PVR) polypeptide, Choline transporter-like protein 4 (SLC44A4) polypeptide, TGF-beta receptor type-2 (TGFBR2) polypeptide, Tumor necrosis factor receptor superfamily member 4 (TNFRSF4) polypeptide, Tumor necrosis factor ligand superfamily member 11 (TNFSF11) polypeptide, Vascular endothelial growth factor C (VEGFC) polypeptide, Zinc-alpha-2-glycoprotein (AZGP1) polypeptide, Beta-galactosidase-1-like protein 2 (GLB1L2) polypeptide, Polyadenylate-binding protein 1-like 2 (PABC1L2A) polypeptide, Testican-1 (SPOCK1) polypeptide, Protein-glutamine gamma-glutamyltransferase 4 (TGM4) polypeptide, (Tn) antigen, (SialylTn (sTn)) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof.

In some embodiments, a target biomarker included in a target biomarker signature of prostate cancer is or comprises a surface biomarker selected from the group consisting of: ATP-binding cassette sub-family C member 4 (ABCC4) polypeptide, AP-1 complex subunit mu-2 (AP1M2) polypeptide, Brefeldin A-inhibited guanine nucleotide-exchange protein 3 (ARFGEF3) polypeptide, Soluble calcium-activated nucleotidase 1 (CANT1) polypeptide, ADP-ribosyl cyclase/cyclic ADP-ribose hydrolase 1 (CD38) polypeptide, Cadherin-1 (CDH1) polypeptide, Claudin-3 (CLDN3) polypeptide, Claudin-4 (CLDN4) polypeptide, Calmegin (CLGN) polypeptide, Ectonucleotide pyrophosphatase/phosphodiesterase family member 5 (ENPP5) polypeptide, Glutamate carboxypeptidase 2 (FOLH1) polypeptide, Golgi membrane protein 1 (GOLM1) polypeptide, Grainyhead-like protein 2 homolog (GRHL2) polypeptide, Ensconsin (MAP7) polypeptide, MARCKS-related protein (MARCKSL1) polypeptide, Mucin-1 (MUC1) polypeptide, Protein TMEPAI (PMEPA1) polypeptide, Podocalyxin-like protein 2 (PODXL2) polypeptide, Serine/threonine-protein phosphatase 2B catalytic subunit alpha isoform (PPP3CA) polypeptide, Prostate stem cell antigen (PSCA) polypeptide, Ras-related protein Rab-3B (RAB3B) polypeptide, Ras-related protein Rab-3D (RAB3D) polypeptide, Retinol dehydrogenase 11 (RDH11) polypeptide, Zinc transporter ZIP6 (SLC39A6) polypeptide, Electrogenic sodium bicarbonate cotransporter 1 (SLC4A4) polypeptide, Acid sphingomyelinase-like phosphodiesterase 3b (SMPDL3B) polypeptide, Sorbitol dehydrogenase (SORD) polypeptide, Metalloreductase STEAP1 (STEAP1) polypeptide, Metalloreductase STEAP2 (STEAP2) polypeptide, Synaptotagmin-7 (SYT7) polypeptide, Transmembrane protease serine 2 (TMPRSS2) polypeptide, Transient receptor potential cation channel subfamily M member 4 (TRPM4) polypeptide, Tetraspanin-1 (TSPAN1) polypeptide, Protein unc-13 homolog B (UNC13B) polypeptide, Lewis Y antigen, SialylTn (sTn)) antigen, Sialyl Lewis X (sLex) antigen (also known as Sialyl SSEA-1 (SLX)), T antigen, Tn antigen, and combinations thereof.

In some embodiments, a target biomarker signature comprises one or more extracellular vesicle-associated surface biomarkers and/or one or more surface biomarkers, each independently selected from a list consisting of an ABCC4 polypeptide, an ABHD17C polypeptide, an AD11 polypeptide, an AGTRAP polypeptide, an AP1M2 polypeptide, an APOO polypeptide, an ARFGEF3 polypeptide, an ATP2C1 polypeptide, a BCAM polypeptide, a CADM4 polypeptide, a CANT1 polypeptide, a CDH1 polypeptide, a CHMP4C polypeptide, a CLDN3 polypeptide, a CLDN4 polypeptide, a CLGN polypeptide, a CLN5 polypeptide, a CYB561 polypeptide, a DNAJC30 polypeptide, an ENPP5 polypeptide, an EPCAM polypeptide, an ERGIC1 polypeptide, a FAAH polypeptide, a FOLH1 polypeptide, a GALNT3 polypeptide, a GNG4 polypeptide, a GNPNAT1 polypeptide, a GOLM1 polypeptide, a GRHL2 polypeptide, a HID1 polypeptide, a HOMER2 polypeptide, a HPN polypeptide, a LCP1 polypeptide, a LRIG1 polypeptide, a MAP7 polypeptide, a MARCKSL1 polypeptide, a MARVELD2 polypeptide, a MBOAT2 polypeptide, a MIA3 polypeptide, a MUC1 polypeptide, a NAAA polypeptide, a NDUFA2 polypeptide, a PMEPA1 polypeptide, a PODXL2 polypeptide, a PPP3CA polypeptide, a PRSS8 polypeptide, a RAB3B polypeptide, a RAB3D polypeptide, a RAP1GAP polypeptide, a RDH11 polypeptide, a SCARB2 polypeptide, a SERINC5 polypeptide, a SFXN2 polypeptide, a SHROOM2 polypeptide, a SHROOM3 polypeptide, a SLC35F2 polypeptide, a SLC39A6 polypeptide, a SLC39A7 polypeptide, a SLC4A4 polypeptide, a SMPDL3B polypeptide, a SORD polypeptide, a STEAP1 polypeptide, a STEAP2 polypeptide, a SYNGR2 polypeptide, a SYT7 polypeptide, a TMC5 polypeptide, a TMED3 polypeptide, a TMEM141 polypeptide, a TMEM192 polypeptide, a TMEM9 polypeptide, a TMPRSS2 polypeptide, a TRPM4 polypeptide, a TSPAN1 polypeptide, an UNC13B polypeptide, a VWA1 polypeptide, a YIPF1 polypeptide, an ADAM17 polypeptide, a CCL2 polypeptide, a CD274 polypeptide, a CD38 polypeptide, a CLEC2D polypeptide, an ERBB2 polypeptide, a FLNA polypeptide, a FLNB polypeptide, a GPC1 polypeptide, an IL6 polypeptide, a ITGAV polypeptide, a KLK3 polypeptide, a KLKB1 polypeptide, a PLAC1 polypeptide, a PPP1R3A polypeptide, a PSCA polypeptide, a PVR polypeptide, a SLC44A4 polypeptide, a TGFBR2 polypeptide, a TNFRSF4 polypeptide, a TNFSF11 polypeptide, a VEGFC polypeptide, an AZGP1 polypeptide, a GLB1L2 polypeptide, a PABPC1L2A polypeptide, a SPOCK1 polypeptide, a TGM4 polypeptide, a Tn antigen, a SialylTn (sTn) antigen, a Thomsen-Friedenreich (T, TF) antigen, a Lewis Y antigen (also known as CD174) antigen, a Sialyl Lewis X (sLex) antigen (also known as Sialyl SSEA-1 (SLX)) antigen, and combinations thereof.

In some embodiments, a target biomarker signature comprises one or more extracellular vesicle-associated surface biomarkers and/or one or more surface biomarkers, each independently selected from a list consisting of an ABCC4 polypeptide, an AP1M2 polypeptide, an ARFGEF3 polypeptide, a CANT1 polypeptide, a CD38 polypeptide, a CDH1 polypeptide, a CLDN3 polypeptide, a CLDN4 polypeptide, a CLGN polypeptide, an ENPP5 polypeptide, a FOLH1 polypeptide, a GOLM1 polypeptide, a GRHL2 polypeptide, a MAP7 polypeptide, a MARCKSL1 polypeptide, a MUC1 polypeptide, a PMEPA1 polypeptide, a PODXL2 polypeptide, a PPP3CA polypeptide, a PSCA polypeptide, a RAB3B polypeptide, a RAB3D polypeptide, a RDH11 polypeptide, a SLC39A6 polypeptide, a SLC4A4 polypeptide, a SMPDL3B polypeptide, a SORD polypeptide, a STEAP1 polypeptide, a STEAP2 polypeptide, a SYT7 polypeptide, a TMPRSS2 polypeptide, a TRPM4 polypeptide, a TSPAN1 polypeptide, an UNC13B polypeptide, a Lewis Y antigen, a SialylTn (sTn) antigen, a Sialyl Lewis X (sLex) antigen (also known as Sialyl SSEA-1 (SLX)) antigen, a T antigen, a Tn antigen, and combinations thereof.

In some embodiments, a target biomarker signature comprises one or more extracellular vesicle-associated surface biomarkers and/or one or more surface biomarkers, each selected from a list consisting of: CLDN3 polypeptide, ABCC4 polypeptide, RAB3B polypeptide, FOLH1 polypeptide, PMEPA1 polypeptide, TMPRSS2 polypeptide, TSPAN1 polypeptide, PODXL2 polypeptide, STEAP2 polypeptide, GOLM1 polypeptide, AP1M2 polypeptide, ARFGEF3 polypeptide, SORD polypeptide, SLC39A6 polypeptide, CLGN polypeptide, GRHL2 polypeptide, CANT1 polypeptide, SYT7 polypeptide, SMPDL3B polypeptide, PPP3CA polypeptide, ENPP5 polypeptide, CDH1 polypeptide, RDH11 polypeptide, SLC4A4 polypeptide, STEAP1 polypeptide, MARCKSL1 polypeptide, UNC13B polypeptide, MAP7 polypeptide, RAB3D polypeptide, CLDN4 polypeptide, and combinations thereof.

In some embodiments, a target biomarker signature comprises one or more extracellular vesicle-associated surface biomarkers and/or one or more surface biomarkers, each selected from a list consisting of: CLDN3 polypeptide, ABCC4 polypeptide, RAB3B polypeptide, FOLH1 polypeptide, PMEPA1 polypeptide, TMPRSS2 polypeptide, TSPAN1 polypeptide, PODXL2 polypeptide, STEAP2 polypeptide, GOLM1 polypeptide, AP1M2 polypeptide, ARFGEF3 polypeptide, SORD polypeptide, SLC39A6 polypeptide, CLGN polypeptide, and combinations thereof.

In some embodiments, a target biomarker signature comprises one or more extracellular vesicle-associated surface biomarkers and/or one or more surface biomarkers, each independently selected from a list consisting of: ABCC4 polypeptide, AP1M2 polypeptide, ARFGEF3 polypeptide, CANT1 polypeptide, CD38 polypeptide, CDH1 polypeptide, CLDN3 polypeptide, CLDN4 polypeptide, CLGN polypeptide, ENPP5 polypeptide, FOLH1 polypeptide, GOLM1 polypeptide, GRHL2 polypeptide, MAP7 polypeptide, MARCKSL1 polypeptide, MUC1 polypeptide, PMEPA1 polypeptide, PODXL2 polypeptide, PPP3CA polypeptide, PSCA polypeptide, RAB3B polypeptide, RAB3D polypeptide, RDH11 polypeptide, SLC39A6 polypeptide, SLC4A4 polypeptide, SMPDL3B polypeptide, SORD polypeptide, STEAP1 polypeptide, STEAP2 polypeptide, SYT7 polypeptide. TMPRSS2 polypeptide, TRPM4 polypeptide, TSPAN1 polypeptide, UNC13B polypeptide, Lewis Y antigen, SialylTn (sTn) antigen, Sialyl Lewis X (sLex) antigen (also known as Sialyl SSEA-1 (SLX)) antigen, T antigen, Tn antigen, and combinations thereof.

In some embodiments, a target biomarker signature comprises one or more extracellular vesicle-associated surface biomarkers and/or one or more surface biomarkers, each selected from a list consisting of: CLDN3 polypeptide, ABCC4 polypeptide, RAB3B polypeptide, FOLH1 polypeptide, PMEPA1 polypeptide, TMPRSS2 polypeptide, TSPAN1 polypeptide, PODXL2 polypeptide, GOLM1 polypeptide, SORD polypeptide, CLGN polypeptide, GRHL2 polypeptide, CANT1 polypeptide, SYT17 polypeptide, PPP3CA polypeptide, and combinations thereof.

In some embodiments, a target biomarker in a target biomarker signature of prostate cancer is or comprises an intravesicular biomarker selected from the group consisting of: a ACP5 polypeptide, a ACPP polypeptide, a ACSM1 polypeptide, a ACTA2 polypeptide, a ACTG2 polypeptide, a ADAMTS1 polypeptide, a AGR2 polypeptide, a AIM1 polypeptide, a ALCAM polypeptide, a ALDH1A3 polypeptide, a ALOX15B polypeptide, a ANTXR1 polypeptide, a ANTXR2 polypeptide, a AP1M2 polypeptide, a AR polypeptide, a ARG2 polypeptide, a ARHGAP6 polypeptide, a ARHGEF26 polypeptide, a ARHGEF37 polypeptide, a ASAP3 polypeptide, a ATP8B1 polypeptide, a ATP9A polypeptide, a BAIAP2L1 polypeptide, a BCAM polypeptide, a BHLHA15 polypeptide, a BOK polypeptide, a C15orf48 polypeptide, a C19orf48 polypeptide, a C1S polypeptide, a C1orf116 polypeptide, a C2orf72 polypeptide, a C8orf4 polypeptide, a C9orf152 polypeptide, a CADM4 polypeptide, a CALD1 polypeptide, a CAMSAP3 polypeptide, a CAPN5 polypeptide, a CBLC polypeptide, a CD24 polypeptide, a CD74 polypeptide, a CDC42EP1 polypeptide, a CDC42EP5 polypeptide, a CGN polypeptide, a CGNL1 polypeptide, a CHMP4C polypeptide, a CLGN polypeptide, a CMTM4 polypeptide, a COLCA1 polypeptide, a CORO2A polypeptide, a CPE polypeptide, a CPNE4 polypeptide, a CPQ polypeptide, a CRYM polypeptide, a CTSF polypeptide, a CX3CL1 polypeptide, a CXADR polypeptide, a CXCR4 polypeptide, a CYB561 polypeptide, a DPYSL3 polypeptide, a DSP polypeptide, a EEF1A2 polypeptide, a EFNA1 polypeptide, a EHD2 polypeptide, a EHF polypeptide, a ELF3 polypeptide, a EMP2 polypeptide, a EPHX1 polypeptide, a EPN3 polypeptide, a ERBB3 polypeptide, a ERG polypeptide, a ESRP1 polypeptide, a ESRP2 polypeptide, a F3 polypeptide, a FAAH polypeptide, a FAM129A polypeptide, a FAM129B polypeptide, a FAM210B polypeptide, a FAM3B polypeptide, a FAM3D polypeptide, a FAM83H polypeptide, a FAM84A polypeptide, a FAM84B polypeptide, a FAT1 polypeptide, a FBLIM1 polypeptide, a FBP1 polypeptide, a FLNC polypeptide, a FNBP1L polypeptide, a FOS polypeptide, a FOXA1 polypeptide, a GADD45G polypeptide, a GATA2 polypeptide, a GGT1 polypeptide, a GJB1 polypeptide, a GLYATL1 polypeptide, a GNG4 polypeptide, a GNMT polypeptide, a GRHL2 polypeptide, a GUCY1A3 polypeptide, a HGD polypeptide, a HID1 polypeptide, a HIST1H2BK polypeptide, a HIST3H2A polypeptide, a HMGCS2 polypeptide, a HOMER2 polypeptide, a HOXB13 polypeptide, a HSD17B6 polypeptide, a HSPB6 polypeptide, a HSPB8 polypeptide, a ICA1 polypeptide, a ID1 polypeptide, a IQGAP2 polypeptide, a IRF6 polypeptide, a ITGA6 polypeptide, a KIAA1522 polypeptide, a KIF5C polypeptide, a KLK2 polypeptide, a KRT14 polypeptide, a KRT15 polypeptide, a KRT17 polypeptide, a KRT18 polypeptide, a KRT19 polypeptide, a KRT5 polypeptide, a KRT7 polypeptide, a KRT8 polypeptide, a LCP1 polypeptide, a LIFR polypeptide, a LIMCH1 polypeptide, a LMOD1 polypeptide, a LRP11 polypeptide, a LRP3 polypeptide, a LSR polypeptide, a MAL2 polypeptide, a MAOA polypeptide, a MAP7 polypeptide, a MARC1 polypeptide, a MARCKSL1 polypeptide, a MB polypeptide, a MESP1 polypeptide, a MLPH polypeptide, a MME polypeptide, a MPZL2 polypeptide, a MT1G polypeptide, a MYBPC1 polypeptide, a MYL9 polypeptide, a MYLK polypeptide, a NCAPD3 polypeptide, a NEDD4L polypeptide, a NEFH polypeptide, a NFIX polypeptide, a NKX3-1 polypeptide, a NPDC1 polypeptide, a NUPR1 polypeptide, a OBSL1 polypeptide, a PALLD polypeptide, a PARVA polypeptide, a PCDH1 polypeptide, a PCP4 polypeptide, a PDIA5 polypeptide, a PEBP4 polypeptide, a PGM5 polypeptide, a PKP1 polypeptide, a PKP3 polypeptide, a PPL polypeptide, a PPM1H polypeptide, a PPP1R1B polypeptide, a PPP3CA polypeptide, a PRAC1 polypeptide, a PRR15L polypeptide, a PSAT1 polypeptide, a PTPRF polypeptide, a PYCR1 polypeptide, a RAB3B polypeptide, a RAMP1 polypeptide, a RAP1GAP polypeptide, a RASEF polypeptide, a RBM47 polypeptide, a RCAN3 polypeptide, a RDH11 polypeptide, a REEP6 polypeptide, a REPS2 polypeptide, a RGS10 polypeptide, a RHPN2 polypeptide, a RNF144B polypeptide, a RORC polypeptide, a RPS4Y1 polypeptide, a S100A16 polypeptide, a SELENBP1 polypeptide, a SERINC5 polypeptide, a SH3BGRL2 polypeptide, a SH3BP4 polypeptide, a SHC2 polypeptide, a SLC1A5 polypeptide, a SLC22A3 polypeptide, a SLC2A12 polypeptide, a SLC40A1 polypeptide, a SLC4A4 polypeptide, a SLC7A8 polypeptide, a SLC9A3R2 polypeptide, a SMOC2 polypeptide, a SORD polypeptide, a SPDEF polypeptide, a SPINT1 polypeptide, a SPINT2 polypeptide, a SPTBN2 polypeptide, a ST14 polypeptide, a STAP2 polypeptide, a STK39 polypeptide, a SULT2B1 polypeptide, a SYNE4 polypeptide, a SYT7 polypeptide, a SYTL1 polypeptide, a TAGLN polypeptide, a TBC1D16 polypeptide, a TBX3 polypeptide, a TC2N polypeptide, a TCEA3 polypeptide, a TEAD3 polypeptide, a TESC polypeptide, a THBS4 polypeptide, a TM4SF1 polypeptide, a TMED3 polypeptide, a TMEM150C polypeptide, a TMEM54 polypeptide, a TMEM98 polypeptide, a TMSB15A polypeptide, a TP53111 polypeptide, a TPD52 polypeptide, a TPM1 polypeptide, a TSPAN13 polypeptide, a TSPAN6 polypeptide, a TSPAN8 polypeptide, a VAMP8 polypeptide, a VSTM2L polypeptide, a WLS polypeptide, a WWC1 polypeptide, a YAP1 polypeptide, a ZBTB42 polypeptide, a ZC3H11A polypeptide, a ZNF217 polypeptide, and combinations thereof. In some embodiments, an intravesicular biomarker described herein may comprise at least one post-translational modification.

In some embodiments, a target biomarker signature comprises one or more intravesicular RNA biomarkers selected from a list consisting of a ABCC4 RNA, a ACP5 RNA, a ACPP RNA, a ACSM1 RNA, a ACTA2 RNA, a ACTG2 RNA, a ADAMTS1 RNA, a AGR2 RNA, a AIM1 RNA, a ALCAM RNA, a ALDH1A3 RNA, a ALOX15B RNA, a ANO7 RNA, a ANPEP RNA, a ANTXR1 RNA, a ANTXR2 RNA, a AP1M2 RNA, a APCDD1 RNA, a AQP3 RNA, a AR RNA, a ARG2 RNA, a ARHGAP6 RNA, a ARHGEF26 RNA, a ARHGEF37 RNA, a ASAP3 RNA, a ASTN2 RNA, a ATP8B1 RNA, a ATP9A RNA, a BAIAP2L1 RNA, a BCAM RNA, a BHLHA15 RNA, a BIK RNA, a BMPR1B RNA, a BOK RNA, a C15orf48 RNA, a C19orf48 RNA, a C1S RNA, a C1orf116 RNA, a C2orf72 RNA, a C8orf4 RNA, a C9orf152 RNA, a CADM4 RNA, a CALD1 RNA, a CAMSAP3 RNA, a CAPN5 RNA, a CBLC RNA, a CD24 RNA, a CD74 RNA, a CDC42EP1 RNA, a CDC42EP5 RNA, a CDH1 RNA, a CGN RNA, a CGNL1 RNA, a CHMP4C RNA, a CHRM1 RNA, a CHRNA2 RNA, a CLDN3 RNA, a CLDN4 RNA, a CLDN7 RNA, a CLDN8 RNA, a CLGN RNA, a CMTM4 RNA, a COLCA1 RNA, a COLEC12 RNA, a CORO2A RNA, a CPE RNA, a CPNE4 RNA, a CPQ RNA, a CRB3 RNA, a CREB3L1 RNA, a CREB3L4 RNA, a CRYM RNA, a CTSF RNA, a CWH43 RNA, a CX3CL1 RNA, a CXADR RNA, a CXCR4 RNA, a CYB561 RNA, a DPP4 RNA, a DPYSL3 RNA, a DSG2 RNA, a DSP RNA, a EEF1A2 RNA, a EFNA1 RNA, a EHD2 RNA, a EHF RNA, a ELF3 RNA, a ELOVL7 RNA, a EMB RNA, a EMP2 RNA, a ENPP5 RNA, a EPCAM RNA, a EPHX1 RNA, a EPN3 RNA, a ERBB3 RNA, a ERG RNA, a ESRP1 RNA, a ESRP2 RNA, a F3 RNA, a FAAH RNA, a FAM129A RNA, a FAM129B RNA, a FAM189A2 RNA, a FAM210B RNA, a FAM3B RNA, a FAM3D RNA, a FAM83H RNA, a FAM84A RNA, a FAM84B RNA, a FAT1 RNA, a FBLIM1 RNA, a FBP1 RNA, a FLNC RNA, a FNBP1L RNA, a FOLH1 RNA, a FOS RNA, a FOXA1 RNA, a FXYD3 RNA, a GADD45G RNA, a GALNT3 RNA, a GALNT7 RNA, a GATA2 RNA, a GCNT1 RNA, a GGT1 RNA, a GJB1 RNA, a GLYATL1 RNA, a GNG4 RNA, a GNMT RNA, a GOLM1 RNA, a GPR160 RNA, a GREB1 RNA, a GRHL2 RNA, a GUCY1A3 RNA, a HGD RNA, a HID1 RNA, a HIST1H2BK RNA, a HIST3H2A RNA, a HMGCS2 RNA, a HOMER2 RNA, a HOXB13 RNA, a HPN RNA, a HSD17B6 RNA, a HSPB6 RNA, a HSPB8 RNA, a ICA1 RNA, a ID1 RNA, a IQGAP2 RNA, a IRF6 RNA, a ITGA6 RNA, a KCNN4 RNA, a KIAA1324 RNA, a KIAA1522 RNA, a KIF5C RNA, a KLK2 RNA, a KRT14 RNA, a KRT15 RNA, a KRT17 RNA, a KRT18 RNA, a KRT19 RNA, a KRT5 RNA, a KRT7 RNA, a KRT8 RNA, a KRTCAP3 RNA, a LCP1 RNA, a LIFR RNA, a LIMCH1 RNA, a LMOD1 RNA, a LPAR3 RNA, a LRP11 RNA, a LRP3 RNA, a LRRC26 RNA, a LSR RNA, a MAL2 RNA, a MAOA RNA, a MAP7 RNA, a MARC1 RNA, a MARCKSL1 RNA, a MB RNA, a MBOAT2 RNA, a MESP1 RNA, a MLPH RNA, a MME RNA, a MPZL2 RNA, a MT1G RNA, a MYBPC1 RNA, a MYL9 RNA, a MYLK RNA, a NCAPD3 RNA, a NEDD4L RNA, a NEFH RNA, a NFIX RNA, a NKX3-1 RNA, a NPDC1 RNA, a NUPR1 RNA, a OBSL1 RNA, a OR51E1 RNA, a OR51E2 RNA, a PALLD RNA, a PARM1 RNA, a PARVA RNA, a PCDH1 RNA, a PCP4 RNA, a PDIA5 RNA, a PDLIM5 RNA, a PDZK11P1 RNA, a PDZRN3 RNA, a PEBP4 RNA, a PGM5 RNA, a PIGR RNA, a PKP1 RNA, a PKP3 RNA, a PLA2G2A RNA, a PMEPA1 RNA, a PODXL2 RNA, a PPL RNA, a PPM1H RNA, a PPP1R1B RNA, a PPP3CA RNA, a PRAC1 RNA, a PROM2 RNA, a PRR15L RNA, a PRSS8 RNA, a PSAT1 RNA, a PSCA RNA, a PTPRF RNA, a PTPRN2 RNA, a PYCR1 RNA, a RAB25 RNA, a RAB27B RNA, a RAB3B RNA, a RAMP1 RNA, a RAP1GAP RNA, a RASEF RNA, a RBM47 RNA, a RCAN3 RNA, a RDH11 RNA, a REEP6 RNA, a REPS2 RNA, a RGS10 RNA, a RHPN2 RNA, a RNF144B RNA, a RORC RNA, a RPS4Y1 RNA, a S100A16 RNA, a SCNN1A RNA, a SDC1 RNA, a SELENBP1 RNA, a SERINC2 RNA, a SERINC5 RNA, a SEZ6L2 RNA, a SH3BGRL2 RNA, a SH3BP4 RNA, a SHC2 RNA, a SLC15A2 RNA, a SLC1A5 RNA, a SLC22A3 RNA, a SLC2A10 RNA, a SLC2A12 RNA, a SLC30A4 RNA, a SLC35F2 RNA, a SLC40A1 RNA, a SLC44A4 RNA, a SLC45A3 RNA, a SLC4A4 RNA, a SLC7A8 RNA, a SLC9A3R2 RNA, a SMIM22 RNA, a SMOC2 RNA, a SORD RNA, a SPDEF RNA, a SPINT1 RNA, a SPINT2 RNA, a SPTBN2 RNA, a ST14 RNA, a STAP2 RNA, a STEAP1 RNA, a STEAP2 RNA, a STEAP4 RNA, a STK39 RNA, a SULT2B1 RNA, a SYNE4 RNA, a SYT7 RNA, a SYTL1 RNA, a TACSTD2 RNA, a TAGLN RNA, a TBC1D16 RNA, a TBX3 RNA, a TC2N RNA, a TCEA3 RNA, a TEAD3 RNA, a TESC RNA, a THBS4 RNA, a TM4SF1 RNA, a TMC4 RNA, a TMC5 RNA, a TMED3 RNA, a TMEFF2 RNA, a TMEM125 RNA, a TMEM150C RNA, a TMEM30B RNA, a TMEM45B RNA, a TMEM54 RNA, a TMEM98 RNA, a TMPRSS2 RNA, a TMSB15A RNA, a TP53111 RNA, a TPD52 RNA, a TPM1 RNA, a TRPM4 RNA, a TRPM8 RNA, a TRPV6 RNA, a TSPAN1 RNA, a TSPAN13 RNA, a TSPAN6 RNA, a TSPAN8 RNA, a TUSC3 RNA, a UPK3A RNA, a VAMP8 RNA, a VSIG2 RNA, a VSTM2L RNA, a WLS RNA, a WWC1 RNA, a YAP1 RNA, a ZBTB42 RNA, a ZC3H11A RNA, a ZNF217 RNA, and combinations thereof.

In some embodiments, a target biomarker signature for prostate cancer comprises at least two or more (e.g., 2, 3, 4, 5, 6, 7, 8, or more) surface biomarkers (e.g., ones described herein) present on the surface of nanoparticles having a size range of interest that includes extracellular vesicles, e.g., in some embodiments, nanoparticles having a size within the range of about 30 nm to about 1000 nm.) In some embodiments, the two or more surface biomarkers are the same. In some embodiments, the two or more surface biomarkers are distinct.

In some embodiments, a target biomarker signature for prostate cancer comprises at least one or more (e.g., 1, 2, 3, 4, 5, 6, 7, 8, or more) extracellular vesicle-associated surface biomarkers (e.g., ones described herein) and at least one or more (e.g., 1, 2, 3, 4, 5, 6, 7, 8, or more) surface biomarkers (e.g., ones described herein). In some embodiments, at least one extracellular vesicle-associated surface biomarker and at least one surface biomarker are the same.

In some embodiments, at least one extracellular vesicle-associated surface biomarker and at least one surface biomarker(s) of a target biomarker signature for prostate cancer are distinct. For example, in some embodiments, a target biomarker signature for prostate cancer comprises at least one extracellular vesicle-associated surface biomarker and at least one surface biomarker.

In some embodiments, a target biomarker signature for prostate cancer comprises at least one or more (e.g., 1, 2, 3, 4, 5, 6, 7, 8, or more) surface biomarker (e.g., ones described herein) present on the surface of nanoparticles having a size range of interest that includes extracellular vesicles, e.g., in some embodiments, nanoparticles having a size within the range of about 30 nm to about 1000 nm.) and at least one or more (e.g., 1, 2, 3, 4, 5, 6, 7, 8, or more) intravesicular biomarkers (e.g., ones described herein). In some such embodiments, the surface biomarker(s) and the intravesicular biomarker(s) can be encoded by the same gene, while the former is present on the surface of the nanoparticles and the latter is contained within the extracellular vesicle (e.g. cargo). In some such embodiments, the surface biomarker(s) and the intravesicular biomarker(s) can be encoded by different genes.

In some embodiments, a target biomarker signature for prostate cancer comprises at least one or more (e.g., 1, 2, 3, 4, 5, 6, 7, 8, or more) extracellular vesicle-associated surface biomarkers (e.g., ones described herein) and at least one or more (e.g., 1, 2, 3, 4, 5, 6, 7, 8, or more) intravesicular biomarkers (e.g., ones described herein). In some such embodiments, the extracellular vesicle-associated surface biomarker(s) and the intravesicular biomarker(s) can be encoded by the same gene, while the former is expressed in the membrane of the extracellular vesicle and the latter is contained within the extracellular vesicle (e.g., cargo). In some such embodiments, the extracellular vesicle-associated surface biomarker(s) and the intravesicular biomarker(s) can be encoded by different genes.

In some embodiments, a target biomarker signature for prostate cancer comprises at least one or more (e.g., 1, 2, 3, 4, 5, 6, 7, 8, or more) surface biomarkers (e.g., ones described herein) and at least one or more (e.g., 1, 2, 3, 4, 5, 6, 7, 8, or more) intravesicular RNA (e.g., mRNA) biomarkers (e.g., ones described herein). In some such embodiments, the surface biomarker(s) and the intravesicular RNA (e.g., but not limited to mRNA and noncoding RNA such as, e.g., orphan noncoding RNA, long noncoding RNA, piwi-interacting RNA, microRNA, circular RNA, etc.) biomarker(s) can be encoded by the same gene. In some such embodiments, the surface biomarker(s) and the intravesicular RNA (e.g., but not limited to mRNA and noncoding RNA such as, e.g., orphan noncoding RNA, long noncoding RNA, piwi-interacting RNA, microRNA, circular RNA, etc.) biomarker(s) can be encoded by different genes.

In some embodiments, a target biomarker signature for prostate cancer comprises at least one or more (e.g., 1, 2, 3, 4, 5, 6, 7, 8, or more) extracellular vesicle-associated surface biomarkers (e.g., ones described herein) and at least one or more (e.g., 1, 2, 3, 4, 5, 6, 7, 8, or more) intravesicular RNA (e.g., mRNA) biomarkers (e.g., ones described herein). In some such embodiments, the extracellular vesicle-associated surface biomarker(s) and the intravesicular RNA (e.g., mRNA) biomarker(s) can be encoded by the same gene. In some such embodiments, the extracellular vesicle-associated surface biomarker(s) and the intravesicular RNA (e.g., mRNA) biomarker(s) can be encoded by different genes.

In some embodiments, any one of provided biomarkers can be detected and/or measured by protein and/or RNA (e.g., mRNA) expression levels in wild-type form.

In some embodiments, any one of provided biomarkers can be detected and/or measured by protein and/or RNA (e.g., mRNA) expression levels in mutant form. Thus, in some embodiments, mutant-specific detection of provided biomarkers (e.g., proteins and/or RNA such as, e.g., mRNAs) can be included.

As noted herein, in some embodiments, a biomarker is or comprises a particular form of one or more polypeptides or proteins (e.g., a pro-form, a truncated form, a modified form such as a glycosylated, phosphorylated, acetylated, methylated, ubiquitylated, lipidated form, etc). In some embodiments, detection of such form detects a plurality (and, in some embodiments, substantially all) polypeptides present in that form (e.g., containing a particular modification such as, for example, a particular glycosylation, e.g., sialyl-Tn (sTn) glycosylation, e.g., a truncated O-glycan containing a sialic acid α-2,6 linked to GalNAc α-O-Ser/Thr.

Accordingly, in some embodiments, a surface biomarker can be or comprise a glycosylation moiety (e.g., an sTn antigen moiety, a Tn antigen moiety, or a T antigen moiety). Thompsen-nouvelle (Tn) antigen is an O-linked glycan that is thought to be associated with a broad array of tumors. Tn is a single alpha-linked GalNAc added to Ser or Thr as the first step of a major O-linked glycosylation pathway. A skilled artisan will understand that in certain embodiments. T antigen typically refers to an O-linked glycan with the structure Galβ1-3GalNAc—.

In some embodiments, a surface protein biomarker can be or comprise a tumor-associated post-translational modification. In some embodiments, such a post-translational modification can be or comprise tumor-specific glycosylation patterns such as mucins with glycans aberrantly truncated at the initial GalNAc (e.g., Tn), or combinations thereof. In some embodiments, a surface protein biomarker can be or comprise a tumor-specific proteoform of mucin resulting from altered splicing and/or translation (isoforms) or proteolysis (cancer specific protease activity resulting in aberrant cleavage products).

In some embodiments, a target biomarker signature comprises a combination of at least two biomarkers, which combination can be selected from the following: a ABCC4 polypeptide and a RAB3B polypeptide; or a ABCC4 polypeptide and a FOLH1 polypeptide; or a ABCC4 polypeptide and a GOLM1 polypeptide; or a ABCC4 polypeptide and a PODXL2 polypeptide; or a FOLH1 polypeptide and a TSPAN1 polypeptide; or a GOLM1 polypeptide and a RAB3B polypeptide; or a ABCC4 polypeptide and a ARFGEF3 polypeptide; or a ABCC4 polypeptide and a TMPRSS2 polypeptide; or a ABCC4 polypeptide and a CLGN polypeptide; or a ABCC4 polypeptide and a SYT7 polypeptide; or a FOLH1 polypeptide and a GOLM1 polypeptide; or a ABCC4 polypeptide and a PMEPA1 polypeptide; or a FOLH1 polypeptide and a SMPDL3B polypeptide; or a FOLH1 polypeptide and a PODXL2 polypeptide; or a PMEPA1 polypeptide and a TSPAN1 polypeptide; or a CLDN4 polypeptide and a FOLH1 polypeptide; or a RAB3B polypeptide and a SLC4A4 polypeptide; or a PODXL2 polypeptide and a RAB3B polypeptide; or a ABCC4 polypeptide and a RDH11 polypeptide; or a CANT1 polypeptide and a FOLH1 polypeptide; or a FOLH1 polypeptide and a PPP3CA polypeptide; or a ARFGEF3 polypeptide and a RAB3B polypeptide; or a AP1M2 polypeptide and a FOLH1 polypeptide; or a ENPP5 polypeptide and a RAB3B polypeptide; or a FOLH1 polypeptide and a SLC4A4 polypeptide; or a RAB3B polypeptide and a SMPDL3B polypeptide; or a MARCKSL1 polypeptide and a RAB3B polypeptide; or a PPP3CA polypeptide and a RAB3B polypeptide; or a CLGN polypeptide and a GOLM1 polypeptide; or a RAB3B polypeptide and a TSPAN1 polypeptide; or a GOLM1 polypeptide and a SORD polypeptide; or a SLC39A6 polypeptide and a TSPAN1 polypeptide; or a PPP3CA polypeptide and a TSPAN1 polypeptide; or a STEAP2 polypeptide and a TSPAN1 polypeptide; or a CLGN polypeptide and a TSPAN1 polypeptide; or a CLDN3 polypeptide and a PMEPA1 polypeptide; or a SLC39A6 polypeptide and a TMPRSS2 polypeptide; or a CLGN polypeptide and a SLC4A4 polypeptide; or a HOMER2 polypeptide and a TSPAN1 polypeptide; or a CLGN polypeptide and a PODXL2 polypeptide; or a PPP3CA polypeptide and a TMPRSS2 polypeptide; or a PODXL2 polypeptide and a TMPRSS2 polypeptide; or a ARFGEF3 polypeptide and a CLGN polypeptide; or a CLN5 polypeptide and a STEAP1 polypeptide; or a CLDN3 polypeptide and a SLC39A6 polypeptide; or a CLGN polypeptide and a SMPDL3B polypeptide; or a PODXL2 polypeptide and a SORD polypeptide; or a GOLM1 polypeptide and a TMPRSS2 polypeptide; or a APOO polypeptide and a YIPF1 polypeptide; or a APOO polypeptide and a SYNGR2 polypeptide; or a SYT7 polypeptide and a TSPAN1 polypeptide; or a GOLM1 polypeptide and a GRHL2 polypeptide; or a HPN polypeptide and a PMEPA1 polypeptide; or a ENPP5 polypeptide and a TMPRSS2 polypeptide; or a STEAP1 polypeptide and a TSPAN1 polypeptide; or a CLDN3 polypeptide and a PPP3CA polypeptide; or a CLDN3 polypeptide and a STEAP2 polypeptide; or a APOO polypeptide and a MIA3 polypeptide; or a MARCKSL1 polypeptide and a TMPRSS2 polypeptide; or a SYNGR2 polypeptide and a YIPF1 polypeptide; or a AD11 polypeptide and a SMPDL3B polypeptide; or a GOLM1 polypeptide and a RDH11 polypeptide; or a AP1M2 polypeptide and a CLGN polypeptide; or a RAP1GAP polypeptide and a SLC39A7 polypeptide; or a GOLM1 polypeptide and a PMEPA1 polypeptide; or a STEAP1 polypeptide and a TMPRSS2 polypeptide; or a APOO polypeptide and a TMEM192 polypeptide; or a PMEPA1 polypeptide and a PODXL2 polypeptide; or a CADM4 polypeptide and a MARCKSL1 polypeptide; or a CLGN polypeptide and a PPP3CA polypeptide; or a GOLM1 polypeptide and a SLC39A6 polypeptide; or a PODXL2 polypeptide and a STEAP2 polypeptide; or a ARFGEF3 polypeptide and a SORD polypeptide; or a GRHL2 polypeptide and a SLC4A4 polypeptide; or a ARFGEF3 polypeptide and a TMPRSS2 polypeptide; or a SLC39A7 polypeptide and a SMPDL3B polypeptide; or a PODXL2 polypeptide and a STEAP1 polypeptide; or a SLC4A4 polypeptide and a SORD polypeptide; or a CANT1 polypeptide and a CLGN polypeptide; or a SLC4A4 polypeptide and a TMPRSS2 polypeptide; or a STEAP1 polypeptide and a STEAP2 polypeptide; or a APOO polypeptide and a TMEM9 polypeptide; or a MARCKSL1 polypeptide and a PMEPA1 polypeptide; or a AGTRAP polypeptide and a SMPDL3B polypeptide; or a SMPDL3B polypeptide and a SORD polypeptide; or a MARCKSL1 polypeptide and a STEAP2 polypeptide; or a HPN polypeptide and a SLC39A6 polypeptide; or a HPN polypeptide and a STEAP2 polypeptide; or a ARFGEF3 polypeptide and a CLDN3 polypeptide; or a AP1M2 polypeptide and a CANT1 polypeptide; or a APOO polypeptide and a BCAM polypeptide; or a APOO polypeptide and a SMPDL3B polypeptide; or a HPN polypeptide and a PPP3CA polypeptide; or a SMPDL3B polypeptide and a SYNGR2 polypeptide; or a ARFGEF3 polypeptide and a PMEPA1 polypeptide; or a EPCAM polypeptide and a STEAP1 polypeptide; or a ARFGEF3 polypeptide and a HPN polypeptide; or a GNPNAT1 polypeptide and a RAP1GAP polypeptide; or a APOO polypeptide and a CLN5 polypeptide; or a APOO polypeptide and a ATP2C1 polypeptide; or a ABCC4 polypeptide and a CLDN3 polypeptide; or a ABCC4 polypeptide and a TSPAN1 polypeptide; or a CLDN3 polypeptide and a FOLH1 polypeptide; or a PODXL2 polypeptide and a TSPAN1 polypeptide; or a GOLM1 polypeptide and a PODXL2 polypeptide; or a PODXL2 polypeptide and a PPP3CA polypeptide; or a CLGN polypeptide and a MARCKSL1 polypeptide; or a CLGN polypeptide and a PMEPA1 polypeptide; or a PMEPA1 polypeptide and a PPP3CA polypeptide; or a PPP3CA polypeptide and a STEAP1 polypeptide; or a PMEPA1 polypeptide and a SMPDL3B polypeptide; or a GRHL2 polypeptide and a HPN polypeptide; or a AGTRAP polypeptide and a HPN polypeptide; or a CANT1 polypeptide and a SLC39A6 polypeptide; or a SLC39A6 polypeptide and a SLC4A4 polypeptide; or a CADM4 polypeptide and a STEAP1 polypeptide; or a CLDN3 polypeptide and a ENPP5 polypeptide; or a CLDN3 polypeptide and a MARCKSL1 polypeptide; or a SLC39A6 polypeptide and a SYNGR2 polypeptide; or a APOO polypeptide and a HOMER2 polypeptide; or a APOO polypeptide and a SCARB2 polypeptide; or a CANT1 polypeptide and a SORD polypeptide; or a MARCKSL1 polypeptide and a STEAP1 polypeptide; or a APOO polypeptide and a SLC39A6 polypeptide; or a LRIG1 polypeptide and a PRSS8 polypeptide; or a ABCC4 polypeptide and a SORD polypeptide; or a ABCC4 polypeptide and a PPP3CA polypeptide; or a ABCC4 polypeptide and a ENPP5 polypeptide; or a FOLH1 polypeptide and a PMEPA1 polypeptide; or a FOLH1 polypeptide and a TRPM4 polypeptide; or a ENPP5 polypeptide and a FOLH1 polypeptide; or a CLDN3 polypeptide and a CLDN4 polypeptide; or a CLDN3 polypeptide and a GOLM1 polypeptide; or a CDH1 polypeptide and a FOLH1 polypeptide; or a EPCAM polypeptide and a PMEPA1 polypeptide; or a CLDN4 polypeptide and a GOLM1 polypeptide; or a GOLM1 polypeptide and a PPP3CA polypeptide; or a CDH1 polypeptide and a GOLM1 polypeptide; or a ENPP5 polypeptide and a PMEPA1 polypeptide; or a AD11 polypeptide and a TSPAN1 polypeptide; or a ENPP5 polypeptide and a PPP3CA polypeptide; or a RDH11 polypeptide and a TSPAN1 polypeptide; or a SORD polypeptide and a TSPAN1 polypeptide; or a EPCAM polypeptide and a PPP3CA polypeptide; or a GOLM1 polypeptide and a TSPAN1 polypeptide; or a BCAM polypeptide and a TSPAN1 polypeptide; or a CLGN polypeptide and a ENPP5 polypeptide; or a CLDN4 polypeptide and a PPP3CA polypeptide; or a CLDN4 polypeptide and a HPN polypeptide; or a CLDN3 polypeptide and a SCARB2 polypeptide; or a CLDN3 polypeptide and a LCP1 polypeptide; or a CDH1 polypeptide and a CLGN polypeptide; or a BCAM polypeptide and a CLDN3 polypeptide; or a BCAM polypeptide and a CLGN polypeptide; or a HID1 polypeptide and a RAP1GAP polypeptide; or a BCAM polypeptide and a EPCAM polypeptide; or a CLDN4 polypeptide and a CLGN polypeptide; or a EPCAM polypeptide and a RDH11 polypeptide; or a CLGN polypeptide and a TRPM4 polypeptide; or a RAP1GAP polypeptide and a SORD polypeptide; or a ENPP5 polypeptide and a PRSS8 polypeptide; or a ENPP5 polypeptide and a RAP1GAP polypeptide; or a ENPP5 polypeptide and a EPCAM polypeptide; or a EPCAM polypeptide and a HPN polypeptide; or a ENPP5 polypeptide and a TRPM4 polypeptide; or a EPCAM polypeptide and a SORD polypeptide; or a BCAM polypeptide and a PRSS8 polypeptide; or a CDH1 polypeptide and a HPN polypeptide; or a SCARB2 polypeptide and a SORD polypeptide; or a RAP1GAP polypeptide and a SCARB2 polypeptide; or a CLDN4 polypeptide and a RAP1GAP polypeptide; or a CLDN4 polypeptide and a PRSS8 polypeptide; or a CLDN4 polypeptide and a RDH11 polypeptide; or a CDH1 polypeptide and a RDH11 polypeptide; or a RAP1GAP polypeptide and a TRPM4 polypeptide; or a CLDN4 polypeptide and a TRPM4 polypeptide; or a CDH1 polypeptide and a RAP1GAP polypeptide; or a RAP1GAP polypeptide and a RDH11 polypeptide; or a CADM4 polypeptide and a PRSS8 polypeptide; or a BCAM polypeptide and a HPN polypeptide; or a HPN polypeptide and a RDH11 polypeptide; or a CDH1 polypeptide and a SCARB2 polypeptide; or a AD11 polypeptide and a PRSS8 polypeptide; or a LCP1 polypeptide and a RAP1GAP polypeptide; or a HPN polypeptide and a PRSS8 polypeptide; or a CADM4 polypeptide and a TRPM4 polypeptide; or a SORD polypeptide and a TRPM4 polypeptide; or a CADM4 polypeptide and a CDH1 polypeptide; or a CDH1 polypeptide and a TRPM4 polypeptide; or a BCAM polypeptide and a CDH1 polypeptide; or a BCAM polypeptide and a RDH11 polypeptide; or a RDH11 polypeptide and a TRPM4 polypeptide; or a CADM4 polypeptide and a HPN polypeptide; or a HID1 polypeptide and a HPN polypeptide; or a PRSS8 polypeptide and a SCARB2 polypeptide; or a FAAH polypeptide and a RAP1GAP polypeptide; or a CADM4 polypeptide and a RDH11 polypeptide; or a AD11 polypeptide and a HID1 polypeptide; or a LCP1 polypeptide and a TRPM4 polypeptide; or a HPN polypeptide and a LCP1 polypeptide; or and combinations thereof. In some embodiments, a target biomarker in the foregoing combinations may be used as a target of a capture probe and/or a target of a detection probe of assays described herein.

In some embodiments, a target biomarker signature comprises at least two biomarkers, which is or comprises a ABCC4 polypeptide and a RAB3B polypeptide. In some embodiments, a target biomarker signature comprises at least two biomarkers, which is or comprises a ABCC4 polypeptide and a FOLH1 polypeptide. In some embodiments, a target biomarker signature comprises at least two biomarkers, which is or comprises a ABCC4 polypeptide and a GOLM1 polypeptide. In some embodiments, a target biomarker signature comprises at least two biomarkers, which is or comprises a ABCC4 polypeptide and a PODXL2 polypeptide. In some embodiments, a target biomarker signature comprises at least two biomarkers, which is or comprises a FOLH1 polypeptide and a TSPAN1 polypeptide. In some embodiments, a target biomarker signature comprises at least two biomarkers, which is or comprises a GOLM1 polypeptide and a RAB3B polypeptide. In some embodiments, a target biomarker signature comprises at least two biomarkers, which is or comprises a ABCC4 polypeptide and a ARFGEF3 polypeptide. In some embodiments, a target biomarker signature comprises at least two biomarkers, which is or comprises a ABCC4 polypeptide and a TMPRSS2 polypeptide. In some embodiments, a target biomarker signature comprises at least two biomarkers, which is or comprises a ABCC4 polypeptide and a CLGN polypeptide. In some embodiments, a target biomarker signature comprises at least two biomarkers, which is or comprises a ABCC4 polypeptide and a SYT7 polypeptide. In some embodiments, a target biomarker signature comprises at least two biomarkers, which is or comprises a FOLH1 polypeptide and a GOLM1 polypeptide. In some embodiments, a target biomarker signature comprises at least two biomarkers, which is or comprises a ABCC4 polypeptide and a PMEPA1 polypeptide. In some embodiments, a target biomarker signature comprises at least two biomarkers, which is or comprises a FOLH1 polypeptide and a SMPDL3B polypeptide. In some embodiments, a target biomarker signature comprises at least two biomarkers, which is or comprises a FOLH1 polypeptide and a PODXL2 polypeptide. In some embodiments, a target biomarker signature comprises at least two biomarkers, which is or comprises a PMEPA1 polypeptide and a TSPAN1 polypeptide. In some embodiments, a target biomarker signature comprises at least two biomarkers, which is or comprises a ABCC4 polypeptide and a CLDN3 polypeptide. In some embodiments, a target biomarker signature comprises at least two biomarkers, which is or comprises a ABCC4 polypeptide and a RDH11 polypeptide. In some embodiments, a target biomarker signature comprises at least two biomarkers, which is or comprises a ABCC4 polypeptide and a SORD polypeptide. In some embodiments, a target biomarker signature comprises at least two biomarkers, which is or comprises a ABCC4 polypeptide and a TSPAN1 polypeptide. In some embodiments, a target biomarker signature comprises at least two biomarkers, which is or comprises a FOLH1 polypeptide and a PPP3CA polypeptide. In some embodiments, a target biomarker signature comprises at least two biomarkers, which is or comprises a PPP3CA polypeptide and a TSPAN1 polypeptide. In some embodiments, a target biomarker signature comprises at least two biomarkers, which is or comprises a CLDN4 polypeptide and a FOLH1 polypeptide. In some embodiments, a target biomarker signature comprises at least two biomarkers, which is or comprises a ABCC4 polypeptide and a PPP3CA polypeptide. In some embodiments, a target biomarker signature comprises at least two biomarkers, which is or comprises a ABCC4 polypeptide and a ENPP5 polypeptide. In some embodiments, a target biomarker signature comprises at least two biomarkers, which is or comprises a FOLH1 polypeptide and a PMEPA1 polypeptide. In some embodiments, a target biomarker signature comprises at least two biomarkers, which is or comprises a CLGN polypeptide and a GOLM1 polypeptide. In some embodiments, a target biomarker signature comprises at least two biomarkers, which is or comprises a CLGN polypeptide and a TSPAN1 polypeptide. In some embodiments, a target biomarker signature comprises at least two biomarkers, which is or comprises a FOLH1 polypeptide and a TRPM4 polypeptide. In some embodiments, a target biomarker signature comprises at least two biomarkers, which is or comprises a ENPP5 polypeptide and a FOLH1 polypeptide. In some embodiments, a target biomarker signature comprises at least two biomarkers, which is or comprises a CLDN3 polypeptide and a FOLH1 polypeptide. In some embodiments, a target biomarker signature comprises at least two biomarkers, which is or comprises a CLDN3 polypeptide and a CLDN4 polypeptide. In some embodiments, a target biomarker signature comprises at least two biomarkers, which is or comprises a GOLM1 polypeptide and a SORD polypeptide. In some embodiments, a target biomarker signature comprises at least two biomarkers, which is or comprises a CLDN3 polypeptide and a PMEPA1 polypeptide. In some embodiments, a target biomarker signature comprises at least two biomarkers, which is or comprises a PODXL2 polypeptide and a RAB3B polypeptide. In some embodiments, a target biomarker signature comprises at least two biomarkers, which is or comprises a RAB3B polypeptide and a SLC4A4 polypeptide. In some embodiments, a target biomarker signature comprises at least two biomarkers, which is or comprises a CANT1 polypeptide and a FOLH1 polypeptide. In some embodiments, a target biomarker signature comprises at least two biomarkers, which is or comprises a RAB3B polypeptide and a SMPDL3B polypeptide. In some embodiments, a target biomarker signature comprises at least two biomarkers, which is or comprises a ENPP5 polypeptide and a RAB3B polypeptide. In some embodiments, a target biomarker signature comprises at least two biomarkers, which is or comprises a FOLH1 polypeptide and a SLC4A4 polypeptide. In some embodiments, a target biomarker signature comprises at least two biomarkers, which is or comprises a SLC39A6 polypeptide and a TSPAN1 polypeptide. In some embodiments, a target biomarker signature comprises at least two biomarkers, which is or comprises a ARFGEF3 polypeptide and a RAB3B polypeptide. In some embodiments, a target biomarker signature comprises at least two biomarkers, which is or comprises a AP1M2 polypeptide and a FOLH1 polypeptide. In some embodiments, a target biomarker in the foregoing combinations may be used as a target of a capture probe and/or a target of a detection probe of assays described herein.

In some embodiments, a target biomarker signature comprises at least two biomarkers, which is or comprises a MUC1 polypeptide and an ABCC4 polypeptide. In some embodiments, a target biomarker signature comprises at least two biomarkers, which is or comprises a MUC1 polypeptide and an AP1M2 polypeptide. In some embodiments, a target biomarker signature comprises at least two biomarkers, which is or comprises a MUC1 polypeptide and an ARFGEF3 polypeptide. In some embodiments, a target biomarker signature comprises at least two biomarkers, which is or comprises a MUC1 polypeptide and a CANT1 polypeptide. In some embodiments, a target biomarker signature comprises at least two biomarkers, which is or comprises a MUC1 polypeptide and a CD38 polypeptide. In some embodiments, a target biomarker signature comprises at least two biomarkers, which is or comprises a MUC1 polypeptide and a CDH1 polypeptide. In some embodiments, a target biomarker signature comprises at least two biomarkers, which is or comprises a MUC1 polypeptide and a CLDN3 polypeptide. In some embodiments, a target biomarker signature comprises at least two biomarkers, which is or comprises a MUC1 polypeptide and a CLDN4 polypeptide. In some embodiments, a target biomarker signature comprises at least two biomarkers, which is or comprises a MUC1 polypeptide and a CLGN polypeptide. In some embodiments, a target biomarker signature comprises at least two biomarkers, which is or comprises a MUC1 polypeptide and a ENPP5 polypeptide. In some embodiments, a target biomarker signature comprises at least two biomarkers, which is or comprises a MUC1 polypeptide and a FOLH1 polypeptide. In some embodiments, a target biomarker signature comprises at least two biomarkers, which is or comprises a MUC1 polypeptide and a GOLM1 polypeptide. In some embodiments, a target biomarker signature comprises at least two biomarkers, which is or comprises a MUC1 polypeptide and a GRHL2 polypeptide. In some embodiments, a target biomarker signature comprises at least two biomarkers, which is or comprises a MUC1 polypeptide and a MAP7 polypeptide. In some embodiments, a target biomarker signature comprises at least two biomarkers, which is or comprises a MUC1 polypeptide and a MARCKSL1 polypeptide. In some embodiments, a target biomarker signature comprises at least two biomarkers, which is or comprises a MUC1 polypeptide and a PMEPA1 polypeptide. In some embodiments, a target biomarker signature comprises at least two biomarkers, which is or comprises a MUC1 polypeptide and a PODXL2 polypeptide. In some embodiments, a target biomarker signature comprises at least two biomarkers, which is or comprises a MUC1 polypeptide and a PPP3CA polypeptide. In some embodiments, a target biomarker signature comprises at least two biomarkers, which is or comprises a MUC1 polypeptide and a PSCA polypeptide. In some embodiments, a target biomarker signature comprises at least two biomarkers, which is or comprises a MUC1 polypeptide and a RAB3B polypeptide. In some embodiments, a target biomarker signature comprises at least two biomarkers, which is or comprises a MUC1 polypeptide and a RAB3D polypeptide. In some embodiments, a target biomarker signature comprises at least two biomarkers, which is or comprises a MUC1 polypeptide and a RDH11 polypeptide. In some embodiments, a target biomarker signature comprises at least two biomarkers, which is or comprises a MUC1 polypeptide and a SLC39A6 polypeptide. In some embodiments, a target biomarker signature comprises at least two biomarkers, which is or comprises a MUC1 polypeptide and a SLC4A4 polypeptide. In some embodiments, a target biomarker signature comprises at least two biomarkers, which is or comprises a MUC1 polypeptide and a SMPDL3B polypeptide. In some embodiments, a target biomarker signature comprises at least two biomarkers, which is or comprises a MUC1 polypeptide and a SORD polypeptide. In some embodiments, a target biomarker signature comprises at least two biomarkers, which is or comprises a MUC1 polypeptide and a STEAP1 polypeptide. In some embodiments, a target biomarker signature comprises at least two biomarkers, which is or comprises a MUC1 polypeptide and a STEAP2 polypeptide. In some embodiments, a target biomarker signature comprises at least two biomarkers, which is or comprises a MUC1 polypeptide and a SYT7 polypeptide. In some embodiments, a target biomarker signature comprises at least two biomarkers, which is or comprises a MUC1 polypeptide and a TMPRSS2 polypeptide. In some embodiments, a target biomarker signature comprises at least two biomarkers, which is or comprises a MUC1 polypeptide and a TRPM4 polypeptide. In some embodiments, a target biomarker signature comprises at least two biomarkers, which is or comprises a MUC1 polypeptide and a TSPAN1 polypeptide. In some embodiments, a target biomarker signature comprises at least two biomarkers, which is or comprises a MUC1 polypeptide and an UNC13B polypeptide. In some embodiments, a target biomarker in the foregoing combinations may be used as a target of a capture probe and/or a target of a detection probe of assays described herein.

In some embodiments, a target biomarker signature comprises at least two biomarkers, which is or comprises a sTn antigen and an ABCC4 polypeptide. In some embodiments, a target biomarker signature comprises at least two biomarkers, which is or comprises a sTn antigen and an AP1M2 polypeptide. In some embodiments, a target biomarker signature comprises at least two biomarkers, which is or comprises a sTn antigen and an ARFGEF3 polypeptide. In some embodiments, a target biomarker signature comprises at least two biomarkers, which is or comprises a sTn antigen and a CANT1 polypeptide. In some embodiments, a target biomarker signature comprises at least two biomarkers, which is or comprises a sTn antigen and a CD38 polypeptide. In some embodiments, a target biomarker signature comprises at least two biomarkers, which is or comprises a sTn antigen and a CDH1 polypeptide. In some embodiments, a target biomarker signature comprises at least two biomarkers, which is or comprises a sTn antigen and a CLDN3 polypeptide. In some embodiments, a target biomarker signature comprises at least two biomarkers, which is or comprises a sTn antigen and a CLDN4 polypeptide. In some embodiments, a target biomarker signature comprises at least two biomarkers, which is or comprises a sTn antigen and a CLGN polypeptide. In some embodiments, a target biomarker signature comprises at least two biomarkers, which is or comprises a sTn antigen and a ENPP5 polypeptide. In some embodiments, a target biomarker signature comprises at least two biomarkers, which is or comprises a sTn antigen and a FOLH1 polypeptide. In some embodiments, a target biomarker signature comprises at least two biomarkers, which is or comprises a sTn antigen and a GOLM1 polypeptide. In some embodiments, a target biomarker signature comprises at least two biomarkers, which is or comprises a sTn antigen and a GRHL2 polypeptide. In some embodiments, a target biomarker signature comprises at least two biomarkers, which is or comprises a sTn antigen and a MAP7 polypeptide. In some embodiments, a target biomarker signature comprises at least two biomarkers, which is or comprises a sTn antigen and a MARCKSL1 polypeptide. In some embodiments, a target biomarker signature comprises at least two biomarkers, which is or comprises a sTn antigen and a PMEPA1 polypeptide. In some embodiments, a target biomarker signature comprises at least two biomarkers, which is or comprises a sTn antigen and a PODXL2 polypeptide. In some embodiments, a target biomarker signature comprises at least two biomarkers, which is or comprises a sTn antigen and a PPP3CA polypeptide. In some embodiments, a target biomarker signature comprises at least two biomarkers, which is or comprises a sTn antigen and a PSCA polypeptide. In some embodiments, a target biomarker signature comprises at least two biomarkers, which is or comprises a sTn antigen and a RAB3B polypeptide. In some embodiments, a target biomarker signature comprises at least two biomarkers, which is or comprises a sTn antigen and a RAB3D polypeptide. In some embodiments, a target biomarker signature comprises at least two biomarkers, which is or comprises a sTn antigen and a RDH11 polypeptide. In some embodiments, a target biomarker signature comprises at least two biomarkers, which is or comprises a sTn antigen and a SLC39A6 polypeptide. In some embodiments, a target biomarker signature comprises at least two biomarkers, which is or comprises a sTn antigen and a SLC4A4 polypeptide. In some embodiments, a target biomarker signature comprises at least two biomarkers, which is or comprises a sTn antigen and a SMPDL3B polypeptide. In some embodiments, a target biomarker signature comprises at least two biomarkers, which is or comprises a sTn antigen and a SORD polypeptide. In some embodiments, a target biomarker signature comprises at least two biomarkers, which is or comprises a sTn antigen and a STEAP1 polypeptide. In some embodiments, a target biomarker signature comprises at least two biomarkers, which is or comprises a sTn antigen and a STEAP2 polypeptide. In some embodiments, a target biomarker signature comprises at least two biomarkers, which is or comprises a sTn antigen and a SYT7 polypeptide. In some embodiments, a target biomarker signature comprises at least two biomarkers, which is or comprises a sTn antigen and a TMPRSS2 polypeptide. In some embodiments, a target biomarker signature comprises at least two biomarkers, which is or comprises a sTn antigen and a TRPM4 polypeptide. In some embodiments, a target biomarker signature comprises at least two biomarkers, which is or comprises a sTn antigen and a TSPAN1 polypeptide. In some embodiments, a target biomarker signature comprises at least two biomarkers, which is or comprises a sTn antigen and an UNC13B polypeptide. In some embodiments, a target biomarker in the foregoing combinations may be used as a target of a capture probe and/or a target of a detection probe of assays described herein.

In some embodiments, a target biomarker signature comprises at least two biomarkers, which is or comprises a sLex antigen and an ABCC4 polypeptide. In some embodiments, a target biomarker signature comprises at least two biomarkers, which is or comprises a sLex antigen and an AP1M2 polypeptide. In some embodiments, a target biomarker signature comprises at least two biomarkers, which is or comprises a sLex antigen and an ARFGEF3 polypeptide. In some embodiments, a target biomarker signature comprises at least two biomarkers, which is or comprises a sLex antigen and a CANT1 polypeptide. In some embodiments, a target biomarker signature comprises at least two biomarkers, which is or comprises a sLex antigen and a CD38 polypeptide. In some embodiments, a target biomarker signature comprises at least two biomarkers, which is or comprises a sLex antigen and a CDH1 polypeptide. In some embodiments, a target biomarker signature comprises at least two biomarkers, which is or comprises a sLex antigen and a CLDN3 polypeptide. In some embodiments, a target biomarker signature comprises at least two biomarkers, which is or comprises a sLex antigen and a CLDN4 polypeptide. In some embodiments, a target biomarker signature comprises at least two biomarkers, which is or comprises a sLex antigen and a CLGN polypeptide. In some embodiments, a target biomarker signature comprises at least two biomarkers, which is or comprises a sLex antigen and a ENPP5 polypeptide. In some embodiments, a target biomarker signature comprises at least two biomarkers, which is or comprises a sLex antigen and a FOLH1 polypeptide. In some embodiments, a target biomarker signature comprises at least two biomarkers, which is or comprises a sLex antigen and a GOLM1 polypeptide. In some embodiments, a target biomarker signature comprises at least two biomarkers, which is or comprises a sLex antigen and a GRHL2 polypeptide. In some embodiments, a target biomarker signature comprises at least two biomarkers, which is or comprises a sLex antigen and a MAP7 polypeptide. In some embodiments, a target biomarker signature comprises at least two biomarkers, which is or comprises a sLex antigen and a MARCKSL1 polypeptide. In some embodiments, a target biomarker signature comprises at least two biomarkers, which is or comprises a sLex antigen and a PMEPA1 polypeptide. In some embodiments, a target biomarker signature comprises at least two biomarkers, which is or comprises a sLex antigen and a PODXL2 polypeptide. In some embodiments, a target biomarker signature comprises at least two biomarkers, which is or comprises a sLex antigen and a PPP3CA polypeptide. In some embodiments, a target biomarker signature comprises at least two biomarkers, which is or comprises a sLex antigen and a PSCA polypeptide. In some embodiments, a target biomarker signature comprises at least two biomarkers, which is or comprises a sLex antigen and a RAB3B polypeptide. In some embodiments, a target biomarker signature comprises at least two biomarkers, which is or comprises a sLex antigen and a RAB3D polypeptide. In some embodiments, a target biomarker signature comprises at least two biomarkers, which is or comprises a sLex antigen and a RDH11 polypeptide. In some embodiments, a target biomarker signature comprises at least two biomarkers, which is or comprises a sLex antigen and a SLC39A6 polypeptide. In some embodiments, a target biomarker signature comprises at least two biomarkers, which is or comprises a sLex antigen and a SLC4A4 polypeptide. In some embodiments, a target biomarker signature comprises at least two biomarkers, which is or comprises a sLex antigen and a SMPDL3B polypeptide. In some embodiments, a target biomarker signature comprises at least two biomarkers, which is or comprises a sLex antigen and a SORD polypeptide. In some embodiments, a target biomarker signature comprises at least two biomarkers, which is or comprises a sLex antigen and a STEAP1 polypeptide. In some embodiments, a target biomarker signature comprises at least two biomarkers, which is or comprises a sLex antigen and a STEAP2 polypeptide. In some embodiments, a target biomarker signature comprises at least two biomarkers, which is or comprises a sLex antigen and a SYT7 polypeptide. In some embodiments, a target biomarker signature comprises at least two biomarkers, which is or comprises a sLex antigen and a TMPRSS2 polypeptide. In some embodiments, a target biomarker signature comprises at least two biomarkers, which is or comprises a sLex antigen and a TRPM4 polypeptide. In some embodiments, a target biomarker signature comprises at least two biomarkers, which is or comprises a sLex antigen and a TSPAN1 polypeptide. In some embodiments, a target biomarker signature comprises at least two biomarkers, which is or comprises a sLex antigen and an UNC13B polypeptide. In some embodiments, a target biomarker in the foregoing combinations may be used as a target of a capture probe and/or a target of a detection probe of assays described herein.

In some embodiments, a target biomarker signature comprises a combination of at least three biomarkers, which combination can be selected from the following: a ABCC4 polypeptide, a CLDN3 polypeptide, and a PMEPA1 polypeptide; or a ABCC4 polypeptide, a APOO polypeptide, and a RAB3B polypeptide; or a ABCC4 polypeptide, a MARCKSL1 polypeptide, and a RAB3B polypeptide; or a ABCC4 polypeptide, a GOLM1 polypeptide, and a PODXL2 polypeptide; or a ABCC4 polypeptide, a RAB3B polypeptide, and a SMPDL3B polypeptide; or a ABCC4 polypeptide, a GOLM1 polypeptide, and a RAB3B polypeptide; or a ABCC4 polypeptide, a CLDN3 polypeptide, and a SLC39A6 polypeptide; or a ABCC4 polypeptide, a CLDN3 polypeptide, and a HOMER2 polypeptide; or a ABCC4 polypeptide, a AP1M2 polypeptide, and a PMEPA1 polypeptide; or a ABCC4 polypeptide, a CLDN3 polypeptide, and a PPP3CA polypeptide; or a PMEPA1 polypeptide, a PPP3CA polypeptide, and a TSPAN1 polypeptide; or a PMEPA1 polypeptide, a SLC39A6 polypeptide, and a TSPAN1 polypeptide; or a PPP3CA polypeptide, a RAB3B polypeptide, and a SMPDL3B polypeptide; or a CLDN4 polypeptide, a FOLH1 polypeptide, and a PODXL2 polypeptide; or a FOLH1 polypeptide, a PODXL2 polypeptide, and a TSPAN1 polypeptide; or a GOLM1 polypeptide, a PODXL2 polypeptide, and a SORD polypeptide; or a FOLH1 polypeptide, a MARCKSL1 polypeptide, and a TSPAN1 polypeptide; or a AP1M2 polypeptide, a FOLH1 polypeptide, and a PODXL2 polypeptide; or a CLN5 polypeptide, a FOLH1 polypeptide, and a TSPAN1 polypeptide; or a PMEPA1 polypeptide, a PODXL2 polypeptide, and a TSPAN1 polypeptide; or a GOLM1 polypeptide, a PPP3CA polypeptide, and a RAB3B polypeptide; or a GOLM1 polypeptide, a PODXL2 polypeptide, and a RAB3B polypeptide; or a CLDN4 polypeptide, a FOLH1 polypeptide, and a MARCKSL1 polypeptide; or a PODXL2 polypeptide, a SLC39A6 polypeptide, and a TSPAN1 polypeptide; or a ENPP5 polypeptide, a RAB3B polypeptide, and a SMPDL3B polypeptide; or a CLDN3 polypeptide, a PODXL2 polypeptide, and a SLC39A6 polypeptide; or a GOLM1 polypeptide, a MARCKSL1 polypeptide, and a RAB3B polypeptide; or a GOLM1 polypeptide, a RAB3B polypeptide, and a SLC39A6 polypeptide; or a FOLH1 polypeptide, a MBOAT2 polypeptide, and a SMPDL3B polypeptide; or a FOLH1 polypeptide, a PODXL2 polypeptide, and a SMPDL3B polypeptide; or a PPP3CA polypeptide, a SLC39A6 polypeptide, and a TSPAN1 polypeptide; or a FOLH1 polypeptide, a HPN polypeptide, and a SMPDL3B polypeptide; or a PMEPA1 polypeptide, a RAB3D polypeptide, and a TSPAN1 polypeptide; or a MARCKSL1 polypeptide, a SLC39A6 polypeptide, and a TMPRSS2 polypeptide; or a GOLM1 polypeptide, a SLC39A6 polypeptide, and a TMPRSS2 polypeptide; or a ENPP5 polypeptide, a MARCKSL1 polypeptide, and a TMPRSS2 polypeptide; or a MARCKSL1 polypeptide, a PPP3CA polypeptide, and a TMPRSS2 polypeptide; or a GOLM1 polypeptide, a MARCKSL1 polypeptide, and a SORD polypeptide; or a CLGN polypeptide, a GOLM1 polypeptide, and a PPP3CA polypeptide; or a APOO polypeptide, a SLC39A6 polypeptide, and a TMPRSS2 polypeptide; or a MARCKSL1 polypeptide, a STEAP1 polypeptide, and a TMPRSS2 polypeptide; or a AP1M2 polypeptide, a CLGN polypeptide, and a PPP3CA polypeptide; or a CLGN polypeptide, a PPP3CA polypeptide, and a SMPDL3B polypeptide; or a AP1M2 polypeptide, a HPN polypeptide, and a STEAP2 polypeptide; or a CLGN polypeptide, a SLC39A6 polypeptide, and a SMPDL3B polypeptide; or a GRHL2 polypeptide, a HPN polypeptide, and a PPP3CA polypeptide; or a CLGN polypeptide, a GRHL2 polypeptide, and a MARCKSL1 polypeptide; or a AP1M2 polypeptide, a CLGN polypeptide, and a SLC4A4 polypeptide; or a CLGN polypeptide, a GRHL2 polypeptide, and a SMPDL3B polypeptide; or a CANT1 polypeptide, a CLGN polypeptide, and a SLC4A4 polypeptide; or a CANT1 polypeptide, a GRHL2 polypeptide, and a HPN polypeptide; or a ARFGEF3 polypeptide, a HPN polypeptide, and a TRPM4 polypeptide; or a EPCAM polypeptide, a HOMER2 polypeptide, and a STEAP2 polypeptide; or a EPCAM polypeptide, a SORD polypeptide, and a STEAP2 polypeptide; or a ARFGEF3 polypeptide, a SLC4A4 polypeptide, and a SORD polypeptide; or a GRHL2 polypeptide, a SLC4A4 polypeptide, and a STEAP1 polypeptide; or a GALNT3 polypeptide, a HPN polypeptide, and a STEAP2 polypeptide; or a GRHL2 polypeptide, a LCP1 polypeptide, and a SLC4A4 polypeptide; or a HPN polypeptide, a MBOAT2 polypeptide, and a RDH11 polypeptide; or a GALNT3 polypeptide, a GNG4 polypeptide, and a HPN polypeptide; or a CANT1 polypeptide, a SLC4A4 polypeptide, and a SYT7 polypeptide; or a EPCAM polypeptide, a HOMER2 polypeptide, and a STEAP1 polypeptide; or a CLDN4 polypeptide, a STEAP1 polypeptide, and a SYT7 polypeptide; or a CANT1 polypeptide, a RDH11 polypeptide, and a SLC4A4 polypeptide; or a MBOAT2 polypeptide, a RDH11 polypeptide, and a SLC4A4 polypeptide; or a GNG4 polypeptide, a HPN polypeptide, and a MBOAT2 polypeptide; or a MBOAT2 polypeptide, a SLC4A4 polypeptide, and a SYT7 polypeptide; or a HPN polypeptide, a RDH11 polypeptide, and a SLC35F2 polypeptide; or a CLDN4 polypeptide, a ENPP5 polypeptide, and a RDH11 polypeptide; or a CDH1 polypeptide, a ENPP5 polypeptide, and a SYT7 polypeptide; or a CANT1 polypeptide, a CDH1 polypeptide, and a ENPP5 polypeptide; or a EPCAM polypeptide, a GNG4 polypeptide, and a HOMER2 polypeptide; or a ENPP5 polypeptide, a EPCAM polypeptide, and a RDH11 polypeptide; or a CLDN4 polypeptide, a ENPP5 polypeptide, and a GNG4 polypeptide; or a CLDN4 polypeptide, a ENPP5 polypeptide, and a STEAP1 polypeptide; or a ENPP5 polypeptide, a MARVELD2 polypeptide, and a RDH11 polypeptide; or a CLDN4 polypeptide, a RDH11 polypeptide, and a STEAP1 polypeptide; or a CLDN4 polypeptide, a GNG4 polypeptide, and a STEAP1 polypeptide; or a GALNT3 polypeptide, a GNG4 polypeptide, and a STEAP1 polypeptide; or a EPCAM polypeptide, a HOMER2 polypeptide, and a LCP1 polypeptide; or a HOMER2 polypeptide, a NAAA polypeptide, and a TMC5 polypeptide; or a CADM4 polypeptide, a SERINC5 polypeptide, and a TMC5 polypeptide; or a MIA3 polypeptide, a SERINC5 polypeptide, and a TMC5 polypeptide; or a BCAM polypeptide, a FAAH polypeptide, and a TMC5 polypeptide; or a RAP1GAP polypeptide, a SERINC5 polypeptide, and a SLC35F2 polypeptide; or a SERINC5 polypeptide, a TMC5 polypeptide, and a TMEM192 polypeptide; or a SERINC5 polypeptide, a TMC5 polypeptide, and a VWA1 polypeptide; or a BCAM polypeptide, a SERINC5 polypeptide, and a TMC5 polypeptide; or a RAP1GAP polypeptide, a SHROOM2 polypeptide, and a SLC35F2 polypeptide; or a BCAM polypeptide, a NAAA polypeptide, and a PRSS8 polypeptide; or a ATP2C1 polypeptide, a RAP1GAP polypeptide, and a SLC35F2 polypeptide; or a BCAM polypeptide, a FAAH polypeptide, and a PRSS8 polypeptide; or a APOO polypeptide, a CYB561 polypeptide, and a FAAH polypeptide; or a BCAM polypeptide, a CYB561 polypeptide, and a PRSS8 polypeptide; or a CYB561 polypeptide, a RAP1GAP polypeptide, and a SLC35F2 polypeptide; or a CYB561 polypeptide, a FAAH polypeptide, and a SERINC5 polypeptide; or a BCAM polypeptide, a MIA3 polypeptide, and a PRSS8 polypeptide; or a FAAH polypeptide, a RAP1GAP polypeptide, and a SLC35F2 polypeptide; or a APOO polypeptide, a FAAH polypeptide, and a SERINC5 polypeptide; or a BCAM polypeptide, a PRSS8 polypeptide, and a SERINC5 polypeptide; or and combinations thereof. In some embodiments, at least one target biomarker in the foregoing combinations may be used as a target of a capture probe, and at least two biomarkers may be used as targets of detection probes.

In some embodiments, a target biomarker signature comprises at least three biomarkers, which is or comprises a ABCC4 polypeptide, a CLDN3 polypeptide, and a PMEPA1 polypeptide. In some embodiments, a target biomarker signature comprises at least three biomarkers, which is or comprises a ABCC4 polypeptide, a APOO polypeptide, and a RAB3B polypeptide. In some embodiments, a target biomarker signature comprises at least three biomarkers, which is or comprises a ABCC4 polypeptide, a MARCKSL1 polypeptide, and a RAB3B polypeptide. In some embodiments, a target biomarker signature comprises at least three biomarkers, which is or comprises a ABCC4 polypeptide, a GOLM1 polypeptide, and a PODXL2 polypeptide. In some embodiments, a target biomarker signature comprises at least three biomarkers, which is or comprises a ABCC4 polypeptide, a RAB3B polypeptide, and a SMPDL3B polypeptide. In some embodiments, a target biomarker signature comprises at least three biomarkers, which is or comprises a ABCC4 polypeptide, a GOLM1 polypeptide, and a RAB3B polypeptide. In some embodiments, a target biomarker signature comprises at least three biomarkers, which is or comprises a ABCC4 polypeptide, a CLDN3 polypeptide, and a SLC39A6 polypeptide. In some embodiments, a target biomarker signature comprises at least three biomarkers, which is or comprises a ABCC4 polypeptide, a CLDN3 polypeptide, and a HOMER2 polypeptide. In some embodiments, a target biomarker signature comprises at least three biomarkers, which is or comprises a ABCC4 polypeptide, a AP1M2 polypeptide, and a PMEPA1 polypeptide. In some embodiments, a target biomarker signature comprises at least three biomarkers, which is or comprises a ABCC4 polypeptide, a CLDN3 polypeptide, and a PPP3CA polypeptide. In some embodiments, a target biomarker signature comprises at least three biomarkers, which is or comprises a ABCC4 polypeptide, a PODXL2 polypeptide, and a RAB3B polypeptide. In some embodiments, a target biomarker signature comprises at least three biomarkers, which is or comprises a PMEPA1 polypeptide, a PPP3CA polypeptide, and a TSPAN1 polypeptide. In some embodiments, a target biomarker signature comprises at least three biomarkers, which is or comprises a PMEPA1 polypeptide, a SLC39A6 polypeptide, and a TSPAN1 polypeptide. In some embodiments, a target biomarker signature comprises at least three biomarkers, which is or comprises a PPP3CA polypeptide, a RAB3B polypeptide, and a SMPDL3B polypeptide. In some embodiments, a target biomarker signature comprises at least three biomarkers, which is or comprises a CLDN4 polypeptide, a FOLH1 polypeptide, and a PODXL2 polypeptide. In some embodiments, a target biomarker signature comprises at least three biomarkers, which is or comprises a FOLH1 polypeptide, a PODXL2 polypeptide, and a TSPAN1 polypeptide. In some embodiments, a target biomarker signature comprises at least three biomarkers, which is or comprises a GOLM1 polypeptide, a PODXL2 polypeptide, and a SORD polypeptide. In some embodiments, a target biomarker signature comprises at least three biomarkers, which is or comprises a FOLH1 polypeptide, a MARCKSL1 polypeptide, and a TSPAN1 polypeptide. In some embodiments, a target biomarker signature comprises at least three biomarkers, which is or comprises a CLN5 polypeptide, a FOLH1 polypeptide, and a TSPAN1 polypeptide. In some embodiments, a target biomarker signature comprises at least three biomarkers, which is or comprises a PMEPA1 polypeptide, a PODXL2 polypeptide, and a TSPAN1 polypeptide. In some embodiments, a target biomarker signature comprises at least three biomarkers, which is or comprises a ABCC4 polypeptide, a PMEPA1 polypeptide, and a TSPAN1 polypeptide. In some embodiments, a target biomarker signature comprises at least three biomarkers, which is or comprises a ABCC4 polypeptide, a FOLH1 polypeptide, and a GOLM1 polypeptide. In some embodiments, a target biomarker signature comprises at least three biomarkers, which is or comprises a ABCC4 polypeptide, a CLGN polypeptide, and a GOLM1 polypeptide. In some embodiments, a target biomarker signature comprises at least three biomarkers, which is or comprises a ABCC4 polypeptide, a CLDN3 polypeptide, and a FOLH1 polypeptide. In some embodiments, a target biomarker signature comprises at least three biomarkers, which is or comprises a ABCC4 polypeptide, a GOLM1 polypeptide, and a PMEPA1 polypeptide. In some embodiments, a target biomarker signature comprises at least three biomarkers, which is or comprises a ABCC4 polypeptide, a CDH1 polypeptide, and a PMEPA1 polypeptide. In some embodiments, a target biomarker signature comprises at least three biomarkers, which is or comprises a ABCC4 polypeptide, a CLDN3 polypeptide, and a ENPP5 polypeptide. In some embodiments, a target biomarker signature comprises at least three biomarkers, which is or comprises a ABCC4 polypeptide, a CLGN polypeptide, and a TSPAN1 polypeptide. In some embodiments, a target biomarker signature comprises at least three biomarkers, which is or comprises a EPCAM polypeptide, a FOLH1 polypeptide, and a PMEPA1 polypeptide. In some embodiments, a target biomarker signature comprises at least three biomarkers, which is or comprises a CLGN polypeptide, a PPP3CA polypeptide, and a TSPAN1 polypeptide. In some embodiments, at least one target biomarker in the foregoing combinations may be used as a target of a capture probe, and at least two biomarkers may be used as targets of detection probes.

III. Exemplary Methods of Detecting Provided Markers and/or Target Biomarker Signatures for Prostate Cancer

In general, the present disclosure provides technologies according to which a target biomarker signature is analyzed and/or assessed in a bodily fluid-derived sample (e.g., but not limited to a blood-derived sample, a urine-derived sample, etc.) comprising extracellular vesicles from a subject in need thereof; in some embodiments, a diagnosis or therapeutic decision is made based on such analysis and/or assessment.

In some embodiments, methods of detecting a target biomarker signature include methods for detecting one or more provided markers of a target biomarker signature as proteins, glycans, or proteoglycans (including, e.g., but not limited to a protein with a carbohydrate or glycan moiety). Exemplary protein-based methods of detecting one or more provided markers include, but are not limited to, proximity ligation assay, mass spectrometry (MS) and immunoassays, such as immunoprecipitation; Western blot; ELISA; immunohistochemistry; immunocytochemistry; flow cytometry; and immuno-PCR. In some embodiments, an immunoassay can be a chemiluminescent immunoassay. In some embodiments, an immunoassay can be a high-throughput and/or automated immunoassay platform.

In some embodiments, methods of detecting one or more provided markers as proteins, glycans, or proteoglycans (including, e.g., but not limited to a protein with a carbohydrate or glycan moiety) in a sample comprise contacting a sample with one or more antibody agents directed to the provided markers of interest. In some embodiments, such methods also comprise contacting the sample with one or more detection labels. In some embodiments, antibody agents are labeled with one or more detection labels.

In some embodiments, detecting binding between a biomarker of interest and an antibody agent for the biomarker of interest includes determining absorbance values or emission values for one or more detection agents. For example, the absorbance values or emission values are indicative of amount and/or concentration of biomarker of interest expressed by extracellular vesicles (e.g., higher absorbance is indicative of higher level of biomarker of interest expressed by extracellular vesicles). In some embodiments, absorbance values or emission values for detection agents are above a threshold value. In some embodiments, absorbance values or emission values for detection agents is at least 1.3, at least 1.4, at least 1.5, at least 1.6, at least 1.7, at least 1.8, at least 1.9, at least 2.0, at least 2.5, at least 3.0, at least 3.5 fold or greater than a threshold value. In some embodiments, the threshold value is determined across a population of a control or reference group (e.g., non-cancer subjects).

In some embodiments, methods of detecting one or more provided markers include methods for detecting one or more provided markers as nucleic acids. Exemplary nucleic acid-based methods of detecting one or more provided markers include, but are not limited to, performing nucleic acid amplification methods, such as polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). In some embodiments, a nucleic acid-based method of detecting one or more provided markers includes detecting hybridization between one or more nucleic acid probes and one or more nucleotide sequences that encode a biomarker of interest. In some embodiments, the nucleic acid probes are each complementary to at least a portion of one of the one or more nucleotide sequences that encode the biomarker of interest. In some embodiments, the nucleotide sequences that encode the biomarker of interest include DNA (e.g., cDNA). In some embodiments, the nucleotide sequences that encode the biomarker of interest include RNA. In some embodiments, the nucleotide sequences that encode the biomarker of interest may be or comprise mRNA. In some embodiments, the nucleotide sequences that encode the biomarker of interest may be or comprise microRNA. In some embodiments, the nucleotide sequences that encode the biomarker of interest may be or comprise noncoding RNA, which in some embodiments may be or comprise orphan noncoding RNA (oncRNA). In some embodiments, the nucleotide sequences that encode the biomarker of interest may be or comprise long noncoding RNA (lncRNA). In some embodiments, the nucleotide sequences that encode the biomarker of interest may be or comprise piwi-interacting RNA (piwiRNA). In some embodiments, the nucleotide sequences that encode the biomarker of interest may be or comprise circular RNA (circRNA). In some embodiments, the nucleotide sequences that encode the biomarker of interest may be or comprise small nucleolar RNA (snoRNA).

In some embodiments, methods of detecting one or more provided markers involve proximity-ligation-immuno quantitative polymerase chain reaction (pliq-PCR). Pliq-PCR can have certain advantages over other technologies to profile EVs. For example, pliq-PCR can have a sensitivity three orders of magnitude greater than other standard immunoassays, such as ELISAs (Darmanis et al., 2010; which is incorporated herein by reference for the purpose described herein). In some embodiments, a pliq-PCR reaction can be designed to have an ultra-low LOD, which enables to detect trace levels of tumor-derived EVs, for example, down to a thousand EVs per mL.

In some embodiments, methods for detecting one or more provided markers may involve other technologies for detecting EVs, including, e.g., Nanoplasmic Exosome (nPLEX) Sensor (Im et al., 2014; which is incorporated herein by reference for the purpose described herein) and the Integrated Magnetic-Electrochemical Exosome (iMEX) Sensor (Jeong et al., 2016; which is incorporated herein by reference for the purpose described herein), which have reported LODs of ˜10³ and ˜10⁴ EVs, respectively (Shao et al., 2018; which is incorporated herein by reference for the purpose described herein).

In some embodiments, methods for detecting one or more provided biomarkers in extracellular vesicles can be based on bulk EV sample analysis.

In some embodiments, methods for detecting one or more provided biomarkers in extracellular vesicles can be based on profiling individual EVs (e.g., single-EV profiling assays), which is further discussed in the section entitled “Exemplary Methods for Profiling Individual Nanoparticles Having a Size Range of Interest that Includes Extracellular Vesicles (EVs)” below.

A skilled artisan reading the present disclosure will understand that the assays described herein for detecting or profiling individual EVs can be also used to detect biomarker combinations on the surface of nanoparticles having a size range of interest (e.g., as described herein) that includes extracellular vesicles (e.g., as described herein).

In some embodiments, nanoparticles having a size range of interest that includes extracellular vesicles in a sample may be captured or immobilized on a solid substrate prior to detecting one or more provided biomarkers in accordance with the present disclosure. In some embodiments, nanoparticles having a size range of interest that includes extracellular vesicles may be captured on a solid substrate surface by non-specific interaction, including, e.g., adsorption. In some embodiments, nanoparticles having a size range of interest that includes extracellular vesicles may be selectively captured on a solid substrate surface. For example, in some embodiments, a solid substrate surface may be coated with an agent that specifically binds to nanoparticles having a size range of interest that includes extracellular vesicles (e.g., an antibody agent specifically targeting such nanoparticles, e.g., associated with prostate cancer). In some embodiments, a solid substrate surface may be coated with a member of an affinity binding pair and an entity of interest (e.g., extracellular vesicles) to be captured may be conjugated to a complementary member of the affinity binding pair. In some embodiments, an exemplary affinity binding pair includes, e.g., but is not limited to biotin and avidin-like molecules such as streptavidin. As will be understood by those of skilled in the art, other appropriate affinity binding pairs can also be used to facilitate capture of an entity of interest to a solid substrate surface. In some embodiments, an entity of interest may be captured on a solid substrate surface by application of a current, e.g., as described in Ibsen et al. ACS Nano., 11:6641-6651 (2017) and Lewis et al. ACS Nano., 12:3311-3320 (2018), both of which are incorporated herein by reference for the purpose described herein, and both of which describe use of an alternating current electrokinetic microarray chip device to isolate extracellular vesicles from an undiluted human blood or plasma sample.

A solid substrate may be provided in a form that is suitable for capturing nanoparticles having a size range of interest that includes extracellular vesicles and does not interfere with downstream handling, processing, and/or detection. For example, in some embodiments, a solid substrate may be or comprise a bead (e.g., a magnetic bead). In some embodiments, a solid substrate may be or comprise a surface. For example, in some embodiments, such a surface may be a capture surface of an assay chamber (including, e.g., a tube, a well, a microwell, a plate, a filter, a membrane, a matrix, etc.). Accordingly, in some embodiments, a method described herein comprises, prior to detecting provided biomarkers in a sample, capturing or immobilizing nanoparticles having a size range of interest that includes extracellular vesicles on a solid substrate.

In some embodiments, a sample may be processed, e.g., to remove undesirable entities such as cell debris or cells, prior to capturing nanoparticles having a size range of interest that includes extracellular vesicles on a solid substrate surface. For example, in some embodiments, such a sample may be subjected to centrifugation, e.g., to remove cell debris, cells, and/or other particulates. Additionally or alternatively, in some embodiments, such a sample may be subjected to size-exclusion-based purification or filtration. Various size-exclusion-based purification or filtration are known in the art and those skilled in the art will appreciate that in some cases, a sample may be subjected to a spin column purification based on specific molecular weight or particle size cutoff. Those skilled in the art will also appreciate that appropriate molecular weight or particle size cutoff for purification purposes can be selected, e.g., based on the size of extracellular vesicles. For example, in some embodiments, size-exclusion separation methods may be applied to samples comprising extracellular vesicles to isolate a fraction of nanoparticles that include extracellular vesicles of a certain size (e.g., greater than 30 nm and no more than 1000 nm, or greater than 70 nm and no more than 200 nm). Typically, extracellular vesicles may range from 30 nm to several micrometers in diameter. See, e.g., Chuo et al., “Imaging extracellular vesicles: current and emerging methods” Journal of Biomedical Sciences 25:91 (2018) which is incorporated herein by reference for the purpose described herein, which provides information of sizes for different extracellular vesicle (EV) subtypes: migrasomes (0.5-3 μm), microvesicles (0.1-1 μm), oncosomes (1-10 μm), exomeres (<50 nm), small exosomes (60-80 nm), and large exosomes (90-120 nm). In some embodiments, nanoparticles having a size range of about 30 nm to 1000 nm may be isolated, for example, in some embodiments by one or more size-exclusion separation methods, for detection assay. In some embodiments, specific EV subtype(s) may be isolated, for example, in some embodiments by one or more size-exclusion separation methods, for detection assay.

In some embodiments, nanoparticles having a size range of interest that includes extracellular vesicles in a sample may be processed prior to detecting one or more provided biomarkers of a target biomarker signature for prostate cancer. Different sample processing and/or preparation can be performed, e.g., to stabilize targets (e.g., target biomarkers) in extracellular vesicles to be detected, and/or to facilitate exposure of targets (e.g., intravesicular proteins and/or RNA such as mRNA) to a detection assay (e.g., as described herein), and/or to reduce non-specific binding. Examples of such sample processing and/or preparation are known in the art and include, but are not limited to, crosslinking molecular targets (e.g., fixation), permeabilization of biological entities (e.g., cells or nanoparticles having a size range of interest that includes extracellular vesicles), and/or blocking non-specific binding sites.

In one aspect, the present disclosure provides a method for detecting whether a target biomarker signature of prostate cancer is present or absent in a biological sample from a subject in need thereof, which may be in some embodiments a biological sample (e.g., but not limited to a blood-derived sample, a urine-derived sample, etc.) comprising nanoparticles having a size range of interest that includes extracellular vesicles. In some embodiments, such a method comprises (a) detecting, in a biological sample such as a blood-derived sample (e.g., a plasma sample) from a subject, biological entities of interest (including, e.g., nanoparticles having a size range of interest that includes extracellular vesicles) having a target biomarker signature of prostate cancer; and (b) comparing sample information indicative of the level of the target biomarker signature-expressing biological entities of interest (e.g., nanoparticles having a size range of interest that includes extracellular vesicles) in the biological sample (e.g., blood-derived sample) to reference information including a reference threshold level. In some embodiments, a reference threshold level corresponds to a level of biological entities of interest (e.g., nanoparticles having a size range of interest that includes extracellular vesicles) that express such a target biomarker signature in comparable samples from a population of reference subjects, e.g., non-cancer subjects. In some embodiments, exemplary non-cancer subjects include healthy subjects (e.g., healthy subjects of specified age ranges, such as e.g., below age 55 or above age 55), subjects with non-prostate-related health diseases, disorders, or conditions (including, e.g., subjects having non-prostate cancer such as lung cancer, ovarian cancer, etc., or subjects having symptoms of inflammatory diseases or disorders), subjects having benign prostatic hyperplasia, and combinations thereof.

In some embodiments, a sample is pre-screened for certain characteristics prior to utilization in an assay as described herein. In some embodiments, a sample meeting certain pre-screening criteria is more suitable for diagnostic applications than a sample failing pre-screening criteria. For example, in some embodiments samples are visually inspected for appearance using known standards, e.g., is the sample normal, hemolyzed (red), icteric (yellow), and/or lipemic (whitish/turbid). In some embodiments, samples can then be rated on a known standard scale (e.g., 1, 2, 3, 4, 5) and the results are recorded. In some embodiments, samples are visually inspected for hemolysis (e.g., heme) and rated on a scale from 1-5, where the visual inspection correlates with a known concentration, e.g., where 1 denotes approximately 0 mg/dL, 2 denotes approximately 50 mg/dL, 3 denotes approximately 150 mg/dL, 4 denotes approximately 250 mg/dL, and 5 denotes approximately 525 mg/dL. In some embodiments, samples are visually inspected icteric levels (e.g., bilirubin) and rated on a scale from 1-5, where the visual inspection correlates with a known concentration, e.g., where 1 denotes approximately 0 mg/dL, 2 denotes approximately 1.7 mg/dL. 3 denotes approximately 6.6 mg/dL, 4 denotes approximately 16 mg/dL, and 5 denotes approximately 30 mg/dL. In some embodiments, samples are visually inspected for turbidity (e.g. lipids) and rated on a scale from 1-5, where the visual inspection correlates with a known concentration, e.g., where 1 denotes approximately 0 mg/dL, 2 denotes approximately 125 mg/dL. 3 denotes approximately 250 mg/dL, 4 denotes approximately 500 mg/dL, and 5 denotes approximately 1000 mg/dL.

In some embodiments, samples scoring lower than a certain level on one or more metrics, e.g., equal to or lower than a score of 4, may be utilized in an assay as described herein. In some embodiments, samples scoring lower than a certain level on one or more metrics, e.g., equal to or lower than a score of 3, may be utilized in an assay as described herein. In some embodiments, samples scoring lower than a certain level on one or more metrics, e.g., equal to or lower than a score of 2, may be utilized in an assay as described herein. In some embodiments, samples scoring lower than a certain level on all three metrics (e.g., hemolyzed, icteric, and lipemic) e.g., equal to or lower than a score of 2, may be utilized in an assay as described herein. In some embodiments, low visual inspection scores on pre-screening criteria such as hemolysis, bilirubin, and/or lipemia (e.g., equal to or lower than a score of 2) may have no appreciable effect (e.g., not be correlated with) on diagnostic properties (e.g., Ct values) produced in an assay as described herein.

In some embodiments, a sample is determined to be positive for the presence of a target biomarker signature (e.g., ones described herein) when it shows an elevated level of nanoparticles (having a size range of interest that includes extracellular vesicles) that present the target biomarker signature on their surface, relative to a reference threshold level (e.g., ones described herein). In some embodiments, a sample is determined to be positive for the presence of a target biomarker signature (e.g., as reflected by the level of target biomarker signature-expressing extracellular vesicles) if its level is at least 30% or higher, including, e.g., at least 40%, at least 50%, at least 60%, at least 70%, at least 80%, at least 90%, at least 95% or higher, as compared to a reference threshold level. In some embodiments, a sample is determined to be positive for the presence of a target biomarker signature (e.g., as reflected by the level of target biomarker signature-expressing extracellular vesicles) if its level is at least 2-fold or higher, including, e.g., at least 3-fold, at least 4-fold, at least 5-fold, at least 6-fold, at least 7-fold, at least 8-fold, at least 9-fold, at least 10-fold, at least 50-fold, at least 100-fold, at least 250-fold, at least 500-fold, at least 750-fold, at least 1000-fold, at least 2500-fold, at least 5000-fold, or higher, as compared to a reference threshold level.

In some embodiments, a binary classification system may be used to determine whether a sample is positive for the presence of a target biomarker signature. For example, in some embodiments, a sample is determined to be positive for the presence of a target biomarker signature (e.g., as reflected by the level of target biomarker signature-expressing extracellular vesicles) if its level is at or above a reference threshold level, e.g., a cutoff value. In some embodiments, such a reference threshold level (e.g., a cutoff value) may be determined by selecting a certain number of standard deviations away from an average value obtained from control subjects such that a desired sensitivity and/or specificity of a prostate cancer detection assay (e.g., ones described herein) can be achieved. In some embodiments, such a reference threshold level (e.g., a cutoff value) may be determined by selecting a certain number of standard deviations away from a maximum assay signal obtained from control subjects such that a desired sensitivity and/or specificity of a prostate cancer detection assay (e.g., ones described herein) can be achieved. In some embodiments, such a reference threshold level (e.g., a cutoff value) may be determined by selecting the less restrictive of either (i) a certain number of standard deviations away from an average value obtained from control subjects, or (ii) a certain number of standard deviations away from a maximum assay signal obtained from control subjects, such that a desired sensitivity and/or specificity of a prostate cancer detection assay (e.g., ones described herein) can be achieved. In some embodiments, control subjects for determination of a reference threshold level (e.g., a cutoff value) may include, but are not limited to healthy subjects, subjects with inflammatory conditions (e.g., benign prostatic hyperplasia, prostatitis, etc.), subjects with benign tumors, and combinations thereof. In some embodiments, healthy subjects and subjects with inflammatory conditions (e.g., benign prostatic hyperplasia) are included in determination of a reference threshold level (e.g., a cutoff value). In some embodiments, subjects with benign prostatic hyperplasia are not included in determination of a reference threshold level (e.g., a cutoff value). In some embodiments, a reference threshold level (e.g., a cutoff value) may be determined by selecting at least 1.5 standard deviations (SDs) or higher (including, e.g., at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6 or higher SDs) away from (i) an average value obtained from control subjects, or (ii) a maximum assay signal obtained from control subjects, such that a desired specificity (e.g., at least 95% or higher specificity [including, e.g., at least 96%, at least 97%, at least 98%, at least 99%, or higher specificity] such as in some embodiments at least 99.8% specificity) of a prostate cancer detection assay (e.g., ones described herein) can be achieved. In some embodiments, a reference threshold level (e.g., a cutoff value) may be determined by selecting at least 2.9 SDs (e.g., at least 2.93 SDs) away from (i) an average value obtained from control subjects, or (ii) a maximum assay signal obtained from control subjects, such that a desired specificity (e.g., at least 99%, or higher specificity) of a prostate cancer detection assay (e.g., ones described herein) can be achieved. In some embodiments, a reference threshold level (e.g., a cutoff value) may be determined by selecting at least 2.9 SDs (e.g., at least 2.93 SDs) away from the less restrictive of (i) an average value obtained from control subjects, or (ii) a maximum assay signal obtained from control subjects, such that a desired specificity (e.g., at least 99%, or higher specificity) of a prostate cancer detection assay (e.g., ones described herein) can be achieved. In some embodiments, such a reference threshold level (e.g., a cutoff value) may be determined based on expression level (e.g., transcript level) of a target biomarker in normal healthy tissues vs. in prostate cancer samples such that the specificity and/or sensitivity of interest (e.g., as described herein) can be achieved. In some embodiments, a reference threshold level (e.g., a cutoff value) may vary dependent on, for example, prostate cancer stages and/or subtypes and/or patient characteristics, for example, patient age, risks factors for prostate cancer (e.g., hereditary risk vs, average risk, life-history-associated risk factors), symptomatic/asymptomatic status, and combinations thereof.

In some embodiments, a reference threshold level (e.g., a cutoff value) may be determined based on a log-normal distribution around healthy subjects (e.g., of specified age ranges), and optionally subjects with inflammatory conditions (e.g., benign prostatic hyperplasia) and selection of a level that is necessary to achieve the specificity of interest, e.g., based on prevalence of prostate cancer or a subtype thereof (e.g., including but not limited to prostate adenocarcinoma). In some embodiments, specificity of interest may be at least 70%, including, e.g., at least 75%, at least 80%, at least 85%, at least 90%, at least 95%, at least 98%, at least 99%, at least 99.5% or higher.

The present disclosure, among other things, also provides technologies for determining whether a subject as having or being susceptible to prostate cancer, for example, from a sample comprising nanoparticles with a size range of interest that includes extracellular vesicles. For example, in some embodiments, when a bodily fluid-derived sample (e.g., but not limited to a blood-derived sample, a urine-derived sample, etc.) from a subject in need thereof shows a level of target biomarker signature-expressing extracellular vesicles that is at or above a reference threshold level, e.g., cutoff value (e.g., as determined in accordance with the present disclosure), then the subject is classified as having or being susceptible to prostate cancer. In some such embodiments, a reference threshold level (e.g., cutoff value) may be determined based on a log-normal distribution around healthy subjects (e.g., of specified age ranges), and optionally subjects with inflammatory conditions (e.g., benign prostatic hyperplasia) and selection of a level that is necessary to achieve the specificity of interest, e.g., based on prevalence of prostate cancer or a subtype thereof (e.g., prostate adenocarcinoma). In some embodiments, specificity of interest may be at least 70%, including, e.g., at least 75%, at least 80%, at least 85%, at least 90%, at least 95%, at least 98%, at least 99%, at least 99.5% or higher.

In some embodiments, a reference threshold level (e.g., a cutoff value) may be determined based on expression level (e.g., transcript level) of individual target biomarker(s) of a target biomarker signature in normal healthy tissues vs. in prostate cancer samples such that the specificity and/or sensitivity of interest (e.g., as described herein) can be achieved. In some embodiments, a reference threshold level (e.g., a cutoff value) may vary dependent on, for example, prostate cancer stages, Gleason score, and/or subtypes and/or patient characteristics, for example, patient age, risks factors for prostate cancer (e.g., hereditary risk vs, average risk, life-history-associated risk factors), symptomatic/asymptomatic status, and combinations thereof.

In some embodiments, when a biological sample from a subject in need thereof shows a level of biomarker combination that satisfies a reference threshold level, then the subject is classified as having or being susceptible to prostate cancer. For example, in some embodiments, when a bodily fluid-derived sample (e.g., but not limited to a blood-derived sample, a urine-derived sample, etc.) from a subject in need thereof shows an elevated level of target biomarker signature-expressing extracellular vesicles relative to a reference threshold level, then the subject is classified as having or being susceptible to prostate cancer.

In some embodiments, a subject in need thereof is classified as having or being susceptible to prostate cancer when the subject's bodily fluid-derived sample (e.g., but not limited to a blood-derived sample, a urine-derived sample, etc.) shows a level of target biomarker signature-expressing extracellular vesicles that is at least 30% or higher, including, e.g., at least 40%, at least 50%, at least 60%, at least 70%, at least 80%, at least 90%, at least 95% or higher, as compared to a reference threshold level. In some embodiments, a subject in need thereof is classified as having or being susceptible to prostate cancer when the subject's bodily fluid-derived sample (e.g., but not limited to a blood-derived sample, a urine-derived sample, etc.) shows a level of target biomarker signature-expressing extracellular vesicles that is at least 2-fold or higher, including, e.g., at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 250-fold, at least 500-fold, at least 750-fold, at least 1000-fold, or higher, as compared to a reference threshold level.

When a biological sample from a subject in need thereof shows a comparable level to a reference threshold level, then the subject is classified as not likely to have or as not likely to be susceptible to prostate cancer. In some such embodiments, a reference threshold level corresponds to a level of extracellular vesicles that express a target biomarker signature in comparable samples from a population of reference subjects, e.g., non-cancer subjects. In some embodiments, exemplary non-cancer subjects include healthy subjects (e.g., healthy subjects of specified age ranges, such as e.g., below age 55 or above age 55), subjects with non-prostate related health diseases, disorders, or conditions (including, e.g., subjects having non-prostate cancer such as lung cancer, ovarian cancer, etc., or subjects having symptoms of inflammatory diseases or disorders), subjects having benign prostatic hyperplasia, and combinations thereof.

IV. Exemplary Methods for Profiling Individual Nanoparticles Having a Size Range of Interest that Includes Extracellular Vesicles (EVs)

In some embodiments, assays for profiling individual extracellular vesicles (e.g., single EV profiling assays) can be used to detect one or more provided biomarkers of one or more target biomarker signatures for prostate cancer. For example, in some embodiments, such an assay may involve (i) a capture assay through targeting one or more provided markers of a target biomarker signature for prostate cancer and (ii) a detection assay for at least one or more additional provided markers of such a target biomarker signature for prostate cancer, wherein such a capture assay is performed prior to such a detection assay.

A skilled artisan reading the present disclosure will understand that assays described herein for detecting or profiling individual extracellular vesicles can also detect surface biomarkers present on the surfaces of nanoparticles having a size of interest (e.g., in some embodiments a size within the range of about 30 nm to about 1000 nm) that includes extracellular vesicles.

In some embodiments, a capture assay is performed to selectively capture tumor-associated nanoparticles having a size range of interest that includes extracellular vesicles (e.g., prostate tumor-associated extracellular vesicles) from a bodily fluid-derived sample (e.g., but not limited to a blood-derived sample, a urine-derived sample, etc.) of a subject in need thereof. In some embodiments, a capture assay is performed to selectively capture nanoparticles of a certain size range that includes extracellular vesicles, and/or certain characteristic(s), for example, extracellular vesicles associated with prostate cancer. In some such embodiments, prior to a capture assay, a bodily fluid-derived sample (e.g., but not limited to a blood-derived sample, a urine-derived sample, etc.) may be pre-processed to remove contaminants, including, e.g., but not limited to soluble proteins and interfering entities such as, e.g., cell debris. For example, in some embodiments, nanoparticles having a size range of interest that includes extracellular vesicles are purified from a bodily fluid-derived sample (e.g., but not limited to a blood-derived sample, a urine-derived sample, etc.) of a subject using size exclusion chromatography. In some such embodiments, nanoparticles having a size range of interest that includes extracellular vesicles can be directly purified from a bodily fluid-derived sample (e.g., but not limited to a blood-derived sample, a urine-derived sample, etc.) using size exclusion chromatography, which in some embodiments may remove at least 90% or higher (including, e.g., at least 93%, 95%, 97%, 99% or higher) of soluble proteins and other interfering agents such as, e.g., cell debris.

In some embodiments, a capture assay comprises a step of contacting a bodily fluid-derived sample (e.g., but not limited to a blood-derived sample, a urine-derived sample, etc.) with at least one capture agent comprising a target-capture moiety that binds to at least one or more provided biomarkers of a target biomarker signature for prostate cancer. In some embodiments, a capture assay may be multiplexed, which comprises a step of contacting a bodily fluid-derived sample (e.g., but not limited to a blood-derived sample, a urine-derived sample, etc.) with a set of capture agents, each capture agent comprising a target-capture moiety that binds to a distinct provided biomarker of a target biomarker signature for prostate cancer. In some embodiments, a target-capture moiety is directed to an extracellular vesicle-associated surface biomarker or surface biomarker (e.g., ones as described and/or utilized herein).

In some embodiments, such a target-capture moiety may be immobilized on a solid substrate. Accordingly, in some embodiments, a capture agent employed in a capture assay is or comprises a solid substrate comprising at least one or more (e.g., 1, 2, 3, 4, 5, or more) target-capture moiety conjugated thereto, each target-capture moiety directed to an extracellular vesicle-associated surface biomarker and/or surface biomarker (e.g., ones as described and/or utilized herein). A solid substrate may be provided in a form that is suitable for capturing nanoparticles having a size range of interest that includes extracellular vesicles and does not interfere with downstream handling, processing, and/or detection. For example, in some embodiments, a solid substrate may be or comprise a bead (e.g., a magnetic bead). In some embodiments, a solid substrate may be or comprise a surface. For example, in some embodiments, such a surface may be a capture surface of an assay chamber (including, e.g., a tube, a well, a microwell, a plate, a filter, a membrane, a matrix, etc.). In some embodiments, a capture agent is or comprises a magnetic bead comprising a target-capture moiety conjugated thereto.

In some embodiments, a detection assay is performed to detect one or more provided biomarkers of a target biomarker signature for prostate cancer (e.g., ones that are different from ones targeted in a capture assay) in nanoparticles having a size range of interest that includes extracellular vesicles that are captured by a capture assay (e.g., as described above). In some embodiments, a detection assay may comprise immuno-PCR. In some embodiments, an immuno-PCR may involve at least one probe targeting a single provided biomarker (e.g., ones described herein) of a target biomarker signature for prostate cancer. In some embodiments, an immuno-PCR may involve a plurality of (e.g., at least two, at least three, at least four, or more) probes directed to different epitopes of the same biomarker (e.g., ones described herein) of a target biomarker signature. In some embodiments, an immuno-PCR may involve a plurality of (e.g., at least two, at least three, at least four, or more) probes, each directed to a different provided biomarker described herein.

In some embodiments, a detection assay may comprise reverse transcription polymerase chain reaction (RT-PCR). In some embodiments, an RT-PCR may involve at least one primer/probe set targeting a single provided biomarker described herein. In some embodiments, an RT-PCR may involve a plurality of (e.g., at least two, at least three, at least four, or more) primer/probe sets, each set directed to a different provided biomarker described herein.

In some embodiments, a detection assay may comprise a proximity-ligation-immuno quantitative polymerase chain reaction (pliq-PCR), for example, to determine co-localization of one or more provided biomarkers of a target biomarker signature for prostate cancer within nanoparticles having a size range of interest that includes extracellular vesicles (e.g., captured extracellular vesicles that express at least one extracellular vesicle-associated surface biomarker).

In some embodiments, a detection assay employs a target entity detection system that was developed by Applicant and described in U.S. application Ser. No. 16/805,637 (published as US2020/0299780; issued as U.S. Pat. No. 11,085,089), and International Application PCT/US2020/020529 (published as WO2020180741), both filed Feb. 28, 2020 and entitled “Systems, Compositions, and Methods for Target Entity Detection” (the “'089 patent” and the “'529 application”; both of which are incorporated herein by reference in their entirety) which are, in part, based on interaction and/or co-localization of a target biomarker signature in individual extracellular vesicles. For example, such a target entity detection system (as described in the '089 patent and '529 application and also further described below in the section entitled “Provided Target Entity Detection Systems and Methods Involving the Same”) can detect in a sample (e.g., in a biological, environmental, or other sample), in some embodiments at a single entity level, entities of interest (e.g., biological or chemical entities of interest, such as extracellular vesicles or analytes) comprising at least one or more (e.g., at least two or more) targets (e.g., molecular targets). Those skilled in the art, reading the present disclosure, will recognize that provided target entity detection systems are useful for a wide variety of applications and/or purposes, including, e.g., for detection of prostate cancer. For example, in some embodiments, provided target entity detection systems may be useful for medical applications and/or purposes. In some embodiments, provided target entity detection systems may be useful to screen (e.g., regularly screen) individuals (e.g., in some embodiments which may be asymptomatic individuals, or in some embodiments which may be individuals experiencing one or more symptoms associated with prostate cancer, or in some embodiments which may be individuals at risk for prostate cancer such as, e.g., individuals with a hereditary risk for prostate cancer and/or life-history-associated risk factor, including individuals who smoke and/or are obese) for a disease or condition (e.g., prostate cancer). In some embodiments, provided target entity detection systems may be useful to screen (e.g., regularly screen) individuals (e.g., in some embodiments which may be asymptomatic individuals, or in some embodiments which may be individuals experiencing one or more symptoms associated with prostate cancer, or in some embodiments which may be individuals at risk for prostate cancer such as, e.g., individuals with a hereditary risk for prostate cancer and/or life-history-associated risk factor, including individuals who smoke and/or are obese) for different types of cancer (e.g., for a plurality of different cancers, one of which may be prostate cancer). In some embodiments, provided target entity detection systems are effective even when applied to populations comprising or consisting of asymptomatic individuals (e.g., due to sufficiently high sensitivity and/or low rates of false positive and/or false negative results). In some embodiments, provided target entity detection systems may be useful as a companion diagnostic in conjunction with a disease treatment (e.g., treatment of prostate cancer).

In some embodiments, a plurality of (e.g., at least two or more) detection assays may be performed to detect a plurality of biomarkers (e.g., at least two or more) of one or more target biomarker signatures for prostate cancer (e.g., ones that are different from ones targeted in a capture assay) in nanoparticles having a size range of interest that includes extracellular vesicles, e.g., ones that are captured by a capture assay (e.g., as described above). For example, in some embodiments, a plurality of detection assays may comprise (i) a provided target entity detection system or a system described in the '089 patent and '529 application and/or described herein; and (ii) immuno-PCR. In some embodiments, a plurality of detection assays may comprise (i) a provided target entity detection system or a system described in the '089 patent and '529 application and/or described herein; and (ii) RT-PCR.

For example, in some embodiments, a subject's sample comprising extracellular vesicles may be first subjected to detection of surface biomarkers (e.g., as described herein) using a target entity detection system or a system described in the '089 patent and '529 application and/or described herein and then subjected to a lysis buffer to release intravesicular analytes, followed by a nucleic acid assay (e.g., in some embodiments RT-qPCR) for detection of one or more intravesicular RNA biomarkers. In some embodiments, one or more intravesicular RNA biomarkers may be or comprise an mRNA transcript encoded by a biomarker gene described herein. In some embodiments, one or more intravesicular RNA biomarkers may be or comprise a microRNA. In some embodiments, one or more intravesicular RNA biomarkers may be or comprise an orphan noncoding RNA. In some embodiments, one or more intravesicular RNA biomarkers may be or comprise a long noncoding RNA. In some embodiments, one or more intravesicular RNA biomarkers may be or comprise a piwi-interacting RNA. In some embodiments, one or more intravesicular RNA biomarkers may be or comprise a circular RNA. In some embodiments, one or more intravesicular RNA biomarkers may be or comprise a small nucleolar RNA.

### V. Provided Target Entity Detection Systems and Methods Involving the Same

In some embodiments, a target entity detection system that can be useful in a detection assay for one or more provided biomarkers of one or more target biomarker signatures for prostate cancer includes a plurality of detection probes each for a specific target (e.g., a provided biomarker of a target biomarker signature). In some embodiments, such a system may comprise at least 2, at least 3, at least 4, at least 5, at least 6, at least 7, at least 8, at least 9, at least 10, at least 15, at least 20, at least 25, at least 30, at least 40, at least 50, or more detection probes each for a specific target (e.g., a provided biomarker of a target biomarker signature). In some embodiments, such a system may comprise 2-50 detection probes each for a specific target (e.g., a provided biomarker of a target biomarker signature). In some embodiments, such a system may comprise 2-30 detection probes each for a specific target (e.g., a provided biomarker of a target biomarker signature). In some embodiments, such a system may comprise 2-25 detection probes each for a specific target (e.g., a provided biomarker of a target biomarker signature). In some embodiments, such a system may comprise 5-30 detection probes each for a specific target (e.g., a provided biomarker of a target biomarker signature). In some embodiments, such a system may comprise 5-25 detection probes each for a specific target (e.g., a provided biomarker of a target biomarker signature). In some embodiments, at least two of such detection probes in a set may be directed to the same biomarker of a target biomarker signature. In some embodiments, at least two of such detection probes in a set may be directed to the same epitope of the same biomarker of a target biomarker signature. In some embodiments, at least two of such detection probes in a set may be directed to different epitopes of the same biomarker of a target biomarker signature.

In some embodiments, detection probes appropriate for use in a target entity detection system provided herein may be used for detection of a single disease or condition, e.g., prostate cancer. In some embodiments, detection probes appropriate for use in a target entity detection system provided herein may permit detection of at least two or more diseases or conditions, e.g., one of which is prostate cancer. In some embodiments, detection probes appropriate for use in a target entity detection system provided herein may permit detection of prostate cancer of certain subtypes including but not limited to, e.g., prostate adenocarcinoma, and other specified types of cancer as known in the art (SEER Cancer Statistics Review 1975-2017). In some embodiments, detection probes appropriate for use in a target entity detection system provided herein may permit detection of prostate cancer of certain stages, including, e.g., stage I, stage II, stage III, and/or stage IV. Accordingly, in some embodiments, detection probes appropriate for use in a target entity detection system provided herein may comprise a plurality (e.g., at least 2, at least 3, at least 4, at least 5, at least 6, at least 7, at least 8, at least 9, at least 10, or more) of sets of detection probes, wherein each set is directed to detection of a different disease or a different type of disease or condition. For example, in some embodiments, detection probes appropriate for use in a target entity detection system provided herein may comprise a plurality (e.g., at least 2, at least 3, at least 4, at least 5, at least 6, at least 7, at least 8, at least 9, at least 10, or more) of sets of detection probes, wherein in some embodiments, each set is directed to detection of a different type of cancer, one of which is prostate cancer, or in some embodiments, each set is directed to detection of prostate cancer of various subtypes (e.g., prostate adenocarcinoma) and/or stages.

### Detection Probes

In some embodiments, a detection probe as provided and/or utilized herein comprises a target-binding moiety and an oligonucleotide domain coupled to the target-binding moiety. In some embodiments, an oligonucleotide domain coupled to a target-binding moiety may comprise a double-stranded portion and a single-stranded overhang extended from at least one end of the oligonucleotide domain. In some embodiments, an oligonucleotide domain coupled to a target-binding moiety may comprise a double-stranded portion and a single-stranded overhang extended from each end of the oligonucleotide domain. In some embodiments, detection probes may be suitable for proximity-ligation-immuno quantitative polymerase chain reaction (pliq-PCR) and be referred to as pliq-PCR detection probes.

### A. Target-Binding Moieties

A target-binding moiety that is coupled to an oligonucleotide domain is an entity or an agent that specifically binds to a target (e.g., a provided biomarker of a target biomarker signature; those skilled in the art will appreciate that, where the target biomarker is a particular form or moiety/component, the target-binding moiety specifically binds to that form or moiety/component). In some embodiments, a target-binding moiety may have a binding affinity (e.g., as measured by a dissociation constant) for a target (e.g., molecular target) of at least about 10⁻⁴M, at least about 10⁻⁵M, at least about 10⁻⁶M, at least about 10⁻⁷M, at least about 10⁻⁸M, at least about 10⁻⁹M, or lower. Those skilled in the art will appreciate that, in some cases, binding affinity (e.g., as measured by a dissociation constant) may be influenced by non-covalent intermolecular interactions such as hydrogen bonding, electrostatic interactions, hydrophobic and Van der Waals forces between the two molecules. Alternatively or additionally, binding affinity between a ligand and its target molecule may be affected by the presence of other molecules. Those skilled in the art will be familiar with a variety of technologies for measuring binding affinity and/or dissociation constants in accordance with the present disclosure, including, e.g., but not limited to ELISAs, surface plasmon resonance (SPR) assays, Luminex Single Antigen (LSA) assays, bio-layer interferometry (BLI) (e.g., Octet) assays, grating-coupled interferometry, and spectroscopic assays.

In some embodiments, a target-binding moiety is assessed for off-target interactions. In some embodiments, a target-binding moiety is assessed using immunocapture followed by mass spectrometry (e.g., to reveal off target binding events in a complex sample). In some embodiments, a target-binding moiety is assessed using protein or glycan arrays, e.g., where many thousands of human proteins or glycans are arrayed on a chip and an antibody's binding is profiled across all available targets (e.g., a specific antibody will only bind to a target of interest). In some embodiments, a target-binding moiety is assessed using traditional immunoassays such as western blot. In some embodiments, a target-binding moiety is assessed for generic off-target non-specific binding (e.g., binding to other antibodies, DNA, lipids, etc.). In some embodiments, such generic off-target non-specific binding may be measured and identified using a negative control to identify a false positive signal (e.g., suggesting that one or more antibodies bind non-specifically, and not to a target).

In some embodiments, a target-binding moiety may be or comprise an agent of any chemical class such as, for example, a carbohydrate, a nucleic acid, a lipid, a metal, a polypeptide, a small molecule, etc., and/or a combination thereof. In some embodiments, a target-binding moiety may be or comprise an affinity agent such as an antibody, affimer, aptamer, lectin, siglec, etc. In some embodiments, a target-binding moiety is or comprises an antibody agent, e.g., an antibody agent that specifically binds to a target or an epitope thereof, e.g., a provided biomarker of a target biomarker signature for prostate cancer or an epitope thereof. In some embodiments, a target-binding moiety is or comprises a lectin or siglec that specifically binds to a carbohydrate-dependent marker as provided herein. In some embodiments, a target-binding moiety for a provided biomarker may be a commercially available. In some embodiments, a target-binding moiety for a provided biomarker may be designed and created for the purpose of use in assays as described herein. In some embodiments, a target-binding moiety is or comprises an aptamer, e.g., an aptamer that specifically binds to a target or an epitope thereof, e.g., a provided biomarker of a target biomarker signature for prostate cancer or an epitope thereof. In some embodiments, a target-binding moiety is or comprises an affimer molecule that specifically binds to a target or an epitope thereof, e.g., a provided biomarker of a target biomarker signature for prostate cancer or an epitope thereof. In some embodiments, such an affimer molecule can be or comprise a peptide or polypeptide that binds to a target or an epitope thereof (e.g., as described herein) with similar specificity and affinity to that of a corresponding antibody. In some embodiments, a target may be or comprise a target that is associated with prostate cancer. For example, in some such embodiments, a cancer-associated target can be or comprise a target that is associated with more than one cancer (i.e., at least two or more cancers). In some embodiments, a cancer-associated target can be or comprise a target that is typically associated with cancers. In some embodiments, a cancer-associated target can be or comprise a target that is associated with cancers of a specific tissue, e.g., prostate cancer. In some embodiments, a cancer-associated target can be or comprise a target that is specific to a particular cancer, e.g., a particular prostate cancer and more specifically prostate adenocarcinoma.

In some embodiments, a target-binding moiety recognizes and specifically binds to a target present in a biological entity (including, e.g., but not limited to cells and/or extracellular vesicles). For example, in some embodiments, a target-binding moiety may recognize and specifically bind to a tumor-associated antigen or epitope thereof. In some embodiments, a tumor-associated antigen may be or comprise an antigen that is associated with a cancer such as, for example, skin cancer, brain cancer (including, e.g., glioblastoma), breast cancer, prostate cancer (e.g., prostate adenocarcinoma), liver cancer, lung cancer, ovarian cancer, pancreatic cancer, prostate cancer, and skin cancer. In some embodiments, a target-binding moiety may recognize a tumor antigen associated with prostate cancer (e.g., prostate adenocarcinoma). In some embodiments, a target-binding moiety may recognize a tumor antigen associated with prostate adenocarcinoma.

In some embodiments, a target-binding moiety may specifically bind to an intravesicular target, e.g., a provided intravesicular protein or RNA (e.g., mRNA). In some embodiments, a target-binding moiety may specifically bind to a surface target that is present on/within nanoparticles having a size range of interest that includes extracellular vesicles, e.g., a membrane-bound polypeptide present on prostate cancer-associated extracellular vesicles.

In some embodiments, a target-binding moiety is directed to a biomarker for a specific condition or disease (e.g., cancer), which biomarker is or has been determined, for example, by analyzing a population or library (e.g., tens, hundreds, thousands, tens of thousands, hundreds of thousands, or more) of patient biopsies and/or patient data to identify such a biomarker (e.g., a predictive biomarker).

In some embodiments, a relevant biomarker may be one identified and/or characterized, for example, via data analysis. In some embodiments, for example, a diverse set of data (e.g., in some embodiments comprising one or more of bulk RNA sequencing, single-cell RNA (scRNA) sequencing, mass spectrometry, histology, post-translational modification data, in vitro and/or in vivo experimental data) can be analyzed through machine learning and/or computational modeling to identify biomarkers (e.g., predictive markers) that are highly specific to a disease or condition (e.g., cancer).

In some embodiments, a target-binding moiety is directed to a tissue-specific target, for example, a target that is associated with a specific tissue such as, for example, brain, breast, colon, ovary and/or other tissues associated with a female reproductive system, pancreas, prostate and/or other tissues associated with a male reproductive system, liver, lung, and skin. In some embodiments, such a tissue-specific target may be associated with a normal healthy tissue and/or a diseased tissue, such as a tumor. In some embodiments, a target-binding moiety is directed to a target that is specifically associated with a normal healthy condition of a subject.

In some embodiments, individual target binding entities utilized in a plurality of detection probes (e.g., as described and/or utilized herein) are directed to different targets. In some embodiments, such different targets may represent different marker proteins or polypeptides. In some embodiments, such different targets may represent different epitopes of the same marker proteins or polypeptides. In some embodiments, two or more individual target binding entities utilized in a plurality of detection probes (e.g., as described and/or utilized herein) may be directed to the same target.

In some embodiments, individual target binding entities utilized in a plurality of detection probes for detection of prostate cancer may be directed to different target biomarkers of a target biomarker signature for prostate cancer (e.g., ones as described in the section entitled “Provided Biomarkers and/or Target Biomarker Signatures for Detection of Prostate Cancer” above).

In some embodiments, individual target binding entities utilized in a plurality of detection probes for detection of prostate cancer may be directed to the same target biomarker of a target biomarker signature for prostate cancer (e.g., ones as described in the section entitled “Provided Biomarkers and/or Target Biomarker Signatures for Detection of Prostate Cancer” above). In some embodiments, such target binding entities may be directed to the same or different epitopes of the same target biomarker of such a target biomarker signature for prostate cancer.

### B. Oligonucleotide Domains

In some embodiments, an oligonucleotide domain for use in accordance with the present disclosure (e.g., that may be coupled to a target-binding moiety) may comprise a double-stranded portion and a single-stranded overhang extended from one or both ends of the oligonucleotide domain. In some embodiments where an oligonucleotide domain comprises a single-stranded overhang extended from each end, a single-stranded overhang is extended from a different strand of a double-stranded portion. In some embodiments where an oligonucleotide domain comprises a single-stranded overhang extended from one end of the oligonucleotide domain, the other end of the oligonucleotide domain may be a blunt end.

In some embodiments, an oligonucleotide domain may comprise ribonucleotides, deoxyribonucleotides, synthetic nucleotide residues that are capable of participating in Watson-Crick type or analogous base pair interactions, and any combinations thereof. In some embodiments, an oligonucleotide domain is or comprises DNA. In some embodiments, an oligonucleotide domain is or comprises peptide nucleic acid (PNA).

In some embodiments, an oligonucleotide may have a length that is determined, at least in part, for example, by, e.g., the physical characteristics of an entity of interest (e.g., biological entity such as extracellular vesicles) to be detected, and/or selection and localization of molecular targets in an entity of interest (e.g., biological entity such as extracellular vesicles) to be detected. In some embodiments, an oligonucleotide domain of a detection probe is configured to have a length such that when a first detection probe and a second detection probe bind to an entity of interest (e.g., biological entity such as extracellular vesicles), the first single-stranded overhang and the second single-stranded overhang are in sufficiently close proximity to permit interaction (e.g., hybridization) between the single-stranded overhangs. For example, when an entity of interest (e.g., biological entity) is an extracellular vesicle (e.g., an exosome), oligonucleotide domains of detection probes can each independently have a length such that their respective single-stranded overhangs are in sufficiently close proximity to anneal or interact with each other when the corresponding detection probes are bound to the same extracellular vesicle. For example, in some embodiments, oligonucleotide domains of detection probes for use in detecting extracellular vesicles (e.g., an exosome) may each independently have a length of about 20 nm to about 200 nm, about 40 nm to about 500 nm, about 40 nm to about 300 nm, or about 50 nm to about 150 nm. In some embodiments, oligonucleotide domains of detection probes for use in detecting extracellular vesicles (e.g., an exosome) may each independently have a length of about 20 nm to about 200 nm. In some embodiments, lengths of oligonucleotide domains of detection probes in a set can each independently vary to increase and/or maximize the probability of them finding each other when they simultaneously bind to the same entity of interest. Such oligonucleotide domains designed for use in detection probes for detecting extracellular vesicles can also be used in detection probes for detecting nanoparticles having a size range of interest that includes extracellular vesicles.

Accordingly, in some embodiments, an oligonucleotide domain for use in technologies provided herein may have a length in the range of about 20 up to about 1000 nucleotides. In some embodiments, an oligonucleotide domain may have a length in the range of about 30 up to about 1000 nucleotides, In some embodiments, an oligonucleotide domain may have a length in the range of about 30 to about 500 nucleotides, from about 30 to about 250 nucleotides, from about 30 to about 200 nucleotides, from about 30 to about 150 nucleotides, from about 40 to about 150 nucleotides, from about 40 to about 125 nucleotides, from about 40 to about 100 nucleotides, from about 40 to about 60 nucleotides, from about 50 to about 90 nucleotides, from about 50 to about 80 nucleotides. In some embodiments, an oligonucleotide domain may have a length of at least 20 or more nucleotides, including, e.g., at least 30, at least 40, at least 50, at least 60, at least 70, at least 80, at least 90, at least 100, at least 250, at least 500, at least 750, at least 1000 nucleotides or more. In some embodiments, an oligonucleotide domain may have a length of no more than 1000 nucleotides or lower, including, e.g., no more than 900, no more than 800, no more than 700, no more than 600, no more than 500, no more than 400, no more than 300, no more than 200, no more than 100, no more than 90, no more than 80, no more than 70, no more than 60, no more than 50, no more than 40 nucleotides, no more than 30 nucleotides, no more than 20 nucleotides or lower.

In some embodiments, an oligonucleotide domain may have a length of about 20 nm to about 500 nm. In some embodiments, an oligonucleotide domain may have a length of about 20 nm to about 400 nm, about 30 nm to about 200 nm, about 50 nm to about 100 nm, about 30 nm to about 70 nm, or about 40 nm to about 60 nm. In some embodiments, an oligonucleotide domain may have a length of at least about 20 nm or more, including, e.g., at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm or more. In some embodiments, an oligonucleotide domain may have a length of no more than 1000 nm or lower, including, e.g., no more than 900 nm, no more than 800 nm, no more than 700 nm, no more than 600 nm, no more than 500 nm, no more than 400 nm, no more than 300 nm, no more than 200 nm, no more than 100 nm or lower.

In some embodiments, a double-stranded portion of an oligonucleotide domain for use in technologies provided herein may have a length in the range of about 30 up to about 1000 nucleotides. In some embodiments, a double-stranded portion of an oligonucleotide domain may have a length in the range of about 30 to about 500 nucleotides, from about 30 to about 250 nucleotides, from about 30 to about 200 nucleotides, from about 30 to about 150 nucleotides, from about 40 to about 150 nucleotides, from about 40 to about 125 nucleotides, from about 40 to about 100 nucleotides, from about 50 to about 90 nucleotides, from about 50 to about 80 nucleotides. In some embodiments, a double-stranded portion of an oligonucleotide domain may have a length of at least 30 or more nucleotides, including, e.g., at least 40, at least 50, at least 60, at least 70, at least 80, at least 90, at least 100, at least 250, at least 500, at least 750, at least 1000 nucleotides or more. In some embodiments, a double-stranded portion of an oligonucleotide domain may have a length of no more than 1000 nucleotides or lower, including, e.g., no more than 900, no more than 800, no more than 700, no more than 600, no more than 500, no more than 400, no more than 300, no more than 200, no more than 100, no more than 90, no more than 80, no more than 70, no more than 60, no more than 50, no more than 40 nucleotides or lower.

In some embodiments, a double-stranded portion of an oligonucleotide domain may have a length of about 20 nm to about 500 nm. In some embodiments, a double-stranded portion of an oligonucleotide domain may have a length of about 20 nm to about 400 nm, about 30 nm to about 200 nm, about 50 nm to about 100 nm, about 30 nm to about 70 nm, or about 40 nm to about 60 nm. In some embodiments, a double-stranded portion of an oligonucleotide domain may have a length of at least about 20 nm or more, including, e.g., at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm or more. In some embodiments, a double-stranded portion of an oligonucleotide domain may have a length of no more than 1000 nm or lower, including, e.g., no more than 900 nm, no more than 800 nm, no more than 700 nm, no more than 600 nm, no more than 500 nm, no more than 400 nm, no more than 300 nm, no more than 200 nm, no more than 100 nm or lower.

In some embodiments, a double-stranded portion of an oligonucleotide domain is characterized in that when detection probes are connected to each other through hybridization of respective complementary single-stranded overhangs (e.g., as described and/or utilized herein), the combined length of the respective oligonucleotide domains (including, if any, a linker that links a target-binding moiety to an oligonucleotide domain) is long enough to allow respective target binding entities to substantially span the full characteristic length (e.g., diameter) of an entity of interest (e.g., an extracellular vesicle). For example, in some embodiments where extracellular vesicles are entities of interest, a combined length of oligonucleotide domains (including, if any, a linker that links a target-binding moiety to an oligonucleotide domain) of detection probes may be approximately 50 to 200 nm, when the detection probes are fully connected to each other.

In some embodiments, a double-stranded portion of an oligonucleotide domain may comprise a binding site for a primer. In some embodiments, such a binding site for a primer may comprise a nucleotide sequence that is designed to reduce or minimize the likelihood for miss-priming or primer dimers. Such a feature, in some embodiments, can decrease the lower limit of detection and thus increase the sensitivity of systems provided herein. In some embodiments, a binding site for a primer may comprise a nucleotide sequence that is designed to have a similar annealing temperature as another primer binding site.

In some embodiments, a double-stranded portion of an oligonucleotide domain may comprise a nucleotide sequence designed to reduce or minimize overlap with nucleic acid sequences (e.g., DNA and/or RNA sequences) typically associated with genome and/or gene transcripts (e.g., genomic DNA and/or RNA, such as mRNA of genes) of a subject (e.g., a human subject). Such a feature, in some embodiments, may reduce or minimize interference of any genomic DNA and/or mRNA transcripts of a subject that may be present (e.g., as contaminants) in a sample during detection.

In some embodiments, a double-stranded portion of an oligonucleotide domain may have a nucleotide sequence designed to reduce or minimize formation of self-dimers, homo-dimers, or hetero-dimers.

In some embodiments, a single-stranded overhang of an oligonucleotide domain for use in technologies provided herein may have a length of about 2 to about 20 nucleotides. In some embodiments, a single-stranded overhang of an oligonucleotide domain may have a length of about 2 to about 15 nucleotides, from about 2 to about 10 nucleotides, from about 3 to about 20 nucleotides, from about 3 to about 15 nucleotides, from about 3 to about 10 nucleotides. In some embodiments, a single-stranded overhang can have at least 1 to 5 nucleotides in length. In some embodiments, a single-stranded overhang of an oligonucleotide domain may have a length of at least 2 or more nucleotides, including, e.g., at least 3, at least 4, at least 5, at least 6, at least 7, at least 8, at least 9, at least 10, at least 11, at least 12, at least 13, at least 14, at least 15, at least 20 nucleotides, or more. In some embodiments, a single-stranded overhang of an oligonucleotide domain may have a length of no more than 20 nucleotides or lower, including, e.g., no more than 15, no more than 14, no more than 13, no more than 12, no more than 11, no more than 10, no more than 9, no more than 8, no more than 7, no more than 6, no more than 5, no more than 4 nucleotides or lower.

In some embodiments, a single-stranded overhang of an oligonucleotide domain may have a length of about 1 nm to about 10 nm. In some embodiments, a single-stranded overhang of an oligonucleotide domain may have a length of about 1 nm to about 5 nm. In some embodiments, a single-stranded overhang of an oligonucleotide domain may have a length of at least about 0.5 nm or more, including, e.g., at least about 1 nm, at least about 1.5 nm, at least about 2 nm, at least about 3 nm, at least about 4 nm, at least about 5 nm, at least about 6 nm, at least about 7 nm, at least about 8 nm, at least about 9 nm, at least about 10 nm or more. In some embodiments, a single-stranded overhang of an oligonucleotide domain may have a length of no more than 10 nm or lower, including, e.g., no more than 9 nm, no more than 8 nm, no more than 7 nm, no more than 6 nm, no more than 5 nm, no more than 4 nm, no more than 3 nm, no more than 2 nm, no more than 1 nm or lower.

A single-stranded overhang of an oligonucleotide domain is designed to comprise a nucleotide sequence that is complementary to at least a portion of a single-stranded overhang of a second detection probe such that a double-stranded complex comprising a first detection probe and a second detection probe can be formed through hybridization of the complementary single-stranded overhangs. In some embodiments, nucleotide sequences of complementary single-stranded overhangs are selected for optimal ligation efficiency in the presence of an appropriate nucleic acid ligase. In some embodiments, a single-stranded overhang has a nucleotide sequence preferentially selected for efficient ligation by a specific nucleic acid ligase of interest (e.g., a DNA ligase such as a T4 or T7 ligase). For example, such a single-stranded overhang may have a nucleotide sequence of GAGT, e.g., as described in Song et al., “Enzyme-guided DNA sewing architecture” Scientific Reports 5:17722 (2015), which is incorporated herein by reference for the purpose described herein.

When two detection probes couple together through hybridization of respective complementary single-stranded overhangs, their respective oligonucleotide domains comprising the hybridized single-stranded overhangs can, in some embodiments, have a combined length of about 90%-110% or about 95%-105% of a characteristic length (e.g., diameter) of an entity of interest (e.g., a biological entity). For example, in some embodiments when a biological entity is an exosome, the combined length can be about 50 nm to about 200 nm, or about 75 nm to about 150 nm, or about 80 nm to about 120 nm.

### C. Coupling Between a Target-Binding Moiety and an Oligonucleotide Domain

An oligonucleotide domain and a target-binding moiety can be coupled together in a detection probe by a covalent linkage, and/or by a non-covalent association (such as, e.g., a protein-protein interaction such as streptavidin-biotin interaction and/or an ionic interaction). In some embodiments, a detection probe appropriate for use in accordance with the present disclosure is a conjugate molecule comprising a target-binding moiety and an oligonucleotide domain, where the two components are typically covalently coupled to each other, e.g., directly through a bond, or indirectly through one or more linkers. In some embodiments, a target-binding moiety is coupled to one of two strands of an oligonucleotide domain by a covalent linkage (e.g., directly through a bond or indirectly through one or more linkers) and/or by a non-covalent association (such as, e.g., a protein-protein interaction such as streptavidin-biotin interaction and/or ionic interaction).

Where linkers are employed, in some embodiments, linkers are chosen to provide for covalent attachment of a target-binding moiety to one or both strands of an oligonucleotide domain through selected linkers. In some embodiments, linkers are chosen such that the resulting covalent attachment of a target-binding moiety to one or both strands of an oligonucleotide domain maintains the desired binding affinity of the target-binding moiety for its target. In some embodiments, linkers are chosen to enhance binding specificity of a target-binding moiety for its target. Linkers and/or conjugation methods of interest may vary widely depending on a target-binding moiety, e.g., its size and/or charges. In some embodiments, linkers are biologically inert.

A variety of linkers and/or methods for coupling a target-binding moiety to an oligonucleotide is known to one of ordinary skill in the art and can be used in accordance with the present disclosure. In some embodiments, a linker can comprise a spacer group at either end with a reactive functional group at either end capable of covalent attachment to a target-binding moiety. Examples of spacer groups that can be used in linkers include, but are not limited to, aliphatic and unsaturated hydrocarbon chains (including, e.g., C4, C5, C6, C7, C8, C9, C10, C11, C12, C13, C14, C15, C16, C17, C18, C19, C20, or longer), spacers containing heteroatoms such as oxygen (e.g., ethers such as polyethylene glycol) or nitrogen (polyamines), peptides, carbohydrates, cyclic or acyclic systems that may contain heteroatoms. Non-limiting examples of a reactive functional group to facilitate covalent attachment include nucleophilic functional groups (e.g., amines, alcohols, thiols, and/or hydrazides), electrophilic functional groups (e.g., aldehydes, esters, vinyl ketones, epoxides, isocyanates, and/or maleimides), functional groups capable of cycloaddition reactions, forming disulfide bonds, or binding to metals. In some embodiments, exemplary reactive functional groups, but are not limited to, primary and secondary amines, hydroxamic acids, N-hydroxysuccinimidyl (NHS) esters, dibenzocyclooctyne (DBCO)—NHS esters, azido-NHS esters, azidoacetic acid NHS ester, propargyl-NHS ester, trans-cyclooctene-NHS esters, N-hydroxysuccinimidyl carbonates, oxycarbonylimidazoles, nitrophenylesters, trifluoroethyl esters, glycidyl ethers, vinylsulfones, maleimides, azidobenzoyl hydrazide, N-[4-(p-azidosalicylamino) butyl]-3′-[2′-pyridyldithio]propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl 4-[N-maleimidomethyl]cyclohexane-1-carboxylate, 3-(2-pyridyldithio) propionic acid N-hydroxysuccinimide ester (SPDP), 4-(N-maleimidomethyl)-cyclohexane-1-carboxylic acid N-hydroxysuccinimide ester (SMCC), and any combinations thereof.

In some embodiments, a target-binding moiety (e.g., a target binding antibody agent) is coupled or conjugated to one or both strands of an oligonucleotide domain using N-hydrosysuccinimide (NHS) ester chemistry. NHS esters react with free primary amines and result in stable covalent attachment. In some embodiments, a primary amino group can be positioned at a terminal end with a spacer group, e.g., but not limited to an aliphatic and unsaturated hydrocarbon chain (e.g., a C6 or C12 spacer group).

In some embodiments, a target-binding moiety (e.g., a target-binding affinity agent) can be coupled or conjugated to one or both strands of an oligonucleotide domain using a site-specific conjugation method known in the art, e.g., to enhance the binding specificity of conjugated target-binding moiety (e.g., conjugated target-binding affinity agent). Examples of a site-specific conjugation method include, but are not limited to coupling or conjugation through a disulfide bond, C-terminus, carbohydrate residue or glycan, and/or unnatural amino acid labeling. In some embodiments where a target-binding moiety is or comprises an affinity agent, an oligonucleotide can be coupled or conjugated to the target-binding moiety via at least one or more free amine groups present in the target-binding moiety. In some embodiments, an oligonucleotide can be coupled or conjugated to a target-binding moiety that is or comprises an affinity agent via at least one or more reactive thiol groups present in the target-binding moiety. In some embodiments, an oligonucleotide can be coupled or conjugated to a target-binding moiety that is or comprises an antibody agent or a peptide aptamer via at least one or more carbohydrate residues present in the target-binding moiety.

In some embodiments, a plurality of oligonucleotides (e.g., at least 2, at least 3, at least 4, at least 5, at least 6, at least 7, at least 8, at least 9, at least ten, or more) can be coupled or conjugated to a target-binding moiety (e.g., a target binding antibody agent).

### Exemplary Duplex Target Entity Detection System

In some embodiments, a target entity detection system as provided by the present disclosure (and useful, for example, for detecting, e.g., at a single entity level, extracellular vesicles associated with prostate cancer) may comprise a first population of first detection probes (e.g., as described and/or utilized herein) for a provided target biomarker (e.g., ones described herein) and a second population of second detection probes (e.g., as described and/or utilized herein) for a provided target biomarker (e.g., ones described herein). In some embodiments, the first detection probes and the second detection probes are directed to the same provided target biomarker. In some embodiments, the first detection probes and the second detection probes are directed to different provided target biomarkers.

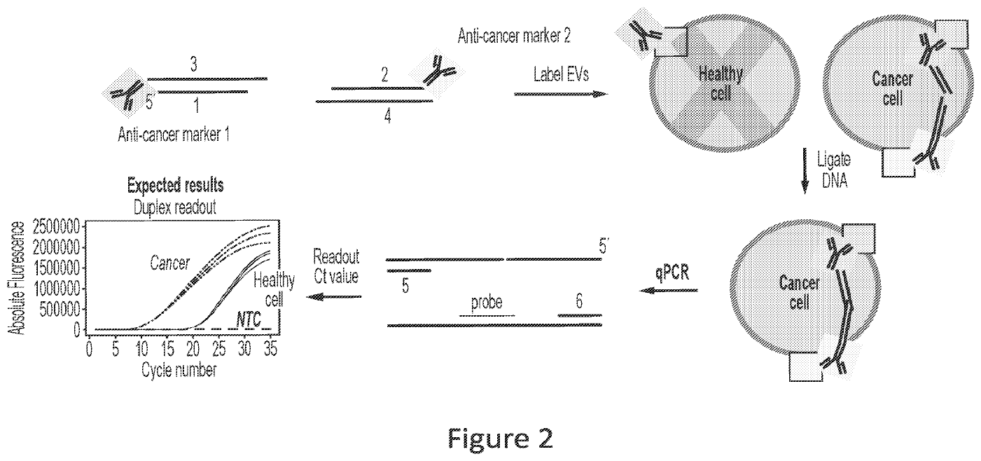

FIG. 2 illustrates an exemplary duplex target entity detection system for detecting, at a single entity level, an entity of interest (e.g., biological entity such as an extracellular vesicle) comprising (i) at least one target (e.g., a provided biomarker of a target biomarker signature for prostate cancer) which expression level is high enough such that two molecules of the same target (e.g., a provided biomarker of a target biomarker signature for prostate cancer) are found in close proximity, or (ii) at least two or more distinct targets (e.g., provided biomarkers of a target biomarker signature for prostate cancer). A first detection probe comprises a first target-binding moiety (e.g., directed to a target cancer marker 1) and a first oligonucleotide domain coupled to the first target-binding moiety, the first oligonucleotide domain comprising a first double-stranded portion and a first single-stranded overhang extended from one end of the first oligonucleotide domain. As shown in FIG. 2, a first oligonucleotide domain may be resulted from hybridization of a longer strand (strand 3) and a shorter strand (strand 1), thereby forming a double-stranded portion and a single-stranded overhang at one end. In some embodiments, a first target-binding moiety (e.g., directed to target cancer marker 1) is coupled (e.g., covalently coupled) to a 5′ end or 3′ end of a strand of a first oligonucleotide domain (e.g., strand 1). In some embodiments, a 5′ end or 3′ end of a strand that is coupled to a first target-binding moiety may be modified with a linker (e.g., as described and/or utilized herein with or without a spacer group). In some embodiments, a 5′ end of another strand of a first oligonucleotide domain (e.g., strand 3) has a free phosphate group.

In the embodiment depicted in FIG. 2, a second detection probe comprises a second target-binding moiety (e.g., directed to a target cancer marker 2) and a second oligonucleotide domain coupled to the second target-binding moiety, the second oligonucleotide domain comprising a second double-stranded portion and a second single-stranded overhang extended from one end of the second oligonucleotide domain. As shown in FIG. 2, a second oligonucleotide domain may be resulted from hybridization of a longer strand (strand 4) and a shorter strand (strand 2), thereby forming a double-stranded portion and a single-stranded overhang at one end. In some embodiments, a second target-binding moiety (e.g., directed to a target cancer marker 2) is coupled (e.g., covalently coupled) to a 5′ end of a strand of a second oligonucleotide domain (e.g., strand 2). In some embodiments, a 5′ end of a strand that is coupled to a second target-binding moiety may be modified with a linker (e.g., as described and/or utilized herein with or without a spacer group). In some embodiments, a 5′ end of another strand of a second oligonucleotide domain (e.g., strand 4) has a free phosphate group.

At least portions of a first single-stranded overhang and a second single-stranded overhang are complementary to each other such that they can hybridize to form a double-stranded complex when they are in sufficiently close proximity, e.g., when a first detection probe and a second detection probe simultaneously bind to the same entity of interest (e.g., biological entity such as extracellular vesicle). In some embodiments, a first single-stranded overhang and a second single-stranded overhang have equal lengths such that when they hybridize to form a double-stranded complex, there is no gap (other than a nick to be ligated) between their respective oligonucleotide domains and each respective target-binding moiety is located at an opposing end of the double-stranded complex. For example, in some embodiments, a double-stranded complex forms before ligation occurs, wherein the double-stranded complex comprises a first detection probe and a second detection probe coupled to each other through direct hybridization of their respective single-stranded overhangs (e.g., having 4 nucleotides in length), wherein each respective target-binding moiety (e.g., directed to a target cancer marker 1 and a target cancer marker 2, respectively) is present at opposing ends of the double-stranded complex. In such embodiments, both strands of the double-stranded complex (comprising a nick between respective oligonucleotide domains) are ligatable, e.g., for amplification and detection. In some embodiments, a double-stranded complex (e.g., before ligation occurs) can comprise an entity of interest (e.g., a biological entity such as an extracellular vesicle), wherein a first target-binding moiety (e.g., directed to a target cancer marker 1) and a second target-binding moiety (e.g., directed to a target cancer marker 2) are simultaneously bound to the entity of interest.

In some embodiments of a duplex target entity detection system for detection of prostate cancer (e.g., prostate adenocarcinoma), a first target-binding moiety of a first detection probe may be directed to a first target surface biomarker (e.g., ones provided in the section entitled “Provided Biomarkers and/or Target Biomarker Signatures for Detection of Prostate Cancer”), while a second target-binding moiety of a second detection probe may be directed to a second target surface biomarker (e.g., ones provided in the section entitled “Provided Biomarkers and/or Target Biomarker Signatures for Detection of Prostate Cancer”). In some embodiments, a first target-binding moiety of a first detection probe may be directed to a first target intravesicular biomarker (e.g., ones provided in the section entitled “Provided Biomarkers and/or Target Biomarker Signatures for Detection of Prostate Cancer”), while a second target-binding moiety of a second detection probe may be directed to a second target intravesicular biomarker (e.g., ones provided in the section entitled “Provided Biomarkers and/or Target Biomarker Signatures for Detection of Prostate Cancer”). In some embodiments, the first target-binding moiety and the second target-binding moiety may be directed to the same or different epitopes of the same target surface biomarker or of the same target intravesicular biomarker. In some embodiments, the first target-binding moiety and the second target-binding moiety may be directed to the different target surface biomarkers or different target intravesicular biomarkers. In some embodiments, the double stranded portion of a first oligonucleotide domain and a second oligonucleotide domain may be the same. In some embodiments, the double-stranded portion of a first oligonucleotide domain and a second oligonucleotide domain may be different.

In some embodiments, a duplex target entity detection system for detection of prostate adenocarcinoma may comprise at least two distinct sets of detection probes. For example, in some embodiments, each set may be directed to a distinct target biomarker signature comprising one or more target biomarkers (e.g., ones described herein).

In some embodiments, a duplex target entity detection system comprising at least two distinct sets of detection probes may also comprise a capture assay comprising a capture agent directed to an extracellular vesicle-associated surface biomarker.

In some embodiments, any combination of biomarker probes (e.g., a biomarker signature) including capture probes or detection probes as described herein may be utilized in combination with any other set of biomarker probes (e.g., a biomarker signature) including capture probes or detection probes as described herein.

Exemplary Triplex or Multiplex (n≥3) Target Entity Detection System

In some embodiments, a target entity detection system as provided by the present disclosure (and useful, for example, for detecting, e.g., at a single entity level, extracellular vesicles associated with prostate cancer) may comprise n populations of distinct detection probes (e.g., as described and/or utilized herein), wherein n≥3. For example, in some embodiments when n=3, a target entity detection system may comprise a first detection probe (e.g., as described and/or utilized herein) for a first target, a population of a second detection probe (e.g., as described and/or utilized herein) for a second target, and a population of a third detection probe (e.g., as described and/or utilized herein) for a third target.

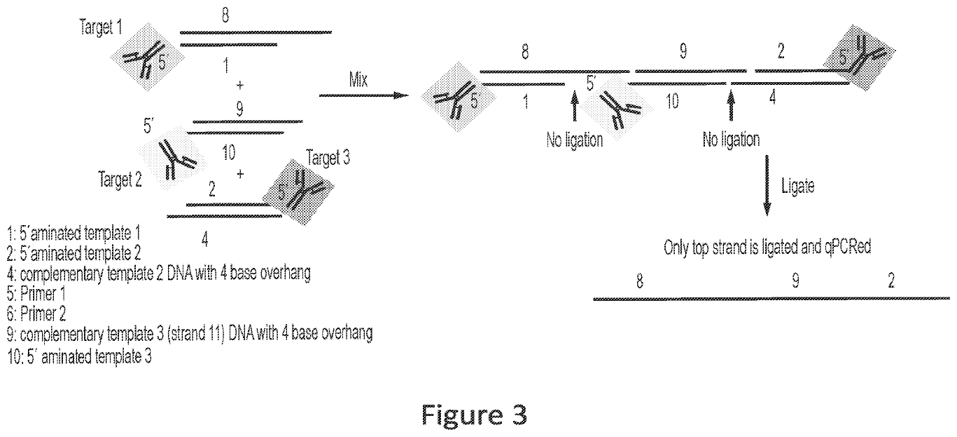

FIG. 3 illustrates an exemplary triplex target entity detection system for detecting, at a single entity level, an entity of interest (e.g., a biological entity such as an extracellular vesicle) comprising three distinct molecular targets. A first detection probe comprises a first target-binding moiety (e.g., anti-cancer marker 1 antibody agent) and a first oligonucleotide domain coupled to the first target-binding moiety, the first oligonucleotide domain comprising a first double-stranded portion and a first single-stranded overhang extended from one end of the first oligonucleotide domain. As shown in FIG. 3, a first oligonucleotide domain may be resulted from hybridization of a longer strand (strand 8) and a shorter strand (strand 1), thereby forming a double-stranded portion and a single-stranded overhang at one end. In some embodiments, a first target-binding moiety (e.g., anti-cancer marker 1 antibody agent) is coupled (e.g., covalently coupled) to a 5′ end of a strand of a first oligonucleotide domain (e.g., strand 1). In some embodiments, a 5′ end of a strand that is coupled to a first target-binding moiety may be modified with a linker (e.g., as described and/or utilized herein with or without a spacer group). In some embodiments, a 5′ end of another strand of a first oligonucleotide domain (e.g., strand 8) has a free phosphate group.

In the embodiment depicted in FIG. 3, a second detection probe comprises a second target-binding moiety (e.g., anti-cancer marker 3 antibody agent) and a second oligonucleotide domain coupled to the second target-binding moiety, the second oligonucleotide domain comprising a second double-stranded portion and a second single-stranded overhang extended from one end of the second oligonucleotide domain. As shown in FIG. 3, a second oligonucleotide domain may be resulted from hybridization of a longer strand (strand 4) and a shorter strand (strand 2), thereby forming a double-stranded portion and a single-stranded overhang at one end. In some embodiments, a second target-binding moiety (e.g., anti-cancer marker 3 antibody agent) is coupled (e.g., covalently coupled) to a 5′ end of a strand of a second oligonucleotide domain (e.g., strand 2). In some embodiments, a 5′ end of a strand that is coupled to a second target-binding moiety may be modified with a linker (e.g., as described and/or utilized herein with or without a spacer group). In some embodiments, a 5′ end of another strand of a second oligonucleotide domain (e.g., strand 4) has no free phosphate group.

A third detection probe comprises a third target-binding moiety (e.g., anti-cancer marker 2 antibody agent) and a third oligonucleotide domain coupled to the third target-binding moiety, the third oligonucleotide domain comprising a third double-stranded portion and a single-stranded overhang extended from each end of the third oligonucleotide domain. For example, a single-stranded overhang is extended from one end of a strand of a third oligonucleotide domain while another single-stranded overhang is extended from an opposing end of a different strand of the third oligonucleotide domain. As shown in FIG. 3, a third oligonucleotide domain may be resulted from hybridization of portions of two strands (e.g., strands 9 and 10), thereby forming a double-stranded portion and a single-stranded overhang at each end. For example, a single-stranded overhang (3A) is formed at a 5′ end of strand 9 of a third detection probe, wherein the 5′ end of strand 9 has a free phosphate group. Additionally, a single-stranded overhang (3B) is formed at a 5′ end of strand 10 of the same third detection probe and a third target-binding moiety (e.g., anti-target 2 antibody agent) is also coupled (e.g., covalently coupled) to the 5′ end of strand 10. In some embodiments, a 5′ end of a strand (e.g., strand 10) that is coupled to a third target-binding moiety may be modified with a linker (e.g., as described and/or utilized herein with or without a spacer group).

When all three detection probes are in sufficiently close proximity, e.g., when all three detection probes simultaneously bind to the same entity of interest (e.g., biological entity), (i) at least a portion of a single-stranded overhang (e.g., 3A) of a third detection probe is hybridized to a corresponding complementary portion of a single-stranded overhang of a second detection probe, and (ii) at least a portion of another single-stranded overhang (e.g., 3B) of the third detection probe is hybridized to a corresponding complementary portion of a single-stranded overhang of a first detection probe. As a result, a double-stranded complex comprising all three detection probes coupled to each other in a linear arrangement is formed by direct hybridization of corresponding single-stranded overhangs. Scc, e.g., FIG. 3.

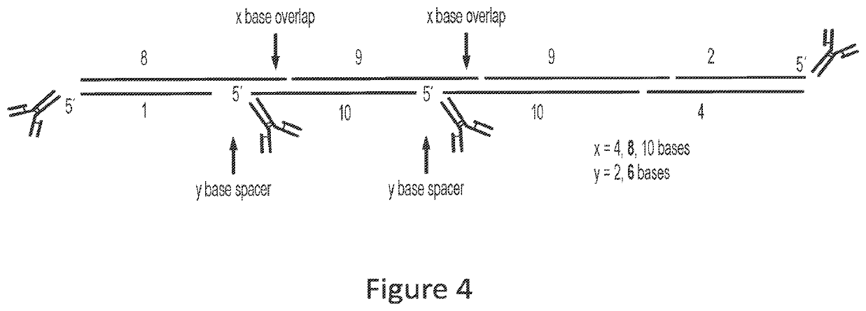

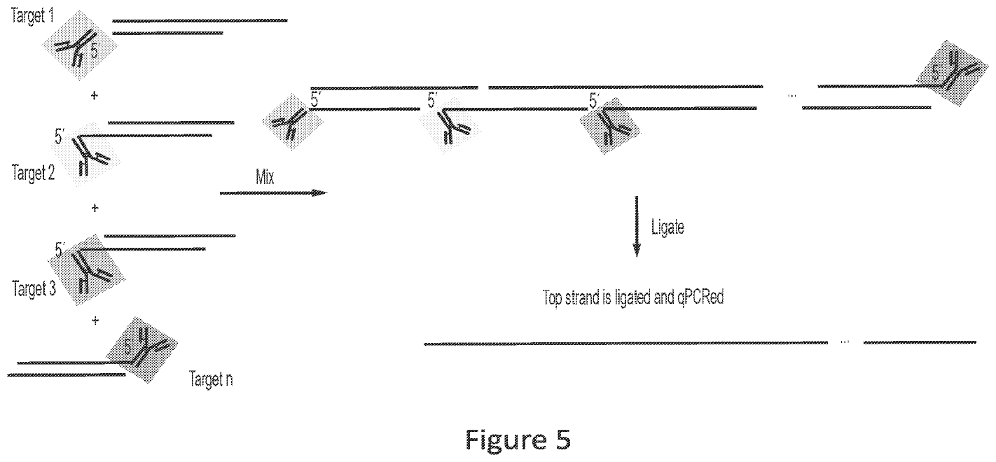

In some embodiments involving use of at least three or more (n ≥3) detection probes in provided technologies, when single-stranded overhangs of detection probes anneal to each respective partner(s) to form a double-stranded complex, at least (n-2) target-binding moiety/moieties is/are present at internal position(s) of the double-stranded complex. In such embodiments, it is desirable to have internal target binding moieties present in a single strand of the double-stranded complex such that another strand of the double-stranded complex is free of any internal target binding moieties and is thus ligatable to form a ligated template, e.g., for amplification and detection. See, e.g., FIG. 3 (using three detection probes), FIG. 4 (using four detection probes), and FIG. 5 (using n detection probes).

In some embodiments where a strand of a double-stranded complex comprises at least one or more internal target binding moieties, the strand comprises a gap between an end of an oligonucleotide strand of a detection probe to which the internal target-binding moiety is coupled and an end of an oligonucleotide strand of another detection probe. The size of the gap is large enough that the strand becomes non-ligatable in the presence of a nucleic acid ligase. In some embodiments, the gap may be 2-8 nucleotides in size or 2-6 nucleotides in size. In some embodiments, the gap is 6 nucleotides in size. In some embodiments, the overlap (hybridization region between single-stranded overhangs) can be 2-15 nucleotides in length or 4-10 nucleotides in length. In some embodiments, the overlap (hybridization region between single-stranded overhangs) is 8 nucleotides in length. The size of the gap and/or hybridization region are selected to provide an optimum signal separation from a ligated template (comprising no internal target binding moieties) and non-ligated template (comprising at least one internal target-binding moiety). It should be noted that while FIGS. 3-5 do not show binding of detection probes to an entity of interest (e.g., a biological entity), a double-stranded complex (e.g., before ligation occurs) can comprise an entity of interest (e.g., a biological entity such as extracellular vesicles), wherein at least three or more target binding moieties are simultaneously bound to the entity of interest.

In some embodiments, selection of a combination (e.g., a set) of detection probes (e.g., number of detection probes and/or specific biomarkers) for use in a target entity detection system provided herein (e.g., a duplex, triplex or multiplex target entity detection system described herein) is based on, for example, a desired specificity and/or a desired sensitivity that is deemed to be optimal for a particular application. For example, in some embodiments, a combination of detection probes is selected for detection of prostate cancer (e.g., for stage I, II, III, or IV) such that it provides a specificity of at least 95% or higher, including, e.g., at least 96%, at least 97%, at least 98%, at least 99%, at least 99.5%, at least 99.7%, at least 99.8% or higher. In some embodiments, a combination of detection probes is selected for detection of prostate cancer (e.g., for stage I, II, III, or IV) such that it provides a sensitivity of at least 30% or higher, including, e.g., at least 40%, at least 50%, at least 60%, at least 70%, at least 80%, at least 90%, at least 95% or higher. In some embodiments, a combination of detection probes is selected for detection of prostate cancer (e.g., for stage I, II, III, or IV) such that it provides a positive predictive value of at least 8% or higher, including, e.g., at least 9%, at least 10%, at least 15%, at least 20%, at least 25%, at least 30%, at least 40%, at least 50%, or higher. In some embodiments, a combination of detection probes is selected for detection of prostate cancer (e.g. prostate adenocarcinoma) (e.g., for stage I, II, III, or IV) such that it provides a positive predictive value of at least 2% or higher, including, e.g., at least 3%, at least 4%, at least 5%, at least 6%, at least 7%, at least 8%, at least 9%, at least 10%, at least 15%, at least 20%, at least 25%, at least 30%, at least 40%, at least 50%, or higher. In some embodiments, a combination of detection probes is selected for detection of prostate cancer (e.g., for stage I, II, III, or IV) such that it provides a limit of detection (LOD) below 1×10⁷ EV/mL sample or lower, including, e.g., below 7×10⁶ EV/mL sample, below 6×10⁶ EV/mL sample, below 5×10⁶ EV/mL sample, below 4×10⁶ EV/mL sample, below 3×10⁶ EV/mL sample, below 2×10⁶ EV/mL sample, below 1×10⁶ EV/mL sample, or lower. In some embodiments, such prostate cancer detection assay may be used to detect different subtypes of prostate cancer including, e.g., prostate adenocarcinoma and other specified types of prostate cancer as known in the art (SEER Cancer Statistics Review 1975-2017). In some embodiments, such prostate cancer detection assay may be used to detect prostate cancer of an epithelial origin. In some embodiments, such prostate cancer detection assay may be used to detect prostate adenocarcinoma.

In some embodiments, a combination (e.g., a set) of detection probes, rather than individual detection probes, confers specificity to detection of a disease, disorder, or condition (e.g., a particular prostate cancer (e.g., prostate adenocarcinoma) and/or a stage of prostate cancer as described herein), for example, one or more individual probes may be directed to a target that itself is not specific to prostate cancer. For example, in some embodiments, a useful combination of detection probes in a target entity detection system provided herein (e.g., a duplex, triplex or multiplex target entity detection system described herein) may comprise at least one detection probe directed to a target specific for the relevant disease, disorder, or condition (i.e., a target that is specific to the relevant disease, disorder, or condition), and may further comprise at least one detection probe directed to a target that is not necessarily or completely specific for the relevant disease, disorder, or condition (e.g., that may also be found on some or all cells that are healthy, are not of the particular disease, disorder, or condition, and/or are not of the particular disease stage of interest). That is, as will be appreciated by those skilled in the art reading the present specification, so long as the set of detection probes utilized in accordance with the present invention is or comprises a plurality of individual detection probes that together are specific for detection of the relevant disease, disorder, or condition (i.e., sufficiently distinguish biological entities for detection that are associated with the relevant disease, disorder, or condition from other biological entities not of interest for detection), the set is useful in accordance with certain embodiments of the present disclosure.

In some embodiments, a target entity detection system provided herein (e.g., a duplex, triplex or multiplex target entity detection system described herein) can comprise at least one or more (e.g., at least 2 or more) control probes (in addition to target-specific detection probes, e.g., as described and/or utilized herein, for example, in some embodiments to recognize disease-specific biomarkers such as cancer-specific biomarkers and/or tissue-specific biomarkers). In some embodiments, a control probe is designed such that its binding to an entity of interest (e.g., a biological entity) inhibits (completely or partially) generation of a detection signal.

In some embodiments, a control probe comprises a control binding moiety and an oligonucleotide domain (e.g., as described and/or utilized herein) coupled to the control binding moiety, the oligonucleotide domain comprising a double-stranded portion and a single-stranded overhang extended from one end of the oligonucleotide domain. A control binding moiety is an entity or moiety that bind to a control reference. In some embodiments, a control reference can be or comprise a biomarker that is preferentially associated with a normal healthy cell. In some embodiments, a control reference can be or comprise a biomarker preferentially associated from a non-target tissue. In some embodiments, inclusion of a control probe can selectively remove or minimize detectable signals generated from false positives (e.g., entities of interest comprising a control reference, optionally in combination with one or more targets to be detected). Other control probes described in U.S. application Ser. No. 16/805,637 (published as US2020/0299780; issued as U.S. Pat. No. 11,085,089), and International Application PCT/US2020/020529 (published as WO2020180741), both filed Feb. 28, 2020 and entitled “Systems, Compositions, and Methods for Target Entity Detection,” the entire contents of each application are incorporated herein by reference in their entirety, can be useful in provided target entity detections systems.

In some embodiments, the present disclosure provides insights, among other things, that detection probes as described or utilized herein may non-specifically bind to a solid substrate surface and some of them may remain in an assay sample even after multiple washes to remove any excess or unbound detection probes; and that such non-specifically bound detection probes may come off from the solid substrate surface and become free-floating in a ligation reaction, thus allowing them to interact with one another to generate a non-specific ligated template that produces an undesirable background signal. Accordingly, in some embodiments, a target entity detection system provided herein (e.g., a duplex, triplex, or multiplex target entity detection described herein) can comprise at least one or more (e.g., at least 2 or more) inhibitor oligonucleotides that are designed to capture residual detection probes that are not bound to an entity of interest but remain as free agents in a ligation reaction, thereby preventing such free-floating detection probes from interacting with other free-floating complementary detection probes to produce an undesirable background signal. In some embodiments, an inhibitor oligonucleotide may be or comprise a single-stranded or double-stranded oligonucleotide comprising a binding domain for a single-stranded overhang of a detection probe (e.g., as described or utilized herein), wherein the inhibitor oligonucleotide does not comprise a primer binding site. The absence of such a primer binding site in an inhibitor oligonucleotide prevents a primer from binding to a non-specific ligated template resulting from ligation of a detectable probe to an inhibitor oligonucleotide, thereby reducing or inhibiting the non-specific ligated template from amplification and/or detection, e.g., by polymerase chain reaction.

In some embodiments, an inhibitor oligonucleotide comprises a binding domain for a single-stranded overhang of a detection probe (e.g., as described or utilized herein), wherein the binding domain is or comprises a nucleotide sequence that is substantially complementary to the single-stranded overhang of the detection probe such that a free, unbound detection probe having a complementary single-stranded overhang can bind to the binding domain of the inhibitor oligonucleotide. In some embodiments, an inhibitor oligonucleotide may have a hairpin at one end. In some embodiments, an inhibitor oligonucleotide may be a single-stranded oligonucleotide comprising at one end a binding domain for a single-stranded overhang of a detection probe, wherein a portion of the single-stranded oligonucleotide can self-hybridize to form a hairpin at another end.

In some embodiments, a target entity detection system provided herein (e.g., a duplex, triplex or multiplex target entity detection system described herein) does not comprise a connector oligonucleotide that associates an oligonucleotide domain of a detection probe with an oligonucleotide domain of another detection probe. In some embodiments, a connector oligonucleotide is designed to bridge oligonucleotide domains of any two detection probes that would not otherwise interact with each other when they bind to an entity of interest. In some embodiments, a connector oligonucleotide is designed to hybridize with at least a portion of an oligonucleotide domain of a detection probe and at least a portion of an oligonucleotide domain of another detection probe. A connector oligonucleotide can be single-stranded, double-stranded, or a combination thereof. A connector oligonucleotide is free of any target-binding moiety (e.g., as described and/or utilized herein) or control binding moiety. In at least some embodiments, no connector oligonucleotides are necessary to indirectly connect oligonucleotide domains of detection probes; in some embodiments, such connector oligonucleotides are not utilized, in part because detection probes as provided and/or utilized herein are designed such that their respective oligonucleotide domains have a sufficient length to reach and interact with each other when they are in sufficiently close proximity, e.g., when the detection probes simultaneously bind to an entity of interest (e.g., a biological entity such as an extracellular vesicle).

### Methods of Using Provided Target Entity Detection Systems

Provided target entity detection systems are useful in detecting an entity of interest (e.g., a biological entity such as extracellular vesicles) in a sample (e.g., in a biological, environmental, or other sample) for various applications and/or purposes associated with detection of prostate cancer. Accordingly, some aspects provided herein relate to methods of using a plurality of (e.g., at least 2, at least 3, or more) detection probes appropriate for use in accordance with the present disclosure. In some embodiments, a method comprises contacting an entity of interest (e.g., a biological entity such as extracellular vesicles) in a sample (e.g., a blood or blood-derived sample from a human subject) with a set of detection probes comprising at least 2 or more (including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more) detection probes as described and/or utilized herein. In some embodiments, a method comprises subjecting a sample comprising an entity of interest (e.g., a biological entity such as extracellular vesicles) to a target entity detection system (e.g., as provided herein). A plurality of detection probes (e.g., at least two or more) can be added to a sample comprising an entity of interest (e.g., a biological entity such as extracellular vesicles) at the same time or at different times (e.g., sequentially). In some embodiments, a method may comprise, prior to contacting with a plurality of detection probes, contacting a sample comprising an entity of interest with at least one capture agent directed to an extracellular vesicle-associated surface biomarker.

In certain embodiments, a provided target entity detection system for use in a method described herein may comprise a plurality of (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more) distinct sets (e.g., combinations) of detection probes (e.g., as described herein). In some embodiments, a method comprises contacting an entity of interest (e.g., a biological entity such as extracellular vesicles) in a sample (e.g., a blood or blood-derived sample from a human subject) with a plurality of sets of detection probes, wherein each set may comprise at least 2 or more (including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more) detection probes as described and/or utilized herein. In some embodiments, a method comprises subjecting a sample comprising an entity of interest (e.g., a biological entity such as extracellular vesicles) to a target entity detection system (e.g., as provided herein). A plurality of detection probes and/or detection probe combinations (e.g., at least two or more) can be added to a sample comprising an entity of interest (e.g., a biological entity such as extracellular vesicles) at the same time or at different times (e.g., sequentially). In some embodiments, a method may comprise, prior to contacting with a plurality of detection probes, contacting a sample comprising an entity of interest with at least one capture agent directed to an extracellular vesicle-associated surface biomarker.

In some embodiments, the relationship between results (e.g., Ct values and/or relative number of ligated nucleic acid templates (e.g., ligated DNA templates)) from profiling one or more biomarker combinations in a sample can be combined with clinical information (including, e.g., but not limited to patient age, past medical history, etc.) and/or other information to better classify patients with or at risk for prostate cancer. Various classification algorithms can be used to interpret the relationship between multiple variables to increase an assay's sensitivity and/or specificity. In some embodiments, such algorithms include, but are not limited to, logistic regression models, support vector machines, gradient boosting machines, random forest algorithms, Naive Bayes algorithms, K-nearest neighborhood algorithms, and combinations thereof. In some embodiments, performance (e.g., accuracy) of assays described herein can be improved, e.g., by selection of biomarker combinations (e.g., as described herein), selection of other factors or variables (e.g., clinical information and/or lifestyle information) to include an algorithm, and/or selection of the type of algorithm itself.

In certain embodiments, technologies described herein utilize a predictive algorithm that is trained and validated using data sets as described herein. In certain embodiments, technologies described herein are utilized to generate a risk score using an algorithm created from training samples which is designed to take into account results from at least two, e.g., at least two, at least 3, at least 4, at least 5, or more than 5 separate assays comprising biomarker signatures (e.g., as described herein). In certain embodiments, an algorithm-generated risk score can be generated at least in part using diagnostic data (e.g., raw and/or normalized Ct values) from at least one individual assay (e.g., individual biomarker signature). In certain embodiments, a reference threshold can be included within a risk score. In certain embodiments, multiple threshold levels denoting multiple different degrees of prostate cancer risk may be included in a risk score. In some embodiments, separate target biomarker signature assays may be performed as individual assays in a series of assays, and individual assays may be weighted equally or differently in a predictive algorithm. In some embodiments, for example, weighting of individual assays combined in an algorithm (e.g., a cohort of biomarker assays) may be determined by a number of factors including but not limited to the sensitivity of an individual assay, the specificity of an individual assay, the reproducibility of an individual assay, the variability of an individual assay, the positive predictive value of an individual assay, and/or the lowest limit of detection of a specific assay. In some embodiments, a cohort of biomarker assays may be ranked according to a characteristic (e.g., sensitivity, specificity, lowest limit of detection etc.) and the biomarker assays may then be weighted based upon their relative rank.

In some embodiments, a risk score generated by an algorithm (as described herein) can be presented in a suitable manner, e.g., on a nominal scale, e.g., on a scale of 0-100 reflecting a number of likelihoods, e.g., including but not limited to the likelihood a subject has prostate cancer, the likelihood a subject will develop prostate cancer, and/or the likely stage of prostate cancer. In some embodiments, a higher risk score can demonstrate that there is an increasing likelihood of disease pathology, e.g., lower to higher values may reflect healthy controls, benign controls, stage I, stage II, stage III, and stage IV prostate cancers. In some embodiments, a risk score can be utilized to reduce the potential of cross reactivity of technologies as described herein when compared with other cancer types.

In some embodiments, a risk score may be generated from a combination of data derived from assays as described herein coupled with other applicable diagnostic data such as age, life history, MRI results, CT scanning, DRE results, urine biomarker test results, other blood biomarker test results, or any combination thereof. In some embodiments, a risk score provides predictive value above and beyond that of conventional standard of care diagnostic assay predictive values, e.g., higher than predictive values provided by abdominal scans, DRE, or other prostate cancer screening assays utilized in isolation or in combination with another diagnostic assay. In some embodiments, a risk score may be generated that has high specificity for prostate cancer (e.g., prostate adenocarcinoma) and has low sensitivity for other cancers.

In some embodiments, a risk score may have an associated clinical cutoff for detection of prostate cancer. For example, in some embodiments, a risk score's clinical cutoff for detection may require an assay that yields at least 40%, e.g., at least 50%, at least 60%, or greater sensitivity for detection of both early and late-stage prostate cancer and has a minimum of 90% specificity, e.g., at least 91%, at least 92%, at least 93%, at least 94%, at least 95%, at least 96%, at least 97%, at least 98%, at least 99% or greater specificity in a generally healthy population of subjects (e.g., aged 40 to 85 years of age) or in a population of subjects with hereditary risk. In some embodiments, sensitivity and specificity targets are the approximate lower bounds of the two-sided 95% confidence interval for the targeted 77% sensitivity and 99.5% specificity.

In some embodiments, a training study is performed to provide the necessary data required to program a risk score algorithm. In some embodiments, such a training study may comprise a cohort of samples from a range of suppliers, including at least commercial suppliers, biobanks, purpose driven studies, and/or physicians. In some embodiments, a training study may comprise positive samples from prostate cancer patients (e.g., stage I, stage II, stage III, and/or stage IV), positive control samples from prostate cancer cell lines, negative samples from benign prostate tumor patients, negative samples from non-prostate cancer patients (e.g., brain cancer, breast cancer, ovarian cancer, endometrial cancer, lung adenocarcinoma, melanoma, non-Hodgkin's lymphoma, pancreatic cancer, skin cancer, etc.), negative samples from inflammatory condition patients (e.g., benign prostatic hyperplasia, etc.), negative samples from healthy patients, or any combination thereof. In some embodiments, a training study may comprise samples from patients of any appropriate age range, e.g., <31 years old, 31-40 years old, 41-50 years old, 51-60 years old, 61-70 years old, 71-80 years old, or >80 years old. In some embodiments, a training study may comprise samples from patients of any race/ethnicity/descent, (e.g., Caucasians, Africans, Asians etc.).

In some embodiments, a validation study is performed to provide the necessary data required to confirm a risk score algorithm's utility. In some embodiments, such a validation study may comprise a cohort of samples from a range of suppliers, including at least commercial suppliers, biobanks, purpose driven studies, and/or physicians. In some embodiments, a validation study may comprise positive samples from prostate cancer patients (e.g., stage I, stage II, stage III, and/or stage IV, with respective Gleason scores), positive control samples from prostate cancer cell lines, negative samples from benign prostate tumor patients, negative samples from non-prostate cancer patients (e.g., brain cancer, breast cancer, ovarian cancer, endometrial cancer, lung adenocarcinoma, melanoma, non-Hodgkin's lymphoma, pancreatic cancer, skin cancer, etc.), negative samples from inflammatory condition patients (e.g., benign prostatic hyperplasia, etc.), negative samples from healthy patients, or any combination thereof. In some embodiments, a validation study may comprise samples from patients of any appropriate age range, e.g., <31 years old, 31-40 years old, 41-50 years old, 51-60 years old, 61-70 years old, 71-80 years old, or >80 years old. In some embodiments, a validation study may comprise samples from patients of any race/ethnicity/descent, (e.g., Caucasians, Africans, Asians etc.).

In certain embodiments, at least one target biomarker signature comprising at least one surface biomarker (e.g., extracellular vesicle-associated surface biomarker) and at least one (including, e.g., at least two, or more) target biomarker (which may be selected from any of surface biomarkers described herein, intravesicular biomarkers described herein, and/or intravesicular RNA biomarkers described herein) may be embodied in a prostate cancer detection assay. In some such embodiments, at least one capture agent is directed to the surface biomarker, and at least one set of detection probes is directed to one or more of such target biomarkers described herein.

In certain embodiments, at least two (including, e.g., at least three or more) distinct target biomarker signatures each comprising at least one surface biomarker (e.g., extracellular vesicle-associated surface biomarker) and at least one (including, e.g., at least two, or more) target biomarker (which may be selected from any of surface biomarkers described herein, intravesicular biomarkers described herein, and/or intravesicular RNA biomarkers described herein) may be embodied in a prostate cancer detection assay.

In some embodiments, each distinct target biomarker signature may have a different pre-determined cutoff value for individually determining whether a sample is positive for prostate cancer. In some embodiments, a sample is determined to be positive for prostate cancer if assay readout is above at least one of cutoff values for a plurality of (e.g., at least 2 or more) target biomarker signatures. In some embodiments, a diagnostic value or a risk score cutoff can be determined based on a plurality of (e.g., at least 2, at least 3 or more) target biomarker signatures.

Accordingly, in some embodiments, a sample can be divided into aliquots such that a different capture agent and/or a different set of detection probes (e.g., each directed to detection of a distinct disease or condition) can be added to a different aliquot. In such embodiments, provided technologies can be implemented with one aliquot at a time or multiple aliquots at a time (e.g., for parallel assays to increase throughput).

In some embodiments, amount of detection probes that is added to a sample provides a sufficiently low concentration of detection probes in a mixture to ensure that the detection probes will not randomly come into close proximity with one another in the absence of binding to an entity of interest (e.g., biological entity), at least not to any great or substantial degree. As such, in many embodiments, when detection probes simultaneously bind to the same entity of interest (e.g., biological entity) through the binding interaction between respective targeting binding moieties of the detection probes and the binding sites of an entity of interest (e.g., a biological entity), the detection probes come into sufficiently close proximity to one another to form double-stranded complex (e.g., as described herein). In some embodiments, the concentration of detection probes in a mixture following combination with a sample may range from about 1 fM to 1 μM, such as from about 1 pM to about 1 nM, including from about 1 pM to about 100 nM.

In some embodiments, the concentration of an entity of interest (e.g., a biological entity) in a sample is sufficiently low such that a detection probe binding to one entity of interest (e.g., a biological entity) will not randomly come into close proximity with another detection probe binding to another entity of interest (e.g., biological entity) in the absence of respective detection probes binding to the same entity of interest (e.g., biological entity), at least not to any great or substantial degree. By way of example only, the concentration of an entity of interest (e.g., biological entity) in a sample is sufficiently low such that a first target detection probe binding to a non-target entity of interest (e.g., a non-cancerous biological entity such as an extracellular vesicle comprising a first target) will not randomly come into close proximity with another different target detection probe that is bound to another non-target entity of interest (e.g., a non-cancerous biological entity such as an extracellular vesicle), at least not to any great or substantial degree, to generate a false positive detectable signal.

Following contacting an entity of interest (e.g., biological entity) in a sample with a set of detection probes, such a mixture may be incubated for a period of time sufficient for the detection probes to bind corresponding targets (e.g., molecular targets), if present, in the entity of interest to form a double-stranded complex (e.g., as described herein). In some embodiments, such a mixture is incubated for a period of time ranging from about 5 min to about 5 hours, including from about 30 min to about 2 hours, at a temperature ranging from about 10 to about 50° C., including from about 20° C., to about 37° C.

A double-stranded complex (resulted from contacting an entity of interest such as a biological entity with detection probes) can then be subsequently contacted with a nucleic acid ligase to perform nucleic acid ligation of a free 3′ end hydroxyl and 5′ end phosphate end of oligonucleotide strands of detection probes, thereby generating a ligated template comprising oligonucleotide strands of at least two or more detection probes. In some embodiments, prior to contacting an assay sample comprising a double-stranded complex with a nucleic acid ligase, at least one or more inhibitor oligonucleotide (e.g., as described herein) can be added to the assay sample such that the inhibitor oligonucleotide can capture any residual free-floating detection probes that may otherwise interact with each other during a ligation reaction.

As is known in the art, ligases catalyze the formation of a phosphodiester bond between juxtaposed 3′-hydroxyl and 5′-phosphate termini of two immediately adjacent nucleic acids when they are annealed or hybridized to a third nucleic acid sequence to which they are complementary. Any known nucleic acid ligase (e.g., DNA ligases) may be employed, including but not limited to temperature sensitive and/or thermostable ligases. Non-limiting examples of temperature sensitive ligases include bacteriophage T4 DNA ligase, bacteriophage T7 ligase, and E. coli ligase. Non-limiting examples of thermostable ligases include Taq ligase, Tth ligase, and Pfu ligase. Thermostable ligase may be obtained from thermophilic or hyper thermophilic organisms, including but not limited to, prokaryotic, eukaryotic, or archaeal organisms. In some embodiments, a nucleic acid ligase is a DNA ligase. In some embodiments, a nucleic acid ligase can be a RNA ligase.

In some embodiments, in a ligation step, a suitable nucleic acid ligase (e.g., a DNA ligase) and any reagents that are necessary and/or desirable are combined with the reaction mixture and maintained under conditions sufficient for ligation of the hybridized ligation oligonucleotides to occur. Ligation reaction conditions are well known to those of skill in the art. During ligation, a reaction mixture, in some embodiments, may be maintained at a temperature ranging from about 20° C., to about 45° C., such as from about 25° C., to about 37° C. for a period of time ranging from about 5 minutes to about 16 hours, such as from about 1 hour to about 4 hours. In yet other embodiments, a reaction mixture may be maintained at a temperature ranging from about 35° C., to about 45° C., such as from about 37° C., to about 42° C., e.g., at or about 38° C. 39° C., 40° C. or 41° C., for a period of time ranging from about 5 minutes to about 16 hours, such as from about 1 hour to about 10 hours, including from about 2 to about 8 hours.

Detection of such a ligated template can provide information as to whether an entity of interest (e.g., a biological entity) in a sample is positive or negative for targets to which detection probes are directed. For example, a detectable level of such a ligated template is indicative of a tested entity of interest (e.g., a biological entity) comprising targets (e.g., molecular targets) of interest. In some embodiments, a detectable level is a level that is above a reference level, e.g., by at least 10% or more, including, e.g., at least 20%, at least 30%, at least 40%, at least 50%, at least 60%, at least 70%, at least 80%, at least 90% or more. In some embodiments, a reference level may be a level observed in a negative control sample, such as a sample in which an entity of interest comprising such targets is absent. Conversely, a non-detectable level (e.g., a level that is below the threshold of a detectable level) of such a ligated template indicates that at least one of targets (e.g., molecular targets) of interest is absent from a tested entity of interest (e.g., a biological entity). Those of skill in the art will appreciate that a threshold that separates a detectable level from a non-detectable level may be determined based on, for example, a desired sensitivity level, and/or a desired specificity level that is deemed to be optimal for each application and/or purpose. For example, in some embodiments, a specificity of 99.7% may be achieved using a system provided herein, for example by setting a threshold that is three standard deviations above a reference level (e.g., a level observed in a negative control sample, such as, e.g., a sample derived from one or more normal healthy individuals). Additionally or alternatively, those of skill in the art will appreciate that a threshold of a detectable level (e.g., as reflected by a detection signal intensity) may be 1 to 100-fold above a reference level.

In some embodiments, a method provided herein comprises, following ligation, detecting a ligated template, e.g., as a measure of the presence and/or amount of an entity of interest in a sample. In various embodiments, detection of a ligated template may be qualitative or quantitative. As such, in some embodiments where detection is qualitative, a method provides a reading or evaluation, e.g., assessment, of whether or not an entity of interest (e.g., a biological entity) comprising at least two or more targets (e.g., molecular targets) is present in a sample being assayed. In other embodiments, a method provides a quantitative detection of whether an entity of interest (e.g., a biological entity) comprising at least two or more targets (e.g., molecular targets) is present in a sample being assayed, e.g., an evaluation or assessment of the actual amount of an entity of interest (e.g., a biological entity) comprising at least two or more targets (e.g., molecular targets) in a sample being assayed. In some embodiments, such quantitative detection may be absolute or relative.

A ligated template formed by using technologies provided herein may be detected by an appropriate method known in the art. Those of skill in the art will appreciate that appropriate detection methods may be selected based on, for example, a desired sensitivity level and/or an application in which a method is being practiced. In some embodiments, a ligated template can be directly detected without any amplification, while in other embodiments, ligated template may be amplified such that the copy number of the ligated template is increased, e.g., to enhance sensitivity of a particular assay. Where detection without amplification is practicable, a ligated template may be detected in a number of different ways. For example, oligonucleotide domains of detection probes (e.g., as described and/or utilized herein) may have been directly labeled, e.g., fluorescently or radioisotopically labeled, such that a ligated template is directly labeled. For example, in some embodiments, an oligonucleotide domain of a detection probe (e.g., as provided and/or utilized herein) can comprise a detectable label. A detectable label may be a composition detectable by spectroscopic, photochemical, biochemical, immunochemical, electrical, optical or chemical means. Such labels include biotin for staining with labeled Streptavidin conjugate, magnetic beads (e.g., Dynabeads®), fluorescent dyes (e.g., fluorescein, Texas red, rhodamine, green fluorescent protein, and the like), radiolabels (e.g., ³H, ¹²⁵I, ³⁴S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. In some embodiments, a directly labeled ligated template may be size separated from the remainder of the reaction mixture, including unligated directly labeled ligation oligonucleotides, in order to detect the ligated template.

In some embodiments, detection of a ligated template can include an amplification step, where the copy number of ligated nucleic acids is increased, e.g., in order to enhance sensitivity of the assay. The amplification may be linear or exponential, as desired, where amplification can include, but is not limited to polymerase chain reaction (PCR); quantitative PCR, isothermal amplification, NASBA, digital droplet PCR, etc.

Various technologies for achieving PCR amplification are known in the art; those skilled in the art will be well familiar with a variety of embodiments of PCR technologies, and will readily be able to select those suitable to amplify a ligated template generated using technologies provided herein. For example, in some embodiments, a reaction mixture that includes a ligated template is combined with one or more primers that are employed in the primer extension reaction, e.g., PCR primers (such as forward and reverse primers employed in geometric (or exponential) amplification, or a single primer employed in a linear amplification). Oligonucleotide primers with which one or more ligated templates are contacted should be of sufficient length to provide for hybridization to complementary template DNA under appropriate annealing conditions. Primers are typically at least 10 bp in length, including, e.g., at least 15 bp in length, at least 20 bp in length, at least 25 bp in length, at least 30 bp in length or longer. In some embodiments, the length of primers can typically range from about 15 to 50 bp in length, from about 18 to 30 bp, or about 20 to 35 bp in length. Ligated templates may be contacted with a single primer or a set of two primers (forward and reverse primers), depending on whether primer extension, linear, or exponential amplification of the template DNA is desired.

In addition to the above components, a reaction mixture comprising a ligated template typically includes a polymerase and deoxyribonucleoside triphosphates (dNTPs). The desired polymerase activity may be provided by one or more distinct polymerase enzymes. In preparing a reaction mixture, e.g., for amplification of a ligated template, various constituent components may be combined in any convenient order. For example, an appropriate buffer may be combined with one or more primers, one or more polymerases and a ligated template to be detected, or all of the various constituent components may be combined at the same time to produce the reaction mixture.

### VI. Uses

In some embodiments, one or more provided biomarkers of one or more target biomarker signatures for prostate cancer can be detected in a sample comprising biological entities (including, e.g., cells, circulating tumor cells, cell-free DNA, extracellular vesicles, etc.), for example, using methods of detecting and/or assays as described herein. In some embodiments, one or more provided biomarkers of one or more target biomarker signatures for prostate cancer can be detected in a sample comprising nanoparticles having a size range of interest that includes extracellular vesicles, for example, using methods of detecting and/or assays as described herein.

In some embodiments, a sample may be or comprise a biological sample. In some embodiments, a biological sample is a bodily fluid sample of a subject (e.g., a human subject). In some embodiments, a biological sample can be derived from a blood or blood-derived sample of a subject (e.g., a human subject) in need of such an assay. In some embodiments, a biological sample can be or comprise a primary sample (e.g., a tissue or tumor sample) from a subject (e.g., a human subject) in need of such an assay. In some embodiments, a biological sample can be processed to separate one or more entities of interest (e.g., biological entity) from non-target entities of interest, and/or to enrich one or more entities of interest (e.g., biological entity). In some embodiments, an entity of interest present in a sample may be or comprise a biological entity, e.g., a cell or a nanoparticle having a size range of interest that includes extracellular vesicles (e.g., an exosome). In some embodiments, such a biological entity (e.g., extracellular vesicle) may be processed or contacted with a chemical reagent, e.g., to stabilize and/or crosslink targets (e.g., provided target biomarkers) to be assayed in the biological entity and/or to reduce non-specific binding with detection probes. In some embodiments, a biological entity is or comprises a cell, which may be optionally processed, e.g., with a chemical reagent for stabilizing and/or crosslinking targets (e.g., molecular targets) and/or for reducing non-specific binding. In some embodiments, a biological entity is or comprises an extracellular vesicle (e.g., an exosome), which may be optionally processed, e.g., with a chemical reagent for stabilizing and/or crosslinking targets (e.g., molecular targets) and/or for reducing non-specific binding.

In some embodiments, technologies provided herein can be useful for managing patient care, e.g., for one or more individual subjects and/or across a population of subjects. By way of example only, in some embodiments, provided technologies may be utilized in screening, which for example, may be performed periodically, such as annually, semi-annually, bi-annually, or with some other frequency as deemed to be appropriate by those skilled in the art. In some embodiments, such a screening may be temporally motivated or incidentally motivated. For example, in some embodiments, provided technologies may be utilized in temporally motivated screening for one or more individual subjects or across a population of subjects (e.g., asymptomatic subjects) who are older than a certain age (e.g., over 40, 45, 50, 55, 60, 65, 70, 75, 80, or older). As will be appreciated by those skilled in the art, in some embodiments, the screening age and/or frequency may be determined based on, for example, but not limited to prevalence of a disease, disorder, or condition (e.g., cancer such as prostate cancer). In some embodiments, provided technologies may be utilized in incidentally-motivated screening for individual subjects who may have experienced an incident or event that motivates screening for a particular disease, disorder, or condition (e.g., cancer such as prostate cancer). For example, in some embodiments, an incidental motivation relating to determination of one or more indicators of a disease, disorder, or condition (e.g., cancer such as prostate cancer) or susceptibility thereto may be or comprise, e.g., an incident based on their family history (e.g., a close relative such as blood-related relative was previously diagnosed for such a disease, disorder, or condition such as prostate cancer), identification of one or more life-history associated risk factors for a disease, disorder, or condition (e.g., prostate cancer) and/or prior incidental findings from genetic tests (e.g., genome sequencing), and/or imaging diagnostic tests (e.g., ultrasound, computerized tomography (CT) and/or magnetic resonance imaging (MRI) scans), development of one or more signs or symptoms characteristic of a particular disease, disorder, or condition (e.g., chronic inflammatory diseases, e.g., benign prostatic hyperplasia), subjects having benign prostate tumors, and combinations thereof, and/or other incidents or events as will be appreciated by those skilled in the art.

In some embodiments, provided technologies for managing patient care can inform treatment and/or payment (e.g., reimbursement for treatment) decisions and/or actions. For example, in some embodiments, provided technologies can provide determination of whether individual subjects have one or more indicators of risk, incidence, or recurrence of a disease disorder, or condition (e.g., cancer such as prostate cancer), thereby informing physicians and/or patients when to provide/receive therapeutic or prophylactic recommendations and/or to initiate such therapy in light of such findings. In some embodiments, such individual subjects may be asymptomatic subjects, who may be temporally-motivated or incidentally-motivated to be screened at a regular frequency (e.g., annually, semi-annually, bi-annually, or other frequency as deemed to be appropriate by those skilled in the art). In some embodiments, such individual subjects may be experiencing one or more symptoms that may be associated with prostate cancer, who may be temporally-motivated or incidentally-motivated to be screened at a regular frequency (e.g., annually, semi-annually, bi-annually, or other frequency as deemed to be appropriate by those skilled in the art). In some embodiments, such individual subjects may be subjects having benign prostatic hyperplasia and/or a chronic inflammatory condition, who may be temporally-motivated or incidentally-motivated screened at a regular frequency (e.g., annually, semi-annually, bi-annually, or other frequency as deemed to be appropriate by those skilled in the art). In some embodiments, such individual subjects may be subjects at hereditary risk for prostate cancer, who may be temporally-motivated or incidentally-motivated to be screened at a regular frequency (e.g., annually, semi-annually, bi-annually, or other frequency as deemed to be appropriate by those skilled in the art). In some embodiments, such individual subjects may be subjects with life-history associated risk, who may be temporally-motivated or incidentally-motivated screened at a regular frequency (e.g., annually, semi-annually, bi-annually, or other frequency as deemed to be appropriate by those skilled in the art). In some embodiments, such individual subjects may be obese and/or smoking subjects (e.g., a BMI over 30 and/or heavy smokers), who may be temporally-motivated or incidentally-motivated screened at a regular frequency (e.g., annually, semi-annually, bi-annually, or other frequency as deemed to be appropriate by those skilled in the art). In some embodiments, such obese and/or smoking subjects may be experiencing abdominal pain.

Additionally or alternatively, in some embodiments, provided technologies can inform physicians and/or patients of treatment selection, e.g., based on findings of specific responsiveness biomarkers (e.g., cancer responsiveness biomarkers). In some embodiments, provided technologies can provide determination of whether individual subjects are responsive to current treatment, e.g., based on findings of changes in one or more levels of molecular targets associated with a disease, thereby informing physicians and/or patients of efficacy of such therapy and/or decisions to maintain or alter therapy in light of such findings. In some embodiments, provided technologies can provide determination of whether individual subjects are likely to be responsive to a recommended treatment, e.g., based on findings of molecular targets (e.g., provided biomarkers of one or more target biomarker signatures for prostate cancer, e.g., in some embodiments prostate adenocarcinoma) that predict therapeutic effects of a recommended treatment on individual subjects, thereby informing physicians and/or patients of potential efficacy of such therapy and/or decisions to administer or alter therapy in light of such findings.

In some embodiments, provided technologies can inform decision making relating to whether health insurance providers reimburse (or not), e.g., for (1) screening itself (e.g., reimbursement available only for periodic/regular screening or available only for temporally- and/or incidentally-motivated screening); and/or for (2) initiating, maintaining, and/or altering therapy in light of findings by provided technologies. For example, in some embodiments, the present disclosure provides methods relating to (a) receiving results of a screening that employs provided technologies and also receiving a request for reimbursement of the screening and/or of a particular therapeutic regimen; (b) approving reimbursement of the screening if it was performed on a subject according to an appropriate schedule (based on, e.g., screening age such as older than a certain age, e.g., over 40, 45, 50, 55, 60, 65, 70, 75, 80, or older, and/or screening frequency such as, e.g., every 3 months, every 6 months, every year, every 2 years, every 3 years or at some other frequencies) or in response to a relevant incident and/or approving reimbursement of the therapeutic regimen if it represents appropriate treatment in light of the received screening results; and, optionally (c) implementing the reimbursement or providing notification that reimbursement is refused. In some embodiments, a therapeutic regimen is appropriate in light of received screening results if the received screening results detect a biomarker that represents an approved biomarker for the relevant therapeutic regimen (e.g., as may be noted in a prescribing information label and/or via an approved companion diagnostic).

Alternatively or additionally, the present disclosure contemplates reporting systems (e.g., implemented via appropriate electronic device(s) and/or communications system(s)) that permit or facilitate reporting and/or processing of screening results (e.g., as generated in accordance with the present disclosure), and/or of reimbursement decisions as described herein. Various reporting systems are known in the art; those skilled in the art will be well familiar with a variety of such embodiments, and will readily be able to select those suitable for implementation.

### Exemplary Uses

### A. Detection of Prostate Cancer Incidence or Recurrence

The present disclosure, among other things, recognizes that detection of a single cancer-associated biomarker in a biological entity (e.g., extracellular vesicle) or a plurality of cancer-associated biomarkers based on a bulk sample, rather than at a resolution of a single biological entity (e.g., individual extracellular vesicles), typically does not provide sufficient specificity and/or sensitivity in determination of whether a subject from whom the biological entity is obtained is likely to be suffering from or susceptible to cancer (e.g., prostate cancer). The present disclosure, among other things, provides technologies, including compositions and/or methods, that solve such problems, including for example by specifically requiring that an entity (e.g., a nanoparticle having a size range of interest that includes an extracellular vesicle) for detection be characterized by presence of a combination of at least two or more targets (e.g., at least two or more provided biomarkers of a target biomarker signature for prostate cancer). In particular embodiments, the present disclosure teaches technologies that require such an entity (e.g., a nanoparticle having a size range of interest that includes an extracellular vesicle) be characterized by presence (e.g., by expression) of a combination of molecular targets that is specific to cancer (i.e., “target biomarker signature” of a relevant cancer, e.g., prostate cancer), while biological entities (e.g., nanoparticles having a size range of interest that includes extracellular vesicles) that do not comprise the targeted combination (e.g., target biomarker signature) do not produce a detectable signal. Accordingly, in some embodiments, technologies provided herein can be useful for detection of risk, incidence, and/or recurrence of cancer in a subject. In some such embodiments, technologies provided herein are useful for detection of risk, incidence, and/or recurrence of prostate cancer in a subject. For example, in some embodiments, a combination of two or more provided biomarkers are selected for detection of a specific cancer (e.g., prostate cancer) or various cancers (one of which includes prostate cancer). In some embodiments, a specific combination of provided biomarkers for detection of prostate cancer can be determined by analyzing a population or library (e.g., tens, hundreds, thousands, tens of thousands, hundreds of thousands, or more) of prostate cancer patient blood samples and/or patient data to identify such a predictive combination. In some embodiments, a relevant combination of biomarkers may be one identified and/or characterized, for example, via data analysis. For example, in some embodiments, data analysis may comprise a bioinformatic analysis, for example, as described in Examples 6-8. In some embodiments, for example, a diverse set of prostate cancer-associated data (e.g., in some embodiments comprising one or more of bulk RNA sequencing, single-cell RNA (scRNA) sequencing, mass spectrometry, histology, post-translational modification data, in vitro and/or in vivo experimental data) can be analyzed through machine learning and/or computational modeling to identify a combination of predictive markers that is highly specific to prostate cancer. In some embodiments, a combination of predictive markers to distinguish stages of cancer (e.g., prostate cancer) can be determined in silico based on comparing and analyzing diverse data (e.g., in some embodiments comprising bulk RNA sequencing, scRNA sequencing, mass spectrometry, histology, post-translational modification data, in vitro and/or in vivo experimental data) relating to different stages of cancer (e.g., prostate cancer). For example, in some embodiments, technologies provided herein can be used to distinguish prostate cancer subjects from non-prostate cancer subjects, including, e.g., healthy subjects, subjects diagnosed with benign tumors or abdominal masses, and subjects with non-prostate-related diseases, disorders, and/or conditions (e.g., subjects with non-prostate cancer, or subjects with inflammatory conditions, e.g., Chron's disease, ulcerative colitis). In some embodiments, technologies provided herein can be useful for early detection of prostate cancer, e.g., detection of prostate cancer of stage I or stage II. In some embodiments, technologies provided herein can be useful for detection of one or more prostate cancer subtypes, including, e.g., prostate adenocarcinoma and other specified types of prostate cancer as known in the art (SEER Cancer Statistics Review 1975-2017). In some embodiments, technologies provided herein can be useful for screening individuals at hereditary risk, life-history associated risk, or average risk for early-stage prostate cancer (e.g., prostate adenocarcinoma).

In some embodiments, technologies provided herein can be useful for screening a subject for risk, incidence, or recurrence of a specific cancer in a single assay. For example, in some embodiments, technologies provided herein is useful for screening a subject for risk, incidence, or recurrence of prostate cancer. In some embodiments, technologies provided herein can be used to screen a subject for risk or incidence of a specific cancer or a plurality of (e.g., at least 2, at least 3, or more) cancers in a single assay. For example, in some embodiments, technologies provided herein can be used to screen a subject for a plurality of cancers in a single assay, one of which includes prostate cancer and other cancers to be screened can be selected from the group consisting of brain cancer (including, e.g., glioblastoma), breast cancer, ovarian cancer, pancreatic cancer, liver cancer, lung cancer, and skin cancer.

In some embodiments, provided technologies can be used periodically (e.g., every year, every two years, every three years, etc.) to screen a human subject for prostate cancer (e.g., early-stage prostate cancer) or cancer recurrence. In some embodiments, a human subject amenable to such screening may be an adult or an elderly. In some embodiments, a human subject amenable to such screening may be older than a specified age, e.g., age 45 and above, age 55 and above, age 65 and above, age 70 and above, at least age 75 above, or age 80 and above. In some embodiments, a human subject amenable to such screening may have an age of about 50 or above. In some embodiments, a human subject amenable to such screening may have an age of 50 or less. In some embodiments, a human subject amenable to such screening may have an age over 35. In some embodiments, a human subject who is determined to have a genetic predisposition to prostate cancer may be screened at a younger age than a human subject who has no family history risk.

In some embodiments, a subject that is amenable to provided technologies for detection of incidence or recurrence of prostate cancer may be a human subject with a smoking or obesity history (e.g., a heavy smoker and/or a BMI over 30), who in some embodiments may be experiencing one or more symptoms associated with prostate cancer or a subset thereof (e.g., prostate adenocarcinoma). In some embodiments, a subject that is amenable to provided technologies for detection of incidence or recurrence of prostate cancer may be a human subject who is at least 50 years old and is determined to have a benign prostate tumor and/or one or more chronic inflammatory conditions (e.g., benign prostatic hyperplasia). In some embodiments, a subject that is amenable to provided technologies for detection of incidence or recurrence of prostate cancer may be a subject who has a family history of prostate cancer (e.g., subjects having one or more first-degree relatives with a history of prostate cancer), who has been previously treated for cancer (e.g., prostate cancer, prostate adenocarcinoma), who is at risk of prostate cancer recurrence after cancer treatment, who is in remission after prostate cancer treatment, and/or who has been previously or periodically screened for prostate cancer, e.g., by screening for the presence of at least one prostate cancer biomarker (e.g., as described herein).

In some embodiments, the present disclosure, among other things, provides insights that technologies described and/or utilized herein may be particularly useful for screening certain populations of subjects, e.g., subjects who are at higher susceptibility to developing prostate cancer. In some embodiments, the present disclosure, among other things, recognizes that the resulting PPVs of technologies described and/or utilized herein for prostate adenocarcinoma detection may be higher in prostate cancer prone or susceptible populations. In some embodiments, the present disclosure, among other things, provides insights that screening of smoking or obese individuals, e.g., regular screening prior to or otherwise in absence of developed symptom(s), can be beneficial, and even important for effective management (e.g., successful treatment) of prostate cancer. In some embodiments, the present disclosure provides prostate cancer screening systems that can be implemented to detect prostate cancer, including early-stage cancer, in some embodiments in obese and/or smoking individuals (e.g., with or without hereditary and/or life-history risks in prostate cancer and/or with or without symptoms associated with prostate cancer). In some embodiments, provided technologies can be implemented to achieve regular screening of obese and/or smoking individuals (e.g., with or without hereditary and/or life-history risks in prostate cancer and/or with or without symptoms associated with prostate cancer). In some embodiments, provided technologies achieve detection (e.g., early detection, e.g., in symptomatic or asymptomatic individual(s) and/or population(s)) of one or more features (e.g., incidence, progression, responsiveness to therapy, recurrence, etc.) of prostate cancer, with sensitivity and/or specificity (e.g., rate of false positive and/or false negative results) appropriate to permit useful application of provided technologies to single-time and/or regular (e.g., periodic) assessment. In some embodiments, provided technologies are useful in conjunction with a subject's periodic physical examination (e.g., every year, every other year, or at an interval approved by the attending physician). In some embodiments, provided technologies are useful in conjunction with treatment regimen(s); in some embodiments, provided technologies may improve one or more characteristics (e.g., rate of success according to an accepted parameter) of such treatment regimen(s).

In some embodiments, a subject that is amenable to provided technologies for detection of incidence or recurrence of prostate cancer may be an asymptomatic human subject and/or across an asymptomatic population of subjects. Such an asymptomatic subject and/or across an asymptomatic population of subjects may be subject(s) who has/have a family history of cancers such as breast and/or ovarian cancer, leukemia, and/or prostate cancer (e.g., individuals having one or more first-degree relatives with a history of cancers known to be associated with genetic risk factors), who has been previously treated for cancer (e.g., prostate cancer), who is at risk of prostate cancer recurrence after cancer treatment, who is in remission after prostate cancer treatment, and/or who has been previously or periodically screened for prostate cancer, e.g., by screening for the presence of at least one prostate cancer biomarker via digital rectal examination (DRE) or other means (e.g., X-ray imaging, low-dose CT scanning, and/or molecular tests based on cell-free nucleic acids, serum biomarkers (e.g. PSA). Alternatively, in some embodiments, an asymptomatic subject may be a subject who has not been previously screened for prostate cancer, who has not been diagnosed for prostate cancer, and/or who has not previously received prostate cancer therapy. In some embodiments, an asymptomatic subject may be a subject with a benign prostate tumor. In some embodiments, an asymptomatic subject may be a subject who is susceptible to prostate cancer (e.g., at an average population risk, at an elevated life-history associated risk, or with hereditary risk for prostate cancer).

In some embodiments, a subject or population of subjects that are amenable to provided technologies for detection of prostate cancer may be selected based on one or more characteristics such as age, race, geographic location, genetic history, medical history, personal history (e.g., smoking, alcohol, drugs, carcinogenic agents, diet, obesity, physical activity, sun exposure, radiation exposure, and/or occupational hazard). For example, in some embodiments, a subject or population of subjects that are amenable to provided technologies for detection of prostate cancer may be a subject or a population of subjects determined to currently be or have been a smoker (e.g., cigarettes, cigars, pipe, and/or hookah) or obese.

In some embodiments, a subject or population of subjects that are amenable to provided technologies for detection of prostate cancer may be a subject or a population of subjects determined to have one or more germline mutations in genes associated with hereditary risk for development of prostate cancer (e.g., BRCA1, BRCA2, CHEK2, ATM, PALB2, RAD51D, MSH2, MSH6, MLH1, PMS2, RNASEL (formerly HPC1), HOXB13, and combinations thereof).

In some embodiments, a subject or population of subjects that are amenable to provided technologies for detection of prostate cancer may be a subject or a population of subjects diagnosed with an imaging-confirmed prostate mass.

In some embodiments, a subject or population of subjects that are amenable to provided technologies for detection of prostate cancer may be a subject or a population of subjects at hereditary risk or life-history associated risk before undergoing a biopsy and/or a surgical procedure (e.g., prostatectomy).

In some embodiments, a subject or population of subjects that are amenable to provided technologies for detection of prostate cancer may be a subject or population of subjects determined to have benign prostatic hyperplasia. In some embodiments, a subject or population of subjects that are amenable to provided technologies for detection of prostate cancer may be a subject or population of subjects with a history of chronic urinary tract issues. In some embodiments, a subject or population of subjects that are amenable to provided technologies for detection of prostate cancer may be a subject or population of subjects with high current or historical alcohol consumption. In some embodiments, a subject or population of subjects that are amenable to provided technologies for detection of prostate cancer may be a subject or population of subjects that are obese. In some embodiments, a subject or population of subjects that are amenable to provided technologies for detection of prostate cancer may be a subject or population of subjects that have been exposed to chemicals (e.g., firefighters or subjects exposed to Agent Orange). In some embodiments, a subject or population that are amenable to provided technologies for detection of prostate cancer may be subject or population of subjects consuming higher than average quantities or red meat (e.g. people residing in the United States). In some embodiments, a subject or population of subjects that are amenable to provided technologies for detection of prostate cancer may be a subject or population of subjects determined to have hereditary mutations in genes associated with hereditary risk for development of prostate cancer (e.g., BRCA1, BRCA2, CHEK2, ATM, PALB2, RAD51D, MSH2, MSH6, MLH1, PMS2, RNASEL (formerly HPC1), HOXB13, and combinations thereof). In some embodiments, a subject or population of subjects that are amenable to provided technologies for detection of prostate cancer may be a subject or population of subjects exposed to radiation therapy and/or chemotherapy.

In some embodiments, a subject or population of subjects that are amenable to provided technologies for detection of prostate cancer may be a subject or a population of subjects with one or more non-specific symptoms of prostate cancer. In some embodiments, exemplary non-specific symptoms of prostate cancer may include symptoms similar to those of diseases of the urinary tract, and/or symptoms such as difficulty urinating, blood in the urine and/or semen, erectile dysfunction, pain in the hips, back (spine), or chest (ribs), weakness or numbness in the legs and/or feet, loss of bladder and/or bowel control, and/or other symptoms associated with the prostate.

In some embodiments, a subject or population of subjects that are amenable to provided technologies for detection of prostate cancer may be a subject or a population of subjects of diverse descendants such as Asians, African Americans, Caucasians, Native Hawaiians or other Pacific Islanders, Hispanics or Latinos, American Indians or Alaska natives, non-Hispanic blacks, or non-Hispanic whites. In some embodiments, a subject or population of subjects that are amenable to provided technologies for detection of prostate cancer may be a subject or a population of subjects of diverse descendants such as Asian Pacific Islanders, Hispanics, American Indian/Alaska natives, non-Hispanic black, or non-Hispanic white. In some embodiments, a subject or population of subjects that are amenable to provided technologies for detection of prostate cancer may be a subject or a population of subjects of any race and/or any ethnicity.

In some embodiments, a subject or population of subjects that are amenable to provided technologies for detection of prostate cancer may have been previously subjected to digital rectal examination (DRE), low-dose CT scanning, and/or molecular tests based on cell-free nucleic acids and/or serum biomarkers (e.g., PSA). In some embodiments, such subjects may have received a negative indication of prostate cancer (e.g., prostate adenocarcinoma) from such diagnostic tests. In some embodiments, such subjects may have received a positive indication of prostate cancer from such diagnostic tests.

In some embodiments, technologies provided herein can be used in combination with other diagnostics assays including, e.g., but not limited to (i) physicals, general practitioner visits, cholesterol/lipid blood tests, urine tests, diabetes (type 2) screening, colonoscopies, blood pressure screening, thyroid function tests, prostate cancer screening, and/or vaccinations; (ii) DRE, abdominal CT scanning, and/or molecular tests based on cell-free nucleic acids from blood, urine, or semen, and/or serum biomarkers (e.g., PSA); (iii) a genetic assay to screen blood plasma for genetic mutations in circulating tumor DNA and/or protein biomarkers linked to cancer; (iv) an assay involving immunofluorescence staining to identify cell phenotype and marker expression, followed by amplification and analysis by next-generation sequencing; and (v) germline and somatic mutation assays, or assays involving cell-free tumor DNA, liquid biopsy, serum protein, cell-free DNA, urine biomarkers (e.g., PCA3), and/or circulating tumor cells.

### B. Selection of Cancer Therapy (e.g., Prostate Cancer Therapy)

In some embodiments, provided technologies can be used for selecting an appropriate treatment for a cancer patient (e.g., a patient suffering from or susceptible to prostate cancer). For example, some embodiments provided herein relate to a companion diagnostic assay for classification of patients for cancer therapy (e.g., prostate cancer and/or adjunct treatment) which comprises assessment in a patient sample (e.g., a blood or blood-derived sample from a prostate cancer patient) of a selected combination of provided biomarkers using technologies provided herein. Based on such an assay outcome, patients who are determined to be more likely to respond to a cancer therapy (e.g., a prostate cancer therapy and/or an adjunct therapy, including, e.g., Abiraterone acetate, Apalutamide, Bicalutamide, Cabazitaxel, Calcitriol, Docetaxel, Enzalutamide, Flutamide, Lenalidomide, Nilutamide, Olaparib, and Relugolix), can be administered such a therapy, or patients who are determined to be non-responsive to a specific such therapy can be administered a different therapy.

### C. Evaluation of Treatment Efficacy (e.g., Cancer Treatment Efficacy)

In some embodiments, technologies provided herein can be used for monitoring and/or evaluating efficacy of an anti-cancer therapy administered to a cancer patient (e.g., prostate cancer patient). For example, a bodily fluid sample (e.g., but not limited to a blood sample, a urine sample, etc.) can be collected from a prostate cancer patient prior to or receiving an anti-cancer therapy (e.g., Abiraterone acetate, Apalutamide, Bicalutamide, Cabazitaxel, Calcitriol, Docetaxel, Enzalutamide, Flutamide, Lenalidomide, Nilutamide, Olaparib, and Relugolix) at a first time point to detect or measure tumor burdens, e.g., by detecting presence or amount of nanoparticles having a size range of interest that includes extracellular vesicles comprising a selected combination of biomarkers that is specific to detection of prostate cancer. After a period of treatment, a second bodily fluid sample (e.g., but not limited to a blood sample, a urine sample, etc.) can be collected from the same prostate cancer patient to detect changes in tumor burdens, e.g., by detecting absence or reduction in amount of nanoparticles having a size range of interest that includes extracellular vesicles comprising a selected combination of biomarkers that is specific to detection of prostate cancer. By monitoring levels and/or changes in tumor burdens over the course of treatment, appropriate course of action, e.g., increasing or decreasing the dose of a therapeutic agent, and/or administering a different therapeutic agent, can be taken.

### VII. Kits

Also provided are kits that find use in practicing technologies as described above. In some embodiments, a kit comprises a plurality of detection probes (e.g., as described and/or utilized herein). In some embodiments, a provided kit may comprise two or more (e.g., 3, 4, 5, 6, 7, 8, 9, 10, 11, 12, 13, 14, 15, 16, 17, 18, 19, 20 or more) detection probes. In some embodiments, individual detection probes may be directed at different targets. In some embodiments, two or more individual detection probes may be directed to the same target. In some embodiments, a provided kit comprises two or more different detection probes directed at different targets, and optionally may include at least one additional detection probe also directed at a target to which another detection probe is directed. In some embodiments, a provided kit comprises a plurality of subsets of detection probes, each of which comprises two or more detection probes directed at the same target. In some embodiments, a plurality of detection probes may be provided as a mixture in a container. In some embodiments, multiple subsets of detection probes may be provided as individual mixtures in separate containers. In some embodiments, each detection probe is provided individually in a separate container.

In some embodiments, a kit for detection of prostate cancer comprises: (a) a capture agent comprising a target-capture moiety directed to an extracellular vesicle-associated surface biomarker; and (b) a set of detection probes, which set comprises at least two detection probes each directed to a target biomarker of a target biomarker signature for prostate cancer, wherein the detection probes each comprise: (i) a target binding moiety directed the target biomarker of the target biomarker signature for prostate cancer; and (ii) an oligonucleotide domain coupled to the target binding moiety, the oligonucleotide domain comprising a double-stranded portion and a single-stranded overhang portion extended from one end of the oligonucleotide domain, wherein the single-stranded overhang portions of the at least two detection probes are characterized in that they can hybridize to each other when the at least two detection probes are bound to the same extracellular vesicle.

In some embodiments, the present disclosure describes a kit for detection of prostate cancer comprising: (a) a capture agent comprising a target-capture moiety directed to a first surface biomarker; and (b) at least one set of detection probes, which set comprises at least two detection probes each directed to a second surface biomarker, wherein the detection probes each comprise: (i) a target binding moiety directed at the second surface biomarker; and (ii) an oligonucleotide domain coupled to the target binding moiety, the oligonucleotide domain comprising a double-stranded portion and a single-stranded overhang portion extended from one end of the oligonucleotide domain, wherein the single-stranded overhang portions of the at least two detection probes are characterized in that they can hybridize to each other when the at least two detection probes are bound to the same nanoparticle having the size within the range of about 30 nm to about 1000 nm; wherein at least the first surface biomarker and the second surface biomarker form a target biomarker signature determined to be associated with prostate cancer, and wherein the first and second surface biomarkers are each independently selected from: (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4; and/or (ii) carbohydrate-dependent markers as follows: Tn antigen, SialylTn (sTn) antigen, Thomsen-Friedenreich (T. TF) antigen, Lewis Y antigen (also known as CD174), Sialyl Lewis X (sLex) antigen (also known as Sialyl SSEA-1 (SLX)), and combinations thereof.

In some embodiments, the first and second surface biomarkers are each independently selected from: (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof. In some embodiments, the first and the second surface biomarkers are different. In some embodiments, the first and the second surface biomarkers are the same (with the same or different epitopes).

In many embodiments described herein, a target biomarker signature for prostate cancer comprises: at least one extracellular vesicle-associated surface biomarker and at least one target biomarker selected from the group consisting of: surface biomarkers, intravesicular biomarkers, and intravesicular RNA biomarkers, wherein:
the surface biomarkers are selected from (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, and combinations thereof; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof;
the intravesicular biomarkers are selected from polypeptides encoded by human genes as follows: ACP5, ACPP, ACSM1, ACTA2, ACTG2, ADAMTS1, AGR2, AIM1, ALCAM, ALDH1A3, ALOX15B, ANTXR1, ANTXR2, AP1M2, AR, ARG2, ARHGAP6, ARHGEF26, ARHGEF37, ASAP3, ATP8B1, ATP9A, BAIAP2L1, BCAM, BHLHA15, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CGN, CGNL1, CHMP4C, CLGN, CMTM4, COLCA1, CORO2A, CPE, CPNE4, CPQ, CRYM, CTSF, CX3CL1, CXADR, CXCR4, CYB561, DPYSL3, DSP, EEF1A2, EFNA1, EHD2, EHF, ELF3, EMP2, EPHX1, EPN3, ERBB3, ERG, ESRP1, ESRP2, F3, FAAH, FAM129A, FAM129B, FAM210B, FAM3B, FAM3D, FAM83H, FAM84A, FAM84B, FAT1, FBLIM1, FBP1, FLNC, FNBP1L, FOS, FOXA1, GADD45G, GATA2, GGT1, GJB1, GLYATL1, GNG4, GNMT, GRHL2, GUCY1A3, HGD, HID1, HIST1H2BK, HIST3H2A, HMGCS2, HOMER2, HOXB13, HSD17B6, HSPB6, HSPB8, ICA1, ID1, IQGAP2, IRF6, ITGA6, KIAA1522, KIF5C, KLK2, KRT14, KRT15, KRT17, KRT18, KRT19, KRT5, KRT7, KRT8, LCP1, LIFR, LIMCH1, LMOD1, LRP11, LRP3, LSR, MAL2, MAOA, MAP7, MARC1, MARCKSL1, MB, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, PALLD, PARVA, PCDH1, PCP4, PDIA5, PEBP4, PGM5, PKP1, PKP3, PPL, PPM1H, PPP1R1B, PPP3CA, PRAC1, PRR15L, PSAT1, PTPRF, PYCR1, RAB3B, RAMP1, RAP1GAP, RASEF, RBM47, RCAN3, RDH11, REEP6, REPS2, RGS10, RHPN2, RNF144B, RORC, RPS4Y1, S100A16, SELENBP1, SERINC5, SH3BGRL2, SH3BP4, SHC2, SLC1A5, SLC22A3, SLC2A12, SLC40A1, SLC4A4, SLC7A8, SLC9A3R2, SMOC2, SORD, SPDEF, SPINT1, SPINT2, SPTBN2, ST14, STAP2, STK39, SULT2B1, SYNE4, SYT7, SYTL1, TAGLN, TBC1D16, TBX3, TC2N, TCEA3, TEAD3, TESC, THBS4, TM4SF1, TMED3, TMEM150C, TMEM54, TMEM98, TMSB15A, TP53111, TPD52, TPM1, TSPAN13, TSPAN6, TSPAN8, VAMP8, VSTM2L, WLS, WWC1, YAP1, ZBTB42, ZC3H11A, ZNF217, ACSL3, CADH7, KCNN2, OPRK1, PDE9A, PLPP1, SPON2, TMEM121B, and combinations thereof; in some embodiments, an intravesicular biomarker described herein may comprise at least one post-translational modification;
the intravesicular RNA biomarkers are selected from: RNA transcripts (e.g., mRNA transcripts) encoded by human genes as follows: ABCC4, ACP5, ACPP, ACSM1, ACTA2, ACTG2, ADAMTS1, AGR2, AIM1, ALCAM, ALDH1A3, ALOX15B, ANO7, ANPEP, ANTXR1, ANTXR2, AP1M2, APCDD1, AQP3, AR, ARG2, ARHGAP6, ARHGEF26, ARHGEF37, ASAP3, ASTN2, ATP8B1, ATP9A, BAIAP2L1, BCAM, BHLHA15, BIK, BMPR1B, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CDH1, CGN, CGNL1, CHMP4C, CHRM1, CHRNA2, CLDN3, CLDN4, CLDN7, CLDN8, CLGN, CMTM4, COLCA1, COLEC12, CORO2A, CPE, CPNE4, CPQ, CRB3, CREB3L1, CREB3L4, CRYM, CTSF, CWH43, CX3CL1, CXADR, CXCR4, CYB561, DPP4, DPYSL3, DSG2, DSP, EEF1A2, EFNA1, EHD2, EHF, ELF3, ELOVL7, EMB, EMP2, ENPP5, EPCAM, EPHX1, EPN3, ERBB3, ERG, ESRP1, ESRP2, F3, FAAH, FAM129A, FAM129B, FAM189A2, FAM210B, FAM3B, FAM3D, FAM83H, FAM84A, FAM84B, FAT1, FBLIM1, FBP1, FLNC, FNBP1L, FOLH1, FOS, FOXA1, FXYD3, GADD45G, GALNT3, GALNT7, GATA2, GCNT1, GGT1, GJB1, GLYATL1, GNG4, GNMT, GOLM1, GPR160, GREB1, GRHL2, GUCY1A3, HGD, HID1, HIST1H2BK, HIST3H2A, HMGCS2, HOMER2, HOXB13, HPN, HSD17B6, HSPB6, HSPB8, ICA1, ID1, IQGAP2, IRF6, ITGA6, KCNN4, KIAA1324, KIAA1522, KIF5C, KLK2, KRT14, KRT15, KRT17, KRT18, KRT19, KRT5, KRT7, KRT8, KRTCAP3, LCP1, LIFR, LIMCH1, LMOD1, LPAR3, LRP11, LRP3, LRRC26, LSR, MAL2, MAOA, MAP7, MARC1, MARCKSL1, MB, MBOAT2, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, OR51E1, OR51E2, PALLD, PARM1, PARVA, PCDH1, PCP4, PDIA5, PDLIM5, PDZK1IP1, PDZRN3, PEBP4, PGM5, PIGR, PKP1, PKP3, PLA2G2A, PMEPA1, PODXL2, PPL, PPM1H, PPP1R1B, PPP3CA, PRAC1, PROM2, PRR15L, PRSS8, PSAT1, PSCA, PTPRF, PTPRN2, PYCR1, RAB25, RAB27B, RAB3B, RAMP1, RAP1GAP, RASEF, RBM47, RCAN3, RDH11, REEP6, REPS2, RGS10, RHPN2, RNF144B, RORC, RPS4Y1, S100A16, SCNN1A, SDC1, SELENBP1, SERINC2, SERINC5, SEZ6L2, SH3BGRL2, SH3BP4, SHC2, SLC15A2, SLC1A5, SLC22A3, SLC2A10, SLC2A12, SLC30A4, SLC35F2, SLC40A1, SLC44A4, SLC45A3, SLC4A4, SLC7A8, SLC9A3R2, SMIM22, SMOC2, SORD, SPDEF, SPINT1, SPINT2, SPTBN2, ST14, STAP2, STEAP1, STEAP2, STEAP4, STK39, SULT2B1, SYNE4, SYT7, SYTL1, TACSTD2, TAGLN, TBC1D16, TBX3, TC2N, TCEA3, TEAD3, TESC, THBS4, TM4SF1, TMC4, TMC5, TMED3, TMEFF2, TMEM125, TMEM150C, TMEM30B, TMEM45B, TMEM54, TMEM98, TMPRSS2, TMSB15A, TP53111, TPD52, TPM1, TRPM4, TRPM8, TRPV6, TSPAN1, TSPAN13, TSPAN6, TSPAN8, TUSC3, UPK3A, VAMP8, VSIG2, VSTM2L, WLS, WWC1, YAP1, ZBTB42, ZC3H11A, ZNF217, and combinations thereof.

In some embodiments, a kit for detection of prostate cancer comprises: (a) a capture agent comprising a target-capture moiety directed to an extracellular vesicle-associated surface biomarker; and (b) a set of detection probes, which set comprises at least two detection probes each directed to a target biomarker of a target biomarker signature for prostate cancer, wherein the detection probes each comprise: (i) a target binding moiety directed the target biomarker of the target biomarker signature for prostate cancer; and (ii) an oligonucleotide domain coupled to the target binding moiety, the oligonucleotide domain comprising a double-stranded portion and a single-stranded overhang portion extended from one end of the oligonucleotide domain, wherein the single-stranded overhang portions of the at least two detection probes are characterized in that they can hybridize to each other when the at least two detection probes are bound to the same extracellular vesicle. In these embodiments, such a target biomarker signature for prostate cancer comprises at least one extracellular vesicle-associated surface biomarker (e.g., as described herein) and at least one target biomarker selected from the group consisting of: surface biomarkers (e.g., as described herein), intravesicular biomarkers (e.g., as described herein), and intravesicular RNA biomarkers (e.g., as described herein). In some embodiments, one or more surface biomarkers utilized in a provided kit are selected from: (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, and combinations thereof; and/or (ii) carbohydrate-dependent markers as follows: Tn antigen, SialylTn (sTn) antigen, Thomsen-Friedenreich (T. TF) antigen, Lewis Y antigen (also known as CD174), Sialyl Lewis X (sLex) antigen (also known as Sialyl SSEA-1 (SLX)), and combinations thereof.

In some embodiments, one or more surface biomarkers utilized in a provided kit are selected from: (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, a first surface biomarker utilized in a provided kit is selected from: (i) a polypeptide encoded by human gene MUC1; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof; and a second surface biomarker utilized in a provided kit is selected from: polypeptides encoded by human genes as follows: ABCC4, AP1M2, ARFGEF3, CANT1, CDH1, CLDN3, CLDN4, CLGN, ENPP5, FOLH1, GOLM1, GRHL2, MAP7, MARCKSL1, PMEPA1, PODXL2, PPP3CA, RAB3B, RAB3D, RDH11, SLC39A6, SLC4A4, SMPDL3B, SORD, STEAP1, STEAP2, SYT7, TMPRSS2, TSPAN1, UNC13B, and combinations thereof.

In some embodiments, one or more intravesicular biomarkers utilized in a provided kit are selected from polypeptides encoded by human genes as follows: ACP5, ACPP, ACSM1, ACTA2, ACTG2, ADAMTS1, AGR2, AIM1, ALCAM, ALDH1A3, ALOX15B, ANTXR1, ANTXR2, AP1M2, AR, ARG2, ARHGAP6, ARHGEF26, ARHGEF37, ASAP3, ATP8B1, ATP9A, BAIAP2L1, BCAM, BHLHA15, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CGN, CGNL1, CHMP4C, CLGN, CMTM4, COLCA1, CORO2A, CPE, CPNE4, CPQ, CRYM, CTSF, CX3CL1, CXADR, CXCR4, CYB561, DPYSL3, DSP, EEF1A2, EFNA1, EHD2, EHF, ELF3, EMP2, EPHX1, EPN3, ERBB3, ERG, ESRP1, ESRP2, F3, FAAH, FAM129A, FAM129B, FAM210B, FAM3B, FAM3D, FAM83H, FAM84A, FAM84B, FAT1, FBLIM1, FBP1, FLNC, FNBP1L, FOS, FOXA1, GADD45G, GATA2, GGT1, GJB1, GLYATL1, GNG4, GNMT, GRHL2, GUCY1A3, HGD, HID1, HIST1H2BK, HIST3H2A, HMGCS2, HOMER2, HOXB13, HSD17B6, HSPB6, HSPB8, ICA1, ID1, IQGAP2, IRF6, ITGA6, KIAA1522, KIF5C, KLK2, KRT14, KRT15, KRT17, KRT18, KRT19, KRT5, KRT7, KRT8, LCP1, LIFR, LIMCH1, LMOD1, LRP11, LRP3, LSR, MAL2, MAOA, MAP7, MARC1, MARCKSL1, MB, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, PALLD, PARVA, PCDH1, PCP4, PDIA5, PEBP4, PGM5, PKP1, PKP3, PPL, PPM1H, PPP1R1B, PPP3CA, PRAC1, PRR15L, PSAT1, PTPRF, PYCR1, RAB3B, RAMP1, RAP1GAP, RASEF, RBM47, RCAN3, RDH11, REEP6, REPS2, RGS10, RHPN2, RNF144B, RORC, RPS4Y1, S100A16, SELENBP1, SERINC5, SH3BGRL2, SH3BP4, SHC2, SLC1A5, SLC22A3, SLC2A12, SLC40A1, SLC4A4, SLC7A8, SLC9A3R2, SMOC2, SORD, SPDEF, SPINT1, SPINT2, SPTBN2, ST14, STAP2, STK39, SULT2B1, SYNE4, SYT7, SYTL1, TAGLN, TBC1D16, TBX3, TC2N, TCEA3, TEAD3, TESC, THBS4, TM4SF1, TMED3, TMEM150C, TMEM54, TMEM98, TMSB15A, TP53111, TPD52, TPM1, TSPAN13, TSPAN6, TSPAN8, VAMP8, VSTM2L, WLS, WWC1, YAP1, ZBTB42, ZC3H11A, ZNF217, ACSL3, CADH7, KCNN2, OPRK1, PDE9A, PLPP1, SPON2, TMEM121B, and combinations thereof. In some embodiments, an intravesicular biomarker described herein may comprise at least one post-translational modification. In some embodiments, one or more intravesicular RNA biomarkers utilized in a provided kit are selected from: RNA transcripts (e.g., mRNA transcripts) encoded by human genes as follows: ABCC4, ACP5, ACPP, ACSM1, ACTA2, ACTG2, ADAMTS1, AGR2, AIM1, ALCAM, ALDH1A3, ALOX15B, ANO7, ANPEP, ANTXR1, ANTXR2, AP1M2, APCDD1, AQP3, AR, ARG2, ARHGAP6, ARHGEF26, ARHGEF37, ASAP3, ASTN2, ATP8B1, ATP9A, BAIAP2L1, BCAM, BHLHA15, BIK, BMPR1B, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CDH1, CGN, CGNL1, CHMP4C, CHRM1, CHRNA2, CLDN3, CLDN4, CLDN7, CLDN8, CLGN, CMTM4, COLCA1, COLEC12, CORO2A, CPE, CPNE4, CPQ, CRB3, CREB3L1, CREB3L4, CRYM, CTSF, CWH43, CX3CL1, CXADR, CXCR4, CYB561, DPP4, DPYSL3, DSG2, DSP, EEF1A2, EFNA1, EHD2, EHF, ELF3, ELOVL7, EMB, EMP2, ENPP5, EPCAM, EPHX1, EPN3, ERBB3, ERG, ESRP1, ESRP2, F3, FAAH, FAM129A, FAM129B, FAM189A2, FAM210B, FAM3B, FAM3D, FAM83H, FAM84A, FAM84B, FAT1, FBLIM1, FBP1, FLNC, FNBP1L, FOLH1, FOS, FOXA1, FXYD3, GADD45G, GALNT3, GALNT7, GATA2, GCNT1, GGT1, GJB1, GLYATL1, GNG4, GNMT, GOLM1, GPR160, GREB1, GRHL2, GUCY1A3, HGD, HID1, HIST1H2BK, HIST3H2A, HMGCS2, HOMER2, HOXB13, HPN, HSD17B6, HSPB6, HSPB8, ICA1, ID1, IQGAP2, IRF6, ITGA6, KCNN4, KIAA1324, KIAA1522, KIF5C, KLK2, KRT14, KRT15, KRT17, KRT18, KRT19, KRT5, KRT7, KRT8, KRTCAP3, LCP1, LIFR, LIMCH1, LMOD1, LPAR3, LRP11, LRP3, LRRC26, LSR, MAL2, MAOA, MAP7, MARC1, MARCKSL1, MB, MBOAT2, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, OR51E1, OR51E2, PALLD, PARM1, PARVA, PCDH1, PCP4, PDIA5, PDLIM5, PDZK1IP1, PDZRN3, PEBP4, PGM5, PIGR, PKP1, PKP3, PLA2G2A, PMEPA1, PODXL2, PPL, PPM1H, PPP1R1B, PPP3CA, PRAC1, PROM2, PRR15L, PRSS8, PSAT1, PSCA, PTPRF, PTPRN2, PYCR1, RAB25, RAB27B, RAB3B, RAMP1, RAP1GAP, RASEF, RBM47, RCAN3, RDH11, REEP6, REPS2, RGS10, RHPN2, RNF144B, RORC, RPS4Y1, S100A16, SCNN1A, SDC1, SELENBP1, SERINC2, SERINC5, SEZ6L2, SH3BGRL2, SH3BP4, SHC2, SLC15A2, SLC1A5, SLC22A3, SLC2A10, SLC2A12, SLC30A4, SLC35F2, SLC40A1, SLC44A4, SLC45A3, SLC4A4, SLC7A8, SLC9A3R2, SMIM22, SMOC2, SORD, SPDEF, SPINT1, SPINT2, SPTBN2, ST14, STAP2, STEAP1, STEAP2, STEAP4, STK39, SULT2B1, SYNE4, SYT7, SYTL1, TACSTD2, TAGLN, TBC1D16, TBX3, TC2N, TCEA3, TEAD3, TESC, THBS4, TM4SF1, TMC4, TMC5, TMED3, TMEFF2, TMEM125, TMEM150C, TMEM30B, TMEM45B, TMEM54, TMEM98, TMPRSS2, TMSB15A, TP53111, TPD52, TPM1, TRPM4, TRPM8, TRPV6, TSPAN1, TSPAN13, TSPAN6, TSPAN8, TUSC3, UPK3A, VAMP8, VSIG2, VSTM2L, WLS, WWC1, YAP1, ZBTB42, ZC3H11A, ZNF217, and combinations thereof.

In some embodiments, when at least one target biomarker is selected from one or more of the provided surface biomarkers, the selected surface biomarker(s) and the at least one extracellular vesicle-associated surface biomarker are different. In some embodiments, when at least one target biomarker is selected from one or more of the provided surface biomarkers, the selected surface biomarker(s) and the at least one extracellular vesicle-associated surface biomarker are the same (with the same or different epitopes).

In some embodiments, a capture agent provided in a kit comprises a target-capture moiety directed to an extracellular vesicle-associated surface biomarker, which is or comprises one or more of (i) a polypeptide encoded by a human gene as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, or combinations thereof; and/or one or more of (ii) a carbohydrate antigen as follows: Tn antigen, SialylTn (sTn) antigen, Thomsen-Friedenreich (T. TF) antigen, Lewis Y antigen (also known as CD174), Sialyl Lewis X (sLex) antigen (also known as Sialyl SSEA-1 (SLX)), or combinations thereof.

In some embodiments, a target binding moiety of at least two detection probes provided in a kit is each directed to the same target biomarker of a target biomarker signature. In some such embodiments, an oligonucleotide domain of such at least two detection probes are different

In some embodiments, a target binding moiety of at least two detection probes provided in a kit is each directed to a distinct target biomarker of a target biomarker signature.

In some embodiments, a target binding moiety of a detection probe may be or comprise an affinity agent, which in some embodiments may be or comprise an antibody (e.g., a monoclonal antibody). In some embodiments, a target binding moiety of a detection probe may be or comprise an affinity agent, which in some embodiments may be or comprise a lectin or siglec.

In some embodiments, a kit may comprise at least one enzymatic and/or chemical reagent such as an enzyme, a fixation agent, a permeabilization agent, and/or a blocking agent.

In some embodiments, a kit may comprise one or more nucleic acid ligation reagents (e.g., a nucleic acid ligase such as a DNA ligase and/or a buffer solution).

In some embodiments, a kit may comprise at least one or more amplification reagents such as PCR amplification reagents. In some embodiments, a kit may comprise one or more nucleic acid polymerases (e.g., DNA polymerases), one or more pairs of primers, nucleotides, and/or a buffered solution.

In some embodiments, a kit may comprise a solid substrate for capturing an entity (e.g., biological entity) of interest. For example, such a solid substrate may be or comprise a bead (e.g., a magnetic bead). In some embodiments, such a solid substrate may be or comprise a surface. In some embodiments, a surface may be or comprise a capture surface (e.g., an entity capture surface) of an assay chamber, such as, e.g., a filter, a matrix, a membrane, a plate, a tube, a well (e.g., but not limited to a microwell), etc. In some embodiments, a surface (e.g., a capture surface) of a solid substrate can be coated with a capture agent (e.g., affinity agent) for an entity (e.g., biological entity) of interest.

In some embodiments, a set of detection probes provided in a kit may be selected for diagnosis of prostate cancer.

In some embodiments, a set of detection probes provided in a kit may be selected for diagnosis of prostate adenocarcinoma.

In some embodiments, a kit may comprise a plurality of sets of detection probes, wherein each set of detection probes is directed for detection of a specific cancer and comprises at least 2 or more detection probes. For example, such a kit can be used to screen a subject for various cancers, one of which is prostate cancer (e.g., prostate adenocarcinoma) while other cancers may be selected from skin cancer, lung cancer, breast cancer, ovarian cancer, pancreatic cancer, prostate cancer, brain cancer, and liver cancer in a single assay.

In some embodiments, kits provided herein may include instructions for practicing methods described herein. These instructions may be present in kits in a variety of forms, one or more of which may be present in the kits. One form in which these instructions may be present is as printed information on a suitable medium or substrate, e.g., a piece or pieces of paper on which the information is printed, in the packaging of kits, in a package insert, etc. Yet another means may be a computer readable medium, e.g., diskette, CD, USB drive, etc., on which instructional information has been recorded. Yet another means that may be present is a website address which may be used via the internet to access instructional information. Any convenient means may be present in the kits.

In some embodiments where kits are for use as companion diagnostics, such kits can include instructions for identifying patients that are likely to respond to a therapeutic agent (e.g., identification of biomarkers that are indicative of patient responsiveness to the therapeutic agent). In some embodiments, such kits can comprise a therapeutic agent for use in tandem with the companion diagnostic test.

Other features of the invention will become apparent in the course of the following description of exemplary embodiments, which are given for illustration of the invention and are not intended to be limiting thereof.

### EXEMPLIFICATION

### Example 1: Detection of an Exemplary Target Biomarker Signature in Individual Extracellular Vesicles Associated with Prostate Adenocarcinoma

The present Example describes synthesis of detection probes for targets (e.g., target biomarker(s)) each comprising a target-binding moiety and an oligonucleotide domain (comprising a double-stranded portion and a single stranded overhang) coupled to the target-binding moiety. The present Example further demonstrates that use of such detection probes to detect the presence or absence of biological entities (e.g., extracellular vesicles) comprising two or more distinct targets.

In some embodiments, a detection probe can comprise a double-stranded oligonucleotide with an antibody agent specific to a target cancer biomarker at one end and a single stranded overhang at another end. When two or more detection probes are bound to the same biological entity (e.g., an extracellular vesicle), the single-stranded overhangs of the detection probes are in close proximity such that they can hybridize to each other to form a double-stranded complex, which can be subsequently ligated and amplified for detection.

This study employed at least two detection probes in a set. In some embodiments, such at least two detection probes are directed to the same target biomarker. In some embodiments, such at least two detection probes directed to the same target, which may be directed to different epitopes of the same target or to the same epitope of the same target. In some embodiments, such at least two detection probes are directed to distinct targets. A skilled artisan reading the present disclosure will understand that two detection probes can be directed to different target biomarkers, or that three or more detection probes, each directed towards a distinct target protein, may be used. Further, compositions and methods described in this Example can be extended to applications in different biological samples (e.g., comprising extracellular vesicles).

### Overview of an Exemplary Assay

In some embodiments, a target entity detection system described herein is a duplex system. In some embodiments, such a duplex system, e.g., as illustrated in FIG. 2, utilizes two antibodies that each recognize a different epitope. Paired double-stranded template DNAs are also utilized in qPCR, each of which has specific four-base 5′ overhangs complementary to the 5′ overhang on its partner. Each antibody may be conjugated with one of the two double-stranded DNA templates. When the antibodies bind their target epitopes, the sticky ends of the respective templates can hybridize. These sticky ends may then be ligated together by T7 ligase, prior to PCR amplification. For hybridization between the two DNA templates to occur, the two antibodies need to be bound close enough to each other (within 50 to 60 nm, the length of the DNA linker and antibody). Any templates that bind but remain unligated will not produce PCR product, as shown in FIG. 2.

### Exemplary Methods:

### Oligonucleotides

In some embodiments, oligonucleotides can have the following sequence structure and modifications. It is noted that the strand numbers below correspond to the numerical values associated with strands shown in FIG. 2.

Strand 1 v1:
/5AzideN/CAGTCTGACACAGCAGTCGTTAATCGTCGCTGCTACCCTTG
ACATCCGTGACTGGCTAGACAGAGGTGT, where /5AzideN/
refers to an azide group linked to the 5′
oligonucleotide terminus via a NHS ester linker,
or
/5AmMC12/CAGTCTGACACAGCAGTCGTTAATCGTCGCTGCTACCCTTG
ACATCCGTGACTGGCTAGACAGAGGTGT, where /5AmMC12/
refers to an amine group (e.g., a primary amino
group) linked to the 5′ oligonucleotide terminus
via a 12-carbon spacer, or
/5ThiolMC6/CAGTCTGACACAGCAGTCGTTAATCGTCGCTGCTACCCT
TGACATCCGTGACTGGCTAGACAGAGGTGT, where /5ThiolMC6/
refers to a thiol linked to the 5′
oligonucleotide terminus via a 6-carbon spacer.
Strand 2 v1:
/5AzideN/GACCTGACCTACAGTGACCATAGCCTTGCCTGATTAGCCAC
TGTCCAGTTTGGCTCCTGGTCTCACTAG, where /5AzideN/
refers to an azide group linked to the 5′
oligonucleotide terminus via a NHS ester linker,
or
/5AmMC12/GACCTGACCTACAGTGACCATAGCCTTGCCTGATTAGCCAC
TGTCCAGTTTGGCTCCTGGTCTCACTAG, where /5AmMC1/
refers to an amine group (e.g., a primary
amino group) linked to the 5' oligonucleotide
terminus via a 12-carbon spacer, or
/5ThiolMC6/GACCTGACCTACAGTGACCATAGCCTTGCCTGATTAGCC
ACTGTCCAGTTTGGCTCCTGGTCTCACTAG, where /5ThiolMC6/
refers to a thiol linked to the 5′
oligonucleotide terminus via a 6-carbon spacer
Strand 3 v1:
/5Phos/GAGTACACCTCTGTCTAGCCAGTCACGGATGTCAAGGGTAGCA
GCGACGATTAACGACTGCTGTGTCAGACTG, wherein /5Phos/
refers to a phosphate group linked to the
5′ oligonucleotide terminus
Strand 4 v1:
/5Phos/ACTCCTAGTGAGACCAGGAGCCAAACTGGACAGTGGCTAATCA
GGCAAGGCTATGGTCACTGTAGGTCAGGTC, wherein /5Phos/
refers to a phosphate group linked to the
5′ oligonucleotide terminus
Strand 5 v1:
CAGTCTGACACAGCAGTCGT
Strand 6 v1:
GACCTGACCTACAGTGACCA
Strand 7 (Probe) v1:
/56-FAM/TGGCTAGAC/ZEN/AGAGGTGTACTCCTAGTGAGA/
3IABkFQ/, wherein /56-FAM/ refers to a
fluorescein (e.g., 6-FAM) at the 5′
oligonucleotide terminus; and /3IABkFQ/
refers to a fluorescein quencher
at the 3′ oligonucleotide terminus

In some embodiments, oligonucleotides can have the following sequence structure and modifications. It is noted that the strand numbers below correspond to the numerical values associated with strands shown in FIG. 2.

Strand 1 v2:
/5AzideN/CAGTCTGACTCACCACTCGTTAATCGTCGCTGCTACCCTTG
ACATCCGTGACTGGCTAGACAGAGGTGT, where /5AzideN/
refers to an azide group linked to the 5′
oligonucleotide terminus via a NHS ester linker,
or
/5AmMC12/CAGTCTGACTCACCACTCGTTAATCGTCGCTGCTACCCTTG
ACATCCGTGACTGGCTAGACAGAGGTGT, where /5AmMC12/
refers to an amine group (e.g., a primary amino
group) linked to the 5′ oligonucleotide terminus
via a 12-carbon spacer, or
/5ThiolMC6/CAGTCTGACTCACCACTCGTTAATCGTCGCTGCTACCCT
TGACATCCGTGACTGGCTAGACAGAGGTGT, where /5ThiolMC6/
refers to a thiol linked to the 5′
oligonucleotide terminus via a 6-carbon spacer
Strand 2 v2:
/5AzideN/CACCAGACCTACGAAGTCCATAGCCTTGCCTGATTAGCCAC
TGTCCAGTTTGGCTCCTGGTCTCACTAG, where /5AzideN/
refers to an azide group linked to the 5′
oligonucleotide terminus via a NHS ester linker,
or
/5AmMC12/CACCAGACCTACGAAGTCCATAGCCTTGCCTGATTAGCCAC
TGTCCAGTTTGGCTCCTGGTCTCACTAG, where /5AmMC1/
refers to an amine group (e.g., a primary
amino group) linked to the 5′ oligonucleotide
terminus via a 12-carbon spacer, or
/5ThiolMC6/CACCAGACCTACGAAGTCCATAGCCTTGCGCCTGATTAG
CCACTGTCCAGTTTGGCTCCTGGTCTCACTAG, where
/5ThiolMC6/refers to a thiol linked to the 5′
oligonucleotide terminus via a 6-carbon spacer
Strand 3 v2:
/5Phos/GAGTACACCTCTGTCTAGCCAGTCACGGATGTCAAGGGTAGCA
GCGACGATTAACGAGTGGTGAGTCAGACTG, wherein /5Phos/
refers to a phosphate group linked to the
5′ oligonucleotide terminus
Strand 4 v2:
/5Phos/ACTCCTAGTGAGACCAGGAGCCAAACTGGACAGTGGCTAATCA
GGCAAGGCTATGGACTTCGTAGGTCTGGTG, wherein /5Phos/
refers to a phosphate group linked to the
5′ oligonucleotide terminus
Strand 5 v2:
CAGTCTGACTCACCACTCGT
Strand 6 v2:
CACCAGACCTACGAAGTCCA
Strand 7 (Probe) v2:
/56-FAM/TGGCTAGAC/ZEN/AGAGGTGTACTCCTAGTGAGA/
3IABkFQ/, wherein /56-FAM/ refers to a
fluorescein (e.g., 6-FAM) at the 5′
oligonucleotide terminus; and
/3IABkFQ/ refers to a fluorescein quencher
at the 3′ oligonucleotide terminus.

In some embodiments, oligonucleotides can have the following sequence structure and modifications. It is noted that the strand numbers below correspond to the numerical values associated with strands shown in FIG. 2.

Strand 1 v1-med:
/5AzideN/CAGTCTGACACAGCAGTCGTGACTGGCTAGACAGAGGTGT,
where /5AzideN/ refers to an azide group linked
to the 5′ oligonucleotide terminus via a NHS
ester linker, or
/5AmMC12/CAGTCTGACACAGCAGTCGTGACTGGCTAGACAGAGGTGT,
where /5AmMC12/ refers to an amine group (e.g., a
primary amino group) linked to the 5′
oligonucleotide terminus via a 12-carbon spacer,
or
/5ThiolMC6/CAGTCTGACACAGCAGTCGTGACTGGCTAGACAGAGGTG
T, where /5ThiolMC6/ refers to a thiol linked
to the 5′ oligonucleotide terminus via a 6-
carbon spacer.
Strand 2 v1-med:
/5AzideN/GACCTGACCTACAGTGACCATTGGCTCCTGGTCTCACTAG,
where /5AzideN/ refers to an azide group linked
to the 5′ oligonucleotide terminus via a NHS
ester linker, or
/5AmMC12/GACCTGACCTACAGTGACCATTGGCTCCTGGTCTCACTAG,
where /5AmMC1/ refers to an amine group (e.g.,
a primary amino group) linked to the 5'
oligonucleotide terminus via a 12-carbon spacer,
or
/5ThiolMC6/GACCTGACCTACAGTGACCATTGGCTCCTGGTCTCACTA
G, where /5ThiolMC6/ refers to a thiol linked to
the 5′ oligonucleotide terminus via a 6-carbon
spacer
Strand 3 v1-med:
/5Phos/GAGTACACCTCTGTCTAGCCAGTCACGACTGCTGTGTCAGACT
G, wherein /5Phos/ refers to a phosphate group
linked to the 5′ oligonucleotide terminus
Strand 4 v1-med:
/5Phos/ACTCCTAGTGAGACCAGGAGCCAATGGTCACTGTAGGTCAGGT
C, wherein /5Phos/ refers to a phosphate group
linked to the 5′ oligonucleotide terminus
Strand 5 v1:
CAGTCTGACACAGCAGTCGT
Strand 6 v1:
GACCTGACCTACAGTGACCA
Strand 7 (Probe) v1:
/56-FAM/TGGCTAGAC/ZEN/AGAGGTGTACTCCTAGTGAGA/
3IABkFQ/, wherein /56-FAM/ refers to a
fluorescein (e.g., 6-FAM) at the 5′
oligonucleotide terminus; and /3IABkFQ/
refers to a fluorescein quencher
at the 3′ oligonucleotide terminus.

### Antibody-Oligonucleotide (e.g., Antibody-DNA) Conjugation:

Antibody aliquots ranging from 25-100 μg may be conjugated with oligonucleotide strands. For example, 60 μg aliquots of antibodies may be conjugated with hybridized strands 1+3 and 2+4, for example, using copper-free click chemistry. The first step may be to prepare DBCO-functionalized antibodies to participate in the conjugation reaction with azide-modified oligonucleotide domain (e.g., DNA domain). This may begin with reacting the antibodies with the DBCO-PEG5-NHS heterobifunctional cross linker. The reaction between the NHS ester and available lysine groups may be allowed to take place at room temperature for 2 hours, after which unreacted crosslinker may be removed using centrifugal ultrafiltration. To complete the conjugation, azide-modified oligonucleotide domains (e.g., DNA domain) and the DBCO-functionalized antibodies may be allowed to react overnight at room temperature. The concentration of conjugated antibody may be measured, for example, using the Qubit protein assay.

### Cell Culture

Negative control cells (e.g., non-prostate cancer cells such as melanoma cells or healthy cells) may be grown in Eagle's Minimum Essential Medium (EMEM) with 10% exosome-free FBS and 50 units of penicillin/streptomycin per mL. Prostate cancer cells may be grown in Roswell Park Memorial Institute (RPMI 1640) with 10% exosome-free FBS and 50 units of penicillin/streptomycin per mL. There are currently dozens, if not more, exemplary prostate cancer cell lines that may be useful to develop an assay for detection of prostate cancer. Cell lines may be grown in complete media supplemented with exosome-depleted fetal bovine serum per the recommendation of the cell line supplier or inventor.

Purification of Extracellular Vesicles from Cell Culture Medium

In some embodiments, prostate cancer cells and negative control cells may be grown in their respective media until they reach ˜80% confluence. The cell culture medium may be collected and spun at 300 RCF for 5 minutes at room temperature (RT) to remove cells and debris. The supernatant may then be collected and used in assays as described herein or frozen at −80° C.

### Thawing

If prior to use, samples were stored at −80° C., they are thawed. In brief, 50 mL tubes containing frozen conditioned media placed in plastic racks, the racks are placed in an empty ice bucket. Room temperature (RT) water is added, and samples are allowed to thaw, with periodic inversion/shaking to facilitate thawing. Tubes are consolidated such that all the tubes for each cell line are the same volume. A typical purification volume is approximately 200 mLs of spent medium per cell line. If larger batches are desired, this volume can be increased.

### Clarification

In some embodiments, samples are clarified prior to use. Clarification of media serves to remove cells and debris. In brief, 1) spin at 1300 RCF for 10 mins; transfer supernatant to a new 50 mL conical tube using a pipette, leaving ˜1 cm of medium (to avoid disturbing the pellet), the remaining media is not decanted; 2) spin at 2000 RCF for 30 mins; transfer supernatant to a new 50 mL conical tube using a pipette, leaving ˜1 cm of medium (to avoid disturbing the pellet), the remaining media is not decanted.

### Concentrate Media

In some embodiments, samples are concentrated. In brief: 1) a single 15 mL Amicon 10 kDa MWCO filter is used for approximately 100 mLs of medium (for example, for a 200 mL batch, two10 kDa MWCO ultrafiltration tubes will be needed). In some embodiments, the same ultrafiltration column can be sequentially added to and re-spun to enable the concentration of large volumes of medium. In general, columns were utilized according to the manufacturer's protocol. Columns are spun for 10-12 minutes each time, at maximum speed (2500 to 4,300 RCF). 2) When each of the two tubes containing the same spent medium reaches ˜1500 uL, the two tubes are combined into one, the now empty Amicon tube may be utilized as a balance. 3) When removing the concentrated medium, the sides of the concentration chamber may be flushed to release as many entrapped EVs as possible, while avoiding frothing, the consolidated media may be concentrated until there is 1 mL left. 4) The media is transferred to a 1.5 mL protein LoBind tube, with the 1 mL line marked, if necessary, volume is corrected to 1 mL with 20 nm filtered 1× PBS.

### Final Clarification Spin

To remove any remaining debris, the concentrated media can be centrifuged at 10,000 RCF for 10 minutes at 21° C. in a tabletop Eppendorf centrifuge.

### Run Concentrated Media Through Prepared IZON Columns

Izon columns are washed as described by the manufacturer, 20 nm filtered 1× PBS can be used to both wash the columns and recover the samples. 1 mL of concentrated spent medium can be run through the column and fractions can be collected (e.g., fractions 7, 8, and 9) in 5-mL Eppendorf flip-cap tubes, following the manufacturer's protocol.

### Particle Counts:

Particle counts may be obtained, e.g., using a SpectraDyne particle counting instrument using the TS400 chips, to measure nanoparticle range between 65 and 1000 nm. In some embodiments, a particle size that is smaller than 65 nm or larger than 1000 nm may be desirable.

### Generation of Patient Plasma Pools:

In some embodiments, pooled patient plasma pools may be utilized. In brief, 1 mL aliquots of patient plasma may be thawed at room temperature for at least 30 minutes. The tubes may be vortexed briefly and spun down to consolidate plasma to the bottom of each tube. Plasma samples from a given patient cohort may be combined in an appropriately sized container and mixed thoroughly by end-over-end mixing. Each plasma pool may be split into 1 mL aliquots in Protein Lo-bind 1.5 mL Eppendorf tubes and refrozen at −80° C.

### Whole-Plasma Clarification (Optional):

In some embodiments, prior to nanoparticle purification, samples may be blinded by personnel who would not participate in sample-handling. The patient-identification information may only be revealed after the experiment is completed to enable data analysis. 1 mL aliquots of whole plasma may be removed from storage at −80° C., and subjected to three clarification spins to remove cells, platelets, and debris.

Size-Exclusion Chromatography Purification of EVs from Clarified Plasma:

Each clarified plasma sample (individual samples or pooled samples) may be run through a single-use, size-exclusion purification column to isolate nanoparticles. Nanoparticles having a size range of about 65 nm to about 1000 nm may be collected for each sample. In some embodiments, particle size that is smaller than 65 nm or larger than 1000 nm may be desirable.

### Capture-Antibody Conjugation to Magnetic-Capture Beads:

Antibodies may be conjugated to magnetic beads (e.g., epoxy-functionalized Dynabeads™). Briefly, beads may be weighed in a sterile environment and resuspended in buffer. Antibodies may be, at approximately 8 μg of Ab per mg of bead, mixed with the functionalized beads and the conjugation reaction may take place overnight at 37° C. with end-over-end mixing. The beads may be washed several times using the wash buffer provided by the conjugation kit and may be stored at 4° C. in the provided storage buffer, or at −20° C. in a glycerol-based storage buffer.

### Direct Capture of Purified Plasma EVs Using Antibody-Conjugated Magnetic Beads:

For biomarker capture, a diluted sample of purified plasma EVs may be incubated with magnetic beads conjugated with respective antibodies for an appropriate time period at an appropriate temperature, e.g., at room temperature.

### Binding of Antibody-Oligonucleotide Conjugates to EVs Bound on Magnetic Capture Beads:

Antibody-oligonucleotide conjugates may be diluted in an appropriate buffer at their optimal concentrations. Antibody probes may be allowed to interact with a sample comprising EVs bound on magnetic capture beads.

### Post-Binding Washes:

In some embodiments, samples may be washed, e.g., multiple times, in an appropriate buffer.

### Ligation:

After the wash to remove unbound antibody-oligonucleotide conjugates, the beads with bound extracellular vesicles and bound antibody-oligonucleotide conjugates may be contacted with a ligation mix. The mixtures may then be incubated for 20 minutes at room temperature.

### PCR:

Following ligation, the beads with bound extracellular vesicles and bound antibody-oligonucleotide conjugates may be contacted with a PCR mix. PCR may be performed in a 96-well plate, e.g., on the Quant Studio 3, with the following exemplary PCR protocol: hold at 95° C. for 1 minute, perform 50 cycles of 95° C. for 5 seconds and 62° C. for 15 seconds. The rate of temperature change may be chosen to be standard (e.g., 2° C. per second). A single qPCR reaction may be performed for each experimental replicate and ROX may be used as the passive reference to normalize the qPCR signals. Data may then be downloaded from the Quant Studio 3 machine and analyzed and plotted in Python 3.7.

### Data Analysis:

In some embodiments, a binary classification system can be used for data analysis. In some embodiments, signals from a detection assay may be normalized based on a reference signal. For example, in some embodiments, normalized signals for a single antibody duplex may be calculated by choosing a reference sample. In some embodiments, the equations used to calculate the normalized signal for an arbitrary sample i are given below, where Signalₘₐₓ is the signal from the highest concentration cell-line EVs standard.

$$Δ {Ct}_{i} = {Ct}_{ref} - {Ct}_{i}$$

$${Signal}_{i} = {2}^{Δ {Ct}_{i}}$$

$$Norm {Signal}_{i} = \frac{{Signal}_{i}}{{Signal}_{max}}$$

### DISCUSSION

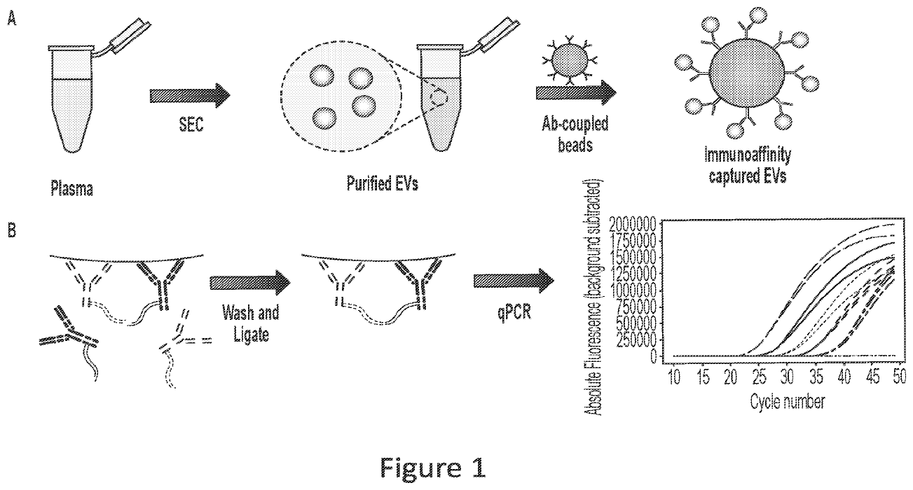

The present Example describes the use of biomarker combinations in the assay described in FIGS. 1 and 2 (e.g. the biomarkers used in combination with a duplex assay). The assay may be capable of detecting prostate cancer with >99% specificity. In some embodiments, a biomarker combination includes capture and detection probes. In some embodiments, use of two or more biomarker combinations in an assay may increase the specificity of the assay.

In some embodiments, a dendron, which can add up to 16 strands of oligonucleotide domain (e.g., DNA) per antibody, can be used instead of one or two strands of DNA per antibody, for example, to enhance signal-to-noise.

### Example 2: Assessment of Extracellular Vesicle (EV) Surface Biomarkers as Prostate Adenocarcinoma Biomarkers

In some embodiments, prostate cancer detection includes detection of at least EV surface biomarker(s) following immunoaffinity capture of extracellular vesicles.

In some embodiments, one or more surface biomarkers or extracellular rmembrane biomarkers that are present on extracellular vesicles (“capture biomarkers”) can be used for immunoaffinity capture of prostate cancer-associated extracellular vesicles. Examples of such capture biomarkers may include, but are not limited to (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, and combinations thereof; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof.

In some embodiments, EV immunoassay methodology (e.g., ones described herein such as in Example 1) and biomarker-validation process (e.g., ones described herein such as in Example 1) can be used to assess additional surface biomarkers as biomarkers for prostate cancer. In some embodiments, an antibody directed to a capture biomarker (e.g., a surface biomarker present on prostate cancer-associated EVs) is conjugated to magnetic beads and evaluated, optionally first on cell-line EVs then on patient samples, for its ability to bind the specific target biomarker. The antibody-coated bead is assessed for its ability to capture prostate cancer-associated EVs and the captured EVs by the antibody-coated bead is read out using a target entity detection system (e.g., a duplex system as described herein involving a set of two detection probes (e.g., as described herein), each directed to a target marker that is distinct from the capture biomarker.

In some embodiments, captured EVs can be read out using at least one (e.g., 1, 2, 3, or more) surface biomarker, which is or comprises (i) at least one polypeptide encoded by a human gene as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, or combinations thereof; and/or (ii) at least one carbohydrate-dependent marker as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof. In some embodiments, captured EVs can be read out using a set of detection probes (e.g., as utilized and/or described herein), at least two of which are directed to one or more (e.g., 1, 2, 3, or more) surface biomarkers, which are or comprise (i) one or more polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4, or combinations thereof; and/or (ii) one or more carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), or combinations thereof. In some embodiments, a set of detection probes comprises two detection probes each directed to the same surface biomarker. In some embodiments, a set of detection probes comprises two detection probes each directed to a distinct surface biomarker.

### Example 3: Assessment of mRNA in Extracellular Vesicles (Intravesicular mRNA) as Prostate Cancer Biomarkers

In some embodiments, prostate cancer detection includes detection of at least intravesicular mRNA(s) following immunoaffinity capture of extracellular vesicles.

In some embodiments, one or more surface proteins or extracellular membrane proteins that are present on extracellular vesicles (“capture proteins”) can be used for immunoaffinity capture of prostate cancer-associated extracellular vesicles. Examples of such capture protein biomarkers may include, but are not limited to polypeptides encoded by human genes as described in Example 2 and carbohydrate-dependent markers as described in Example 2.

In some embodiments, EV nucleic acid detection assay (e.g., reverse transcription PCR using primer-probe sets) and biomarker-validation process (e.g., ones described herein such as in Example 1) can be used to assess mRNA biomarker candidates for prostate cancer. In some embodiments, an antibody directed to a capture biomarker (e.g., a surface biomarker present in prostate cancer-associated EVs) is conjugated to magnetic beads and evaluated, optionally first on cell-line EVs then on patient samples, for its ability to bind the specific target biomarker. The antibody-coated bead is assessed for its ability to capture prostate cancer-associated EVs and the captured EVs by the antibody-coated bead is profiled for their mRNA contents, for example, using one-step quantitative reverse transcription PCR (RT-qPCR) master mix.

In some embodiments, captured EVs can be read out by detection of at least one (e.g., 1, 2, 3, or more) of the following mRNA biomarkers encoded by human genes as follows: ABCC4, ACP5, ACPP, ACSM1, ACTA2, ACTG2, ADAMTS1, AGR2, AIM1, ALCAM, ALDH1A3, ALOX15B, ANO7, ANPEP, ANTXR1, ANTXR2, AP1M2, APCDD1, AQP3, AR, ARG2, ARHGAP6, ARHGEF26, ARHGEF37, ASAP3, ASTN2, ATP8B1, ATP9A, BAIAP2L1, BCAM, BHLHA15, BIK, BMPR1B, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CDH1, CGN, CGNL1, CHMP4C, CHRM1, CHRNA2, CLDN3, CLDN4, CLDN7, CLDN8, CLGN, CMTM4, COLCA1, COLEC12, CORO2A, CPE, CPNE4, CPQ, CRB3, CREB3L1, CREB3L4, CRYM, CTSF, CWH43, CX3CL1, CXADR, CXCR4, CYB561, DPP4, DPYSL3, DSG2, DSP, EEF1A2, EFNA1, EHD2, EHF, ELF3, ELOVL7, EMB, EMP2, ENPP5, EPCAM, EPHX1, EPN3, ERBB3, ERG, ESRP1, ESRP2, F3, FAAH, FAM129A, FAM129B, FAM189A2, FAM210B, FAM3B, FAM3D, FAM83H, FAM84A, FAM84B, FAT1, FBLIM1, FBP1, FLNC, FNBP1L, FOLH1, FOS, FOXA1, FXYD3, GADD45G, GALNT3, GALNT7, GATA2, GCNT1, GGT1, GJB1, GLYATL1, GNG4, GNMT, GOLM1, GPR160, GREB1, GRHL2, GUCY1A3, HGD, HID1, HIST1H2BK, HIST3H2A, HMGCS2, HOMER2, HOXB13, HPN, HSD17B6, HSPB6, HSPB8, ICA1, ID1, IQGAP2, IRF6, ITGA6, KCNN4, KIAA1324, KIAA1522, KIF5C, KLK2, KRT14, KRT15, KRT17, KRT18, KRT19, KRT5, KRT7, KRT8, KRTCAP3, LCP1, LIFR, LIMCH1, LMOD1, LPAR3, LRP11, LRP3, LRRC26, LSR, MAL2, MAOA, MAP7, MARC1, MARCKSL1, MB, MBOAT2, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, OR51E1, OR51E2, PALLD, PARM1, PARVA, PCDH1, PCP4, PDIA5, PDLIM5, PDZK1IP1, PDZRN3, PEBP4, PGM5, PIGR, PKP1, PKP3, PLA2G2A, PMEPA1, PODXL2, PPL, PPM1H, PPP1R1B, PPP3CA, PRAC1, PROM2, PRR15L, PRSS8, PSAT1, PSCA, PTPRF, PTPRN2, PYCR1, RAB25, RAB27B, RAB3B, RAMP1, RAP1GAP, RASEF, RBM47, RCAN3, RDH11, REEP6, REPS2, RGS10, RHPN2, RNF144B, RORC, RPS4Y1, S100A16, SCNN1A, SDC1, SELENBP1, SERINC2, SERINC5, SEZ6L2, SH3BGRL2, SH3BP4, SHC2, SLC15A2, SLC1A5, SLC22A3, SLC2A10, SLC2A12, SLC30A4, SLC35F2, SLC40A1, SLC44A4, SLC45A3, SLC4A4, SLC7A8, SLC9A3R2, SMIM22, SMOC2, SORD, SPDEF, SPINT1, SPINT2, SPTBN2, ST14, STAP2, STEAP1, STEAP2, STEAP4, STK39, SULT2B1, SYNE4, SYT7, SYTL1, TACSTD2, TAGLN, TBC1D16, TBX3, TC2N, TCEA3, TEAD3, TESC, THBS4, TM4SF1, TMC4, TMC5, TMED3, TMEFF2, TMEM125, TMEM150C, TMEM30B, TMEM45B, TMEM54, TMEM98, TMPRSS2, TMSB15A, TP53111, TPD52, TPM1, TRPM4, TRPM8, TRPV6, TSPAN1, TSPAN13, TSPAN6, TSPAN8, TUSC3, UPK3A, VAMP8, VSIG2, VSTM2L, WLS, WWC1, YAP1, ZBTB42, ZC3H11A, ZNF217, and combinations thereof.

In some embodiments, captured EVs can be read out by detection of at least one (e.g., 1, 2, 3, or more) intravesicular RNA biomarkers (e.g., mRNA biomarkers described above); and at least one (e.g., 1, 2, 3, or more) surface biomarkers (e.g., as described in Example 2). Such biomarker combination is prostate cancer-specific. For example, in some embodiments, an intravesicular RNA biomarker may be or comprise an mRNA transcript encoded by a human gene described herein. In some embodiments, an intravesicular RNA biomarker may be or comprise a microRNA. In some embodiments, an intravesicular RNA biomarker may be or comprise long noncoding RNA. In some embodiments, an intravesicular RNA biomarker may be or comprise piwi-interacting RNA. In some embodiments, an intravesicular RNA biomarker may be or comprise circular RNA. In some embodiments, an intravesicular RNA biomarker may be or comprise small nucleolar RNA. In some embodiments, an intravesicular RNA biomarker may be or comprise an orphan noncoding RNA.

In some embodiments, captured EVs can be read out (i) by detection of one or more (e.g., 1, 2, 3, or more) intravesicular RNA biomarkers described herein using RT-qPCR (“intravesicular biomarker detection); and (ii) by using a set of detection probes (e.g., as utilized and/or described herein), at least one of which are directed to one or more (e.g., 1, 2, 3, or more) of EV surface biomarkers described in Example 2 (“surface biomarker detection”). In some embodiments, intravesicular biomarker detection is performed after surface biomarker detection. For example, in some embodiments, captured EVs after intravesicular biomarker detection can be contacted with a lysing agent to release intravascular analytes (including, e.g., intravesicular RNA biomarkers) for detection and analysis.

In some embodiments for surface biomarker detection, a set of detection probes comprises at least one detection probe directed to an EV surface biomarker. In some such embodiments, a set of detection probes comprises at least two detection probes directed to the same EV surface biomarker (with the same or different epitopes). In some such embodiments, a set of detection probes comprises at least two detection probes directed to distinct EV surface biomarkers.

In some embodiments, a set of detection probes comprises at least one detection probe directed to an EV surface biomarker. In some such embodiments, a set of detection probes comprises at least two detection probes directed to the same EV surface biomarker (with the same or different epitopes). In some such embodiments, a set of detection probes comprises at least two detection probes directed to distinct EV surface biomarkers. In some embodiments, a sample comprising an EV surface biomarker and intravesicular mRNA can be contacted with an anti-EV surface biomarker affinity agent (e.g., an antibody directed to EV surface biomarker as described in Example 2) conjugated to a single-stranded oligonucleotide (e.g., DNA) that serves as one of two primers in a pair for an intravesicular mRNA biomarker (e.g., described in Example 3) such that the anti-EV surface biomarker affinity agent is bound to the EV surface biomarker while the conjugated single-stranded oligonucleotide is hybridized with the intravesicular mRNA biomarker present in the same sample. A second primer of the pair and an RT-qPCR probe are then added to perform an RT-qPCR for detection of the presence of an intravesicular mRNA and an EV surface biomarker in a single sample.

In some embodiments, captured EVs can be read out by detection of at least one (e.g., 1, 2, 3, or more) mRNA biomarker described above; and at least one (e.g., 1, 2, 3, or more) EV intravesicular biomarkers described in Example 4. In some such embodiments, captured EVs can be read out (i) by detection of one or more (e.g., 1, 2, 3, or more) mRNAs; and (ii) by using a set of detection probes (e.g., as utilized and/or described herein), at least one of which are directed to one or more (e.g., 1, 2, 3, or more) intravesicular biomarkers described in Example 4. In some embodiments, a set of detection probes comprises at least one detection probe directed to an intravesicular biomarker (e.g., as described herein). In some embodiments, a set of detection probes comprises at least two detection probes each directed to the same intravesicular biomarker (e.g., with the same epitope or different epitopes). In some embodiments, a set of detection probes comprises at least two detection probes each directed to a distinct intravesicular biomarker (e.g., as described herein). In some embodiments, a sample comprising EV intravesicular biomarker and intravesicular mRNA can be contacted with an anti-EV intravesicular biomarker affinity agent (e.g., an antibody directed to EV intravesicular biomarker as described in Example 5) conjugated to a single-stranded oligonucleotide (e.g., DNA) that serves as one of two primers in a pair for an intravesicular mRNA biomarker (e.g., described in Example 4) such that the anti-EV intravesicular biomarker affinity agent is bound to the EV intravesicular biomarker while the conjugated single-stranded oligonucleotide is hybridized with the intravesicular mRNA biomarker present in the same sample. A second primer of the pair and an RT-qPCR probe are then added to perform an RT-qPCR for detection of the presence of an intravesicular mRNA and an intravesicular biomarker in a single sample.

The present Example further demonstrates exemplary methods for detection of at least one (e.g., 1, 2, 3, or more) intravesicular RNA biomarker in extracellular vesicles derived from cancer cell lines. In some embodiments, such a method comprises immunoaffinity capture of extracellular vesicles as described herein (e.g., via a surface-bound protein such as a surface biomarker described herein), followed by detection of intravesicular RNA, for example, by reverse-transcription qPCR (RT-qPCR). In some embodiments, extracellular vesicles are captured by a cancer-associated surface biomarker, e.g., in some embodiments using antibody-functionalized solid substrate (e.g., magnetic beads). In some embodiments, captured extracellular vesicles are lysed to release their nucleic acid cargo prior to detection of intravesicular RNA. In some embodiments, intravesicular RNA is or comprises mRNA.

In some embodiments, cell lines were selected that originate from or are associated with cancer (e.g., a particular cancer type). In some embodiments, such cell lines were selected that originate from or are associated with colon/colorectal cancer, leukemia, melanoma, ovarian cancer, or sarcoma (e.g., rhabdoid tumor). In some embodiments, G-401, K562, NIH: OVCAR-3, SK-MEL-1, or T84 cell lines were selected.

In some embodiments, extracellular vesicles were purified from conditioned cell culture medium, counted, immunoaffinity captured, and washed via methods as described herein (e.g., as described in Example 1).

Each RT-qPCR reaction mixture included a PCR reaction mixture (e.g., 50% (volume) Luna One-Step reaction mix, 5% (volume) Luna WarmStart RT enzyme mix, 5% (volume) primer-TaqMan probe mixture), and a variable combination of water, captured extracellular vesicles, and lysing agent. RT-qPCR was performed, for example, on the Quant Studio 3, with a suitable PCR protocol, e.g., hold at 55° C. for 10 minutes, hold at 95° C. for 1 minute, perform 50 cycles of 95° C. for 5 seconds and 62° C. for 15 seconds, and standard melt curve. The rate of temperature change was chosen to be standard (2° C. per second). All qPCRs were performed in doublets or triplets and ROX was used as the passive reference to normalize the qPCR signals. Data was then downloaded from the Quant Studio 3 machine and analyzed and plotted in Python 3.7. Primers and TaqMan probes for each gene were purchased from Integrated DNA Technologies (IDT) as a 20× concentrate.

As an initial experiment, MIF mRNA was found to be detected in 5e7 bulk extracellular vesicles that were lysed with 1% IGEPAL. Table 1 shows MIF expression in transcript per million (TPM) from different cell lines.

TABLE 1
Summary of MIF mRNA expression for cancer cell lines.
MIF RT-PCR signal (45-Ct) for a panel of bulk cell line
EVs with varying gene expression. Samples were tested
in singlicate and 5e7 EVs were used per reaction.

| Cell Line | MIF Expression (TPM) |
|---|---|
| SK-MEL-1 | 1302.5 |
| NIH:OVCAR-3 | 1175.3 |
| T84 | 196.8 |
| G-401 | 0.8 |

A similar experiment was performed to further demonstrate this approach across different intravesicular RNA biomarkers. Table 2 shows mRNA transcript expression levels in 5e7 bulk extracellular vesicles from different cell lines and shows that mRNA is detectable in cell-line EVs at levels that are dependent on cell gene expression.

TABLE 2
Summary of expression of four different
mRNA transcripts for cancer cell lines

| mRNA | | Expression | RT-PCR signal of 5e7 lysed EVs | |
|---|---|---|---|---|
| transcript | Cell Line | (TPM) | Replicate 1 | Replicate 2 |
| CLDN6 | OVCAR-3 | 203.4 | 34.0 | 33.9 |
| | SK-MEL-1 | 0.1 | 40.5 | Undetected |
| | No template | — | Undetected | Undetected |
| | control | | | |
| FAM83A | K-562 | 220.1 | 29.9 | 29.7 |
| | G-401 | 0.1 | Undetected | Undetected |
| | No template | — | Undetected | Undetected |
| | control | | | |
| HMGB3 | NCI-H1781 | 759.5 | 27.4 | 27.3 |
| | OVCAR-3 | 293.6 | 37.4 | 37.5 |
| | No template | — | Undetected | Undetected |
| | control | | | |
| B3GNT3 | NCI-H1781 | 134.9 | 33.6 | 33.9 |
| | G-401 | 0 | Undetected | Undetected |
| | No template | — | Undetected | Undetected |
| | control | | | |

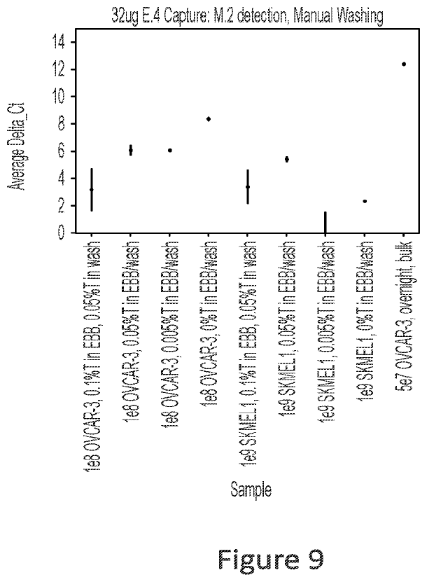

Additionally, an experiment was performed to detect the colocalization of at least one intravesicular RNA biomarkers with at least one surface biomarker (e.g., a surface marker that is associated with extracellular vesicles). In some embodiments, extracellular vesicles are captured using antibody-functionalized beads directed to a surface biomarker that is present on the surface of the extracellular vesicles. For example, in the present Example, EPCAM-targeted beads were used to capture extracellular vesicles. Bound extracellular vesicles were lysed and MIF mRNA content was quantified via RT-qPCR. Results are shown in FIG. 9. In some embodiments, a positive control cell line is selected that expresses a surface biomarker for capture and/or an intravesicular RNA biomarker for detection (e.g., EPCAM+, MIF+), while a negative control cell line is selected that does not express a target biomarker for capture and/or detection (e.g., EPCAM−, MIF+). NIH:OVCAR-3 was selected as a positive control cell line and SK-MEL-1 was selected as a negative control cell line. Multiple detergent conditions were also assessed in this experiment to assess the effect of detergent (e.g., Tween-20) concentration on assay performance. These data indicate that reducing detergent (e.g., Tween-20) concentration can improve assay performance. Without wishing to be bound by a particular theory, this effect may likely be due to preservation of membrane integrity during extracellular vesicle capture, as Tween-20 may permeabilize membranes.

The present Example demonstrates that intravesicular RNA can be detected via RT-qPCR. In particular, the present Example demonstrates that colocalization of surface biomarkers and intravesicular RNA in extracellular vesicles can be detected by immunoaffinity capture via a surface biomarker followed by RT-qPCR analysis of intravascular RNA.

### Example 4: Assessment of Intravesicular Biomarkers as Prostate Cancer Biomarkers

In some embodiments, prostate cancer detection includes detection of at least intravesicular protein(s) following immunoaffinity capture of extracellular vesicles.

In some embodiments, one or more surface proteins or extracellular membrane biomarkers that are present on extracellular vesicles (“capture biomarkers”) can be used for immunoaffinity capture of prostate cancer-associated extracellular vesicles. Examples of such capture biomarkers may include, but are not limited to polypeptides encoded by human genes as described in Example 2 and carbohydrate biomarkers as described in Example 2.

In some embodiments, EV immunoassay methodology (e.g., ones described herein such as in Example 1) and biomarker-validation process (e.g., ones described herein such as in Example 1) can be used to assess intravesicular proteins as biomarkers for prostate cancer. In some embodiments, an antibody directed to a capture biomarker (e.g., a surface protein present in prostate cancer-associated EVs) is conjugated to magnetic beads and evaluated, first on cell-line EVs then on patient samples, for its ability to bind the specific target protein biomarker. The antibody-coated bead is assessed for its ability to capture prostate cancer-associated EVs and the captured EVs by the antibody-coated beads are fixed and/or permeabilized prior to being profiled for their intravesicular proteins using a target entity detection system (e.g., a duplex system as described herein involving a set of two detection probes, each directed to a target marker that is distinct from the capture protein).

In some embodiments, captured EVs after fixation and/or permeabilization can be read out using at least one (e.g., 1, 2, 3, or more) intravesicular biomarker, which is or comprises a polypeptide encoded by a human gene as follows: ACP5, ACPP, ACSM1, ACTA2, ACTG2, ADAMTS1, AGR2, AIM1, ALCAM, ALDH1A3, ALOX15B, ANTXR1, ANTXR2, AP1M2, AR, ARG2, ARHGAP6, ARHGEF26, ARHGEF37, ASAP3, ATP8B1, ATP9A, BAIAP2L1, BCAM, BHLHA15, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CGN, CGNL1, CHMP4C, CLGN, CMTM4, COLCA1, CORO2A, CPE, CPNE4, CPQ, CRYM, CTSF, CX3CL1, CXADR, CXCR4, CYB561, DPYSL3, DSP, EEF1A2, EFNA1, EHD2, EHF, ELF3, EMP2, EPHX1, EPN3, ERBB3, ERG, ESRP1, ESRP2, F3, FAAH, FAM129A, FAM129B, FAM210B, FAM3B, FAM3D, FAM83H, FAM84A, FAM84B, FAT1, FBLIM1, FBP1, FLNC, FNBP1L, FOS, FOXA1, GADD45G, GATA2, GGT1, GJB1, GLYATL1, GNG4, GNMT, GRHL2, GUCY1A3, HGD, HID1, HIST1H2BK, HIST3H2A, HMGCS2, HOMER2, HOXB13, HSD17B6, HSPB6, HSPB8, ICA1, ID1, IQGAP2, IRF6, ITGA6, KIAA1522, KIF5C, KLK2, KRT14, KRT15, KRT17, KRT18, KRT19, KRT5, KRT7, KRT8, LCP1, LIFR, LIMCH1, LMOD1, LRP11, LRP3, LSR, MAL2, MAOA, MAP7, MARC1, MARCKSL1, MB, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, PALLD, PARVA, PCDH1, PCP4, PDIA5, PEBP4, PGM5, PKP1, PKP3, PPL, PPM1H, PPP1R1B, PPP3CA, PRAC1, PRR15L, PSAT1, PTPRF, PYCR1, RAB3B, RAMP1, RAP1GAP, RASEF, RBM47, RCAN3, RDH11, REEP6, REPS2, RGS10, RHPN2, RNF144B, RORC, RPS4Y1, S100A16, SELENBP1, SERINC5, SH3BGRL2, SH3BP4, SHC2, SLC1A5, SLC22A3, SLC2A12, SLC40A1, SLC4A4, SLC7A8, SLC9A3R2, SMOC2, SORD, SPDEF, SPINT1, SPINT2, SPTBN2, ST14, STAP2, STK39, SULT2B1, SYNE4, SYT7, SYTL1, TAGLN, TBC1D16, TBX3, TC2N, TCEA3, TEAD3, TESC, THBS4, TM4SF1, TMED3, TMEM150C, TMEM54, TMEM98, TMSB15A, TP53111, TPD52, TPM1, TSPAN13, TSPAN6, TSPAN8, VAMP8, VSTM2L, WLS, WWC1, YAP1, ZBTB42, ZC3H11A, ZNF217, ACSL3, CADH7, KCNN2, OPRK1, PDE9A, PLPP1, SPON2, TMEM121B, or combinations thereof. In some embodiments, an intravesicular biomarker described herein may comprise at least one post-translational modification. In some embodiments, captured EVs after fixation and/or permeabilization can be read out using a set of detection probes (e.g., as utilized and/or described herein), at least two of which are directed to one or more (e.g., 1, 2, 3, or more) intravesicular biomarkers described above. In some embodiments, a set of detection probes comprises two detection probes each directed to the same intravesicular biomarker. In some embodiments, a set of detection probes comprises two detection probes each directed to a distinct intravesicular biomarker.

In some embodiments, captured EVs after fixation and/or permeabilization can be read out using (i) at least one (e.g., 1, 2, 3, or more) intravesicular marker described above; and (ii) at least one (e.g., 1, 2, 3, or more) EV surface biomarkers described in Example 2. In some embodiments, captured EVs after fixation and/or permeabilization can be read out using a set of detection probes (e.g., as utilized and/or described herein), which comprises (i) a first detection probe directed to one or more (e.g., 1, 2, 3, or more) intravesicular markers described above; and (ii) a second detection probe directed to one or more (e.g., 1, 2, 3, or more) of EV surface biomarkers described in Example 2. In some embodiments, captured EVs after fixation and/or permeabilization can be read out by detecting an EV intravesicular marker and an EV intravesicular mRNA together in a single sample as described in Example 3 above.

### Example 5: Development of a Prostate Cancer Liquid Biopsy Assay

The present Example describes development of a prostate cancer liquid biopsy assay, for example, for screening hereditary- and average-risk individuals. Despite the success of digital rectal examination (DRE) for diagnosis of prostate cancer, it may be desirable to develop a non-invasive prostate cancer screening test from blood that may exhibit two features to provide clinical utility: (1) ultrahigh specificity (>99.5%) to minimize the number of false positives, and (2) high sensitivity (>40%) for stage I and II (Gleason score <7) prostate cancer when prognosis is most favorable. The development of such a test has the potential to save tens of thousands of lives each year.

Several different biomarker classes have been studied for a prostate cancer liquid biopsy assay including circulating tumor DNA (ctDNA), circulating tumor cells (CTCs), bulk proteins, and extracellular vesicles (EVs). EVs are particularly promising due to their abundance and stability in the bloodstream relative to ctDNA and CTCs, suggesting improved sensitivity for early-stage cancers. Moreover, EVs contain cargo (e.g., proteins, RNA, metabolites) that originated from the same cell, providing superior specificity over bulk protein measurements. While the diagnostic utility EVs has been studied, much of this work has pertained to bulk EV measurements or low-throughput single-EV analyses.

This present Example describes one aspect of an exemplary approach for early-stage prostate cancer detection through the profiling of individual extracellular vesicles (EVs) in human plasma. EVs, including exosomes and microvesicles, contain co-localized proteins, RNAs, metabolites, and other compounds representative of their cell of origin (Kosaka et al., 2019; which is incorporated herein by reference for the purpose described herein). The detection of strategically chosen co-localized markers within a single EV can enable the identification of cell type with ultrahigh specificity, including the ability to distinguish cancer cells from normal tissues. As opposed to other cancer diagnostic approaches that rely on cell death for biomarkers to enter the blood (i.e., cfDNA), EVs are released at a high rate by functioning cells. Single cells have been shown to release as many as 10,000 EVs per day in vitro (Balaj et al., 2011; which is incorporated herein by reference for the purpose described herein). In addition, it is widely accepted that cancer cells release EVs at a higher rate than healthy cells (Bebelman et al. 2018; which is incorporated herein by reference for the purpose described herein).

In one aspect, the present disclosure provides insights and technologies involving identification of genes that are upregulated in prostate cancer versus healthy tissues using Applicant's proprietary bioinformatic biomarker discovery process. From a list of upregulated biomarkers, biomarker combinations that are predicted to exhibit high sensitivity and specificity for prostate cancer are designed. Using an exemplary individual EV assay (see, e.g., illustrated in FIG. 1 or 2 and/or described herein), co-localization of such biomarkers on an individual vesicle may be detected, indicating that the grouping of biomarkers originated from the same cell. This provides superior specificity to bulk biomarker measurements, including bulk EV assays, given that many upregulated cancer biomarkers are expressed by one or more healthy tissues. Through the careful design of biomarker combinations, signals from competing tissues can be reduced or eliminated, including those closely related to prostate cancer. In some embodiments, the present disclosure provides technologies with ultrahigh specificity that is particularly helpful as a prostate cancer screening test for which the prevalence of disease is low and a high positive-predictive value (>10%) is required (Seltzer et al., 1995; which is incorporated herein by reference for the purpose described herein).

### Biomarker Discovery

In some embodiments, a biomarker discovery process leverages bioinformatic analysis of large databases and an understanding of the biology of prostate cancer and extracellular vesicles.

### Individual Extracellular Vesicle Analysis

The detection of tumor-derived EVs in the blood requires an assay that has sufficient selectivity and sensitivity to detect relatively few tumor-derived EVs per milliliter of plasma in a background of 10 billion EVs from a diverse range of healthy tissues. The present disclosure, among other things, provides technologies that address this challenge. For example, in some embodiments, an assay for individual extracellular vesicle analysis is illustrated in FIG. 1, which is performed in three key steps as outlined below:
1. EVs are purified from patient plasma using size-exclusion chromatography (SEC), which removes greater than 99% of soluble proteins and other interfering compounds.
2. Tumor-specific EVs are captured using antibody-functionalized magnetic beads specific to a membrane-associated surface biomarker.
3. A modified version of proximity-ligation-immuno quantitative polymerase chain reaction (pliq-PCR) is performed to determine the co-localization of additional biomarkers contained on or within the captured EVs.

In many embodiments of a modified version of a pliq-PCR assay, two or more different antibody-oligonucleotide conjugates are added to the EVs captured by the antibody-functionalized magnetic bead and the antibodies subsequently bind to their biomarker targets. The oligonucleotides are composed of dsDNA with single-stranded overhangs that are complementary, and thus, capable of hybridizing when in close proximity (i.e., when the corresponding biomarker targets are located on the same EV). After washing away unbound antibody-oligonucleotide species, adjacently bound antibody-oligonucleotide species are ligated using a standard DNA ligase reaction. Subsequent qPCR of the ligated template strands enables the detection and relative quantification of co-localized biomarker species. In some embodiments, two to twenty distinct antibody-oligonucleotide probes can be incorporated into such an assay, e.g., as described in U.S. application Ser. No. 16/805,637 (published as US2020/0299780; issued as U.S. Pat. No. 11,085,089), and International Application PCT/US2020/020529 (published as WO2020180741), both filed Feb. 28, 2020 and entitled “Systems, Compositions, and Methods for Target Entity Detection”; which are both incorporated herein by reference in their entirety for any purpose.

pliq-PCR has numerous advantages over other technologies to profile EVs. For example, pliq-PCR has a sensitivity three orders of magnitude greater than other standard immunoassays, such as ELISAs (Darmanis et al., 2010; which is incorporated herein by reference for the purpose described herein). The ultra-low LOD of a well-optimized pliq-PCR reaction enables detection of trace levels of tumor-derived EVs, down to a thousand EVs per mL. This compares favorably with other emerging EV analysis technologies, including the Nanoplasmic Exosome (nPLEX) Sensor (Im et al., 2014; which is incorporated herein by reference for the purpose described herein) and the Integrated Magnetic-Electrochemical Exosome (iMEX) Sensor (Jeong et al., 2016; which is incorporated herein by reference for the purpose described herein), which have reported LODs of ˜10³ and ˜ 10⁴ EVs, respectively (Shao et al., 2018; which is incorporated herein by reference for the purpose described herein). Moreover, in some embodiments, a modified version of pliq-PCR approach does not require complicated equipment and can uniquely detect the co-localization of multiple biomarkers on individual EVs.

In some embodiments, to further improve the sensitivity and specificity of an individual EV profiling assay, other classes of EV biomarkers include mRNA and intravesicular proteins (in addition to EV surface biomarker) can be identified and included in an assay.

### Preliminary Work

Through preliminary studies, a workflow was developed in which biomarker candidates are validated to be present in EVs and capable of being detected by commercially available antibodies or mRNA primer-probe sets. For a given biomarker of interest, one or more cell lines expressing (positive control) and not expressing the biomarker of interest (negative control) can be cultured to harvest their EVs through concentrating their cell culture media and performing purification to isolate nanoparticles having a size range of interest (e.g., using SEC). Typically, extracellular vesicles may range from 30 nm to several micrometers in diameter. See, e.g., Chuo et al., “Imaging extracellular vesicles: current and emerging methods” Journal of Biomedical Sciences 25:91 (2018) which is incorporated herein by reference for the purpose described herein, which provides information of sizes for different extracellular vesicle (EV) subtypes: migrasomes (0.5-3 μm), microvesicles (0.1-1 μm), oncosomes (1-10 μm), exomeres (<50 nm), small exosomes (60-80 nm), and large exosomes (90-120 nm). In some embodiments, nanoparticles having a size range of about 30 nm to 1000 nm may be isolated for detection assay. In some embodiments, specific EV subtype(s) may be isolated for detection assay.

To further improve the performance of an exemplary single EV profile assay (e.g., ones described herein) for detection of prostate cancer, in some embodiments, additional biomarker candidates including membrane-bound proteins and intravesicular mRNAs/proteins can be identified.

In some embodiments, it was previously demonstrated by Applicant the feasibility of EV-mRNA detection using purified cell-line EVs in bulk. Through immunoaffinity capture of a membrane bound protein marker, this approach enables the detection of two co-localized biomarkers. Moreover, EV-mRNA detection requires a simpler protocol because RT-qPCR can be performed directly after immunoaffinity capture. In some embodiments, mRNA detection using EVs can be performed by capturing EVs using capture probes (e.g., as described herein) and detecting a particular prostate cancer mRNA biomarker. EVs that express both capture probe marker and prostate cancer mRNA biomarker are selectively detected.

### Example 6: Bioinformatically-Identified Biomarkers and Biomarker Combinations

The present Example illustrates an exemplary bioinformatically driven approach for identification of certain biomarkers and biomarker combinations that can be useful for prostate cancer diagnosis.

### Bioinformatic Filtering

There are more than 55,000 transcripts captured in the Genotype-Tissue Expression (GTEx) database (e.g., a primary data resource for normal tissue gene expression) and the Cancer Genome Atlas (TCGA) database (e.g., a primary data resource for cancer tissue gene expression). To identify biomarkers that are useful for detection of prostate cancer, two filtering steps were applied to the data.

In some embodiments, UniProt filter was used. Biomarkers that have a valid UniProt entry, which includes evidence that a biomarker protein was found to be associated with a membrane, were considered in the analysis (e.g., proteins with no evidence of being membrane associated were optionally filtered out). Such a filtering step may optionally distinguish between different membranes of interest or level of confidence of the provided evidence.

In some embodiments, Vesiclepedia filter was used. Vesiclepedia (a repository of extracellular vesicle publications) was used to filter the results. Vesiclepedia lists the number of EV related references published for each gene (e.g., Entrez). These references were used as a proxy for presence of a given biomarker in or on EVs. If no EV-related publications exist for a given biomarker, it is less likely to be an actual EV biomarker, and was thus filtered from the list of biomarkers for further consideration.

### Minimum Expression and Differentiation Filtering

In some embodiments, a minimum expression level of a biomarker is considered. Low biomarker expression may produce stochastic noise and make robust signal detection difficult and unreliable. To overcome this challenge, one or more (including all of) of the following expression filters were applied. In particular embodiments, four expression filters were applied.

### Minimal Expression in the Cancer of Interest

In some embodiments, a minimum number of samples were used to show expression levels that were detectable in the cancer of interest, while leaving room for discovery of subtypes that potentially have differential gene expression profiles. To achieve this filter, in some embodiments, the 80th percentile of gene expression in the TCGA cancer of interest (e.g., prostate cancer) was calculated, and in some embodiments, biomarkers that have a transcript per million (TPM) value of >15 at the 80th percentile were considered.

### Minimal Expression Associated Cell-Lines

In some embodiments, positive control cell-lines were utilized for testing of antibodies directed towards bioinformatically-predicted biomarkers. In some embodiments, the Cancer Cell Line Encyclopedia (CCLE) gene expression set, which contains >1000 cell-line profiles, was utilized to reduce biomarker lists to those for which cell-lines expressing a biomarker of interest exist. In some embodiments, the 90th percentile of expression for each biomarker across cancer-specific cell-lines was calculated, and in some embodiments, biomarkers with a TPM>15 at the 90^{th} percentile were considered.

### Minimum Expression in the Cancer of Interest on a Protein Level

One skilled in the art will understand that not all genes that are expressed are ultimately translated into proteins. Accordingly, in some embodiments, mass spectrometry data from the Clinical Proteomic Tumor Analysis Consortium (CPTAC) were utilized to filter for protein-expressing genes. In some embodiments, biomarkers with a spectral count greater than 10 were considered to be expressed.

### Minimum Differentiation Against Normal Tissue

In some embodiments, assays described herein achieved superior specificity by requiring co-expression of at least two biomarkers, and in some embodiments, at least three biomarkers, on the same extracellular vesicle. Simple differential gene expression of normal tissues yielded too many false negative values. Instead, in some embodiments, a biomarker signature comprises a combination of biomarkers that may include biomarkers that were highly expressed in multiple tissue types, but only when they were paired with other biomarkers that provided additional discriminatory power (e.g., highly tissue specific and/or highly cancer specific). However, such an analysis could capture housekeeping genes, such as GAPDH, which were ubiquitously expressed, and accordingly were not necessarily useful as discriminatory biomarkers. To remove such markers, in some embodiments, a z-score comparing cancerous tissue (e.g., prostate cancer) and every tissue type in GTEx for a given biomarker was calculated. In some embodiments, a biomarker with a z-score of 5 at the 80th percentile, in at least one normal tissue type was selected (e.g., at least one normal tissue was clearly excluded by a biomarker candidate).

### Simulation and Stochastic Sampling

Discriminatory power of a biomarker signature candidate or biomarker combination candidate comprising at least two or more (including, e.g., at least three or more) biomarkers can be determined by simulating and comparing expression of such a biomarker signature candidate in normal subjects (e.g., subjects who were determined not to have prostate cancer) to that in cancer subjects. Combinations of at least 2 and at least 3 biomarkers were generated based on filtered biomarker sets. An EV score, which estimated the number of EVs generated by a profiled tissue, was calculated for a given combination by multiplying TPM values of all markers in a given combination.

To simulate a population of normal subjects, a cohort of 5000 plasma samples from 5000 “healthy individuals” was created. Individual samples were created by randomly selecting tissue samples from each of the 54 tissues in the GTEx database and multiplying the TPM values of expressed genes with the estimated weight in grams of each organ based on a healthy individual. EV scores were then summed for an individual across tissues to simulate an individual. EV scores were then summed across tissues for a simulated individual. In addition to a healthy cohort, 5000 samples from “cancer individuals” were created by repeating the “healthy” pool generation technique, but with an added step of adding EV scores of randomly selected prostate cancer (e.g., prostate adenocarcinoma) samples from TCGA, multiplying the sample by 1, 10, or 100, corresponding to a 1 g, a 10 g, or a 100 g tumor. Using these two sample pools of “healthy” and “cancer” individuals, sensitivity for each biomarker combination candidate at 99% specificity was calculated. This metric was then used to rank biomarker combination candidates.

For initial biomarker signature selection, in some embodiments, 1 million combinations of three biomarkers were randomly sampled, and in some embodiments simulations were conducted using a 100 g tumor, and 1000 individuals in each of the cancer and the healthy pool. In some embodiments, biomarker combinations were then ranked based on their sensitivity value at 99% specificity. In some embodiments, single biomarkers were then ranked based on the top 0.5 percentile of their rank in the combination list.

### Example 7: Correlation of Bioinformatically-Identified Biomarkers and Biomarker Combinations with Pathways Known in the Art

The present Example describes a gene set enrichment analysis for determination of overlap between certain bioinformatically-predicted biomarkers and published gene pathways. One skilled in the art will recognize that in certain cases, lists of single genes can be challenging to appropriately interpret. Fortunately, there are resources that provide functional lists of genes, such as, for example, lists of genes that encode proteins that are components of the same biochemical pathway or phenomenon. Comparing a bioinformatically-identified list of biomarkers to known gene sets and biochemical pathways can impose structure on a list of biomarkers.

Table 3 shows an enrichment analysis of certain bioinformatically-identified biomarkers when compared to all gene sets in the Molecular Signature Database Category 2-Cannonical pathways (v.7.4.) from the Broad Institute. This database includes, among other resources, KEGG, Biocarta, and Reactome data. Each p-value is a result of a Chi-square test, comparing a particular gene set with a list of certain bioinformatically-identified biomarkers against the background of all genes in MSigDB C2-CP database. Biomarkers were ranked with the highest overlap first, and in some embodiments, overlaps with a nominal p-value of 0.05 were considered.

Table 3 shows certain molecular pathways that are enriched in a list of bioinformatically identified biomarkers, following correction for multiple testing, several molecular pathways exhibited a false discovery rate (FDR) of less than 0.05. Such molecular pathways provide a biological theme for certain bioinformatically identified biomarkers.

TABLE 3
Enrichment analysis of certain bioinformatically identified biomarkers

| | | | | Exemplary |
|---|---|---|---|---|
| Gene Ontology Pathway, | Raw P | | Pathway | Biomarkers |
| Source and Description | value | FDR | Gene # | Included |
| REACTOME_ZINC_IN- | 0.00E+00 | 2.90E−06 | 10 | SLC39A6, SLC39A7 |
| FLUX_INTO_CELLS_BY_THE_SLC39_GENE_FAMILY | | | | |
| REACTOME_ZINC_TRANSPORTERS | 5.10E−06 | 1.49E−02 | 17 | SLC39A6, SLC39A7 |
| WP_MAMMARY_GLAND_DEVELOP- | 1.01E−05 | 2.96E−02 | 18 | CDH1, CLDN4 |
| MENT_PATHWAY_EMBRYONIC_DE- | | | | |
| VELOPMENT_STAGE_1_OF_4 | | | | |
| REACTOME_METAL_ION_SLC_TRANSPORTERS | 3.76E−04 | 1.00E+00 | 26 | SLC39A6, SLC39A7 |
| REACTOME_CELL_CELL_JUNCTION_ORGANIZATION | 3.83E−04 | 1.00E+00 | 65 | CDH1, CLDN3, CLDN4 |
| REACTOME_TIGHT_JUNCTION_INTERACTIONS | 1.13E−03 | 1.00E+00 | 30 | CLDN3, CLDN4 |
| WP_ZINC_HOMEOSTASIS | 4.42E−03 | 1.00E+00 | 37 | SLC39A6, SLC39A7 |
| REACTOME_FORMATION_OF_XYLU- | 4.74E−03 | 1.00E+00 | 5 | SORD |
| LOSE_5_PHOSPHATE | | | | |
| REACTOME_SIGNALING_BY_MST1 | 4.74E−03 | 1.00E+00 | 5 | HPN |
| REACTOME_ATTACHMENT_AND_ENTRY | 4.74E−03 | 1.00E+00 | 5 | TMPRSS2 |
| REACTOME_CELL_JUNCTION_ORGANIZATION | 5.50E−03 | 1.00E+00 | 92 | CDH1, CLDN3, CLDN4 |
| REACTOME_RNA_POLYMERASE_II_TRANSCRIPTION | 5.94E−03 | 1.00E+00 | 1374 | |
| WP_7Q1123_COPY_NUMBER_VARIA- | 8.18E−03 | 1.00E+00 | 98 | CLDN3, CLDN4, |
| TION_SYNDROME | | | | DNAJC30 |
| REACTOME_METHIONINE_SALVAGE_PATHWAY | 1.08E−02 | 1.00E+00 | 6 | ADI1 |
| REACTOME_MET_RECEPTOR_ACTIVATION | 1.08E−02 | 1.00E+00 | 6 | HPN |
| BIOCARTA_CB1R_PATHWAY | 1.98E−02 | 1.00E+00 | 7 | FAAH |
| WP_SARSCOV2_AND_ANGIOTENSIN | 1.98E−02 | 1.00E+00 | 7 | TMPRSS2 |
| CONVERTING_ENZYME_2_RECEP- | | | | |
| TOR_MOLECULAR_MECHANISMS | | | | |
| REACTOME_FRUCTOSE_METABOLISM | 1.98E−02 | 1.00E+00 | 7 | SORD |
| REACTOME_SARS_COV_1_INFECTION | 2.08E−02 | 1.00E+00 | 50 | CHMP4C, TMPRSS2 |
| REACTOME_NEUROTRANSMITTER_RELEASE_CYCLE | 2.27E−02 | 1.00E+00 | 51 | NAAA, UNC13B |
| KEGG_LYSOSOME | 2.61E−02 | 1.00E+00 | 121 | AP1M2, CLN5, SCARB2 |
| WP_COPPER_HOMEOSTASIS | 2.90E−02 | 1.00E+00 | 54 | STEAP1, STEAP2 |
| REACTOME_REGULATION_OF_INSU- | 2.95E−02 | 1.00E+00 | 124 | GOLM1, MIA3, VWA1 |
| LIN_LIKE_GROWTH_FACTOR_IGF_TRANS- | | | | |
| PORT_AND_UPTAKE_BY_INSU- | | | | |
| LIN_LIKE_GROWTH_FACTOR_BIND- | | | | |
| ING_PROTEINS_IGFBPS | | | | |
| BIOCARTA_CTBP1_PATHWAY | 3.13E−02 | 1.00E+00 | 8 | CDH1 |
| WP_LDLRAD4_AND_WHAT_WE_KNOW_ABOUT_IT | 3.13E−02 | 1.00E+00 | 8 | PMEPA1 |
| REACTOME_SYNTHESIS_OF_UDP_N_ACE- | 3.13E−02 | 1.00E+00 | 8 | GNPNAT1 |
| TYL_GLUCOSAMINE | | | | |
| REACTOME_NEUREXINS_AND_NEUROLIGINS | 3.37E−02 | 1.00E+00 | 56 | HOMER2, SYT7 |
| REACTOME_CELL_CELL_COMMUNICATION | 3.69E−02 | 1.00E+00 | 130 | CDH1, CLDN3, CLDN4 |
| WP_PROXIMAL_TUBULE_TRANSPORT | 3.87E−02 | 1.00E+00 | 58 | ABCC4, SLC4A4 |
| KEGG_TIGHT_JUNCTION | 3.96E−02 | 1.00E+00 | 132 | CLDN3, CLDN4, RAB3B |
| KEGG_CELL_ADHESION_MOLECULES_CAMS | 4.10E−02 | 1.00E+00 | 133 | CDH1, CLDN3, CLDN4 |
| REACTOME_CALCINEURIN_ACTIVATES_NFAT | 4.49E−02 | 1.00E+00 | 9 | PPP3CA |
| REACTOME_INLA_MEDIATED_EN- | 4.49E−02 | 1.00E+00 | 9 | CDH1 |
| TRY_OF_LISTERIA_MONOCYTOGENES_IN- | | | | |
| TO_HOST_CELLS | | | | |
| REACTOME_SERINE_BIOSYNTHESIS | 4.49E−02 | 1.00E+00 | 9 | SERINC5 |
| REACTOME_CA2_PATHWAY | 4.98E−02 | 1.00E+00 | 62 | GNG4, PPP3CA |

### Example 8: Correlation of Bioinformatically-Identified Biomarkers and Biomarker Combinations with Clinical Covariates and Known Somatic Mutational Drivers

The present Example illustrates potential associations between known prostate cancer clinical covariates and certain bioinformatically-predicted biomarkers; and potential associations between known prostate cancer mutational drivers and certain bioinformatically-predicted biomarkers.

In some embodiments, one or more clinical covariates were considered in addition to gene expression of certain bioinformatically-identified biomarkers. Such analysis can be useful to provide an indication on potential subgroups, including staging, lymph node involvement, histological type (e.g., acinar type or other subtype), Gleason score, and others.

In some embodiments, clinical covariates included nodal involvement (e.g., n0, n1, n2), cancer stage, histological type, and/or Gleason score. In some embodiments, cancer stage included stage I, stage II, stage III, or stage IV cancers. In some embodiments, histological type included acinar subtype and other subtype. In some embodiments, Gleason score was in a range from 6 to 10.

This clinical covariate analysis did not identify any strong enrichments within the TCGA sample, demonstrating that certain bioinformatically-identified biomarker combinations can be particularly useful to identify prostate cancer samples (e.g., prostate adenocarcinoma samples) irrespective of a particular clinical covariate (data not shown).

In some embodiments, one or more somatic mutational drivers (including, e.g., mutation and copy number of alteration profiles) were considered in addition to gene expression of certain bioinformatically-identified biomarkers. For example, certain major known mutational drivers of prostate cancer include, but are not limited to mutations in SPOP, IDH1, FOXA1, FLI1, ETV4, ETV1, ERG, or combinations thereof. For each of these drivers, cancer-associated mutations may include copy number alterations (CNAs; including, e.g., but not limited to amplification and/or deletion) and/or mutations (including, e.g., but not limited to in-frame mutation, missense mutation, splice, and/or truncating mutation). A clustering analysis was performed to identify associations between bioinformatically-predicted biomarkers, biomarker combinations, and certain major mutational drivers of prostate cancer.

This mutational driver analysis did not identify any strong enrichments within the TCGA sample, demonstrating that certain bioinformatically-predicted biomarkers and/or biomarker combinations can be particularly useful to identify prostate cancer samples (e.g., prostate adenocarcinoma samples) irrespective of a particular mutational driver (data not shown).

### Example 9: Assessment of Certain Surface Biomarkers and Combinations Thereof as Targets for Capture Probes and/or Detection Probes for Target Entity Detection Systems Described Herein

The present Example describes exemplary characterization of surface biomarkers for use in assays as described herein (e.g., for the detection of prostate cancer, e.g., in some embodiments prostate adenocarcinoma). In some embodiments, a surface biomarker was assessed as a target for a capture probe of assays described herein. In some embodiments, a surface biomarker was assessed as a target for a detection probe of assays described herein.

In this Example where a surface biomarker was assessed as target for a capture probe of assays described herein, a target-capture moiety (e.g., in some embodiments an antibody agent) that binds to a particular surface biomarker of interest was immobilized on a solid substrate to form a capture probe. The capture probe was then added to conditioned media from a selected cell line to capture nanoparticles (i) having a size range of interest (e.g., about 30 nm to about 1000 nm) that included extracellular vesicles, and (ii) having on their surfaces the particular surface biomarker of interest. Captured nanoparticles that included extracellular vesicles were then read out by a set of detection probes (as described herein) each directed to a canonical exosome marker. For example, CD63, CD81, and CD9 are canonical exosome markers that are highly expressed in multiple tissues and cell lines (see, for example, Bobrie et al., Journal of extracellular vesicles 1.1, 2012, incorporated herein by reference). Unconditioned media (e.g., buffer or media which does not contain nanoparticles having a size range of interest (e.g., about 30 nm to about 1000 nm) that included extracellular vesicles) was used as a negative control.

In this Example where a surface biomarker was assessed as target for a detection probe of assays described herein, a target-capture moiety (e.g., in some embodiments an antibody agent) that binds to a canonical exosome marker (e.g., in some embodiments CD63 or CD81) was immobilized on a solid substrate to form a capture probe. The capture probe was then added to conditioned media from a selected cell line to capture nanoparticles (i) having a size range of interest (e.g., about 30 nm to about 1000 nm) that included extracellular vesicles, and (ii) having on their surfaces the particular biomarker of interest. Captured nanoparticles that included extracellular vesicles were then read out by a set of detection probes (as described herein) each directed to a particular surface biomarker of interest. Unconditioned media (e.g., buffer or media which does not contain nanoparticles having a size range of interest (e.g., about 30 nm to about 1000 nm) that included extracellular vesicles) was used as a negative control.

In some embodiments, a positive cell line is selected that expresses a target biomarker of interest, while a negative cell line is selected that does not express a target biomarker of interest. In some embodiments, such positive and negative cell lines are selected that originate from or are associated with a particular cancer type. In some embodiments, such cell lines were selected that originate from or are associated with breast cancer, colon/colorectal cancer, lung cancer, lymphoma, ovarian cancer, pancreatic cancer, sarcoma, or skin cancer. In some embodiments, A549, AsPC-1, AU565, BT-20, BxPC-3, Caov-3, COLO 201, COR-L95, COV413A, COV644, Farage, HCC1419, HCC1500, HCC4006, HT-1080, MCF7, MeWo, NCI-H146, NCI-H1781, NCI-H441, NIH: OVCAR-3, OVISE, OVSAHO, Ramos, SK-OV-3, or SUP-M2 cell lines were selected.

Table 4 shows absolute and delta Ct values for certain surface biomarkers assayed individually as targets for a capture probe. Ct values were read from qPCR where the numeric value corresponds to the number of PCR cycles (i.e., higher values indicate less signal). CD63, CD81, or CD9 were used as a target for a detection probe. As shown in Table 4, certain surface biomarkers may be particularly useful as a target for a capture probe in assays as described herein. For example, surface biomarkers with high delta Ct values (e.g., delta Ct values greater than 2, including, e.g., greater than 3, greater than 4, greater than 5, or higher) may be particularly useful as targets for capture probes. Likewise, such characterization may also be helpful in identifying target-capture moieties that are particularly useful as capture probes. In some embodiments, surface biomarkers MUC1 and other mucins (e.g., MUC4 and MUC16) are particularly useful targets for capture probes. In some embodiments, surface biomarkers that comprise glycosylation, e.g., sTn antigen, sLex antigen, are particularly useful targets for capture probes.

TABLE 4
Characterization of certain surface biomarkers individually as targets for a capture probe.

| | CD63 | | | | CD81 | | | | CD9 | | | |
|---|---|---|---|---|---|---|---|---|---|---|---|---|
| Target | | (+) | (−) | No EV | | (+) | (−) | No EV | | (+) | (−) | No EV |
| Biomarker | dCt | abs Ct | abs Ct | abs Ct | dCt | abs Ct | abs Ct | abs Ct | dCt | abs Ct | abs Ct | abs Ct |
| ABCC4 | — | — | — | — | 3.89 | 26.19 | 30.09 | 31.91 | — | — | — | — |
| CD38 | — | — | — | — | 11.5 | 20.14 | 31.63 | 30.39 | — | — | — | — |
| EPCAM | 8.31 | 20.25 | 28.56 | 28.85 | 6.45 | 22.07 | 28.52 | 28.79 | — | — | — | — |
| ERBB2 | 1.81 | 31.74 | 33.55 | 37.46 | 9.79 | 20.82 | 30.62 | 30.83 | — | — | — | — |
| FOLH1 | — | — | — | — | 2.86 | 26.61 | 29.47 | 30.48 | — | — | — | — |
| ITGAV | — | — | — | — | −2.67 | 24.98 | 22.31 | 40 | 7.99 | 18.16 | 26.15 | 32.31 |
| MARCKSL1 | 1.78 | 30.72 | 32.49 | 31.86 | 3.94 | 29.59 | 33.53 | 34 | — | — | — | — |
| CD274 | 6.59 | 26.69 | 33.28 | 34.05 | 5.77 | 28.64 | 34.42 | 40.52 | — | — | — | — |
| PSCA | — | — | — | — | 1.59 | 28.86 | 30.45 | 31.93 | — | — | — | — |
| sTn antigen | 2.08 | 23.71 | 25.78 | 41.51 | 4.25 | 21.49 | 25.73 | 42.34 | — | — | — | — |
| Tn antigen | 7.67 | 22.52 | 30.19 | 39.2 | 10.03 | 20.29 | 30.32 | 35.58 | — | — | — | — |
| STEAP1 | 3.56 | 28.41 | 31.97 | 30.44 | −0.49 | 26.75 | 26.26 | 26.93 | 1.34 | 27.44 | 28.78 | 29.33 |
| T antigen | 6.34 | 23.46 | 29.8 | 36.7 | 8.17 | 21.89 | 30.06 | 40.57 | — | — | — | — |
| TMPRSS2 | 0.24 | 22.87 | 23.11 | 22.29 | 0.81 | 20.99 | 21.8 | 20.98 | 3.85 | 22.59 | 26.44 | 26.63 |
| TRPM4 | 3.35 | 29.02 | 32.37 | 38.85 | −7.64 | 32.73 | 25.09 | 31.65 | 2.86 | 27.15 | 30.01 | 33.54 |
| TSPAN1 | — | — | — | — | 10.19 | 22.08 | 32.27 | 35.48 | — | — | — | — |

dCt: delta Ct between positive and negative cell-line; abs Ct: absolute Ct; (+): positive cell line; (−): negative cell line.

Table 5 shows absolute and delta Ct values for certain surface biomarkers assayed individually as targets for a detection probe. Ct values were read from qPCR where the numeric value corresponds to the number of PCR cycles (i.e., higher values indicate less signal). CD63 or CD81 were used as a target for a capture probe. As shown in Table 5, certain surface biomarkers may be particularly useful as a target for a detection probe in assays as described herein. For example, surface biomarkers with high delta Ct values (e.g., delta Ct values greater than 2, including, e.g., greater than 3, greater than 4, greater than 5, or higher) may be particularly useful as targets for detection probes. Likewise, such characterization may also be helpful in identifying target-capture moieties that are particularly useful as detection probes. In some embodiments, surface biomarkers shown in Table 5 can be used as targets for detection probes.

TABLE 5
Screening for target biomarkers individually as targets for a detection probe.

| | CD63 | | | | CD81 | | | |
|---|---|---|---|---|---|---|---|---|
| Target | | (+) | (−) | No EV | | (+) | (−) | No EV |
| Biomarker | dCt | abs Ct | abs Ct | abs Ct | dCt | abs Ct | abs Ct | abs Ct |
| BCAM | 1.67 | 29.55 | 31.22 | 30.67 | 7.69 | 27.9 | 35.6 | 34.06 |
| CD38 | — | — | — | — | 17.25 | 21.95 | 39.19 | 39.54 |
| CDH1 | 1.24 | 28.24 | 29.48 | 29.78 | 8.85 | 27.19 | 36.04 | 36.23 |
| CLDN3 | 4.46 | 28.09 | 32.55 | 33.18 | 8.75 | 27.35 | 36.1 | 37.58 |
| EPCAM | 14.94 | 20.69 | 35.62 | 36.47 | 14.59 | 22.06 | 36.65 | 38.6 |
| ERBB2 | — | — | — | — | 16.91 | 23.09 | 40 | 40 |
| GOLM1 | 10.61 | 32.2 | 42.81 | 45 | 7.76 | 31.39 | 39.15 | 37.92 |
| MARCKSL1 | 0.31 | 33.57 | 33.88 | 37.49 | 10.83 | 28.02 | 38.84 | 42.15 |
| PSCA | — | — | — | — | 10.95 | 29.05 | 40 | 39.46 |
| sTn antigen | 9.86 | 29.29 | 39.15 | 37.48 | 10.35 | 26.89 | 37.24 | 41.43 |
| Tn antigen | 3.88 | 32.68 | 36.56 | 37.76 | 6.04 | 30.46 | 36.51 | 37.87 |
| T antigen | 7.54 | 31.78 | 39.33 | 42.6 | 8.78 | 29.12 | 37.91 | 38.5 |
| TSPAN1 | — | — | — | — | 8.71 | 29.07 | 37.78 | 38.06 |
| VWA1 | 12.35 | 29.6 | 41.94 | 42.8 | 9.63 | 28.88 | 38.51 | 42.04 |

dCt: delta Ct between positive and negative cell-line; abs Ct: absolute Ct; (+): positive cell line; (−): negative cell line.

Multiple canonical exosome markers were used for characterization of each surface biomarker as indicated herein because each canonical exosome marker can vary in expression level across exosomes (e.g., exosomes derived from a specific sample). For example, certain exosomes may express a high level of CD63, but not CD81 or CD9, or vice versa. Therefore, as shown in Tables 4 and 5, Ct values may vary between canonical exosome markers for a given surface biomarker.

In some embodiments, certain surface biomarkers were characterized in combination as a target for a capture probe (e.g., as described herein) and as a target for a detection probe (as described herein), of assays described herein. For example, a biomarker combination comprising surface biomarkers of EPCAM and T antigen encompasses combinations where EPCAM is the target for a capture probe and T antigen is the target for a detection probe; and also combinations where T antigen is the target for a capture probe and EPCAM is the target for a detection probe. Such 2-biomarker combinations can be useful for the detection of prostate cancer (e.g., in some embodiments prostate adenocarcinoma).

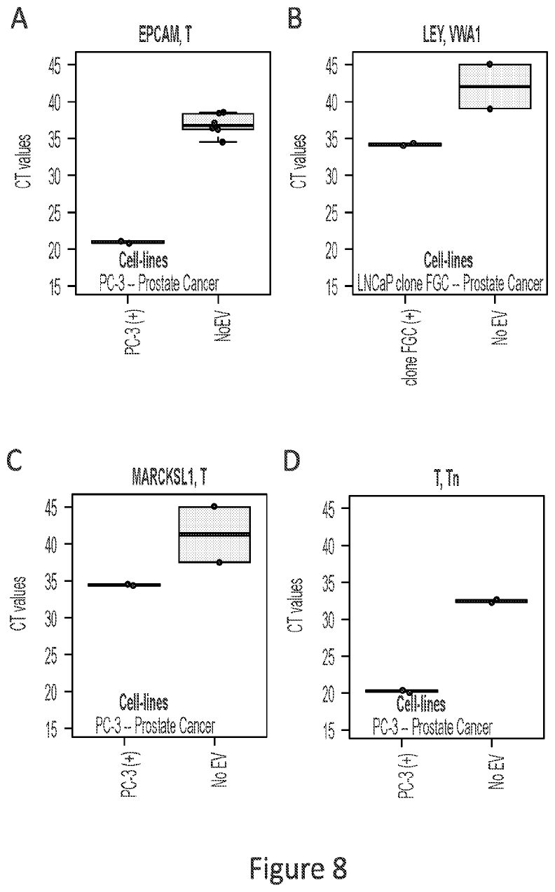

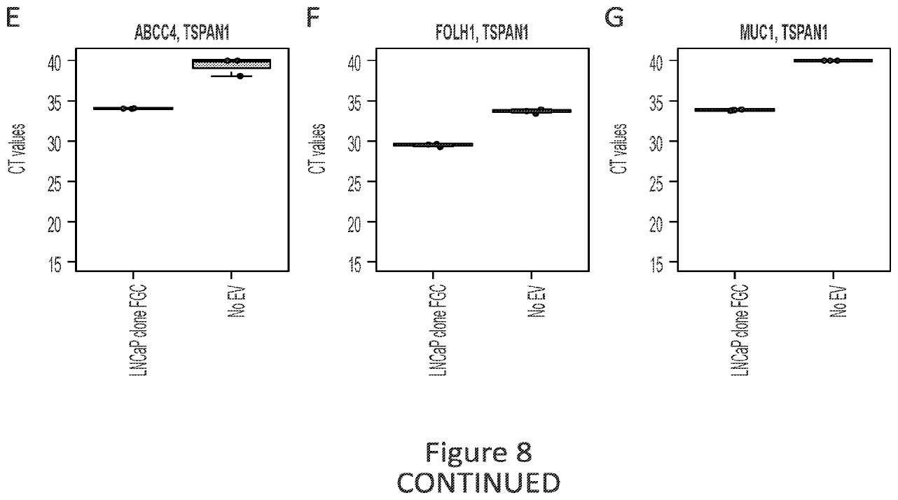

In the present Example, certain biomarker combinations as shown in FIG. 8 were assessed in prostate cancer-specific cell lines. Such biomarker combinations include at least two surface biomarkers, which are (EPCAM, T antigen), (LEY, VWA1), (MARCKSL1, T antigen), (T antigen, Tn antigen), (ABCC4, TSPAN1), (FOLH1, TSPAN1), and (MUC1, TSPAN1). In some embodiments, a prostate cancer-specific cell line utilized for biomarker combination characterization was originated from or associated with prostate adenocarcinoma. In some embodiments, a prostate cancer-specific cell line utilized for biomarker combination characterization was PC-3. In some embodiments, a prostate cancer-specific cell line utilized for biomarker combination characterization was LNCaP clone FGC.

FIG. 8 shows Ct values from characterization of certain 2-biomarker combinations in prostate cancer-specific cell lines and in a negative control group (e.g., no extracellular vesicles). Ct values were read from qPCR where the numeric value corresponds to the number of PCR cycles (i.e., higher values indicate less signal). As shown in FIG. 8, certain 2-biomarker combinations may be particularly useful for detection of prostate cancer. For example, surface biomarkers with high delta Ct values between the prostate cancer-specific cell line and the negative control group (e.g., delta Ct values greater than 2, including, e.g., greater than 3, greater than 4, greater than 5, or higher) may be particularly useful for detection of prostate cancer. In some embodiments, biomarker combinations as shown in FIG. 8 may be useful for detection of prostate cancer.

### REFERENCES CITED

Balaj, L., Lessard, R., Dai, L., Cho, Y. J., Pomeroy, S. L., Breakefield, X. O, and Skog, J., 2011. Tumour microvesicles contain retrotransposon elements and amplified oncogene sequences. Nature communications, 2, p. 180.
Bebelman, M. P., Smit, M. J., Pegtel, D. M., and Baglio, S. R., 2018. Biogenesis and function of extracellular vesicles in cancer. Pharmacology & Therapeutics, 188, pp. 1-11.
Darmanis, S., Nong, R. Y., Hammond, M., Gu, J., Alderborn, A., Vänelid, J., Siegbahn, A., Gustafsdottir, S., Ericsson, O., Landegren, U, and Kamali-Moghaddam, M., 2010. Sensitive plasma protein analysis by microparticle-based proximity ligation assays. Molecular & cellular proteomics, 9 (2), pp. 327-335.
Howlader N, Noone A M, Krapcho M, Miller D, Brest A, Yu M, Ruhl J, Tatalovich Z, Mariotto A, Lewis D R, Chen H S, Feuer E J, Cronin K A (eds). SEER Cancer Statistics Review, 1975-2016, National Cancer Institute. Bethesda, MD, https://seer.cancer.gov/csr/1975_2016/, based on November 2018 SEER data submission, posted to the SEER web site, April 2019.
Im, H., Shao, H., Park, Y. I., Peterson, V. M., Castro, C. M., Weissleder, R, and Lee, H., 2014. Label-free detection and molecular profiling of exosomes with a nano-plasmonic sensor. Nature biotechnology, 32 (5), p. 490.
Jeong, S., Park, J., Pathania, D., Castro, C. M., Weissleder, R, and Lee, H., 2016. Integrated magneto-electrochemical sensor for exosome analysis. ACS nano, 10 (2), pp. 1802-1809.
Shao, H., Im, H., Castro, C. M., Breakefield, X., Weissleder, R, and Lee, H., 2018. New technologies for analysis of extracellular vesicles. Chemical reviews, 118 (4), pp. 1917-1950.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A method comprising steps of:
(a) providing or obtaining a bodily fluid-derived sample (e.g., blood-derived sample) from a subject;
(b) detecting, in the bodily fluid-derived sample (e.g., blood-derived sample), extracellular vesicles expressing a first target biomarker signature (“first target biomarker signature-expressing extracellular vesicles”), the first target biomarker signature comprising:
at least one extracellular vesicle-associated surface biomarker and at least one target biomarker selected from the group consisting of: surface biomarkers, intravesicular biomarkers, and intravesicular RNA biomarkers, wherein:
the surface biomarkers are selected from (i) polypeptides encoded by human genes as follows: ABCC4, ABHD17C, AD11, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPP1R3A, PSCA, PVR, SLC44A4, TGFBR2, TNFRSF4, TNFSF11, VEGFC, AZGP1, GLB1L2, PABPC1L2A, SPOCK1, TGM4; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof;
the intravesicular biomarkers are selected from polypeptides encoded by human genes as follows: ACP5, ACPP, ACSM1, ACTA2, ACTG2, ADAMTS1, AGR2, AIM1, ALCAM, ALDH1A3, ALOX15B, ANTXR1, ANTXR2, AP1M2, AR, ARG2, ARHGAP6, ARHGEF26, ARHGEF37, ASAP3, ATP8B1, ATP9A, BAIAP2L1, BCAM, BHLHA15, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CGN, CGNL1, CHMP4C, CLGN, CMTM4, COLCA1, CORO2A, CPE, CPNE4, CPQ, CRYM, CTSF, CX3CL1, CXADR, CXCR4, CYB561, DPYSL3, DSP, EEF1A2, EFNA1, EHD2, EHF, ELF3, EMP2, EPHX1, EPN3, ERBB3, ERG, ESRP1, ESRP2, F3, FAAH, FAM129A, FAM129B, FAM210B, FAM3B, FAM3D, FAM83H, FAM84A, FAM84B, FAT1, FBLIM1, FBP1, FLNC, FNBP1L, FOS, FOXA1, GADD45G, GATA2, GGT1, GJB1, GLYATL1, GNG4, GNMT, GRHL2, GUCY1A3, HGD, HID1, HIST1H2BK, HIST3H2A, HMGCS2, HOMER2, HOXB13, HSD17B6, HSPB6, HSPB8, ICA1, ID1, IQGAP2, IRF6, ITGA6, KIAA1522, KIF5C, KLK2, KRT14, KRT15, KRT17, KRT18, KRT19, KRT5, KRT7, KRT8, LCP1, LIFR, LIMCH1, LMOD1, LRP11, LRP3, LSR, MAL2, MAOA, MAP7, MARC1, MARCKSL1, MB, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, PALLD, PARVA, PCDH1, PCP4, PDIA5, PEBP4, PGM5, PKP1, PKP3, PPL, PPM1H, PPP1R1B, PPP3CA, PRAC1, PRR15L, PSAT1, PTPRF, PYCR1, RAB3B, RAMP1, RAP1GAP, RASEF, RBM47, RCAN3, RDH11, REEP6, REPS2, RGS10, RHPN2, RNF144B, RORC, RPS4Y1, S100A16, SELENBP1, SERINC5, SH3BGRL2, SH3BP4, SHC2, SLC1A5, SLC22A3, SLC2A12, SLC40A1, SLC4A4, SLC7A8, SLC9A3R2, SMOC2, SORD, SPDEF, SPINT1, SPINT2, SPTBN2, ST14, STAP2, STK39, SULT2B1, SYNE4, SYT7, SYTL1, TAGLN, TBC1D16, TBX3, TC2N, TCEA3, TEAD3, TESC, THBS4, TM4SF1, TMED3, TMEM150C, TMEM54, TMEM98, TMSB15A, TP53111, TPD52, TPM1, TSPAN13, TSPAN6, TSPAN8, VAMP8, VSTM2L, WLS, WWC1, YAP1, ZBTB42, ZC3H11A, ZNF217, ACSL3, CADH7, KCNN2, OPRK1, PDE9A, PLPP1, SPON2, TMEM121B, and combinations thereof;
the intravesicular RNA biomarkers are selected from RNA transcripts (e.g., mRNA transcripts) encoded by human genes as follows: ABCC4, ACP5, ACPP, ACSM1, ACTA2, ACTG2, ADAMTS1, AGR2, AIM1, ALCAM, ALDH1A3, ALOX15B, ANO7, ANPEP, ANTXR1, ANTXR2, AP1M2, APCDD1, AQP3, AR, ARG2, ARHGAP6, ARHGEF26, ARHGEF37, ASAP3, ASTN2, ATP8B1, ATP9A, BAIAP2L1, BCAM, BHLHA15, BIK, BMPR1B, BOK, C15orf48, C19orf48, C1S, C1orf116, C2orf72, C8orf4, C9orf152, CADM4, CALD1, CAMSAP3, CAPN5, CBLC, CD24, CD74, CDC42EP1, CDC42EP5, CDH1, CGN, CGNL1, CHMP4C, CHRM1, CHRNA2, CLDN3, CLDN4, CLDN7, CLDN8, CLGN, CMTM4, COLCA1, COLEC12, CORO2A, CPE, CPNE4, CPQ, CRB3, CREB3L1, CREB3L4, CRYM, CTSF, CWH43, CX3CL1, CXADR, CXCR4, CYB561, DPP4, DPYSL3, DSG2, DSP, EEF1A2, EFNA1, EHD2, EHF, ELF3, ELOVL7, EMB, EMP2, ENPP5, EPCAM, EPHX1, EPN3, ERBB3, ERG, ESRP1, ESRP2, F3, FAAH, FAM129A, FAM129B, FAM189A2, FAM210B, FAM3B, FAM3D, FAM83H, FAM84A, FAM84B, FAT1, FBLIM1, FBP1, FLNC, FNBP1L, FOLH1, FOS, FOXA1, FXYD3, GADD45G, GALNT3, GALNT7, GATA2, GCNT1, GGT1, GJB1, GLYATL1, GNG4, GNMT, GOLM1, GPR160, GREB1, GRHL2, GUCY1A3, HGD, HID1, HIST1H2BK, HIST3H2A, HMGCS2, HOMER2, HOXB13, HPN, HSD17B6, HSPB6, HSPB8, ICA1, ID1, IQGAP2, IRF6, ITGA6, KCNN4, KIAA1324, KIAA1522, KIF5C, KLK2, KRT14, KRT15, KRT17, KRT18, KRT19, KRT5, KRT7, KRT8, KRTCAP3, LCP1, LIFR, LIMCH1, LMOD1, LPAR3, LRP11, LRP3, LRRC26, LSR, MAL2, MAOA, MAP7, MARC1, MARCKSL1, MB, MBOAT2, MESP1, MLPH, MME, MPZL2, MT1G, MYBPC1, MYL9, MYLK, NCAPD3, NEDD4L, NEFH, NFIX, NKX3-1, NPDC1, NUPR1, OBSL1, OR51E1, OR51E2, PALLD, PARM1, PARVA, PCDH1, PCP4, PDIA5, PDLIM5, PDZK1IP1, PDZRN3, PEBP4, PGM5, PIGR, PKP1, PKP3, PLA2G2A, PMEPA1, PODXL2, PPL, PPM1H, PPP1R1B, PPP3CA, PRAC1, PROM2, PRR15L, PRSS8, PSAT1, PSCA, PTPRF, PTPRN2, PYCR1, RAB25, RAB27B, RAB3B, RAMP1, RAP1GAP, RASEF, RBM47, RCAN3, RDH11, REEP6, REPS2, RGS10, RHPN2, RNF144B, RORC, RPS4Y1, S100A16, SCNN1A, SDC1, SELENBP1, SERINC2, SERINC5, SEZ6L2, SH3BGRL2, SH3BP4, SHC2, SLC15A2, SLC1A5, SLC22A3, SLC2A10, SLC2A12, SLC30A4, SLC35F2, SLC40A1, SLC44A4, SLC45A3, SLC4A4, SLC7A8, SLC9A3R2, SMIM22, SMOC2, SORD, SPDEF, SPINT1, SPINT2, SPTBN2, ST14, STAP2, STEAP1, STEAP2, STEAP4, STK39, SULT2B1, SYNE4, SYT7, SYTL1, TACSTD2, TAGLN, TBC1D16, TBX3, TC2N, TCEA3, TEAD3, TESC, THBS4, TM4SF1, TMC4, TMC5, TMED3, TMEFF2, TMEM125, TMEM150C, TMEM30B, TMEM45B, TMEM54, TMEM98, TMPRSS2, TMSB15A, TP53111, TPD52, TPM1, TRPM4, TRPM8, TRPV6, TSPAN1, TSPAN13, TSPAN6, TSPAN8, TUSC3, UPK3A, VAMP8, VSIG2, VSTM2L, WLS, WWC1, YAP1, ZBTB42, ZC3H11A, ZNF217, and combinations thereof;
(c) comparing sample information indicative of level of the first target biomarker signature-expressing extracellular vesicles in the bodily fluid-derived sample (e.g., blood-derived sample) to reference information including a first reference threshold level;
(d) classifying the subject as having or being susceptible to prostate cancer when the bodily fluid-derived sample (e.g., blood-derived sample) shows an elevated level of first target biomarker signature-expressing extracellular vesicles relative to a classification cutoff referencing the first reference threshold level.

2. The method of claim 1, wherein when the at least one target biomarker is selected from one or more of the surface biomarkers, the selected surface biomarker(s) and the at least one extracellular vesicle-associated surface biomarker are different.

3. The method of claim 1, wherein the steps of (b) and (c) are repeated for at least a second target biomarker signature, and wherein the classification cutoff references the first reference threshold level and at least a second reference threshold level corresponding to the at least a second target biomarker signature.

4. The method of claim 1, wherein the extracellular vesicle-associated surface biomarker is or comprises a polypeptide encoded by human genes as follows: CLDN3, ABCC4, RAB3B, FOLH1, PMEPA1, TMPRSS2, TSPAN1, PODXL2, GOLM1, SORD, CLGN, GRHL2, CANT1, SYT17, PPP3CA, or combinations thereof.

5. The method of claim 1, wherein the first and/or second target biomarker signature comprises at least one extracellular vesicle-associated surface biomarker and at least two biomarkers selected from the group consisting of: surface biomarkers, intravesicular biomarkers, and intravesicular RNA biomarkers.

6. The method of claim 1, wherein the at least two biomarkers comprise one of the following combinations:
at least two distinct surface biomarkers;
at least two distinct intravesicular biomarkers;
at least two distinct intravesicular RNA biomarkers;
a surface biomarker and an intravesicular biomarker;
a surface biomarker and an intravesicular RNA biomarker; and
an intravesicular biomarker and an intravesicular RNA biomarker.

7. The method of claim 1, wherein the first or second reference threshold level is determined by levels of target biomarker signature-expressing extracellular vesicles observed in comparable samples from a population of non-cancer subjects.

8. The method of claim 7, wherein the population of non-cancer subjects comprises one or more of the following subject populations: healthy subjects, subjects diagnosed with benign tumors, subjects with prostate-related diseases (e.g., benign prostatic hyperplasia, prostatitis, etc.) and subjects with non-prostate-related diseases, disorders, and/or conditions.

9. The method of claim 1, wherein the bodily fluid-derived sample (e.g., blood-derived sample) has been subjected to size exclusion chromatography to isolate (e.g., directly from the bodily fluid-derived sample (e.g., blood-derived sample) nanoparticles having a size range of interest that includes extracellular vesicles.

10. The method of claim 1, wherein the step of detecting comprises a capture assay.

11. The method of claim 10, wherein the capture assay involves contacting the bodily fluid-derived sample (e.g., blood-derived sample) with a capture agent comprising a target-capture moiety that binds to the at least one extracellular vesicle-associated surface biomarker.

12. The method of claim 11, wherein the capture agent is or comprises a solid substrate comprising the target-capture moiety conjugated thereto.

13. The method of claim 12, wherein the solid substrate comprises a magnetic bead.

14. The method of claim 11, wherein the target-capture moiety is or comprises an antibody agent.

15. The method of claim 1, wherein the step of detecting comprises a detection assay.

16. The method of claim 1, wherein the step of detecting comprises a capture assay and a detection assay, the capture assay being performed prior to the detection assay.

17. The method of claim 1, wherein when the first and/or second target biomarker signature comprises at least one intravesicular RNA biomarker, the detection assay involves reverse transcription qPCR.

18. The method of claim 15, wherein when the first and/or second target biomarker signature comprises at least one intravesicular biomarker, the target biomarker signature-expressing extracellular vesicles are processed involving fixation and/or permeabilization prior to the detection assay.

19. The method of claim 15, wherein when the first and/or second target biomarker signature comprises at least one surface biomarker and/or intravesicular biomarker, the detection assay involves an immunoassay (including, e.g., immuno-PCR, and/or proximity ligation assay).

20. The method of claim 19, wherein the detection assay involves a proximity ligation assay.

21.-114. (canceled)
